(19) United States
(12) Patent Application Publication
(10) Pub. No.: US 2011/0065158 A1
(43) Pub. Date: Mar. 17, 2011

(54) REGULATION OF PLANT METABOLISM

(75) Inventors: Ian Alexander Graham, York (GB); Johanna Elizabeth Cornah, Stratford-upon-Avon (GB)

(73) Assignee: THE UNIVERSITY OF YORK, York (GB)

(21) Appl. No.: 12/517,930

(22) PCT Filed: Dec. 7, 2007
(86) PCT No.: PCT/GB2007/004682

§ 371 Date: Oct. 5, 2010

(30) Foreign Application Priority Data
Dec. 8, 2006 (GB) 0624541.9
Jul. 3, 2007 (GB) 0712908.3

(51) Int. Cl. C12P 7/06; A01H 5/00; A01H 5/10; C11B 1/04; C07H 1/08; C12N 5/10

(52) U.S. Cl. 435/161; 800/298; 800/320.3; 800/320; 800/320.1; 554/8; 536/128; 435/419

(57) ABSTRACT

We describe a plant and plant cell which is modified to enhance the production of plant-derived oil in particular in non-seed tissue and including methods for the processing of plant derived biomass materials.

The invention relates to plant cells and plants that are modified to enhance the production of plant oils and fatty acids and including methods for the processing of plant derived biomass materials.

Plant derived products are currently being widely adopted both as industrial feedstock and as replacement fuels. So called first generation biofuels are either based on bioethanol or biodiesel. Bioethanol production relies on the process of fermentation using microbial organisms to produce ethanol. This ethanol is then used mainly as fuel for transportation. The feedstock for this microbial fermentation is typically sugar obtained from sugar cane or sugar beet or derived from starch obtained from cereal crops such as maize or wheat. Bioethanol production from sugarcane, sugar beet and cereal grains such as maize (corn), wheat and barley feedstock has been widely adopted. Biodiesel is an alternative biofuel to bioethanol. Crops used to produce feedstock for biodiesel production include soybean, castor bean, sunflower, rapeseed, Jatropha and palm.

Biodiesel has some advantages when compared to bioethanol as a fuel source. A recent study based on biofuel production in the USA focussed on environmental, economic, and energetic costs and benefits of biodiesel and ethanol biofuels (Hill et al., PNAS 2006, 103: 11206-10). This study reached a number of conclusions including (1) Soybean derived biodiesel yields 93% more energy than the energy invested in its production whereas Corn-derived ethanol yields only 25% more. (2) Compared with ethanol, biodiesel releases just 1%, 8.3% and 13% of nitrogen, phosphorous and pesticide pollutants respectively per net energy gain. (3) Relative to fossil fuels they displace, combustion of ethanol reduces greenhouse gas emissions by 12% and biodiesel by 41%. (4) Advantages of biodiesel over ethanol are due to lower agricultural inputs and more efficient conversion of feedstocks to fuels. (5) Even dedicating all current production of USA corn and soybean to biofuels would meet only 12% of gasoline and 6% of diesel demand. Therefore, the demand for gasoline and diesel would not be satisfied by current production of bioethanol and biodiesel. There is therefore a continued need to identify means to improve the production of biofuels.

Based on these considerations, attention has turned to non-food derived biofuel from lignocellulosic biomass. Plant biomass is cheap and abundant and typically contains 25% lignin and 75% polysaccharides which represent a rich source of sugars. This biomass can be derived from agricultural residues (leftover material from crops, such as the stalks, leaves, and husks of corn plants), forestry wastes (chips and sawdust from lumber mills, dead trees, and tree branches), municipal solid waste (household garbage and paper products), food processing and other industrial wastes or so called Energy crops' (fast-growing trees and grasses) developed specifically for biomass.

To utilize this biomass however requires much effort to release the sugars from lignocellulose in a process called saccharification.

Because the constituent fatty acids of plant-derived oils are structurally similar to the hydrocarbon chains that give functionality to petrochemicals there is potential for plant-derived oils to act as a sustainable replacement to petrochemicals not only for fuel supply but as industrial feedstock for other applications. These include non-food industrial application areas ranging from lubricants, polymers, paints and solvents to inks and dyes and cosmetics and surfactants typically found in biofuels to facilitate blending.

The major constituents of plant oils are triacylglycerol molecules that contain three fatty acid chains attached to a glycerol backbone. These oils accumulate during seed development. Crops such as soybean, sunflower and oilseed rape have been developed to produce vegetable oil as a major commodity for food and non-food applications. Fatty acid molecules that provide the useful functionality of plant-derived oils are essential constituents of all living cells. Fatty acids are major constituents of membrane lipids which are essential for membrane integrity and cellular activity. Fatty acid biosynthesis therefore occurs throughout the different cells and tissues of a plant whereas triacylglycerol biosynthesis occurs primarily in storage tissues of developing seeds and is not typically found in other tissues of the plant.

Plant-derived oils and their constituent fatty acids also have important food and nutraceutical applications. In particular, 18:2 linoleic acid and 18:3 alpha linolenic acid are so called essential fatty acids that are typically not produced in animals and need to be obtained from plants in the food chain. In addition, gamma linolenic acid and long-chain polyunsaturated fatty acids are recognised as having benefits to human health. There is currently much effort to develop transgenic plants that produce the long chain polyunsaturated fatty acids EPA and DHA that are the active ingredients in fish oil. This effort is typically focussed on engineering developing seed metabolism in order to modify the fatty acid content in seed oil; see PCT/GB03/001099; PCT/GB2004/003057; PCT/GB2005/000549; and PCT/GB2005/003643, each of which is incorporated by reference.

Furthermore, transgenic plants have been engineered to produce unusual fatty acids, (e.g hydroxylated fatty acids). It is known to produce unusual fatty acids such as hydroxylated fatty acids in seeds but the yields are poor; see Thelen J J, Ohlrogge J B, et al Metab Eng. 2002 January; 4(1):12-21) For example, ricinoleic acid is synthesized by oleate-12-hydroxylase the sequence of which is disclosed in U.S. Pat. No. 5,668,292; U.S. Pat. No. 5,801,026; U.S. Pat. No. 6,028,248. and U.S. Pat. No. 6,974,893 (the contents of which are incorporated by reference in their entirety and specifically the sequences of oleate-12-hydroxylase and isoforms thereof). Examples of using such genes to produce ricinoleic and related unusual fatty acids in transgenic plants are known in the art; see Van de Loo F J, Broun P, Turner S, Somerville C. Proc Natl Acad Sci USA. 1995 Jul. 18; 92(15):6743-7; Broun P and Somerville C Plant Physiol. 1997 March; 113(3):933-42).

A further example is the use of cytochrome P450 associated with the synthesis of deltal2-epoxy groups in fatty acids of plants. An example of using such a gene to produce epoxy fatty acids in transgenic plants has been demonstrated (see Cahoon E B, Ripp K G, Hall S E, McGonigle Plant Physiol. 2002 February; 128(2):615-24). In addition the expression of delta 12 fatty acid acetylenase genes in transgenic plants result in the production of acetylenic acid: see Nilsson, R., Liljenberg, C., Dahlqvist, A., Gummeson, P. O., Sjodahl, S. Green, A. and Stymne, S. Science 280 (5365), 915-918 (1998) and Sperling P, Lee M, Girke T, Zahringer U, Stymne S, Heinz Eur J. Biochem. 2000 June; 267(12):3801-11).

An alternative approach to engineering plants to produce fatty acids and/or unusual fatty acids is to transfect plants with genes that encode transcription factors.

In Arabidopsis thaliana, four loci have been identified, namely FUSCA3 (FUS3), ABSCISIC ACID INSENSITIVE3 (ABI3) and LEAFY COTYLEDON1 and 2 (LEC1 and LEC2), that control a wide range of seed-specific characters and play an essential role in seed maturation. These genes all encode transcription factors that have been shown to act synergistically in mediating the response to abscisic acid, the key hormonal regulator of seed maturation. FUS3, ABI3 and LEC2 belong to the B3 family of plant transcription factors, whereas LEC1 encodes an NFY-B factor. The abi3, lec1, lec2 and fus3 mutants share common phenotypes such as reduced accumulation of storage compounds, and exhibit specific phenotypes such as the lack of chlorophyll degradation, anthocyanin accumulation, intolerance to desiccation, or defects in cotyledon identity.

The ectopic expression of LEC2 can confer embryonic characteristics to transgenic seedling, triggering TAG accumulation in developing leaves. An additional regulatory protein called WRINKLED1 (WRI1), a putative AP2/EREBP transcription factor involved in the regulation of seed storage metabolism in Arabidopsis thaliana (Cernac A, Benning C. Plant J. 2004 40:575-85) is also known. Recently Baud et al., (Plant J., 2007, 50:825-838) have demonstrated that WRI1 is a direct target of LEC2 with the implication that it is this interaction that specifies the action of the LEC2 master regulator towards the fatty acid biosynthetic network, such that WRI1 is necessary for LEC2-induced oil accumulation

Ectopic expression of FUS3, and ABI3 which are B3 domain transcription factors closely related to LEC2, causes accumulation of seed protein RNAs in vegetative organs, as is the case with ectopic expression of LEC2 (Parcy et al., Plant Cell. 1994; 6:1567-1582; Gazzarrini et al., Dev. Cell. 2004; 7:373-385; Kagaya et al., Plant Cell Physiol. 2005; 46:300-311). More recently Braybrook et al., Proc Natl Acad Sci USA. 2006 103: 3468-3473 have demonstrated that LEC2 binds with the same DNA element bound by FUS3 and ABI3, the RY motif, which provides a partial explanation for similarities in the gain-of-function phenotypes. These authors point out that LEC2, ABI3, and FUS3 share identical or conserved amino acid residues at positions in the B3 domain implicated as being responsible for DNA-binding specificity based on the solution structure of the B3 domain protein RAV1. Thus, all three transcription factors bind RY motifs through their B3 domains and activate maturation-specific genes. It is possible that the activation of genes associated with oil biosynthesis in all cases could involve an interaction with WRI1. FUS3 together with LEC1 positively regulate the abundance of the ABI3 protein in the seed. Therefore LEC1 may also be expected to lead to elevated expression of seed maturation related genes when ectopically expressed in other tissues.

The exploitation of plants as a source of biofuel and biofuel additives is known in the art. For example, WO2006/002683 describes compositions derived from rapeseed comprising alkyl esters that are formed by treatment of a rapeseed extract in a transesterification reaction that combines the conversion of the oil to its fatty acids followed by an acid catalysis. The composition is high in oleic acid and low in linolenic acid and is claimed to have advantages as a biofuel or biofuel additive. US2005/0069614 describes the extraction of soybean oil that combines mechanical extraction with solvent treatment to substantially extract all the oil in a plant preparation. WO03/085071 describes a process for the production of a mixture of levulinic acid esters and formic acid esters from biomass and olefins. The composition comprising the esters has use as an additive in biofuel to improve performance. WO01/62876 describes a surfactant comprising a mixture of alkanolamide, an alkoxylated alcohol and an alkoxylated fatty acid to facilitate the blending of plant derived fatty acids with diesel. DE19637909 describes a process for the chemical decomposition, saccharification and fermentation of wood that involves a mechanical pre-treatment and chemical digestion of lignin. It is apparent that prior art processes for the production and processing of plant derived products requires both mechanical disruption and severe chemical treatments that are both expensive, labour intensive and involve environmentally damaging chemical treatments.

The present disclosure relates to the production of mono- di- or triacylglycerols in non-seed tissues, for example foliar and vegetative tissues. Advantageously, this provides significant amounts of plant oils in vegetative tissues that can be used as an industrial feedstock or as a feedstock for biodiesel. The oil can be extracted during processing leaving biomass that can then be subjected to saccharification more readily. The disclosure also relates to the inclusion of genes that alter the qualitative and/or quantitative profile of fatty acid production in non-seed tissues.

Additionally, the production of triacylglycerol fatty acids in non-seed tissues could also be used as a source of food, animal feed or neutraceutical. In particular the non-seed tissue producing mono- di- or triacylglycerol fatty acids could be further modified with long-chain fatty acid producing enzymes such as fatty acid desaturases, fatty acid elongases and acyltransferases in order to produce long chain polyunsaturated fatty acids.

Moreover, the production of mono- di- or triacylglycerol fatty acids in non-seed tissues could also be used as a source of unusual fatty acids such as hydroxy fatty acids such as ricinoleic acid, epoxy or conjugated fatty acids. Metabolic engineering to produce these fatty acids in seed oil has met with problems of yield due to apparent bottlenecks in the flux of unusual fatty acids into seed oil and/or breakdown of the unusual fatty acids before they are partitioned to the seed oil. In addition, production of unusual fatty acids in the seed oil of plants that do not naturally accumulate these fatty acids can lead to problems with seed germination and seed viability. Production of unusual fatty acids in non-seed oil could circumvent the problems with seed germination and could also alleviate the problems with yield. The expression of transgenes encoding appropriate enzymes such as hydroxylases, epoxidases and conjugases in non-seed tissue that has been modified to produce mono- di- or triacylglycerol fatty acids could be used to produce unusual fatty acids with important industrial applications.

According to an aspect of the invention there is provided a transgenic plant cell the genome of which is modified by transfection with a nucleic acid molecule selected from the group consisting of:
i) a nucleic acid molecule comprising an expression cassette which cassette comprises a nucleic acid sequence that encodes at least part of a gene that encodes a polypeptide that controls the synthesis, degradation or transport of fatty acids and/or fatty acyl CoAs and therefore synthesis and degradation of mono- di- or triacylglycerols wherein said cassette is adapted such that both sense and antisense nucleic acid molecules are transcribed from said cassette wherein the expression from said cassette produces an interfering RNA molecule that inhibits the expression of said gene;
ii) a nucleic acid molecule comprising an expression cassette which cassette comprises a nucleic acid sequence that encodes at least part of a gene that encodes a polypeptide that controls the synthesis, degradation or transport of fatty acids and/or fatty acyl CoAs and therefore synthesis and degradation of mono- di- or triacylglycerols wherein said cassette is adapted such that an antisense nucleic acid molecule is transcribed from said cassette wherein the expression from said cassette produces an antisense RNA molecule that inhibits the expression of said gene;
iii) a nucleic acid molecule that encodes a polypeptide that controls the synthesis, degradation or transport of fatty acids and/or fatty acyl CoAs and therefore synthesis and degradation of mono- di- or triacylglycerols in a plant cell which polypeptide is a variant polypeptide that varies from a native polypeptide sequence wherein said variant polypeptide is a dominant negative suppressor of the native polypeptide and inhibits the production of fatty acids and/or fatty acyl CoAs therefore synthesis and degradation of mono- di- or triacylglycerols, wherein said nucleic acid molecule in i), ii) and iii) is operably linked to a promoter sequence that is substantially a foliar inducible and/or senescence inducible promoter.

In a preferred embodiment of the invention said gene encodes a polypeptide involved in transport, activation or degradation of fatty acids and/or fatty acyl Co As.

Methods to provide plants that are modified to down regulate or ablate genes are well known in the art and include the use of antisense genes to regulate the expression of specific targets; insertional mutagenesis using T-DNA; the introduction of point mutations and small deletions into open reading frames and regulatory sequences; and double stranded inhibitory RNA (RNAi). RNAi is a technique to specifically ablate gene function through the introduction of double stranded RNA into a cell that results in the destruction of mRNA complementary to the sequence included in the RNAi molecule. The RNAi molecule comprises two complementary strands of RNA (a sense strand and an antisense strand) annealed to each other to form a double stranded RNA molecule. The RNAi molecule is typically derived from exonic or coding sequence of the gene which is to be ablated. Surprisingly, only a few molecules of RNAi are required to block gene expression that implies the mechanism is catalytic. The site of action appears to be nuclear as little if any RNAi is detectable in the cytoplasm of cells indicating that RNAi exerts its effect during mRNA synthesis or processing.

An alternative embodiment of RNAi involves the synthesis of so called “stem loop RNAi” molecules that are synthesised from expression cassettes carried in vectors. The DNA molecule encoding the stem-loop RNA is constructed in two parts, a first part that is derived from a gene the regulation of which is desired. The second part is provided with a DNA sequence that is complementary to the sequence of the first part. The cassette is typically under the control of a promoter that transcribes the DNA into RNA. The complementary nature of the first and second parts of the RNA molecule results in base pairing over at least part of the length of the RNA molecule to form a double stranded hairpin RNA structure or stem-loop. The first and second parts can be provided with a linker sequence. Stem loop RNAi has been successfully used in plants to ablate specific mRNAs and thereby affect the phenotype of the plant, see, Smith et al (2000) Nature 407, 319-320.

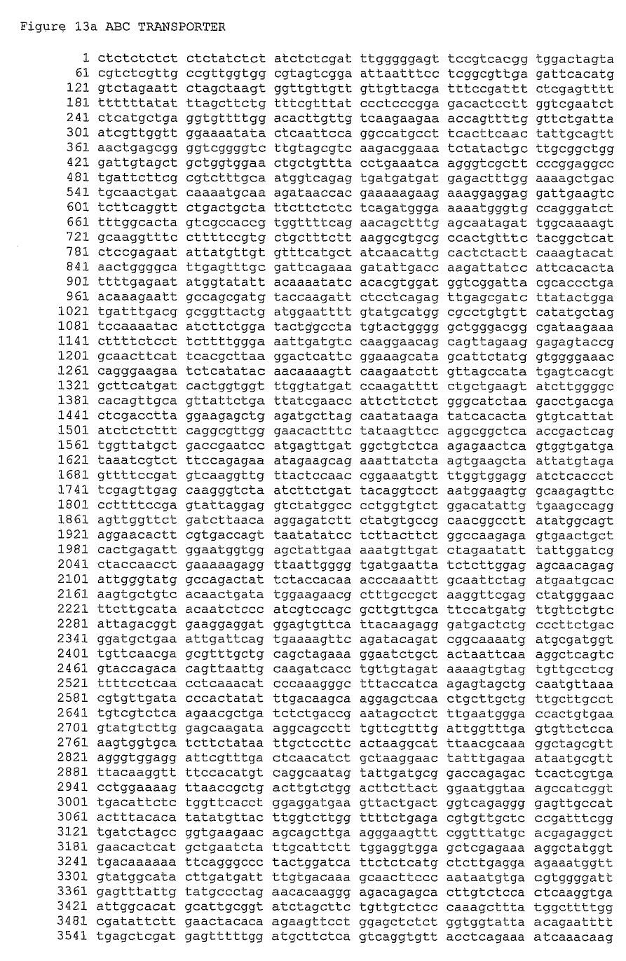

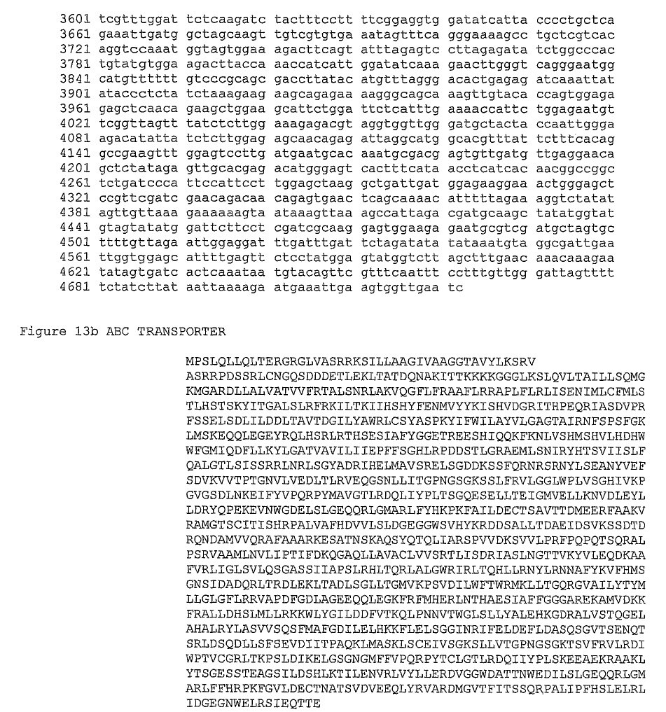

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 13a;
ii) a nucleic acid molecule comprising a nucleic acid sequence that hybridises under stringent hybridisation conditions to a nucleic acid molecule in (i) and which encodes a fatty acid transporter polypeptide;
iii) a nucleic acid molecule that encodes a variant polypeptide that varies from a polypeptide comprising the amino acid sequence as represented in FIG. 13b.

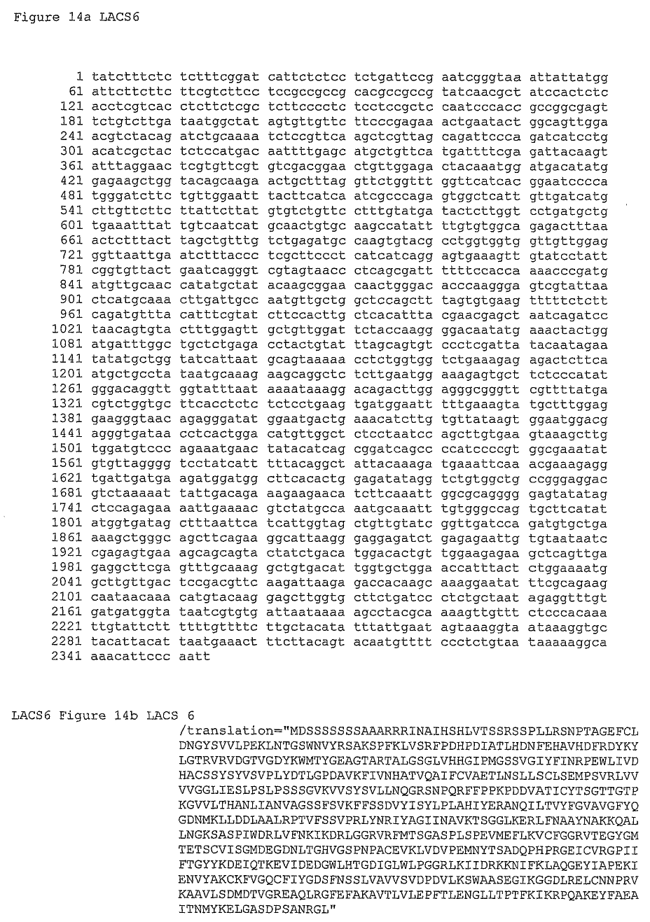

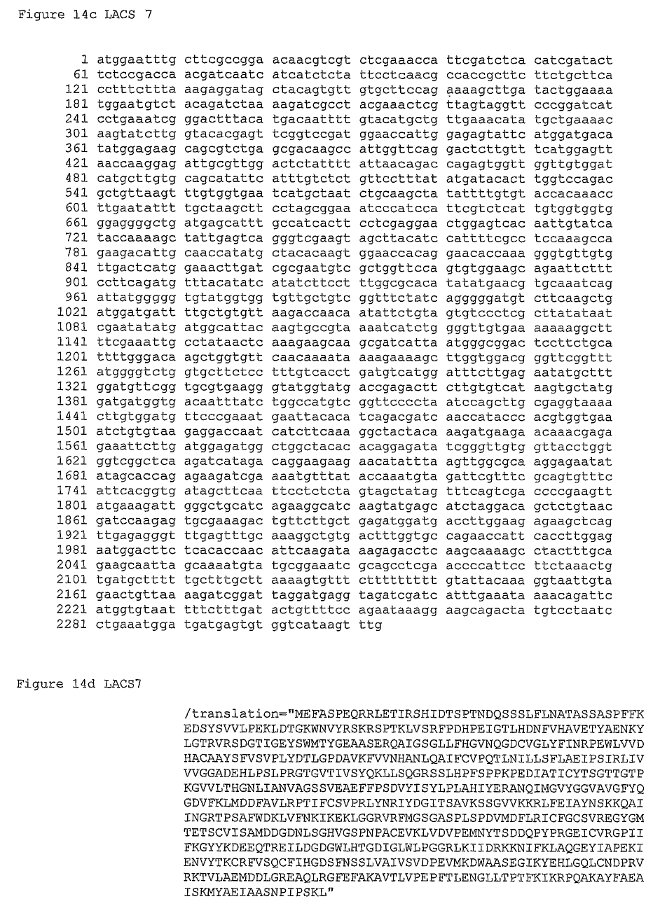

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 14a or 14c;
ii) a nucleic acid molecule comprising a nucleic acid sequence that hybridises under stringent hybridisation conditions to a nucleic acid molecule in (i) and which encodes a long chain acyl Co A synthetase polypeptide;
iii) a nucleic acid molecule that encodes a variant polypeptide that varies from a polypeptide comprising the amino acid sequence as represented in FIG. 14b or 14d.

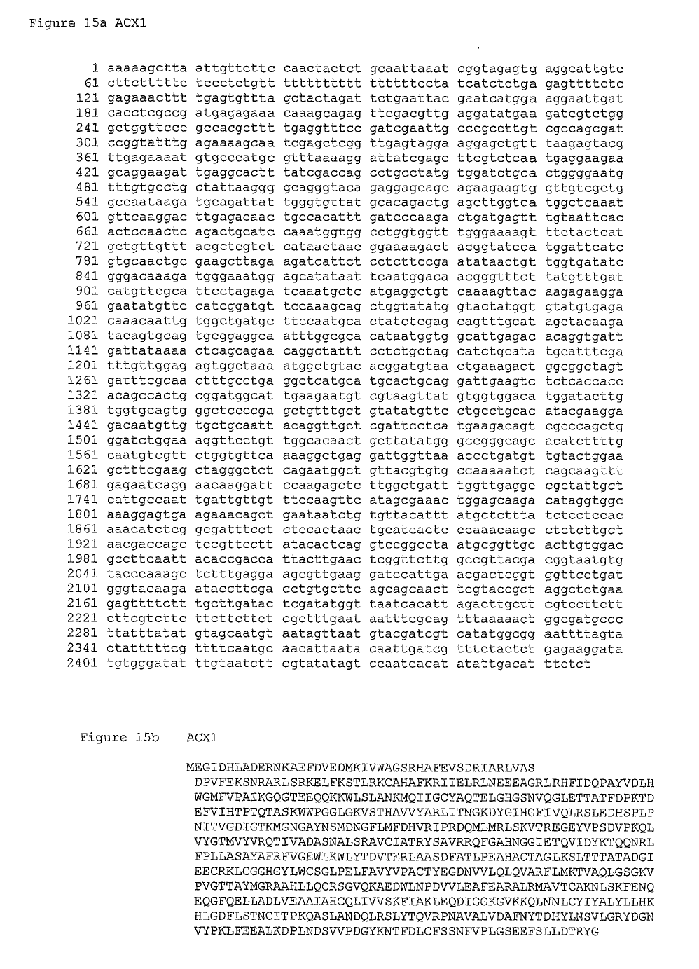

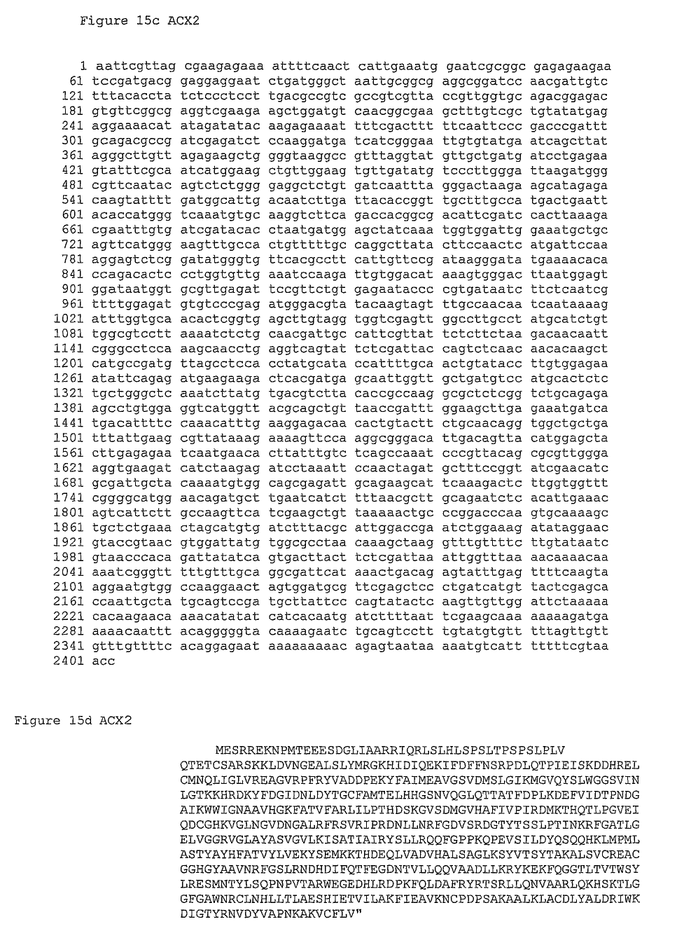

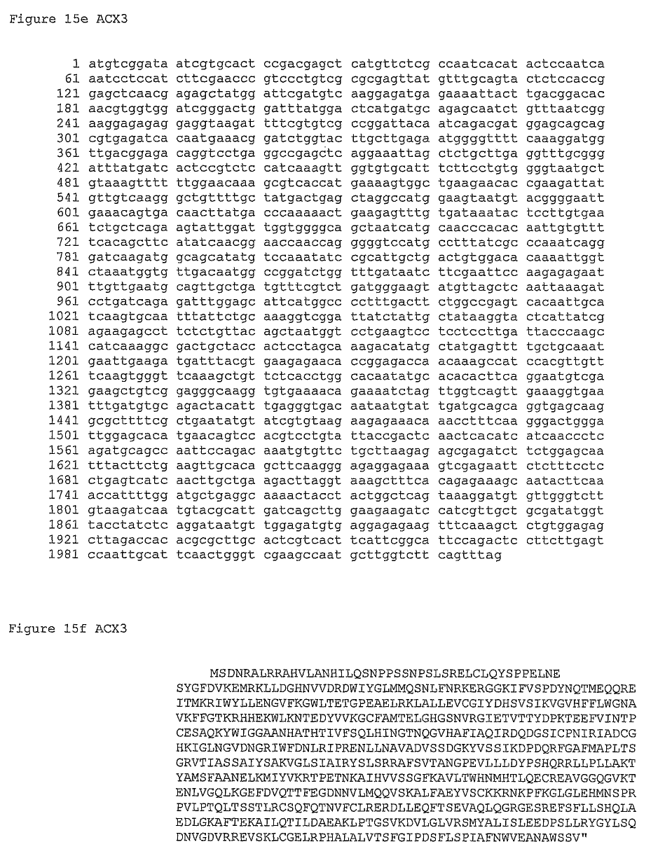

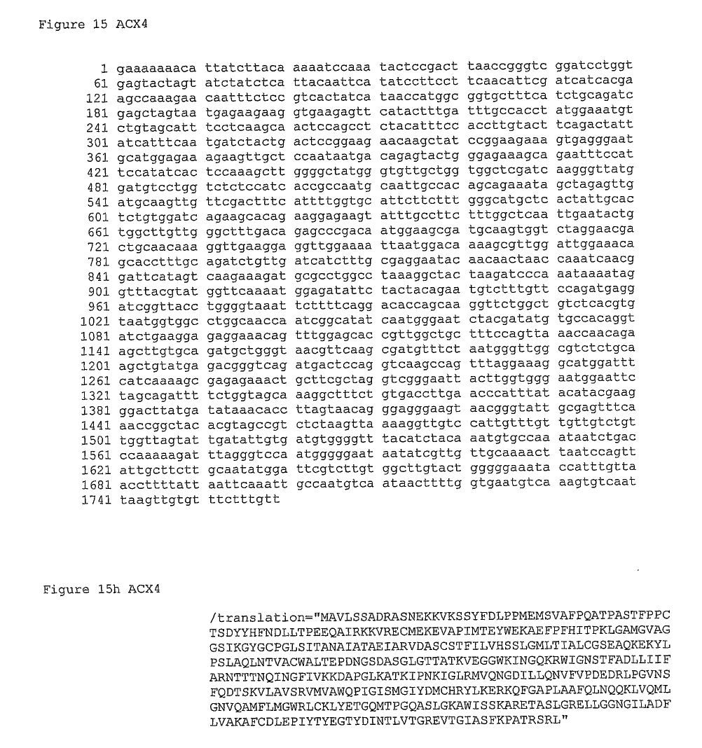

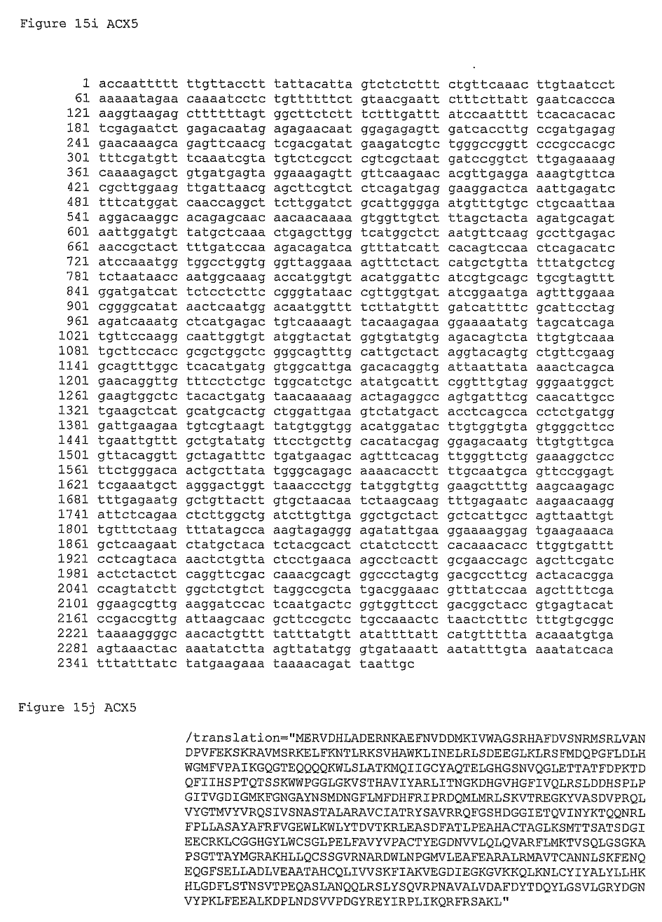

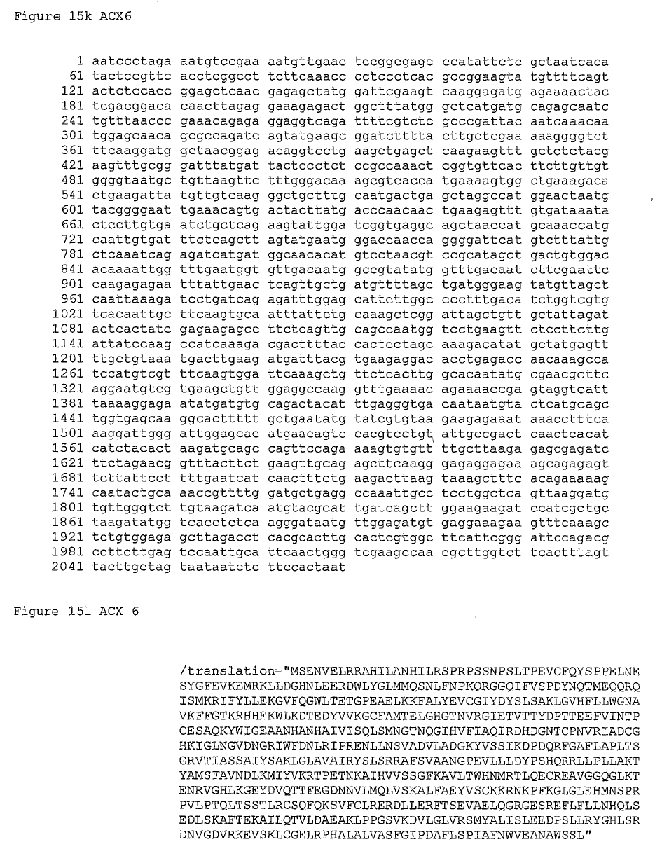

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 15a, 15c, 15e, 15g, 15i or 15k;
ii) a nucleic acid molecule comprising a nucleic acid sequence that hybridises under stringent hybridisation conditions to a nucleic acid molecule in (i) and which encodes an acyl CoA oxidase polypeptide;
iv) a nucleic acid molecule that encodes a variant polypeptide that varies from a polypeptide comprising the amino acid sequence as represented in FIG. 15b, 15d, 15f, 15h, 15j or 15l.

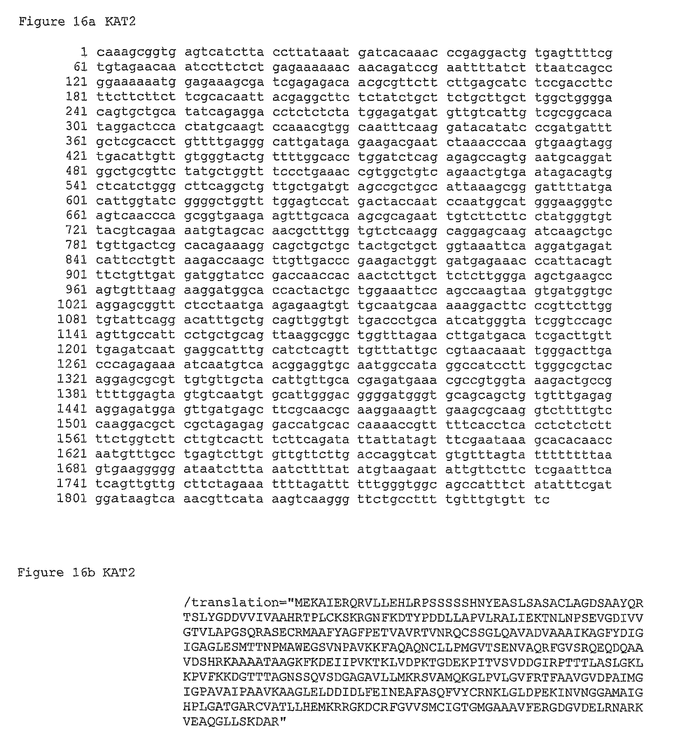

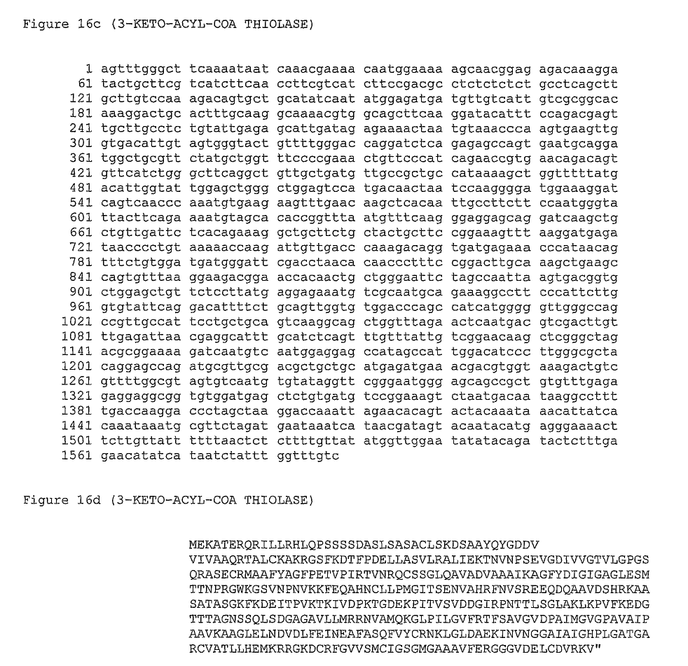

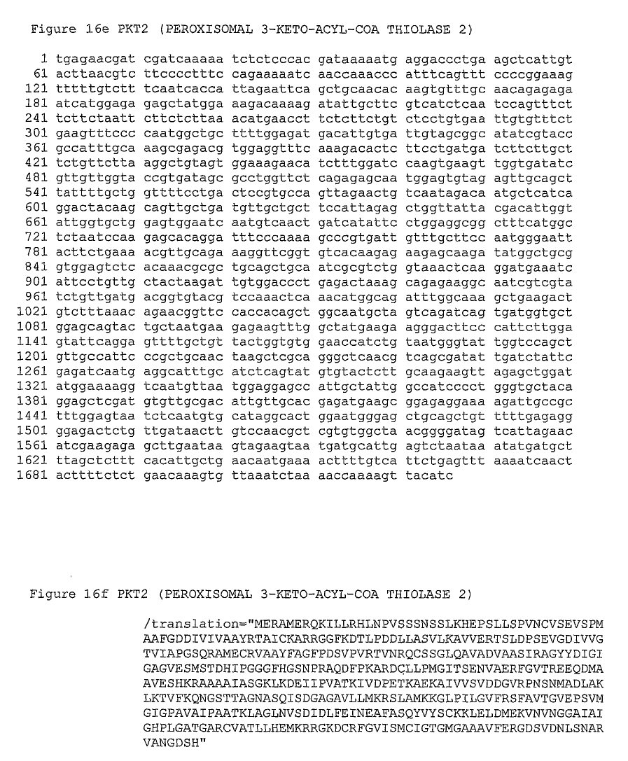

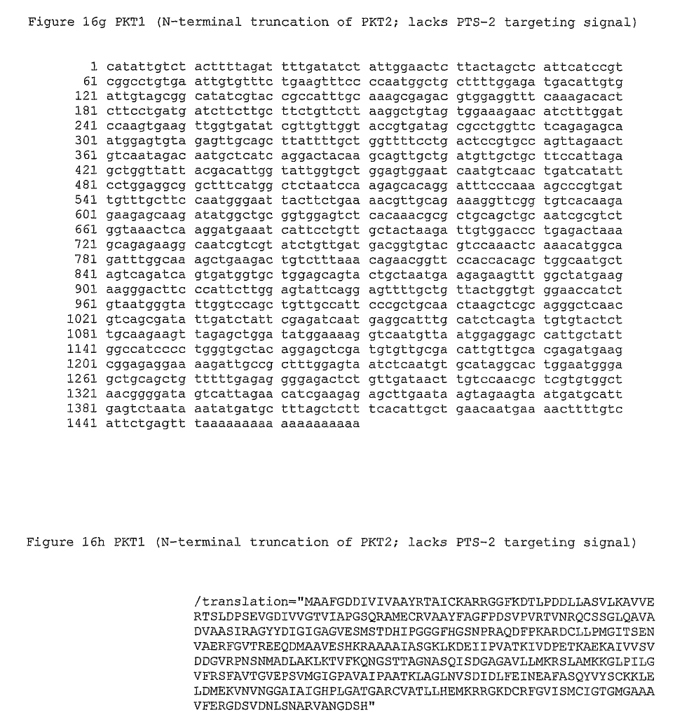

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 16a; 16c,16e or 16g
ii) a nucleic acid molecule comprising a nucleic acid sequence that hybridises under stringent hybridisation conditions to a nucleic acid molecule in (i) and which encodes a Keto-Acyl-CoA thiolase;
iii) a nucleic acid molecule that encodes a variant polypeptide that varies from a polypeptide comprising the amino acid sequence as represented in FIG. 16b, 16d, 16f or 16h.

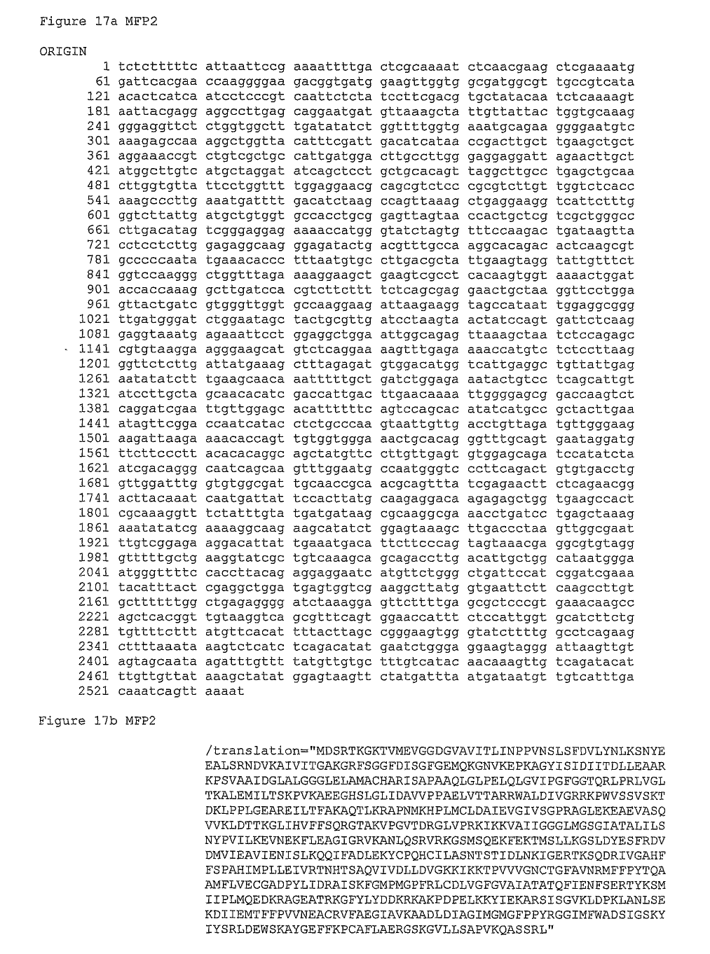

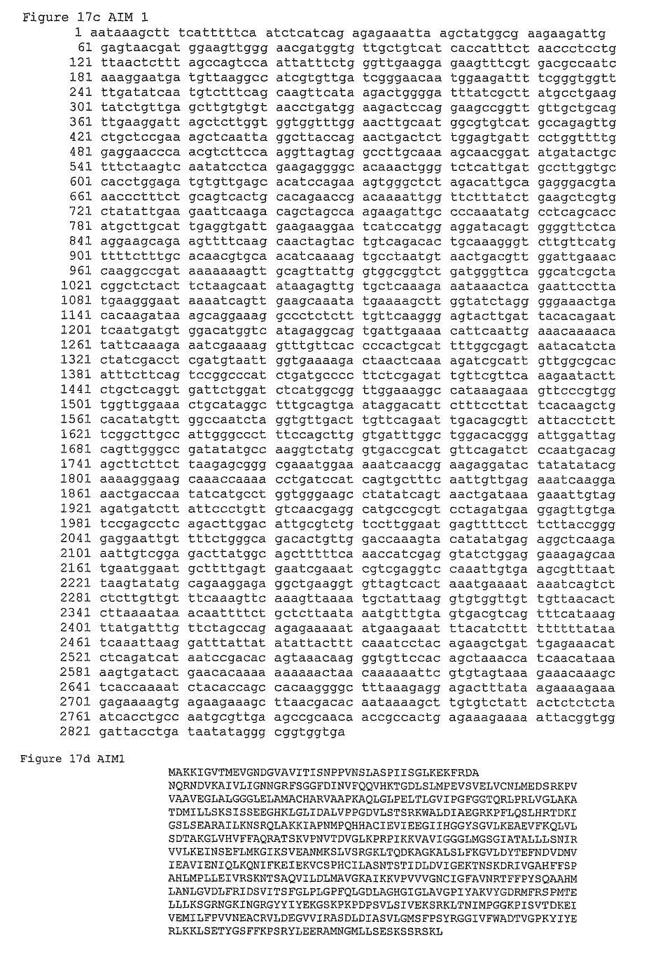

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 17a or 17c
ii) a nucleic acid molecule comprising a nucleic acid sequence that hybridises under stringent hybridisation conditions to a nucleic acid molecule in (i) and which encodes a multifunctional protein involved in peroxisomal β oxidation;
iii) a nucleic acid molecule that encodes a variant polypeptide that varies from a polypeptide comprising the amino acid sequence as represented in FIG. 17b or 17d.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In a further preferred embodiment of the invention said cassette adapted for expression of sense and antisense nucleic acid comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts.

In a further preferred embodiment of the invention said promoter sequence is an inducible foliar specific promoter sequence.

In a further preferred embodiment of the invention said promoter sequence is a senescence inducible promoter sequence.

Foliar and/or senescence specific promoters are known in the art. For example, WO0070061; US2004025205; WO2006102559; US6, 359, 197; WO2006025664 the contents of which are incorporated by reference in their entirety, describe various plant promoters that become activated when senescence is induced. In addition US2002120955 and WO9800533, the contents of which are incorporated by reference, each describe a number of promoter sequences that have leaf or predominantly a leaf specific expression pattern. The present disclosure also describes two promoters that control the expression of genes involved in triacylglycerol metabolism. The genes that encode ACX 1 and KAT 2 are both induced during the induction of senescence and are therefore considered a least in part, senescence inducible.

In a preferred embodiment of the invention said nucleic acid molecule is part of a vector and is operably linked to a promoter.

“Operably linked” means joined as part of the same nucleic acid molecule, suitably positioned and oriented for transcription to be initiated from the promoter. DNA operably linked to a promoter is “under transcriptional initiation regulation” of the promoter.

Particular vectors are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy R R D ed) Oxford, BIOS Scientific Publishers, pp 121-148. Suitable vectors may include plant viral-derived vectors (see e.g. EP-A-194809).

Vectors may also include a selectable genetic marker such as those that confer selectable phenotypes such as resistance to herbicides (e.g. kanamycin, hygromycin, phosphinotricin, chlorsulfuron, methotrexate, gentamycin, spectinomycin, imidazolinones and glyphosate).

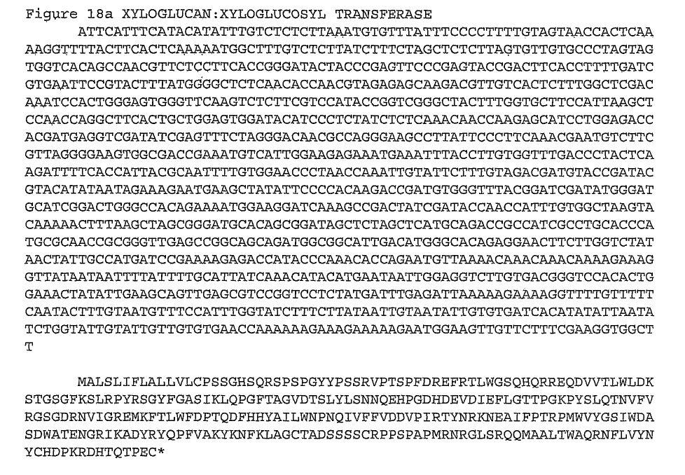

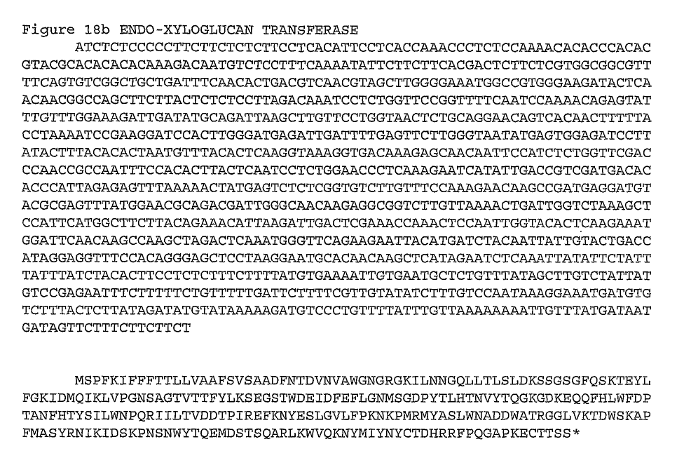

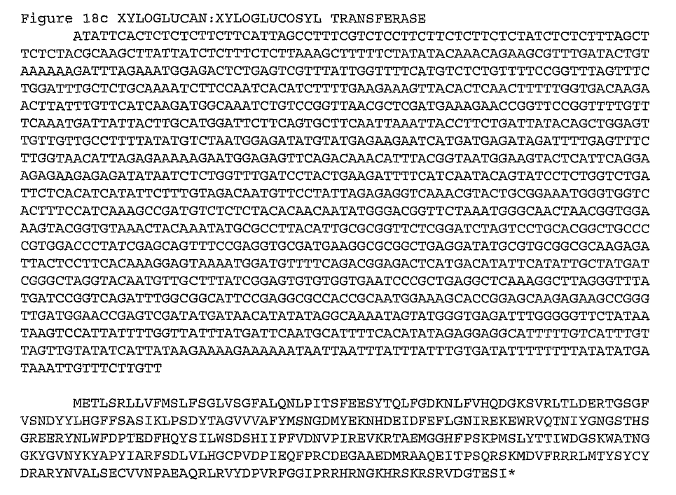

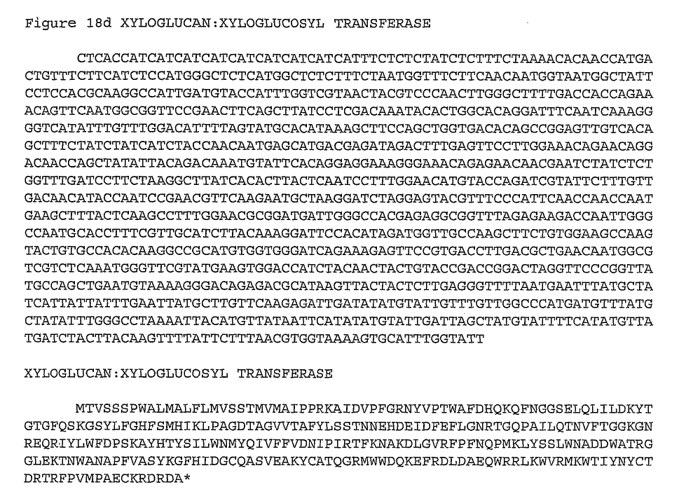

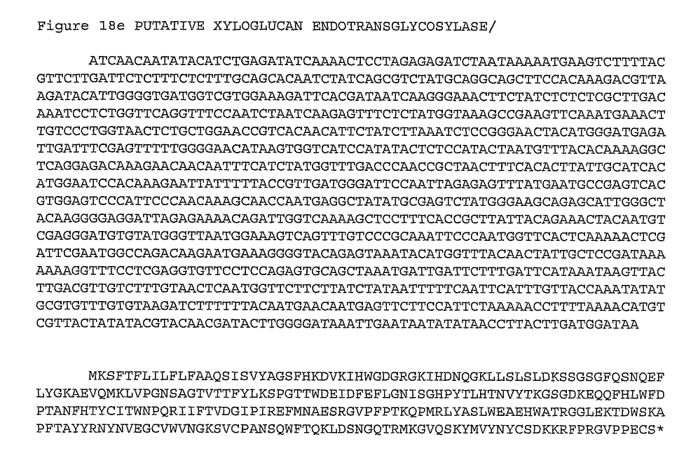

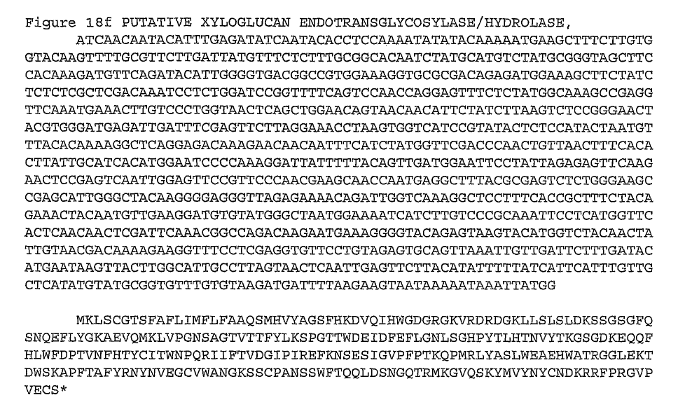

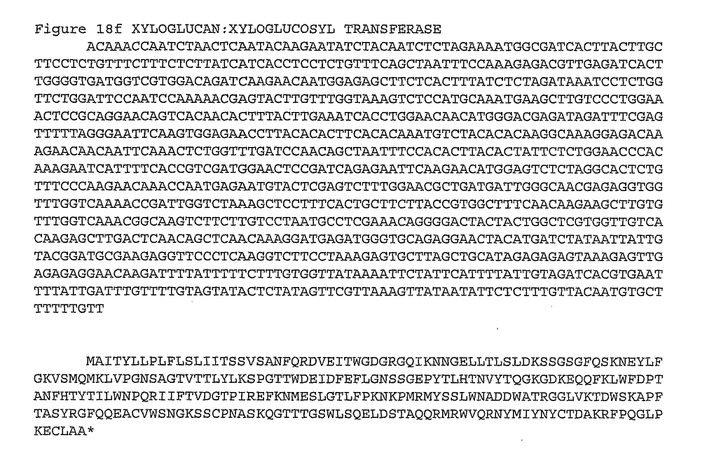

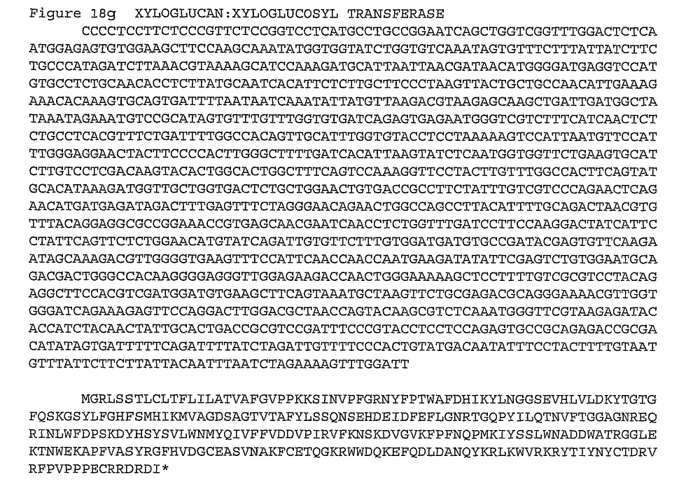

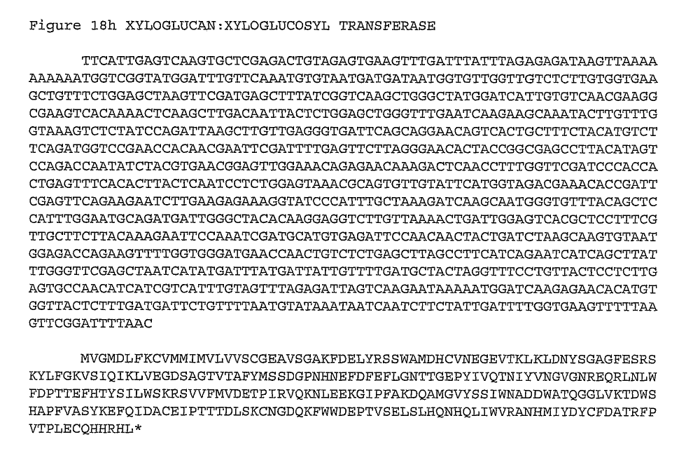

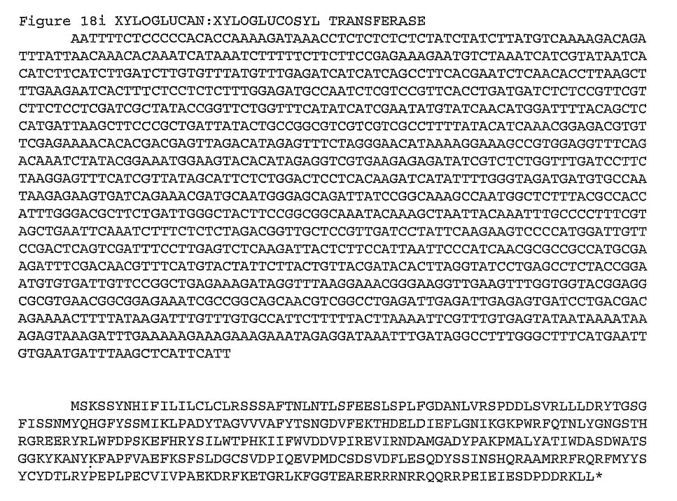

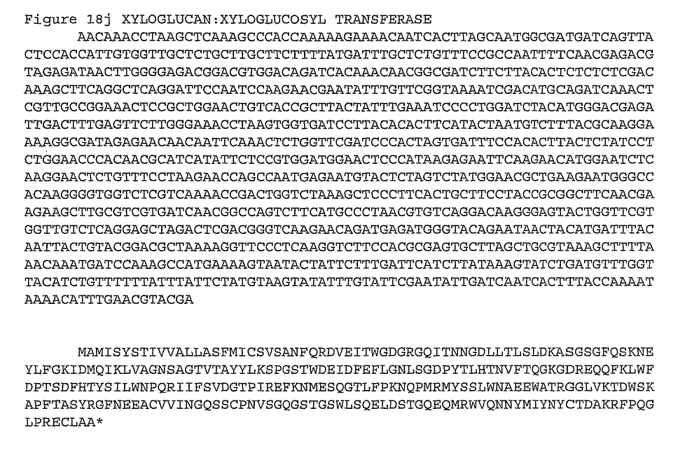

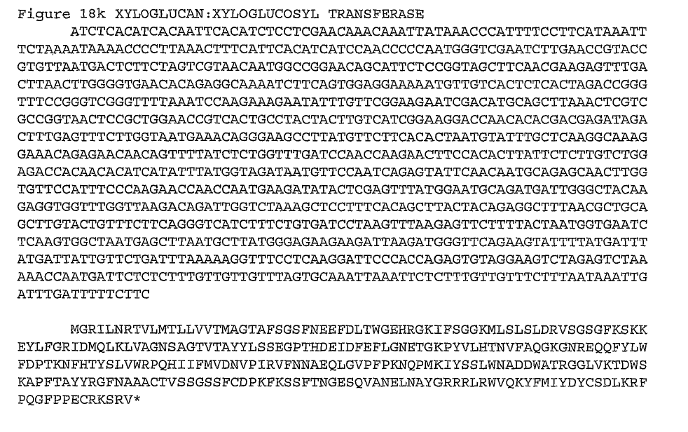

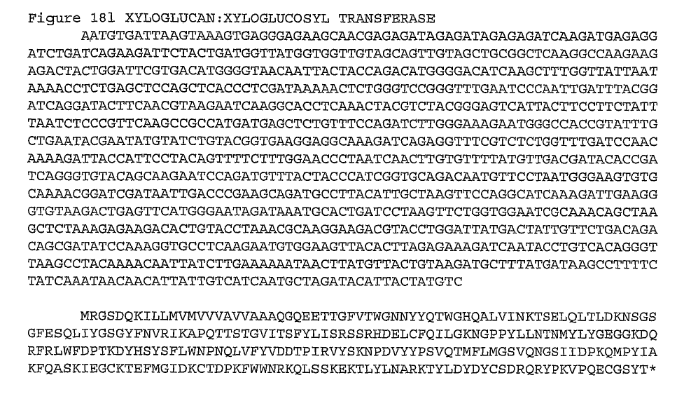

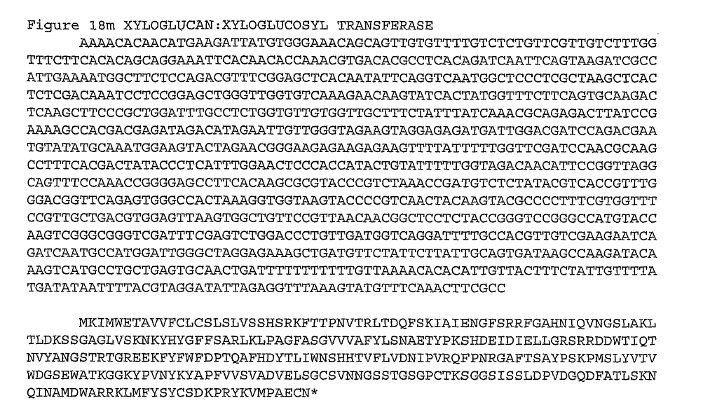

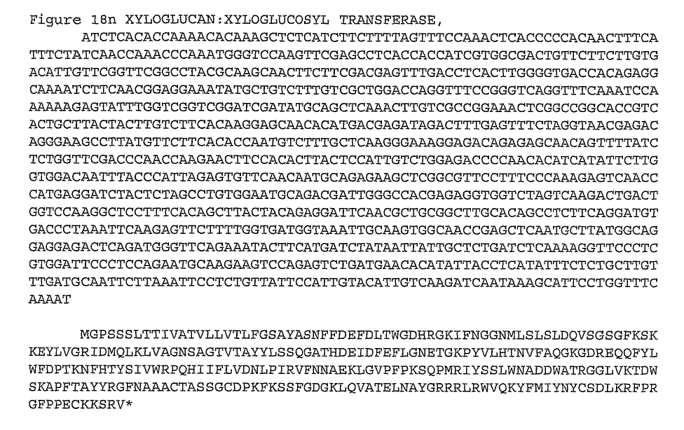

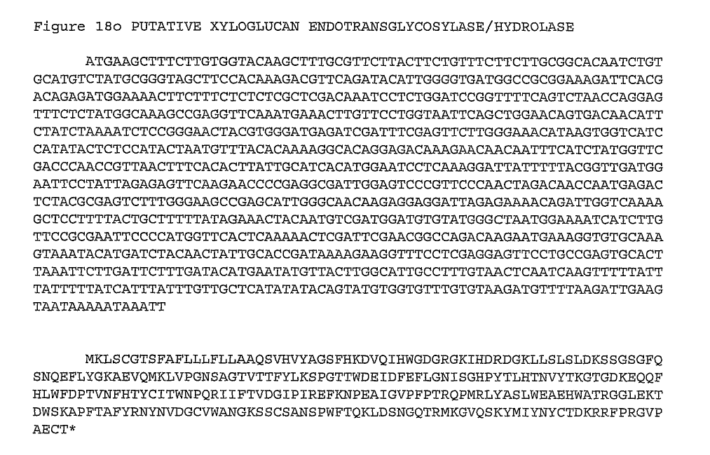

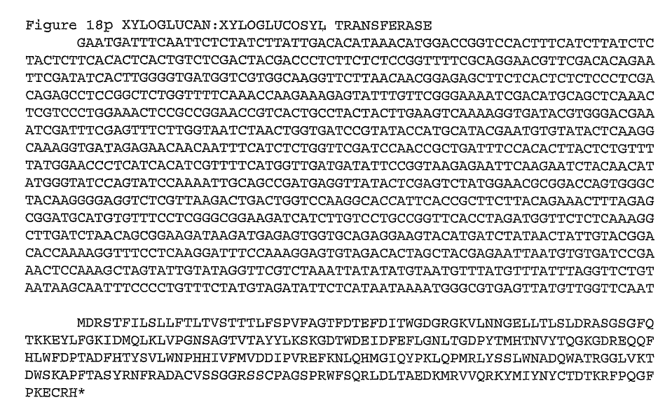

In a yet further preferred embodiment of the invention the genome of said transgenic plant cell is yet further modified by transfection with a nucleic acid selected from the group consisting of:
i) a nucleic acid molecule comprising a nucleic acid sequence as represented in FIG. 18a-18p;
ii) a nucleic acid molecule comprising a nucleic acid sequence that hybridises under stringent hybridisation conditions to a nucleic acid molecule in (i) and which encodes a plant cell modifying polypeptide;
iii) a nucleic acid molecule that encodes a polypeptide comprising the amino acid sequence as represented in FIG. 18a-18p.

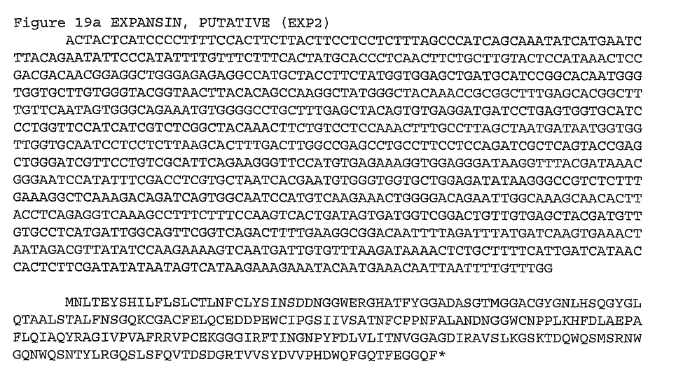

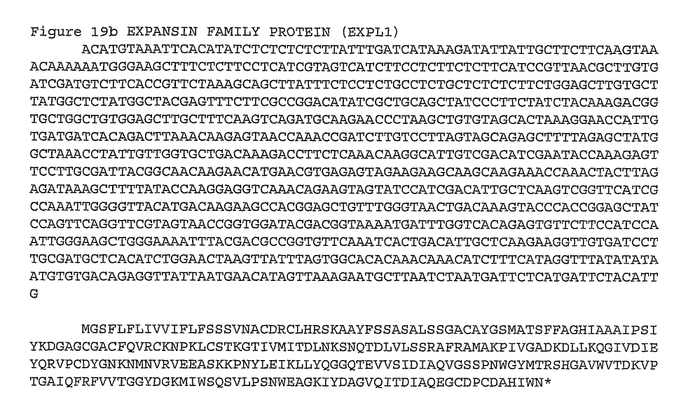

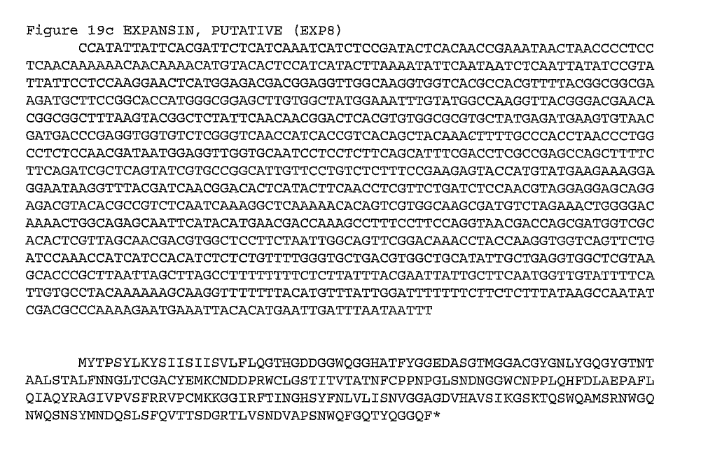

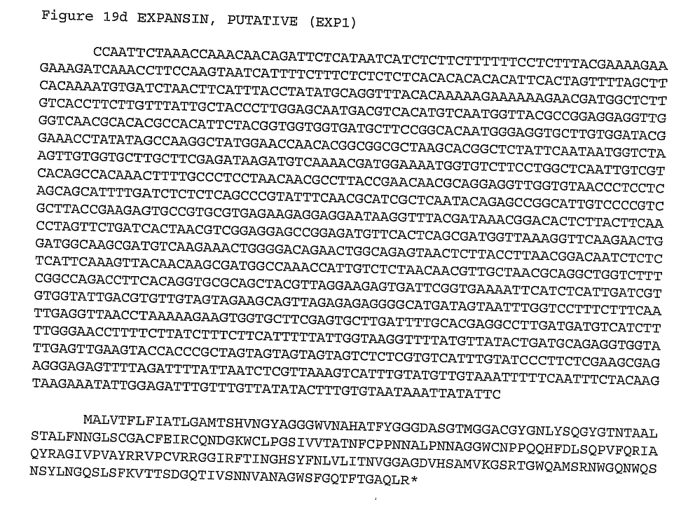

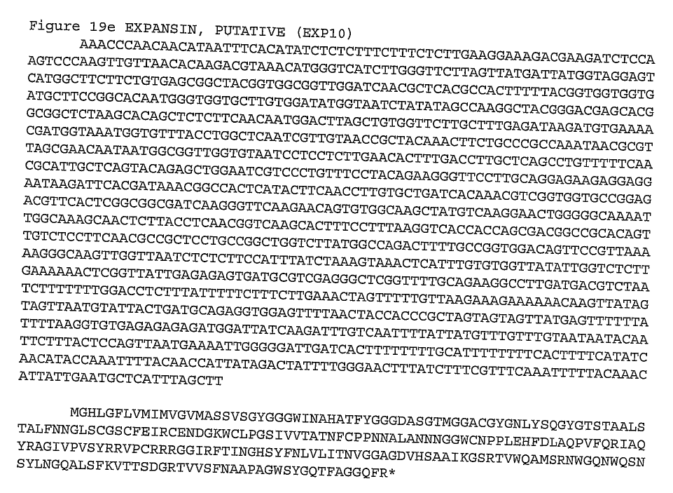

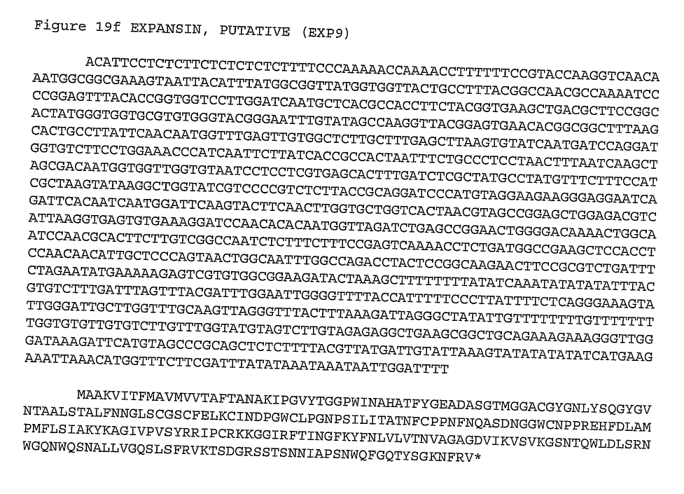

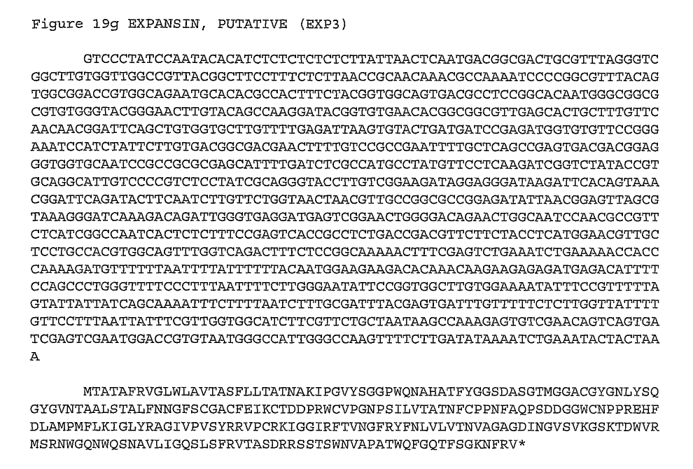

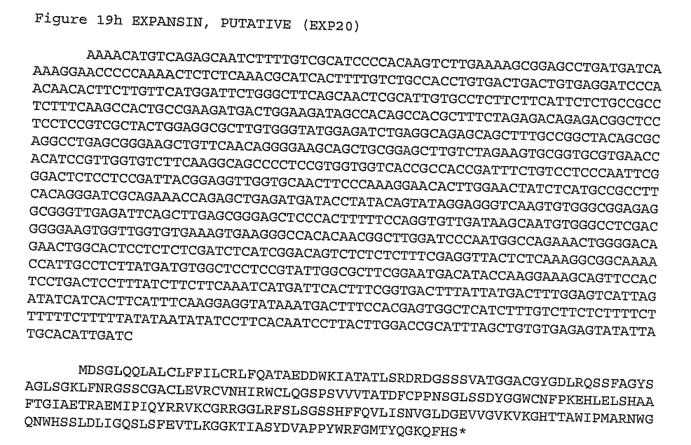

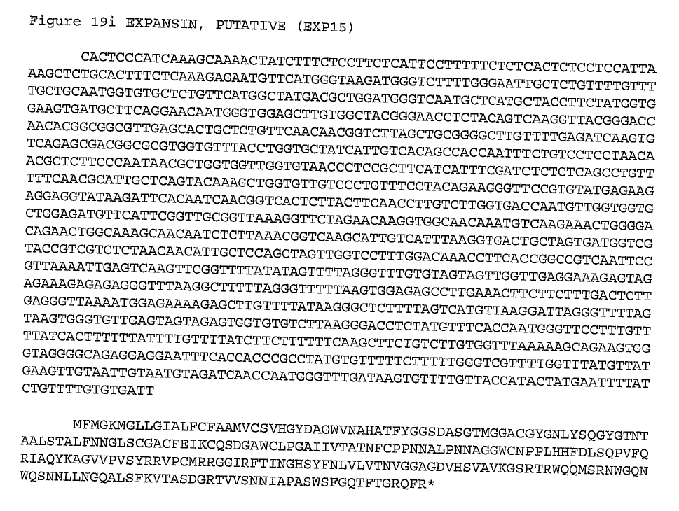

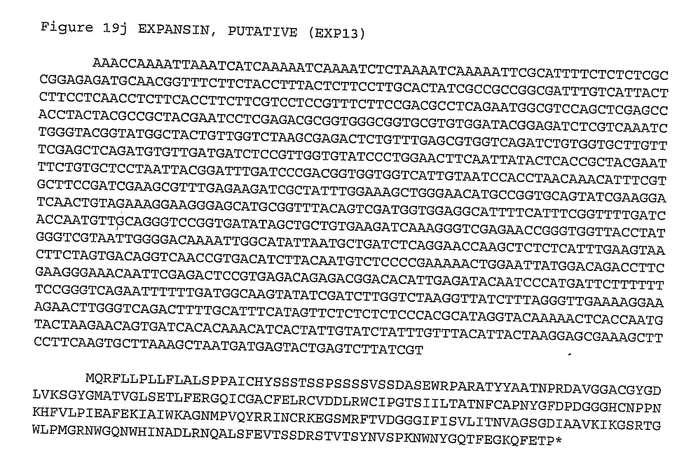

In a yet further preferred embodiment of the invention the genome of said transgenic plant cell is yet further modified by transfection with a nucleic acid selected from the group consisting of:
i) a nucleic acid molecule comprising a nucleic acid sequence as represented in FIG. 19a-19j;
ii) a nucleic acid molecule comprising a nucleic acid sequence that hybridises under stringent hybridisation conditions to a nucleic acid molecule in (i) and which encodes an expansin polypeptide;
iii) a nucleic acid molecule that encodes a polypeptide comprising the amino acid sequence as represented in FIG. 19a-19j.

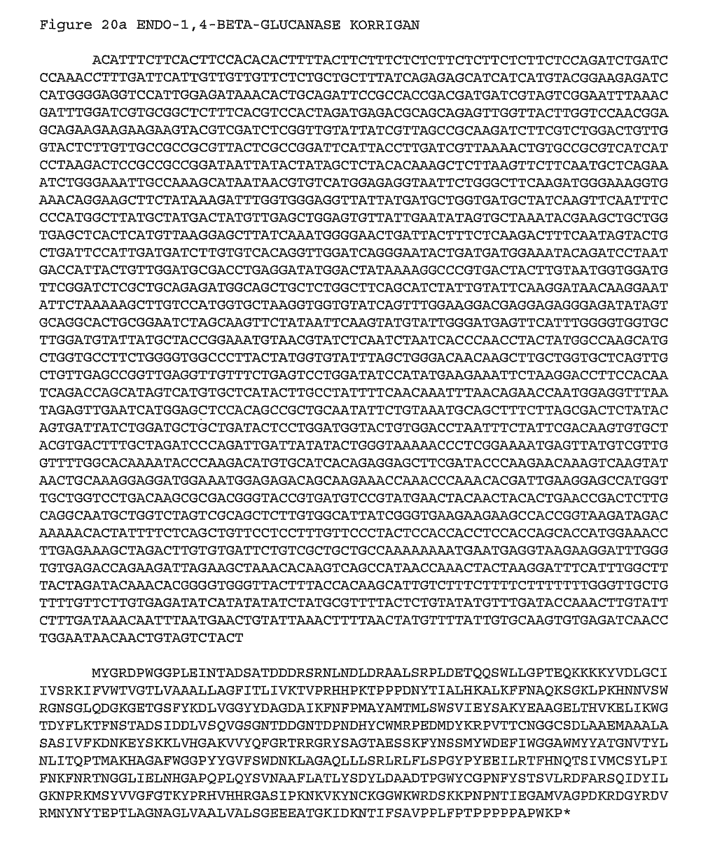

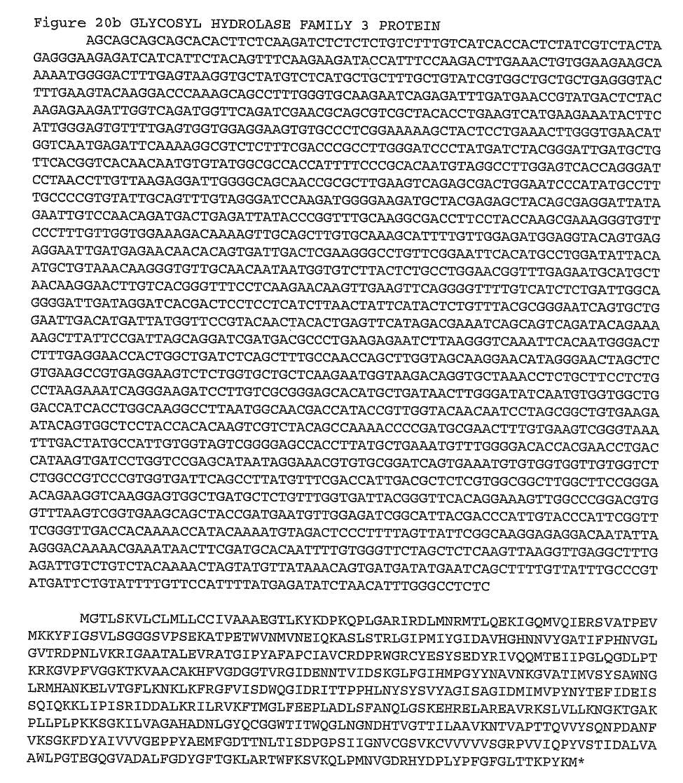

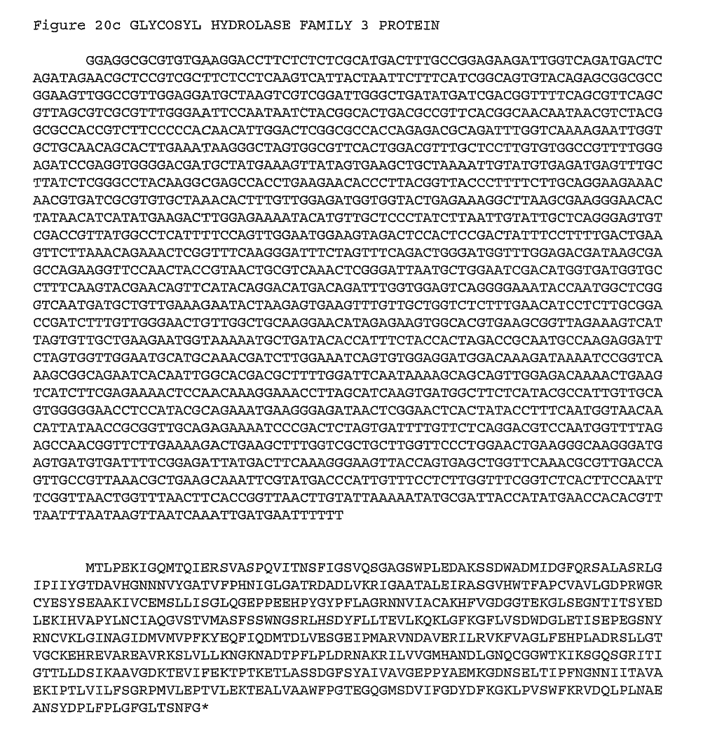

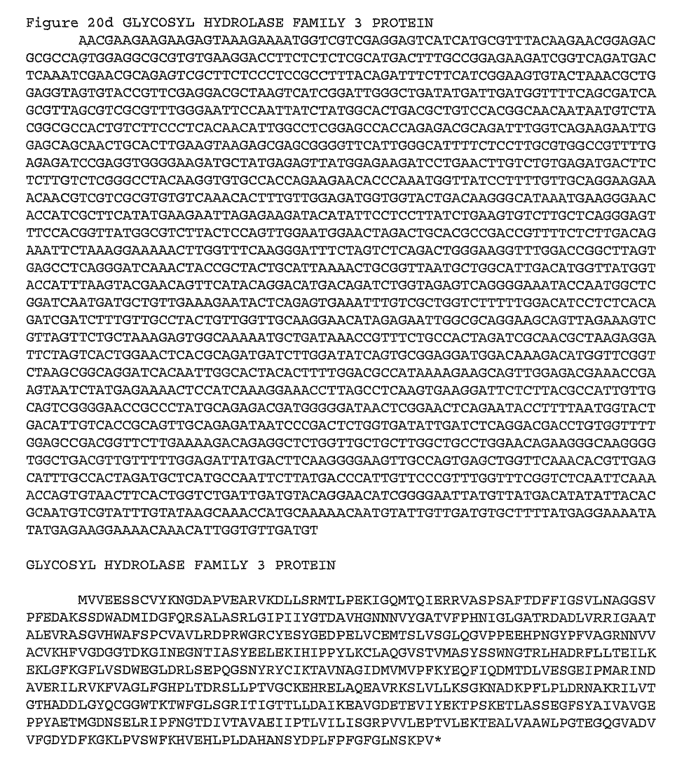

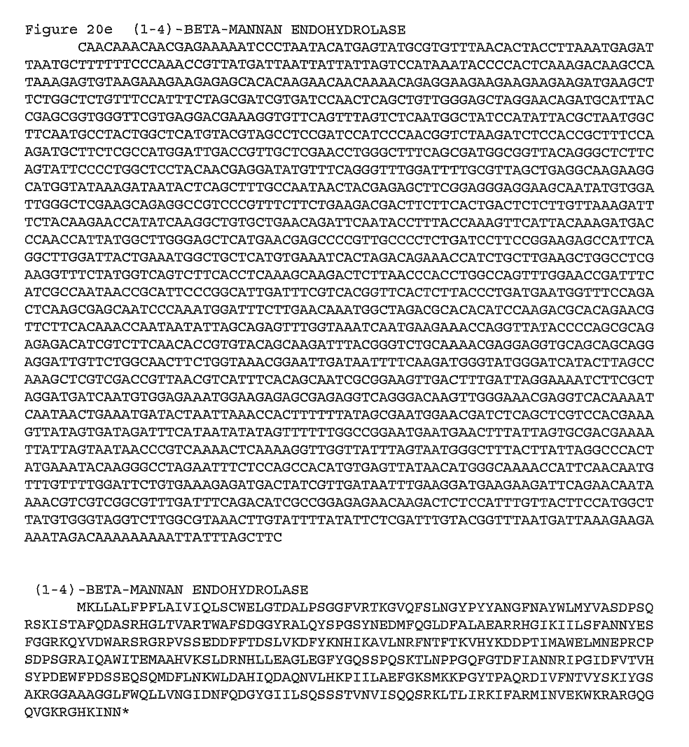

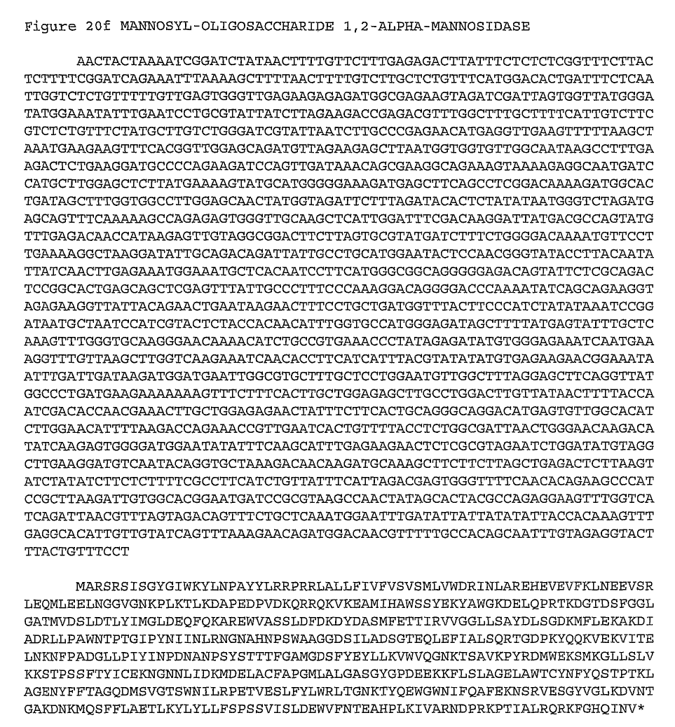

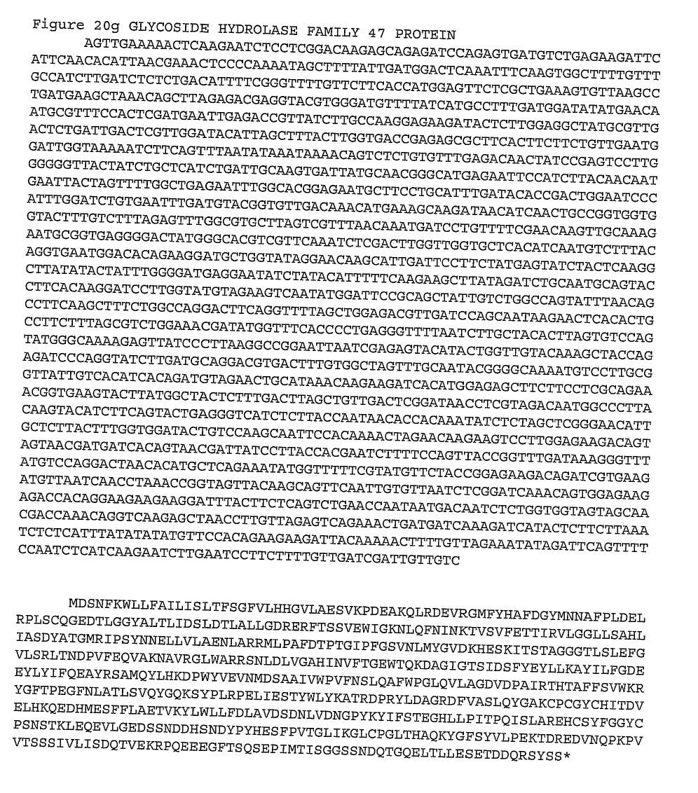

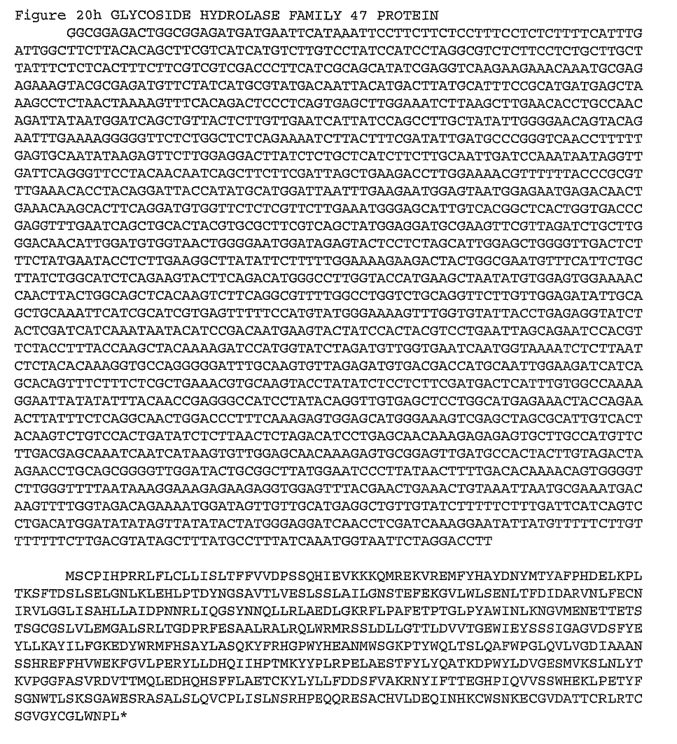

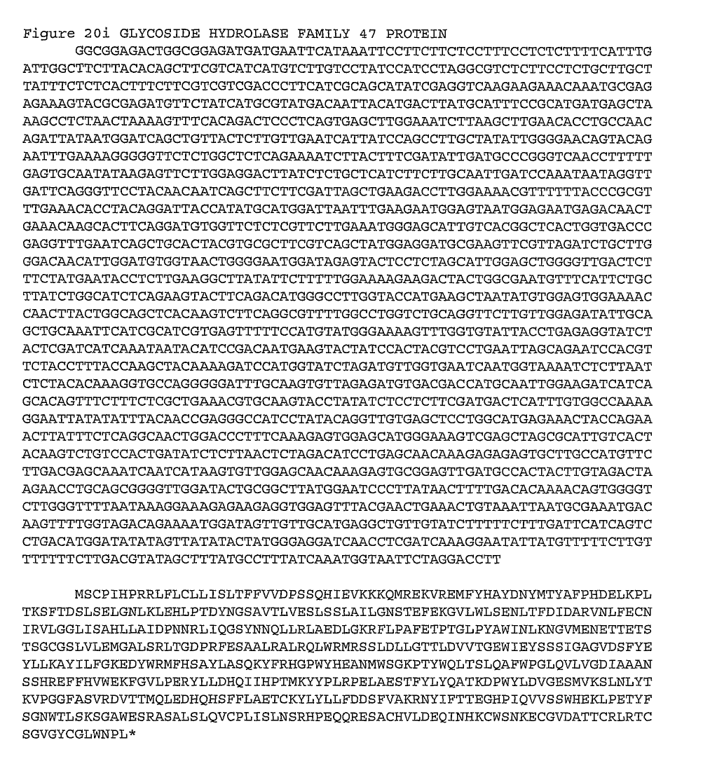

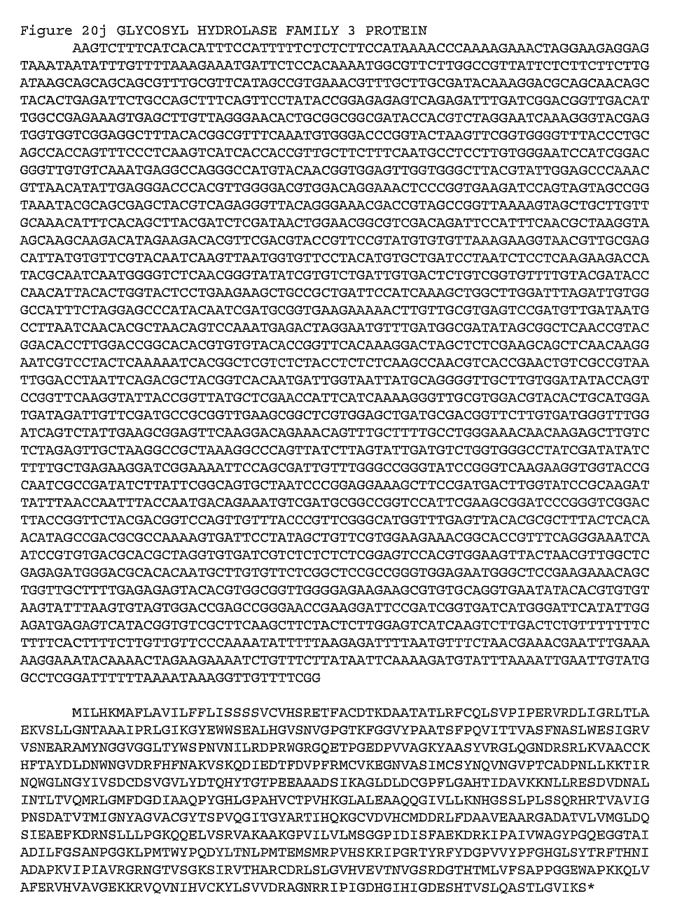

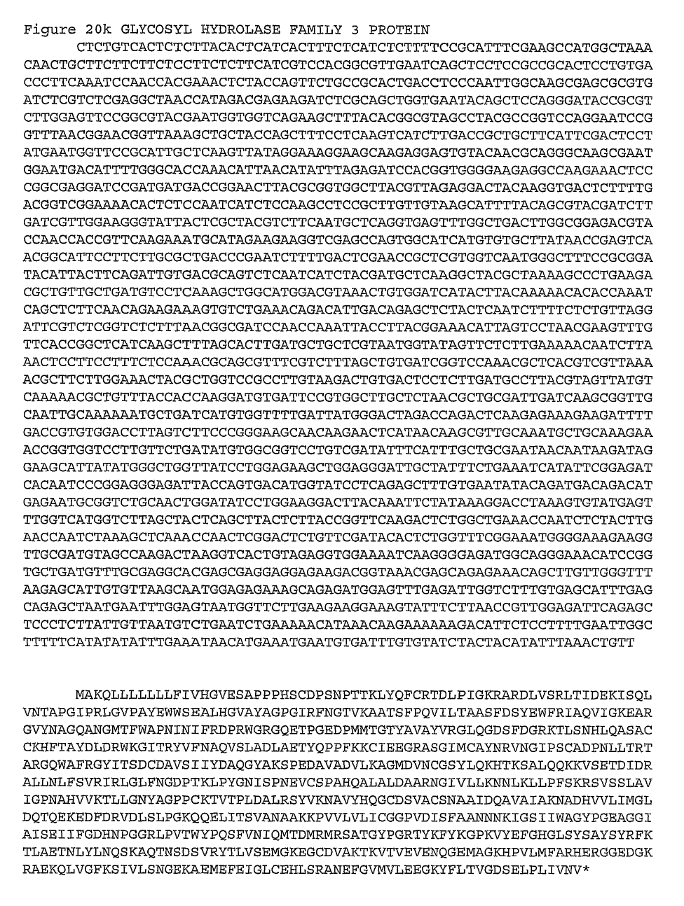

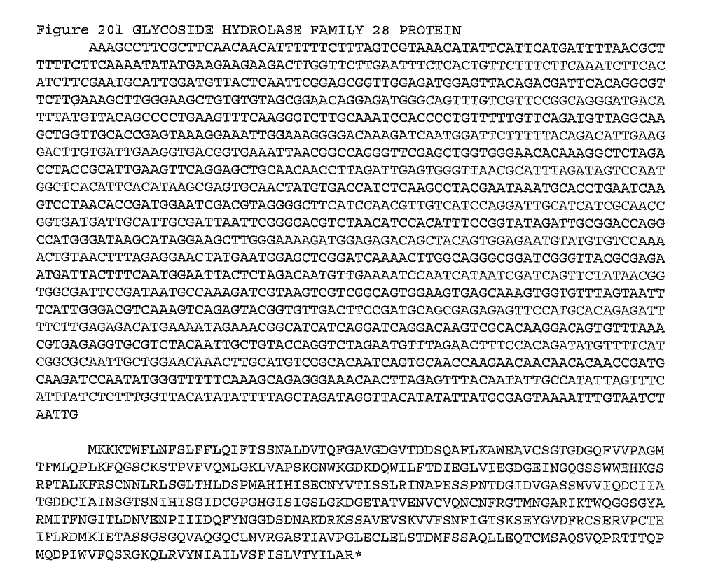

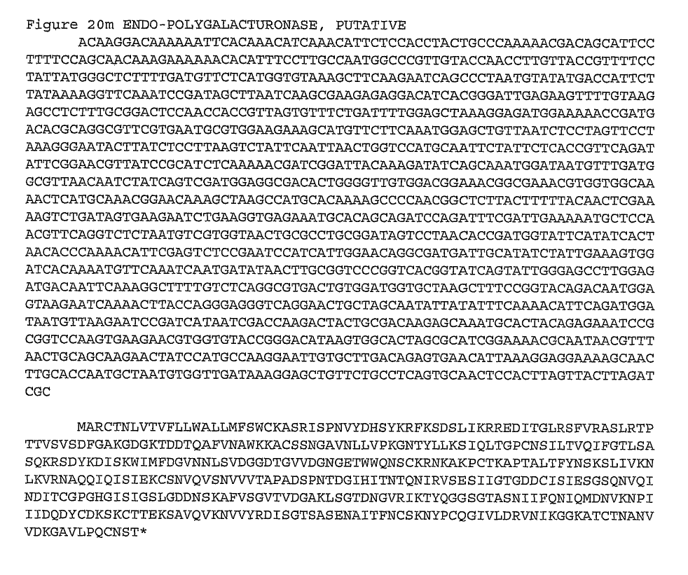

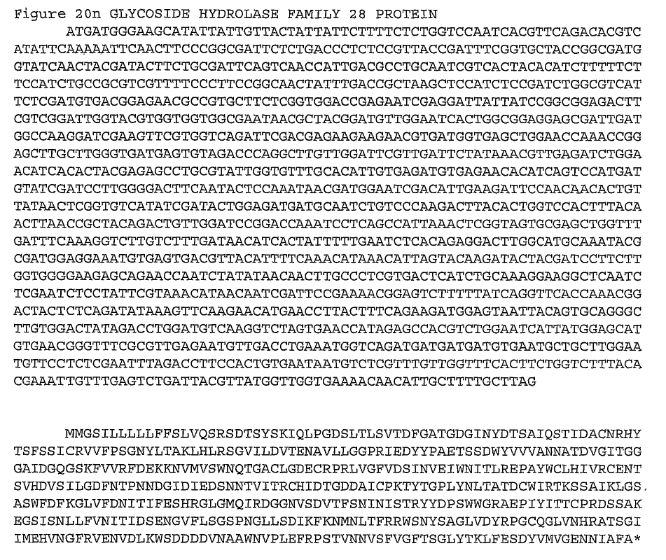

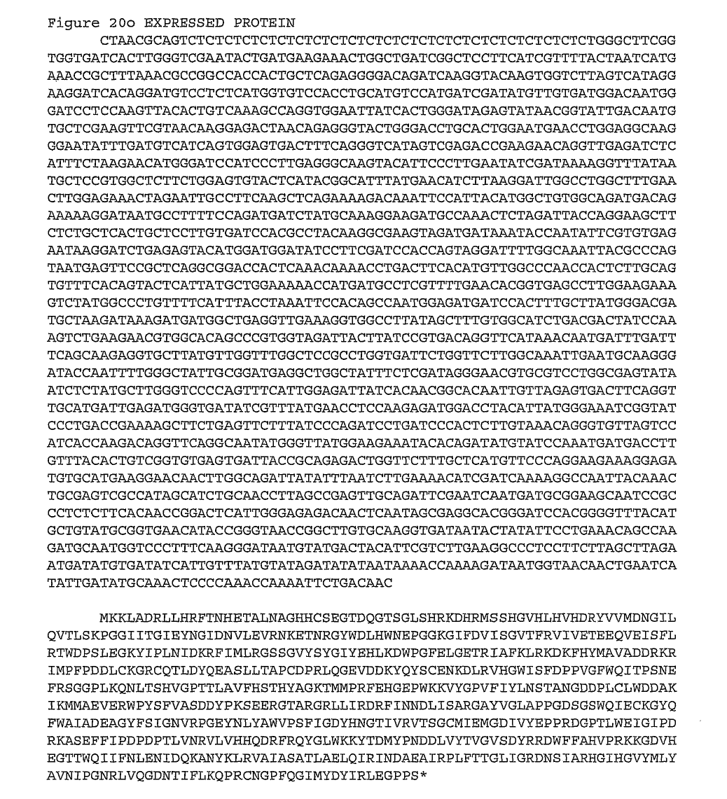

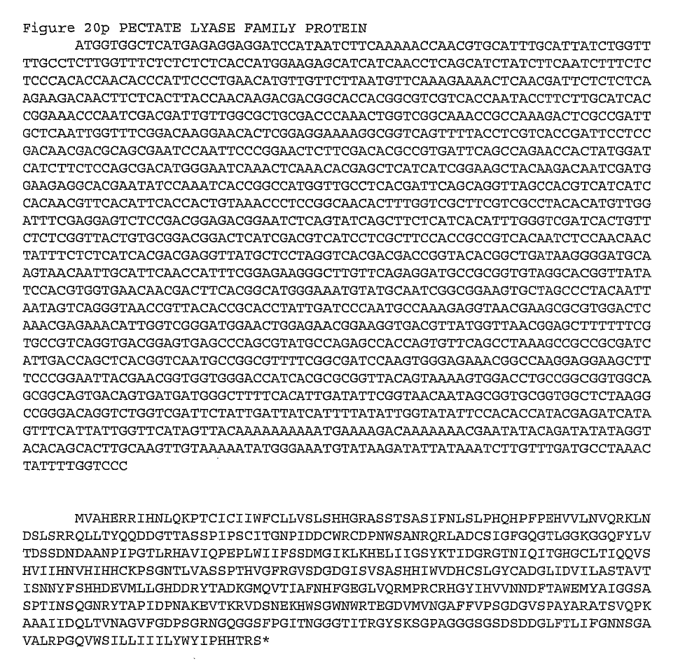

In a yet still further preferred embodiment of the invention the genome of said transgenic plant cell is yet further modified by transfection with a nucleic acid selected from the group consisting of:
i) a nucleic acid molecule comprising a nucleic acid sequence as represented in FIG. 20a-20p;
ii) a nucleic acid molecule comprising a nucleic acid sequence that hybridises under stringent hybridisation conditions to a nucleic acid molecule in (i) and which encodes a plant cell wall hydrolase polypeptide;
iii) a nucleic acid molecule that encodes a polypeptide comprising the amino acid sequence as represented in FIG. 20a-20p.

In tobacco and tomato (Solanum lycopersicum), both of which contain comparatively large endosperms in the mature seed, the endosperm is a major focus in efforts to understand the control of germination. In addition to its function as a storage tissue, in these species and others, the endosperm has been shown to exert control over germination by secreting cell wall loosening enzymes that weaken the mechanical resistance of the micropylar endosperm cap to radicle protrusion (reviewed in Bewley, 1997b). Importantly, the activity of endosperm-expressed cell wall loosening enzymes is controlled by both ABA and GA (Groot et al., 1988; Toorop et al., 2000).The controlled loosening of the micropylar endosperm cell walls to facilitate radicle emergence is achieved by the activity of multiple categories of cell wall—modifying enzymes, including β-mannanase, β-1,4-glucanase, expansins, xyloglucan endotransglycosidases, and polygalacturonases. A transcriptome study of Arabidopsis endosperm 24 hours after seed imbibition was performed and a number of genes associated with cell wall metabolism were identified (Penfield et al., 2006 and table 1).

Expression of these genes in biomass crops will result in cell wall loosening and cell wall breakdown which will be valuable for biomass utilisation by either making the cell walls more available to further breakdown to component sugars by additional enzymes or releasing sugars that can be used as feedstocks for fermentation directly.

In a preferred embodiment of the invention the genome of said transgenic plant cell is modified by transfection with a nucleic acid molecule that encodes a polypeptide the expression of which confers growth enhancing effects on said cell or a plant derived from said cell thereby increasing plant biomass.

In a preferred embodiment of the invention said nucleic acid molecule is over-expressed when compared to a non-transgenic reference plant cell of the same species.

“Plant biomass” refers to living plant tissue and lignocellulosic materials that comprise the plant and includes plant organs (e.g. stems, leaves, flowers, roots, seeds) which may increase in size, number or quality to increase yield. Genes that encode proteins that enhance the growth characteristics of a plant are well known in the art. For example WO92/09685, the content of which is incorporated by reference, describes the plant homologue of the yeast cell-cycle control gene cdc2 referred to as p34Cd 2 and is an important regulator of cell proliferation, particularly in leaf tissue. WO2005/085452, the content of which is incorporated by reference, describes the shoot specific expression of cyclin D3, a cell growth regulator and the enhancement of plant yield. WO2004/087929, the content of which is incorporated by reference, describes the expression of the CCS52 gene, a gene that encodes a cell-cycle regulatory protein, and the enhancement of plant size and increased organ size and number. WO2005/059147, the content of which is incorporated by reference, describes a growth regulatory protein, GRUBX and the effect of over-expression on plant morphology. WO2005/083094 describes a D-type cyclin dependent kinase which when over-expressed results in increased seed yield, also see WOWO2005/085452, WO2005/061702 and WO2006/100112 each of which is incorporated by reference in their entirety.

In a preferred embodiment of the invention said nucleic acid molecule that encodes a polypeptide the expression of which confers growth enhancing effects is selected from the group consisting of:
i) a nucleic acid molecule comprising a nucleic acid sequence as represented in FIG. 21;
ii) a nucleic acid molecule comprising a nucleic acid sequence that hybridises under stringent hybridisation conditions to a nucleic acid molecule in (i) and which encodes a transcription factor.

In our co-pending application (WO2007/063289) and which is incorporated by reference, we describe a transgenic plant that over-expresses a helix turn helix transcription factor referred to as Cesta. The phenotype of over-expressing plant lines is enhanced vegetative growth and an increase in leaf number.

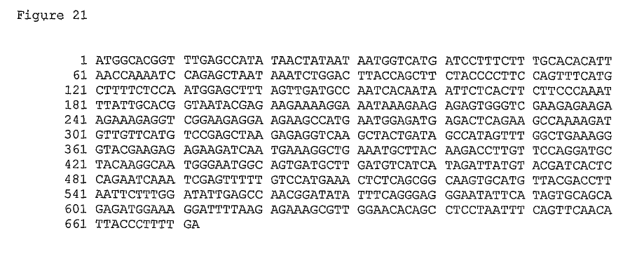

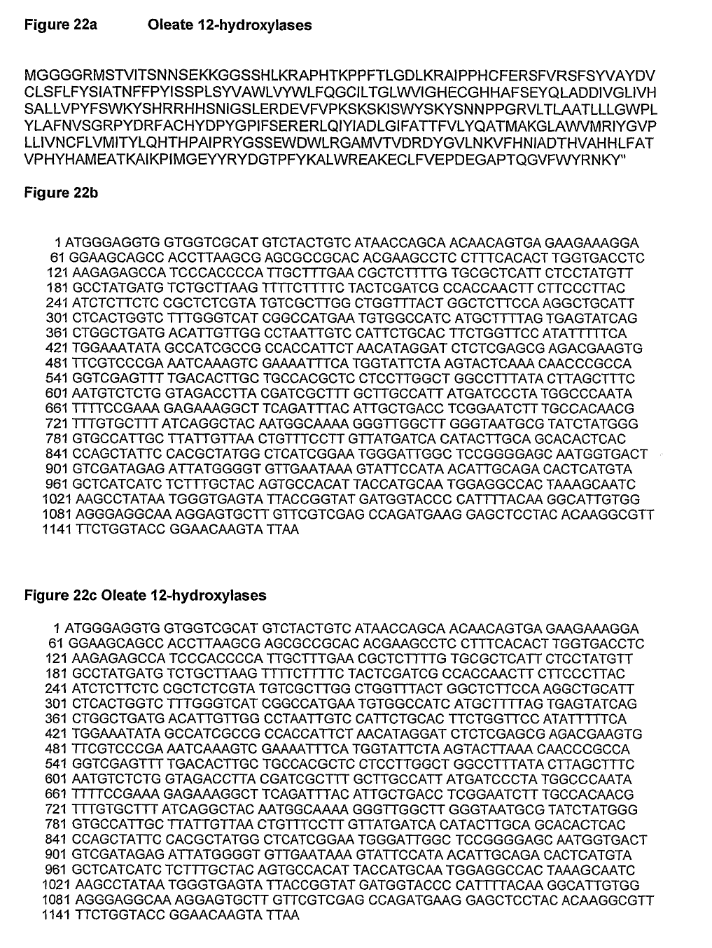

In a further preferred embodiment of the invention said cell is transfected with a nucleic acid molecule selected from:
i) a nucleic acid molecule comprising a nucleic acid sequence as represented in FIG. 22b or 22d;
ii) a nucleic acid molecule comprising a nucleic acid sequence that hybridises under stringent hybridisation conditions to a nucleic acid molecule in (i) and which encodes a oleate 12-hydroxylase.

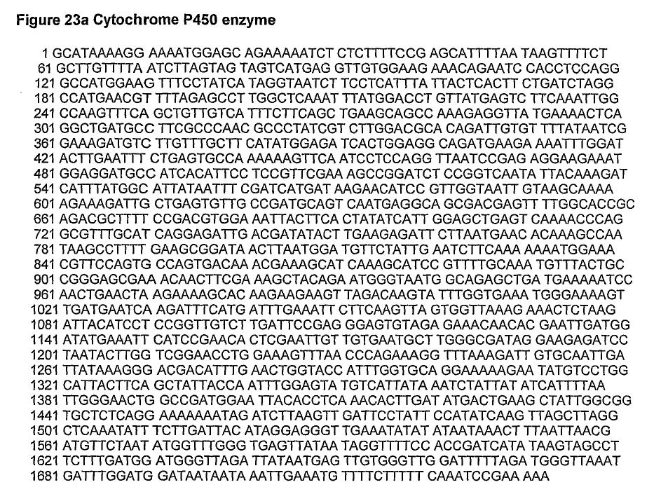

In a preferred embodiment of the invention said cell is transfected with a nucleic acid molecule selected from:
i) a nucleic acid molecule comprising a nucleic acid sequence as represented in FIG. 23a;
ii) a nucleic acid molecule comprising a nucleic acid sequence that hybridises under stringent hybridisation conditions to a nucleic acid molecule in (i) and which encodes a cytochrome P450.

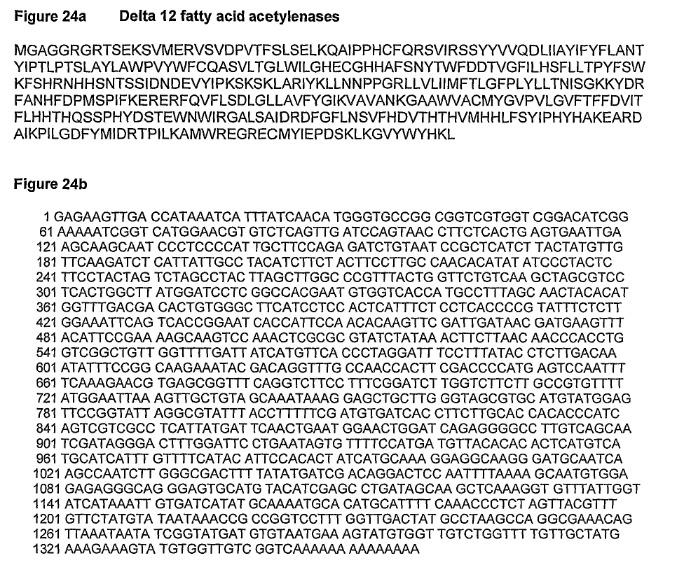

In a preferred embodiment of the invention said cell is transfected with a nucleic acid molecule selected from:
i) a nucleic acid molecule comprising a nucleic acid sequence as represented in FIG. 24a;
ii) a nucleic acid molecule comprising a nucleic acid sequence that hybridises under stringent hybridisation conditions to a nucleic acid molecule in (i) and which encodes a delta 12 fatty acid acetylenase.

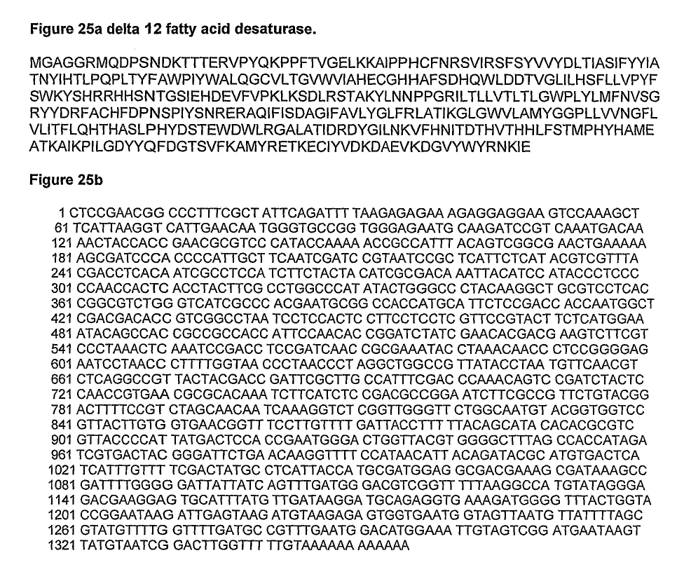

In a preferred embodiment of the invention said cell is transfected with a nucleic acid molecule selected from:
i) a nucleic acid molecule comprising a nucleic acid sequence as represented in FIG. 25b;
ii) a nucleic acid molecule comprising a nucleic acid sequence that hybridises under stringent hybridisation conditions to a nucleic acid molecule in (i) and which encodes a delta 12 fatty acid desaturase.

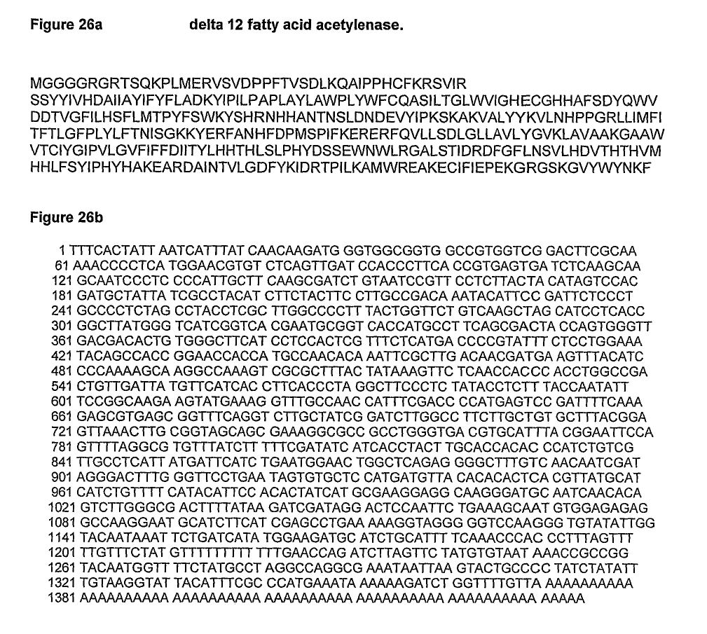

In a preferred embodiment of the invention said cell is transfected with a nucleic acid molecule selected from:
i) a nucleic acid molecule comprising a nucleic acid sequence as represented in FIG. 26b;
ii) a nucleic acid molecule comprising a nucleic acid sequence that hybridises under stringent hybridisation conditions to a nucleic acid molecule in (i) and which encodes a delta 12 fatty acid acetylenase.

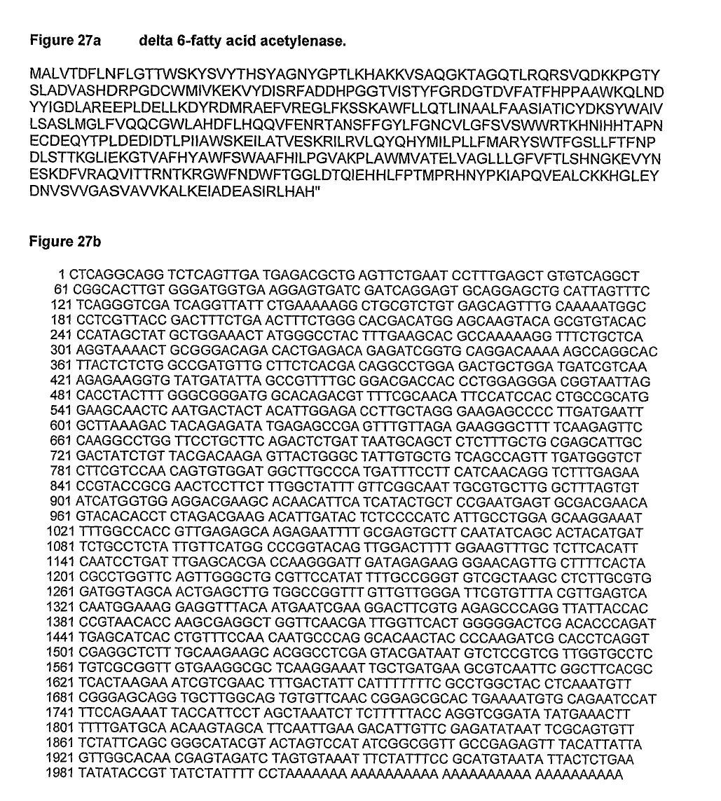

In a preferred embodiment of the invention said cell is transfected with a nucleic acid molecule selected from:
i) a nucleic acid molecule comprising a nucleic acid sequence as represented in FIG. 27b;
ii) a nucleic acid molecule comprising a nucleic acid sequence that hybridises under stringent hybridisation conditions to a nucleic acid molecule in (i) and which encodes a delta 6 fatty acid acetylenase.

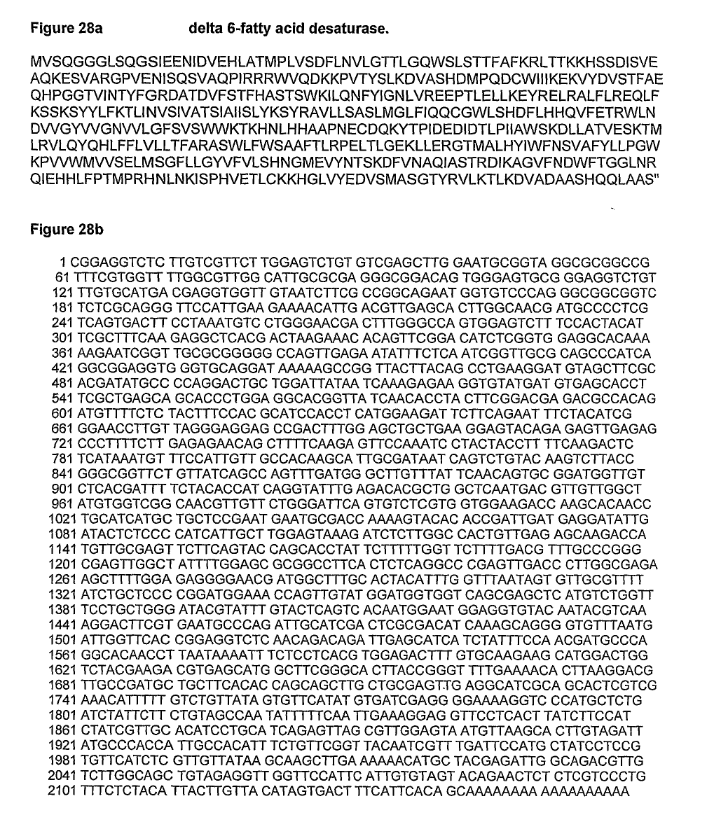

In a preferred embodiment of the invention said cell is transfected with a nucleic acid molecule selected from:
i) a nucleic acid molecule comprising a nucleic acid sequence as represented in FIG. 28b;
ii) a nucleic acid molecule comprising a nucleic acid sequence that hybridises under stringent hybridisation conditions to a nucleic acid molecule in (i) and which encodes a delta 6 fatty acid desaturase.

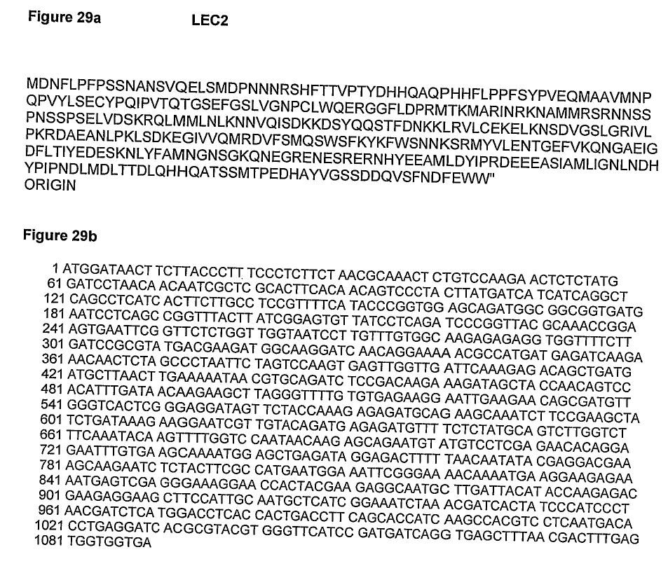

In a preferred embodiment of the invention said cell is transfected with a nucleic acid molecule selected from:
i) a nucleic acid molecule comprising a nucleic acid sequence as represented in FIG. 29b;
ii) a nucleic acid molecule comprising a nucleic acid sequence that hybridises under stringent hybridisation conditions to a nucleic acid molecule in (i) and which encodes a transcription factor.

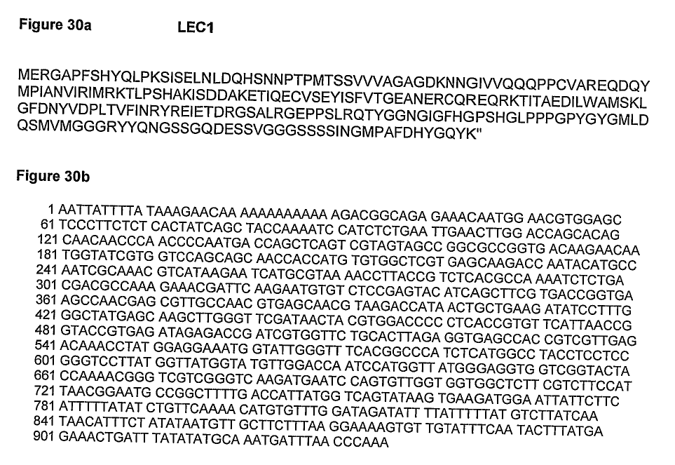

In a preferred embodiment of the invention said cell is transfected with a nucleic acid molecule selected from:
i) a nucleic acid molecule comprising a nucleic acid sequence as represented in FIG. 30b;
ii) a nucleic acid molecule comprising a nucleic acid sequence that hybridises under stringent hybridisation conditions to a nucleic acid molecule in (i) and which encodes a transcription factor.

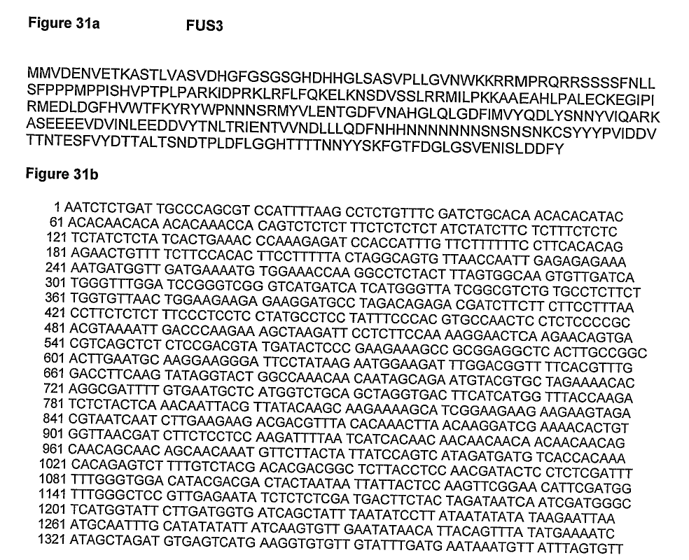

In a preferred embodiment of the invention said cell is transfected with a nucleic acid molecule selected from:
i) a nucleic acid molecule comprising a nucleic acid sequence as represented in FIG. 31b;
ii) a nucleic acid molecule comprising a nucleic acid sequence that hybridises under stringent hybridisation conditions to a nucleic acid molecule in (i) and which encodes a transcription factor.

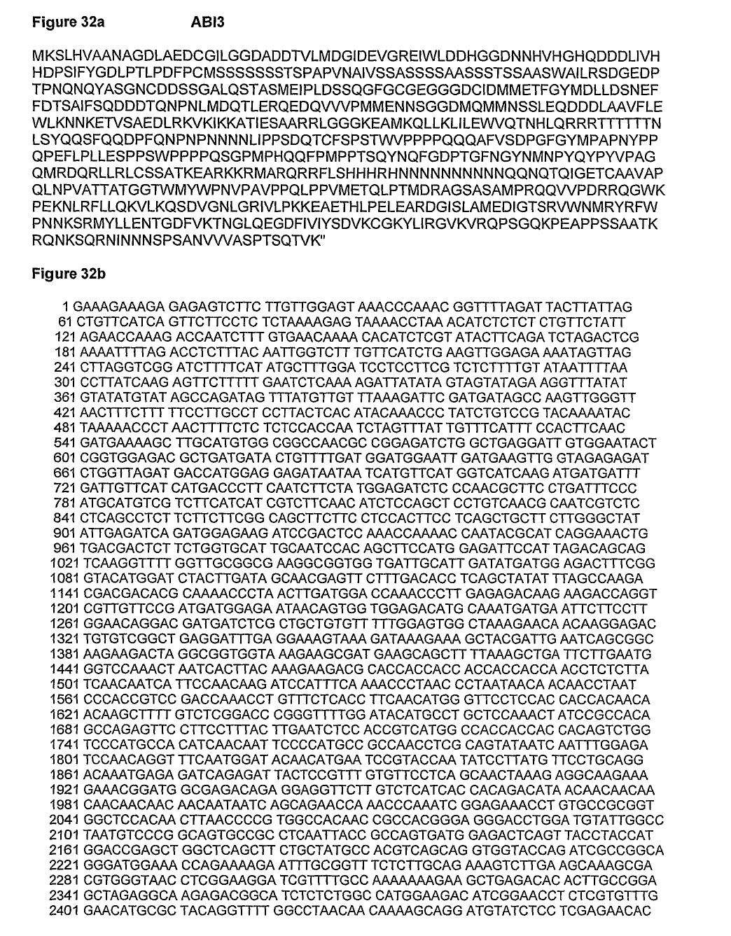

In a preferred embodiment of the invention said cell is transfected with a nucleic acid molecule selected from:
i) a nucleic acid molecule comprising a nucleic acid sequence as represented in FIG. 32b;
ii) a nucleic acid molecule comprising a nucleic acid sequence that hybridises under stringent hybridisation conditions to a nucleic acid molecule in (i) and which encodes a transcription factor.

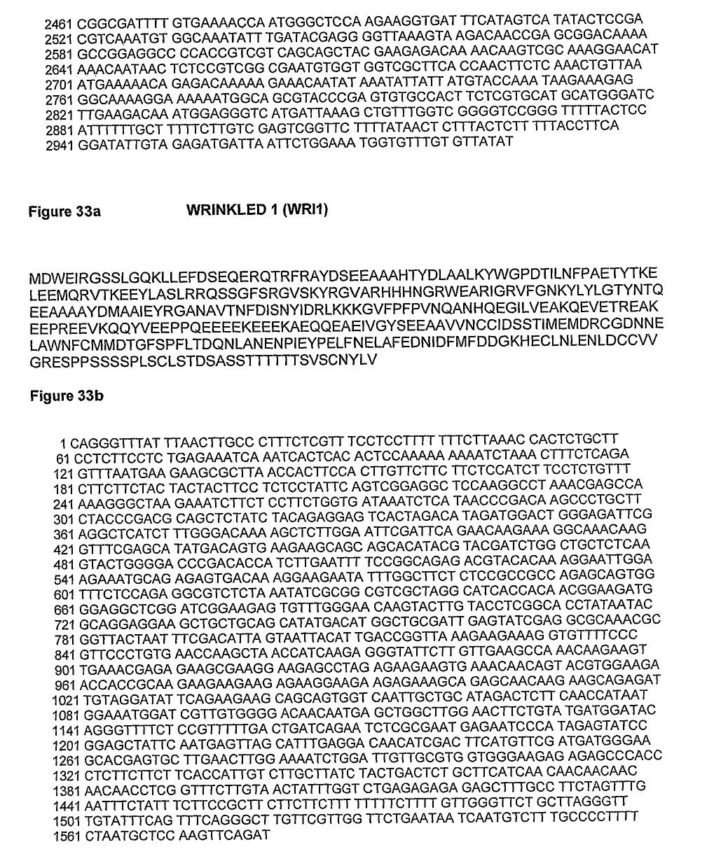

In a preferred embodiment of the invention said cell is transfected with a nucleic acid molecule selected from:
i) a nucleic acid molecule comprising a nucleic acid sequence as represented in FIG. 33b;
ii) a nucleic acid molecule comprising a nucleic acid sequence that hybridises under stringent hybridisation conditions to a nucleic acid molecule in (i) and which encodes a transcription factor.

In a preferred embodiment of the invention there is provided a transgenic plant comprising a cell according to the invention.

In a preferred embodiment of the invention said plant is selected from the group consisting of: corn (Zea mays), canola (Brassica napus, Brassica rapa ssp.), flax (Linum usitatissimum), alfalfa (Medicago sativa), rice (Oryza sativa), rye (Secale cerale), sorghum (Sorghum bicolor, Sorghum vulgare), sunflower (Helianthus annus), wheat (Tritium aestivum), soybean (Glycine max), tobacco (Nicotiana tabacum), potato (Solanum tuberosum), peanuts (Arachis hypogaea), cotton (Gossypium hirsutum), sweet potato (lopmoea batatus), cassaya (Manihot esculenta), coffee (Cofea spp.), coconut (Cocos nucifera), pineapple (Anana comosus), citrus tree (Citrus spp.) cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables and ornamentals.

Preferably, plants of the present invention are biomass crops (switchgrass, alfalfa, willow, poplar, eucalyptus, miscanthus, wheat, maize or barley.), other crop plants (for example, cereals and pulses, maize, wheat, potatoes, tapioca, rice, sorghum, millet, cassaya, barley, pea), and other root, tuber or seed crops. Important seed crops are oil-seed rape, sugar beet, maize, sunflower, soybean, sorghum, and flax (linseed). Horticultural plants to which the present invention may be applied may include lettuce, endive, and vegetable brassica including cabbage, broccoli, and cauliflower. The present invention may be applied in tobacco, cucurbits, carrot, strawberry, sunflower, tomato, pepper.

According to a further aspect of the invention there is provided a seed comprising a plant cell according to the invention.

According to a further aspect of the invention there is provided a method to modulate and extract plant mono- di- or triacylglycerol fatty acids comprising the steps of:
i) providing a transgenic plant the genome of which is modified by transfection with a nucleic acid molecule selected from the group consisting of:
a) a nucleic acid molecule comprising an expression cassette which cassette comprises a nucleic acid sequence that encodes at least part of a gene that encodes a polypeptide that controls the synthesis, degradation or transport of fatty acids and/or fatty acyl CoAs and therefore synthesis and degradation of mono- di- or triacylglycerols, wherein said cassette is adapted such that both sense and antisense nucleic acid molecules are transcribed from said cassette wherein the expression from said cassette produces an interfering RNA molecule that inhibits the expression of said gene;
b) a nucleic acid molecule comprising an expression cassette which cassette comprises a nucleic acid sequence that encodes at least part of a gene that encodes a polypeptide that controls the synthesis, degradation or transport of fatty acids and/or fatty acyl CoAs and therefore synthesis and degradation of mono- di- or triacylglycerols, wherein said cassette is adapted such that an antisense nucleic acid molecule is transcribed from said cassette wherein the expression from said cassette produces an antisense RNA molecule that inhibits the expression of said gene;
c) a nucleic acid molecule that encodes a polypeptide that controls the synthesis, degradation or transport of fatty acids and/or fatty acyl CoAs and therefore synthesis and degradation of mono- di- or triacylglycerols, in a plant cell which polypeptide is a variant polypeptide that varies from a native polypeptide sequence wherein said variant polypeptide is a dominant negative suppressor of the native polypeptide and inhibits the production of mono- di- or triacylglycerol, wherein said nucleic acid molecule in a), b) or c) is operably linked to a promoter sequence;
ii) inducing expression of at least one nucleic acid molecule according to the invention;
iii) harvesting transgenic plant material; and optionally
iv) extracting said harvested plant material to provide a mono- di- or triacylglycerol or free fatty acid fraction and an extracted plant material fraction.

In a preferred method of the invention the induction of expression of said nucleic acid molecules is by induction of senescence.

In a preferred method of the invention the induction of senescence is by growing said plant in reduced light conditions.

In an alternative preferred method of the invention the induction of senescence is by altered day-length.

In a yet further method of the invention senescence is induced by chemical treatment.

In a yet further preferred method of the invention the genome of said transgenic plant cell is yet further modified by transfection with a nucleic acid selected from the group consisting of:
i) a nucleic acid molecule comprising a nucleic acid sequence as represented in FIG. 18a-18p;
ii) a nucleic acid molecule comprising a nucleic acid sequence that hybridises under stringent hybridisation conditions to a nucleic acid molecule in (i) and which encodes a plant cell modifying polypeptide;
iii) a nucleic acid molecule that encodes a polypeptide comprising the amino acid sequence as represented in FIG. 18a-18p.

In a yet further preferred method of the invention the genome of said transgenic plant cell is yet further modified by transfection with a nucleic acid selected from the group consisting of:
i) a nucleic acid molecule comprising a nucleic acid sequence as represented in FIG. 19a-19j;
ii) a nucleic acid molecule comprising a nucleic acid sequence that hybridises under stringent hybridisation conditions to a nucleic acid molecule in (i) and which encodes an expansin polypeptide;
iii) a nucleic acid molecule that encodes a polypeptide comprising the amino acid sequence as represented in FIG. 19a-19j.

In a yet still further preferred method of the invention the genome of said transgenic plant cell is yet further modified by transfection with a nucleic acid selected from the group consisting of:
i) a nucleic acid molecule comprising a nucleic acid sequence as represented in FIG. 20a-20p;

ii) a nucleic acid molecule comprising a nucleic acid sequence that hybridises under stringent hybridisation conditions to a nucleic acid molecule in (i) and which encodes a plant cell wall hydrolase polypeptide;
iii) a nucleic acid molecule that encodes a polypeptide comprising the amino acid sequence as represented in FIG. 20a-20p.

In a preferred method of the invention said extracted plant material is further processed by saccharification to sugar.

Saccharification is a process by which plant lignocellulosic materials (e.g., lignin, cellulose, hemicellulose) are hydrolysed to glucose through chemical and enzymic means. Typically this involves the pre-treatment of plant material with alkali to remove lignin followed by enzyme digestion of cellulose. This typically uses fungal cellulose, for example from the fungus Tichoderma reesei. The present invention utilises plant hydrolases in saccharification thereby simplifying the process.

In a further preferred method of the invention said sugar is used as a feedstock in the production of ethanol by microbial fermentation.

Microorganisms used in the process according to the invention are grown or cultured in the manner with which the skilled worker is familiar, depending on the host organism. As a rule, microorganisms are grown in a liquid medium comprising a carbon source (e.g. sugar as formed during the saccharification process), a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen.

The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The products produced can be isolated from the organisms as described above by processes known to the skilled worker, for example by extraction or distillation. In this process, the pH value is advantageously kept between pH 4 and 12, preferably between pH 6 and 9, especially preferably between pH 7 and 8.

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more, nitrogen sources, inorganic salts, vitamins and/or trace elements.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing microorganisms usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters.

The fermentation broth can then be processed further. The biomass may, according to requirement, be removed completely or partially from the fermentation broth by separation methods such as, for example, centrifugation, filtration, decanting or a combination of these methods or be left completely in said broth.

According to a further aspect of the invention there is provided a composition comprising mono- di- or triacylglycerol formed by the method according to the invention.

In a preferred embodiment of the invention said composition is a biofuel.

In a further preferred embodiment of the invention said composition is a nutraceutical.

In a preferred embodiment of the invention said composition comprises elevated levels of galactolipids.

In a further preferred embodiment of the invention said composition comprises elevated levels of linolenic acid.

An additional method to regulate the expression of plant genes is by virus induced gene silencing (VIGS). A viral infection in a plant induces an RNA mediated defence response against the infecting virus that targets the viral genome and any foreign sequences cloned into the viral genome. The phenomenon is related to RNA interference and only requires a short region of foreign sequence to induce a specific degradation of the RNA that corresponds to the foreign nucleic acid. Advantageously, the method of VIGS does not require the stable genetic modification of the plant genome to effect an ablation effect on gene expression but simply the infection of a plant with a virus that is engineered to include a plant nucleic acid sequence the regulation of which is desired.

According to a further aspect of the invention there is provided a modified plant wherein said plant comprises a virus that includes a nucleic acid molecule wherein said nucleic acid molecule is at least part of a gene that encodes a protein that controls the synthesis, degradation or transport of fatty acids and/or fatty acyl CoAs and therefore the synthesis and degradation of mono- di- or triacylglycerols in a plant cell.

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 13a;
ii) a nucleic acid molecule comprising a nucleic acid sequence that hybridises under stringent hybridisation conditions to a nucleic acid molecule in (i) and which encodes a fatty acid transporter polypeptide;
iii) a nucleic acid molecule that encodes a variant polypeptide that varies from a polypeptide comprising the amino acid sequence as represented in FIG. 13b.

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 14a or 14c;
ii) a nucleic acid molecule comprising a nucleic acid sequence that hybridises under stringent hybridisation conditions to a nucleic acid molecule in (i) and which encodes a long chain acyl Co A synthetase polypeptide;
iii) a nucleic acid molecule that encodes a variant polypeptide that varies from a polypeptide comprising the amino acid sequence as represented in FIG. 14b or 14d.

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 15a, 15c, 15e, 15g, 15i or 15k;
ii) a nucleic acid molecule comprising a nucleic acid sequence that hybridises under stringent hybridisation conditions to a nucleic acid molecule in (i) and which encodes an acyl CoA oxidase polypeptide;
iv) a nucleic acid molecule that encodes a variant polypeptide that varies from a polypeptide comprising the amino acid sequence as represented in FIG. 15b, 15d, 15f, 15h, 15j or 15l.

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 16a; 16c,16e or 16g
ii) a nucleic acid molecule comprising a nucleic acid sequence that hybridises under stringent hybridisation conditions to a nucleic acid molecule in (i) and which encodes a Keto-Acyl-CoA thiolase;
iii) a nucleic acid molecule that encodes a variant polypeptide that varies from a polypeptide comprising the amino acid sequence as represented in FIG. 16b, 16d, 16f or 16h.

In a preferred embodiment of the invention said gene is encoded by a nucleic acid molecule comprising a nucleic acid sequence selected from the group consisting of:
i) a nucleic acid molecule comprising a nucleic acid sequence as represented in FIG. 17a or 17c
ii) a nucleic acid molecule comprising a nucleic acid sequence that hybridises under stringent hybridisation conditions to a nucleic acid molecule in (i) and which encodes a multifunctional protein involved in peroxisomal β oxidation;
iii) a nucleic acid molecule that encodes a variant polypeptide that varies from a polypeptide comprising the amino acid sequence as represented in FIG. 17b or 17d.

In a preferred embodiment of the invention said nucleic acid molecule is between 20-30 base pairs in length.

In a preferred embodiment of the invention said nucleic acid molecule consists of 21-24; pairs in length; preferably about 21 base pairs in length.

According to a further aspect of the invention there is provided a method to inhibit the expression of a plant gene comprising the steps of:
i) contacting a plant with a viral vector that includes a nucleic acid molecule wherein said nucleic acid molecule is at least part of a gene that encodes a protein that controls the synthesis, degradation or transport of fatty acids and/or fatty acyl CoAs; and
ii) cultivating the virally infected plant to allow viral induced gene silencing.

In a preferred method of the invention said infected plant material is harvested.

In a further preferred method of the invention said harvested plant material is extracted to provide a mono- di- or triacylglycerol or free fatty acid fraction.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

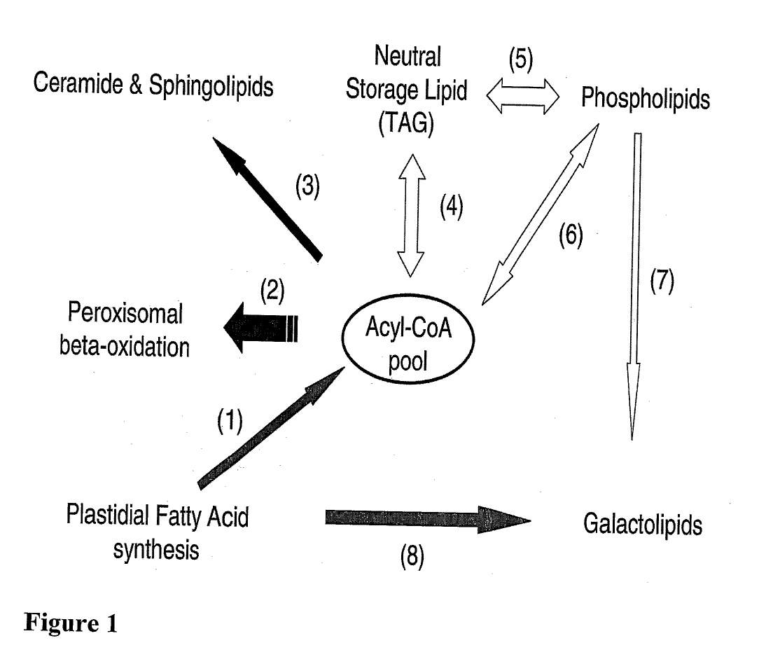

FIG. 1: The central role of the acyl CoA pool in plant lipid metabolism. Arrows represent directional fluxes of cytosolic acyl CoAs in a general model representing all plant tissues. Numbers refer to biochemical routes and genes referenced in the text;

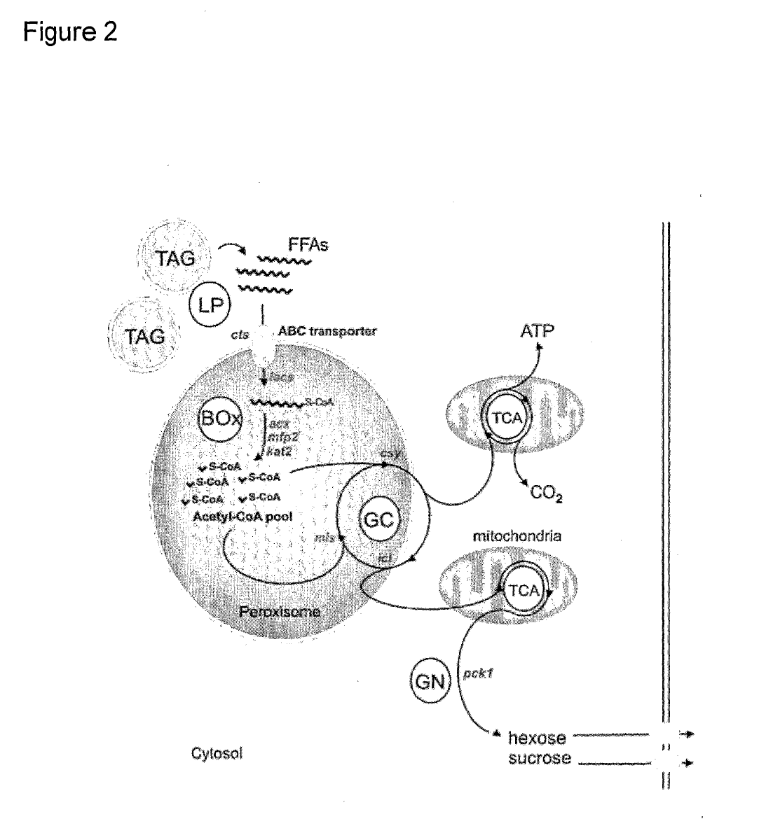

FIG. 2: Overview of the major metabolic pathways required for lipid reserve mobilisation in Arabidopsis seeds;

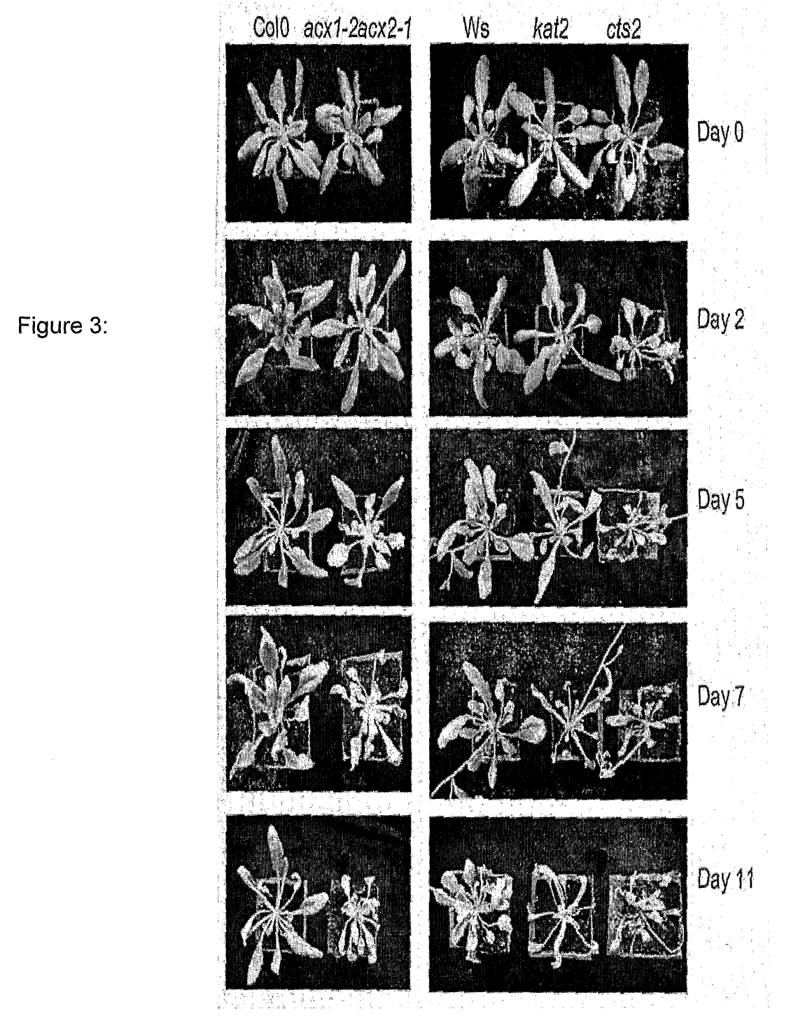

FIG. 3: illustrates that Arabidopsis mutants disrupted in peroxisomal fatty acid beta-oxidation are sensitive to extended dark treatment;

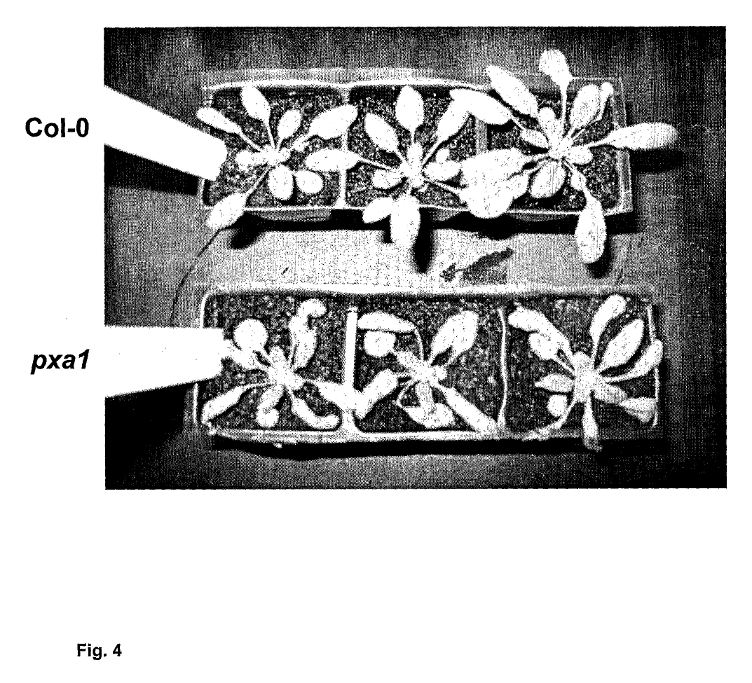

FIG. 4: illustrates the phenotype of pxa1 mutants after 48 hours extended dark compared with Col-0 wild types. Plants were grown in P40 trays for 4 weeks in a Sanyo growth cabinet with a 12 h light/12 h dark cycle;

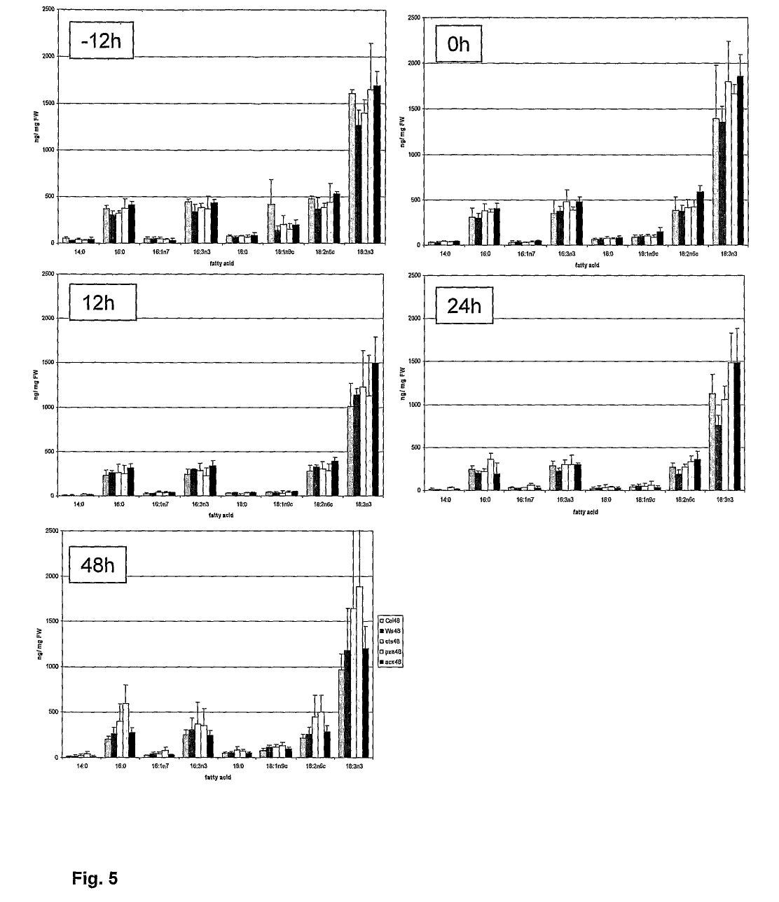

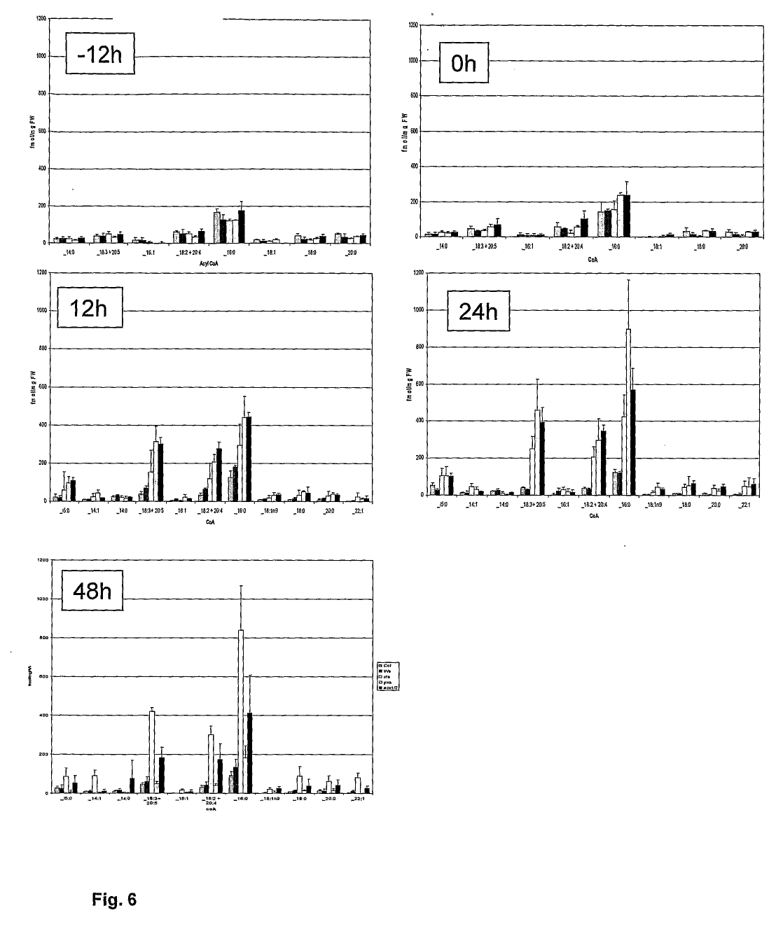

FIG. 5: illustrates total fatty acids in Arabidopsis leaves kept in extended dark for up to 48 hours. −12 h hours in the end of the day and 0 h is the start of the extended dark period. Data is the average plus SD of 4 biological replicates;

FIG. 6: illustrates acyl CoAs in Arabidopsis leaves kept in extended dark for up to 48 hours. −12 h hours in the end of the day and 0 h is the start of the extended dark period. Data is the average plus SD of 4 biological replicates;

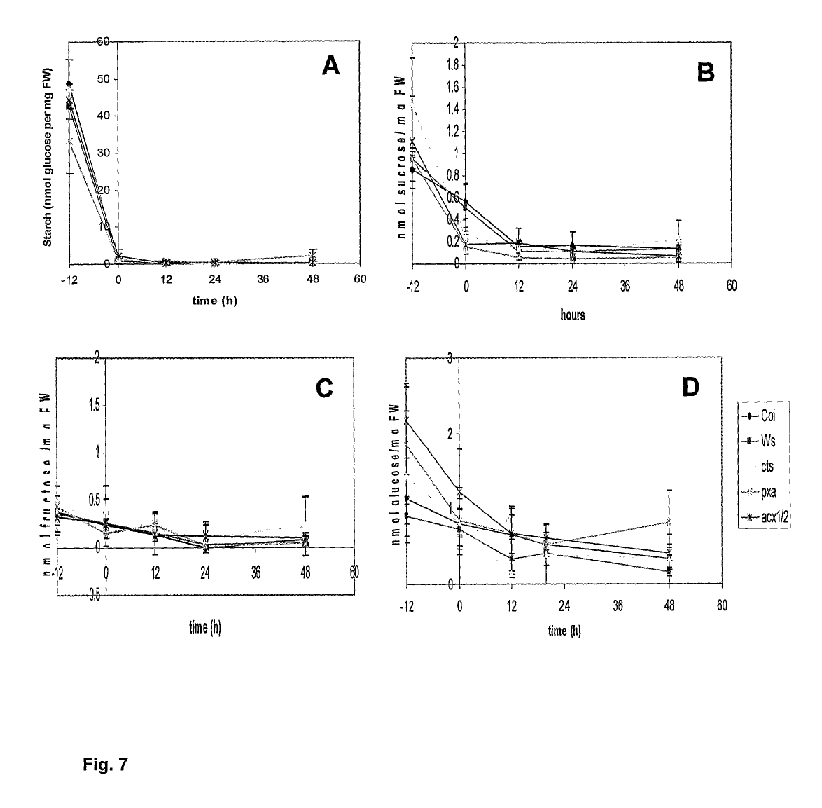

FIG. 7: illustrates the amounts of starch (A), sucrose (B), glucose (C), and fructose (D) in Arabidopsis leaves kept in extended dark for up to 48 hours. −12 h hours in the end of the day and 0 h are the start of the extended dark period. Data is the average plus SD of 4 biological replicates;

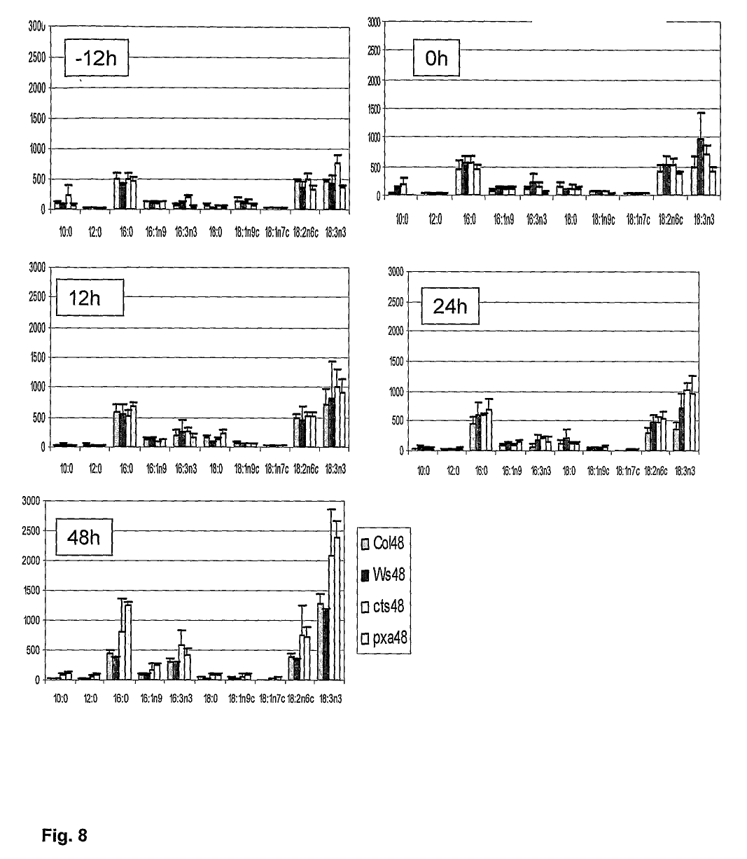

FIG. 8 illustrates total fatty acids in Arabidopsis leaves kept in extended dark for up to 48 hours. Data is the average plus SD of 3 biological replicates;

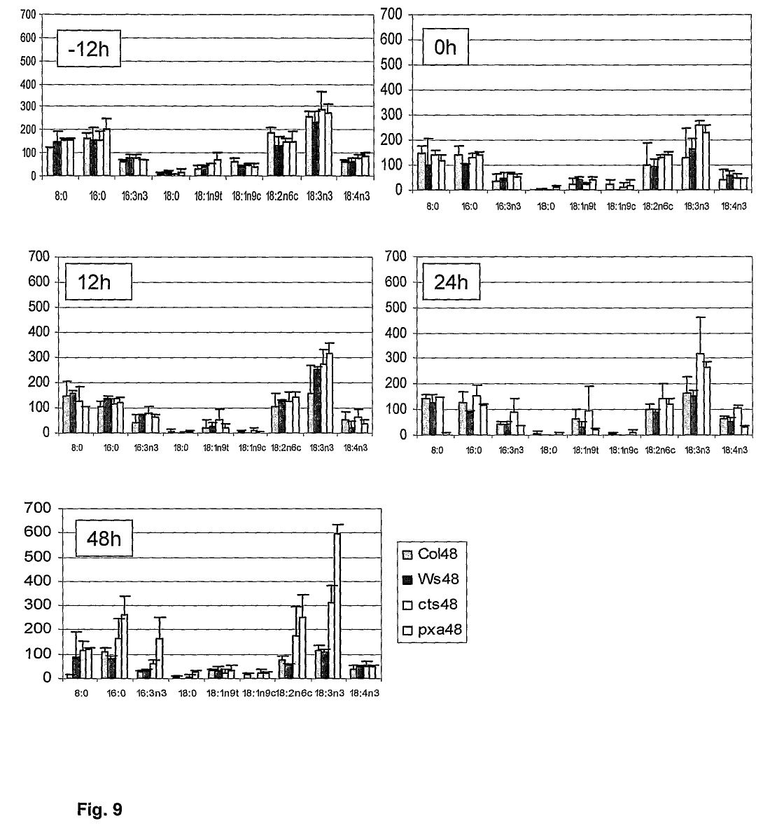

FIG. 9 illustrates non-free fatty acids in Arabidopsis leaves kept in extended dark for up to 48 hours, extracted using the base FAMEs method. Data is the average plus SD of 3 biological replicates;

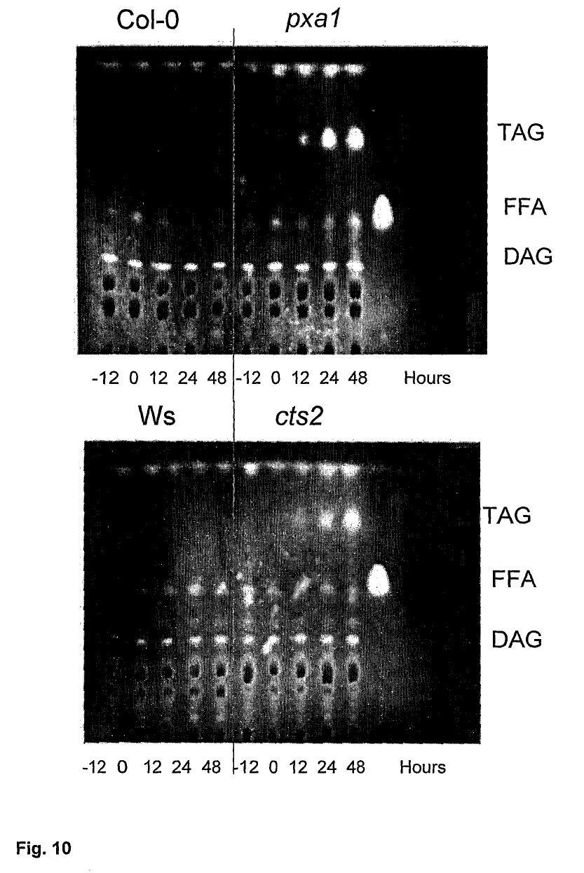

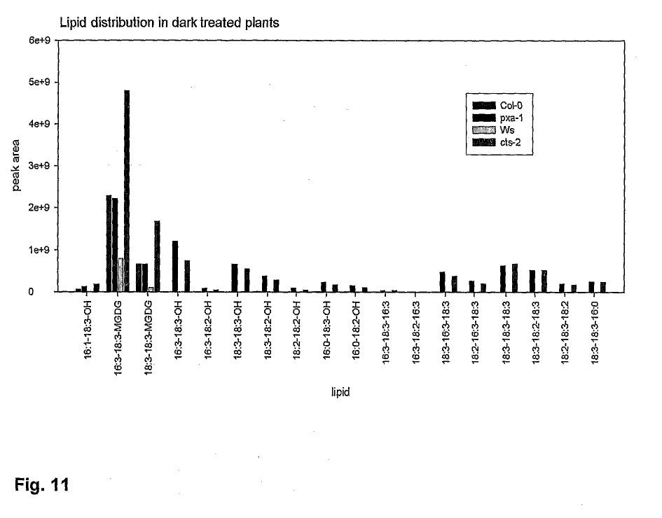

FIG. 10 illustrates thin layer chromatography of total lipid extract from leaves from plants kept in 48 h extended dark. Total lipids were extracted in 3:2 hexane:isopropanol using the standard lab lipid extraction method and developed in the solvent system: hexane:diethylether:acetic acid (70:30:1 v/v). Lipids were visualised by spraying with fluorescein and exposing to UV light;

FIG. 11 illustrates total lipid analysis by LC-MS. A fraction of the total lipid extract from leaves as described in WO2006/018621, the content of which is incorporated by reference, was run on the LCQ using the TAG method developed in the lab. The fatty acid species present in the major galactolipid, DAG and TAG peaks were then identified based on the mass spectra obtained;

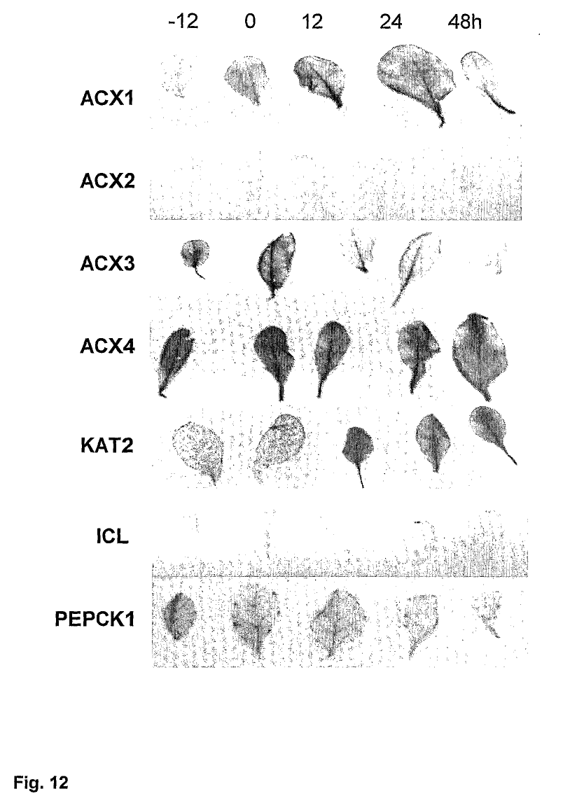

FIG. 12 illustrates histochemical staining of leaves expressing various promoters: GUS constructs kept in extended dark for up to 48 hours;

FIG. 13a is the DNA sequence of an ABC fatty acid transporter; FIG. 13b is the amino acid sequence of the ABC fatty acid transporter;

FIG. 14a is the DNA sequence of a long chain acyl Co A synthetase LACS 6; FIG. 14b is the amino acid sequence of the long chain acyl Co A synthetase LACS 6; FIG. 14c is the DNA sequence of the long chain acyl Co A synthetase LACS 7; FIG. 14d is the amino acid sequence of the long chain acyl Co A synthetase LACS 7;

FIG. 15a is the DNA sequence of a acyl oxidase ACX 1; FIG. 15b is the amino acid sequence of the acyl oxidase ACX 1; FIG. 15c is the DNA sequence of acyl oxidase ACX 2; FIG. 15d is the amino acid sequence of acyl oxidase ACX 2; FIG. 15e is the DNA sequence of the acyl oxidase ACX 3; FIG. 15f is the amino acid sequence of the acyl oxidase ACX 3; FIG. 15g is the DNA sequence of a acyl oxidase ACX 4; FIG. 15h is the amino acid sequence of the acyl oxidase ACX 4; FIG. 15i is the DNA sequence of a acyl oxidase ACX 5; FIG. 15j is the amino acid sequence of the acyl oxidase ACX 5; FIG. 15k is the DNA sequence of a acyl oxidase ACX 6; FIG. 15l is the amino acid sequence of the acyl oxidase ACX 6;

FIG. 16a is the DNA sequence of KAT 2; FIG. 16b is the amino acid sequence of KAT 2; FIG. 16c is the DNA sequence of KAT 1; FIG. 16d is the amino acid sequence of KAT 1; FIG. 16e is the DNA sequence of PKT2; FIG. 16f is the amino acid sequence of PKT2; FIG. 16g is the DNA sequence of PKT1; FIG. 16h is the amino acid sequence of PKT1;

FIG. 17a is the DNA sequence of MFP 2; FIG. 17b is the amino acid sequence of MFP 2; FIG. 17c is the DNA sequence of AIM 1; FIG. 17d is the amino acid sequence of AIM 1;

FIG. 18a-FIG. 18p represents the DNA and amino acid sequences of plant cell wall modifying enzymes;

FIG. 19a-FIG. 19j represents the DNA and amino acid sequences of plant expansin enzymes;

FIG. 20a-FIG. 20p represents the DNA and amino acid sequences of plant cell wall hydrolase enzymes;

FIG. 21 is the DNA sequence of transcription factor Cesta;

FIG. 22a is the amino acid sequence of Ricinus communis oleate 12-hydroxylase;

FIG. 22b is the nucleic acid sequence of Ricinus communis oleate 12-hydroxylase;

FIG. 22c is the nucleic acid sequence of Ricinus communis oleate 12-hydroxylase isoform;

FIG. 23a the nucleic acid sequence of a Euphorbia lagascae cytochrome P450;

FIG. 24a is the amino acid sequence of a Crepis palaestina delta 12 fatty acid epoxygenase; FIG. 24b is the nucleic acid sequence of a Crepis palaestina delta 12 fatty acid epoxygenase;

FIG. 25a is the amino acid sequence of a Crepis palaestina delta 12 fatty acid desaturase; FIG. 25b is the nucleic acid sequence of a Crepis palaestina delta 12 fatty acid epoxygenase;

FIG. 26a is the amino acid sequence of a Crepis palaestina delta 12 fatty acid acetylenase; FIG. 26b is the nucleic acid sequence of a Crepis palaestina delta 12 fatty acid acetylenase;

FIG. 27a is the amino acid sequence of a Ceratodon purpureus delta 6 fatty acid acetylenase; FIG. 27b is the nucleic acid sequence of a Ceratodon purpureus delta 6 fatty acid acetylenase;

FIG. 28a is the amino acid sequence of a Ceratodon purpureus delta 6 fatty acid desaturase; FIG. 28b is the nucleic acid sequence of a Ceratodon purpureus delta 6 fatty acid desaturase;

FIG. 29a is the amino acid sequence of the transcription factor LEC 2; FIG. 29b is the nucleic acid sequence of the transcription factor LEC 2;

FIG. 30a is the amino acid sequence of the transcription factor LEC 1; FIG. 29b is the nucleic acid sequence of the transcription factor LEC 1;

FIG. 31a is the amino acid sequence of the transcription factor FUS 3; FIG. 31b is the nucleic acid sequence of the transcription factor FUS 3;

FIG. 32a is the amino acid sequence of the transcription factor ABI 3; FIG. 32b is the nucleic acid sequence of the transcription factor ABI3; and

FIG. 33a is the amino acid sequence of the transcription factor WRI1; FIG. 33b is the nucleic acid sequence of the transcription factor WRI1.

### MATERIALS AND METHODS

Col-0, Ws, cts2, pxa1 and acx1acx2 plants were grown in P40 trays in a 12 h light/12 h dark regime in a Sanyo growth cabinet with 150 μmol·m⁻²·s⁻¹ light for 4 weeks (rosettes prior to bolting, between growth stages 3.70 and 3.90 according to Boyes et at 2001. Plant Cell 13, 1499), and then the lights were switched off. The following time points were used for material collection: minus 12 h (end of the night before the extended dark period), Oh (start of day and of extended dark), 12 h, 24 h and 48 h. In this experiment samples were collected for analysis of fatty acids and acyl CoAs (2 no.3 size leaf discs per each of 4 reps.), sugars, starch and amino acids (4 no.3 size leaf discs per each of 4 reps), and 2 outer, older leaves for RNA extraction.

Samples were collected, weighed and immediately snap-frozen for subsequent analysis. Fatty acids and CoAs were extracted and analysed using standard lab methods. Amino acids and sugars were extracted from same sample using 80% ethanol and the remaining insoluble material was used to measure starch. Amino acids were derivatised and analysed on the LCQ. Soluble sugars (sucrose, glucose and fructose) and starch (after enzymatic conversion to glucose) were quantified spectrophotometrically, using a Boerhingher Mannheim kit from R-Biopharm Ltd.

The second dark experiment was set up exactly as the first, except the acx1acx2 mutant was not included. As well as repeating the dark experiment, mutant and wild type plants were placed under the following stresses: cold treatment (13° C. and 4° C.), salt and drought. In addition, all the available promoter-GUS lines were grown in the same conditions for subsequent analysis after dark treatment.

Leaf samples were collected from 4 week-old pxa1, cts2, Col-0 and Ws plants kept in the dark for 48 h. Samples were taken at the same time points as in the previous experiment for the analysis of total fatty acids (2 leaf discs), non-free fatty acids (2 leaf discs) and total lipid analysis by thin layer chromatography (2 leaves˜100 mg tissue).

The data for total and non-free fatty acids (alkaline derivatisation) is presented in FIGS. 8 and 9. Total fatty acid data is consistent with experiment 1: after 48 h extended dark, cts2 and pxa1 plants have considerably more total fatty acids than wild types. Remarkably, the levels of some of the major fatty acid species appear to increase in the mutants over the time course (e.g. 18:3n3) introducing the possibility that fatty acids are accumulating in a sink because they cannot be broken down.

The alkaline derivatisation method allows the quantification of non-free fatty acids. Therefore the data from this method can be compared with total fatty acid measurements from the same samples in order to establish the proportions of fatty acids that are free and not free. FIG. 9 shows the levels of non-free fatty acids in the mutants and wild types throughout the time course, and illustrates that a large proportion of the fatty acids in cts2 and pxa1 are not free.

In order to establish the location of the elevated fatty acids in cts2 and pxa1, total lipids were extracted from 2 leaves using standard lipid extraction methods that use 3:2 hexane:isopropanol solvent extraction. The extracts were dried down and resuspended in a minimal volume of chloroform and spotted onto silica TLC plates. The plates were run in a hexane:diethylether:acetic acid (70:30:1 v/v) solvent system and visualised under UV light after spraying with fluorescein. The TLC plates illustrate that the cts2 and pxa1 mutants are accumulating significant amounts of mono- di- or triacylglycerols (TAGs) during the extended dark period, and that free fatty acid levels also increase (FIG. 10).

A small aliquot of the total lipid extraction was run on the LC-MS in order to identify the lipids present in the mutant plants, (see WO2006/018621 the content of which is incorporated by reference), because this sample was prepared for TLC there is no internal standard present and so this data is qualitative rather than quantitative (FIG. 11). This analysis reveals that mutant plants are accumulating triacylglycerols and diacylglycerols. Interestingly, the cts2 mutant also has increased levels of galactolipids. Many of the DAGs and TAGs found in the mutants contain C16:3, which is an exclusively chloroplast fatty acid, not found in seed TAGs. Thus a triacylglycerol sink has been established to cope with fatty acids that are targeted for degradation but blocked due to the lesion in in a specific gene involved in breakdown of fatty acids (in this case either a fatty acid transporter or beta-oxidation gene). Any treatment (for example daylength, temperature) that induces fatty acid turnover in plant material that is unable to breakdown fatty acids is thus expected to result in an increase in triacylglycerol oil accumulation;

### EXAMPLES

The dark-induced phenotype of pxa1 is more severe than that of cts2, such that by 48 h of extended dark, the older leaves have all collapsed and lost turgor (FIG. 4). The fatty acid, acyl CoA, starch and sugar data are presented in FIGS. 5, 6 and 7. The graphs in FIG. 5 show that across the time course fatty acids decrease in wild types but not in mutants, particularly cts2 and pxa1. This is most marked by 48 h of extended dark. The graphs in FIG. 6 show that acyl CoAs accumulate in mutants, particularly 18:3, 18:2 and 16:0 which are the major fatty acid species present in Arabidopsis leaves. In addition, isovaleryl CoA (i5:0), a branched chain amino acid derivative, appears after 12 hours of extended dark which is indicative of protein break down beginning to occur.

FIG. 7 illustrates the levels of soluble sugars and starch during the time course. Starch levels fall to undetectable levels over the night (FIG. 7A). Sucrose levels drop over the 12 h night period, but in wild types sucrose does not disappear completely until 12 h into the extended dark (FIG. 7B). In contrast all 3 mutants show a more rapid decrease in sucrose levels over the night, which is likely to result because fatty acid utilisation, which normally occurs during the night in wild types, cannot occur in the mutants. This indicates that substantial fatty acid turnover occurs during the normal night period in wild type plants and when this is blocked soluble sugars are more rapidly respired. Any treatment that increases fatty acid turnover during the night is therefore likely to increase the flux of carbon into the new triacylglycerol oil sink that is established when fatty acid breakdown is blocked.

The finding that cts2 and pxa1 mutants accumulate TAG is an important discovery. Blocking breakdown leads to accumulation of acyl CoAs and under conditions of dark induced starvation, that most probably also mimic natural senescence, the plants actually induce the process of TAG biosynthesis and divert fatty acids into DAGs and TAGs.

### Beta Oxidation Gene Promoter-GUS Expression

Transgenic plants expressing several promoter-GUS lines were placed in extended dark over the same time course as the beta oxidation mutants, in order to investigate the effect of dark on the gene expression of ACX1, ACX2, ACX3, ACX4, KAT2, ICL and PEPCK1 (FIG. 12). Histochemical staining of dark-starved leaves over the time course suggests that ACX1 and KAT2 are induced by extended dark, while ACX3 is repressed. ACX2 and ICL are not expressed in leaves, and PEPCK1 expression does not change during the time course. The induction of ACX1 and KAT2 demonstrates that the dark treatment is leading to induction of fatty acid beta-oxidation genes. The dark treatment is therefore a convenient experimental treatment to induce fatty acid breakdown and analyse the impact of blocking this process in foliar tissue. This treatment therefore mimics other more physiological conditions such as aging and leaf senescence which would also be expected to result in TAG accumulation when fatty acid breakdown in blocked.

1. A transgenic plant cell the genome of which is modified by transfection with a nucleic acid molecule selected from the group consisting of:
i) a nucleic acid molecule comprising an expression cassette which cassette comprises a nucleic acid sequence that encodes at least part of a gene that encodes a polypeptide that controls the synthesis, degradation or transport of fatty acids and/or fatty acyl CoAs and therefore synthesis and degradation of mono- di- or triacylglycerols wherein said cassette is adapted such that both sense and antisense nucleic acid molecules are transcribed from said cassette wherein the expression from said cassette produces an interfering RNA molecule that inhibits the expression of said gene;
ii) a nucleic acid molecule comprising an expression cassette which cassette comprises a nucleic acid sequence that encodes at least part of a gene that encodes a polypeptide that controls the synthesis, degradation or transport of fatty acids and/or fatty acyl CoAs and therefore synthesis and degradation of mono- di- or triacylglycerols wherein said cassette is adapted such that an antisense nucleic acid molecule is transcribed from said cassette wherein the expression from said cassette produces an antisense RNA molecule that inhibits the expression of said gene; and
iii) a nucleic acid molecule that encodes a polypeptide that controls the synthesis, degradation or transport of fatty acids and/or fatty acyl CoAs and therefore synthesis and degradation of mono- di- or triacylglycerols in a plant cell which polypeptide is a variant polypeptide that varies from a native polypeptide sequence wherein said variant polypeptide is a dominant negative suppressor of the native polypeptide and inhibits the production of fatty acids and/or fatty acyl CoAs therefore synthesis and degradation of mono- di- or triacylglycerols, wherein said nucleic acid molecule in i), ii) and iii) is operably linked to a promoter sequence that is substantially a foliar inducible and/or senescence inducible promoter.

2. A cell according to claim 1 wherein said gene encodes a polypeptide involved in degradation of fatty acids and/or fatty acyl Co As.

3. A cell according to claim 1 wherein said gene is encoded by a nucleic acid molecule comprising a nucleic acid sequence selected from the group consisting of:
i) SEQ ID NO: 1;
ii) a nucleic acid sequence that hybridises under stringent hybridisation conditions to SEQ ID NO: 1 and which encodes a fatty acid transporter polypeptide; and
iii) a nucleic acid sequence that encodes a variant polypeptide that varies from a polypeptide comprising SEQ ID NO: 2.

4. A cell according to claim 1 wherein said gene is encoded by a nucleic acid molecule comprising a nucleic acid sequence selected from the group consisting of:
i) SEQ ID NO: 15 and 17;
ii) a nucleic acid sequence that hybridises under stringent hybridisation conditions to SEQ ID NO: 15 and/or 17 and which encodes a long chain acyl Co A synthetase polypeptide; and
iii) a nucleic acid sequence that encodes a variant polypeptide that varies from a polypeptide comprising SEQ ID NO: 16 or 18.

5. A cell according to claim 1 wherein said gene is encoded by a nucleic acid molecule comprising a nucleic acid sequence selected from the group consisting of:
i) SEQ ID NO: 3, 5, 7, 9, 11 and 13;
ii) a nucleic acid molecule comprising a nucleic acid sequence that hybridises under stringent hybridisation conditions to SEQ ID NO: 3, 5, 7, 9, 11 or 13 and which encodes an acyl CoA oxidase polypeptide; and
iii) a nucleic acid sequence that encodes a variant polypeptide that varies from a polypeptide comprising SEQ ID NO: 4, 6, 8, 10, 12 or 14.

6. A cell according to claim 1 wherein said gene is encoded by a nucleic acid molecule comprising a nucleic acid sequence selected from the group consisting of:
i) SEQ ID NO: 19, 114, 116 and 118;
ii) a nucleic acid sequence that hybridises under stringent hybridisation conditions to SEQ ID NO: 19, 114, 116 or 118 and which encodes a keto-acyl-CoA thiolase; and
iii) a nucleic acid sequence that encodes a variant polypeptide that varies from a polypeptide comprising SEQ ID NO: 20, 115, 117 or 119.

7. A cell according to claim 1 wherein said gene is encoded by a nucleic acid molecule comprising a nucleic acid sequence selected from the group consisting of:
i) SEQ ID NO: 21 and 23.
ii) a nucleic acid sequence that hybridises under stringent hybridisation conditions to a nucleic acid molecule in (i) and which encodes a multifunctional protein involved in peroxisomal β oxidation; and
iii) a nucleic acid sequence that encodes a variant polypeptide that varies from a polypeptide comprising SEQ ID NO: 22 or 24.

8. A cell according to claim 1 wherein said cassette adapted for expression of sense and antisense nucleic acid comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts.

9. A cell according to claim 1 wherein said promoter sequence is an inducible foliar specific promoter sequence.

10. A cell according to claim 1 wherein said promoter sequence is a senescence inducible promoter sequence.

11. A cell according to claim 1 wherein said nucleic acid molecule is part of a vector and is operably linked to a promoter.

12. A cell according to claim 1 wherein the genome of said transgenic plant cell is yet further modified by transfection with at least one nucleic acid sequence selected from the group consisting of:
i) SEQ ID NO: 25, 27, 29, 31, 33, 35, 37, 39, 41, 43, 45, 47, 49, 51, 54, 56 and 58;
ii) a nucleic acid sequence that hybridises under stringent hybridisation conditions to SEQ ID NO: 25, 27, 29, 31, 33, 35, 37, 39, 41, 43, 45, 47, 49, 51, 54, 56 or 58 and which encodes a plant cell wall modifying polypeptide; and
iii) a nucleic acid sequence that encodes a polypeptide comprising SEQ ID NO: 26, 28, 30, 32, 34, 36, 38, 40, 42, 44, 46, 48, 50, 53, 55, 57 or 59.

13. A cell according to claim 1 wherein the genome of said transgenic plant cell is yet further modified by transfection with at least one nucleic acid sequence selected from the group consisting of:
i) SEQ ID NO: 62, 64, 66, 68, 70, 72, 74, 76, 78 and 80;
ii) a nucleic acid sequence that hybridises under stringent hybridisation conditions to SEQ ID NO: 62, 64, 66, 68, 70, 72, 74, 76, 78 or 80 and which encodes an expansin polypeptide; and
iii) a nucleic acid sequence that encodes a polypeptide comprising SEQ ID NO: 63, 65, 67, 69, 71, 73, 75, 77, 79 or 81.

14. A cell according to claim 1 wherein the genome of said transgenic plant cell is yet further modified by transfection with at least one nucleic acid sequence selected from the group consisting of
i) SEQ ID NO: 82, 84, 86, 88, 90, 92, 94, 96, 98, 100, 102, 104, 106, 108, 110 and 112;
ii) a nucleic acid sequence that hybridises under stringent hybridisation conditions to SEQ ID NO: 82, 84, 86, 88, 90, 92, 94, 96, 98, 100, 102, 104, 106, 108, 110 or 112 and which encodes a plant cell wall hydrolase polypeptide; and
iii) a nucleic acid sequence that encodes a polypeptide comprising SEQ ID NO: 83, 85, 87, 89, 91, 93, 95, 97, 99, 101, 103, 105, 107, 109, 111 or 113.

15. A cell according to claim 1 wherein the genome of said transgenic plant cell is modified by transfection with a nucleic acid molecule that encodes a polypeptide the expression of which confers growth enhancing effects on said cell or a plant derived from said cell thereby increasing plant biomass.

16. A cell according to claim 15 wherein said nucleic acid molecule is over-expressed when compared to a non-transgenic reference plant cell of the same species.

17. A cell according to claim 15 wherein said nucleic acid molecule that encodes a polypeptide the expression of which confers growth enhancing effects is selected from the group consisting of:
i) SEQ ID NO: 120; and
ii) a nucleic acid molecule comprising a nucleic acid sequence that hybridises under stringent hybridisation conditions to SEQ ID NO: 120 and which encodes a transcription factor.

18. A transgenic plant comprising a cell according to claim 1.

19. A plant according to claim 18 wherein said plant is selected from the group consisting of switchgrass, alfalfa, willow, poplar, eucalyptus, miscanthus, wheat, maize or barley.

20. A seed comprising a plant cell according to claim 1.

21. A method to modulate and extract plant mono- di- or triacylglycerol fatty acids comprising the steps of:
i) providing a transgenic plant the genome of which is modified by transfection with a nucleic acid molecule selected from the group consisting of:
a) a nucleic acid molecule comprising an expression cassette which cassette comprises a nucleic acid sequence that encodes at least part of a gene that encodes a polypeptide that controls the synthesis, degradation or transport of fatty acids and/or fatty acyl CoAs and therefore synthesis and degradation of mono- di- or triacylglycerols, wherein said cassette is adapted such that both sense and antisense nucleic acid molecules are transcribed from said cassette wherein the expression from said cassette produces an interfering RNA molecule that inhibits the expression of said gene;
b) a nucleic acid molecule comprising an expression cassette which cassette comprises a nucleic acid sequence that encodes at least part of a gene that encodes a polypeptide that controls the synthesis, degradation or transport of fatty acids and/or fatty acyl CoAs and therefore synthesis and degradation of mono- di- or triacylglycerols, wherein said cassette is adapted such that an antisense nucleic acid molecule is transcribed from said cassette wherein the expression from said cassette produces an antisense RNA molecule that inhibits the expression of said gene; and
c) a nucleic acid molecule that encodes a polypeptide that controls the synthesis, degradation or transport of fatty acids and/or fatty acyl CoAs and therefore synthesis and degradation of mono- di- or triacylglycerols, in a plant cell which polypeptide is a variant polypeptide that varies from a native polypeptide sequence wherein said variant polypeptide is a dominant negative suppressor of the native polypeptide and inhibits the production of mono- di- or triacylglycerol, wherein said nucleic acid molecule in a), b) or c) is operably linked to a promoter sequence;
ii) inducing expression of at least one nucleic acid molecule according to the invention;
iii) harvesting transgenic plant material; and
iv) extracting said harvested plant material to provide a mono- di- or triacylglycerol or free fatty acid fraction and an extracted plant material fraction.

22. A method according to claim 21 wherein the induction of expression of said nucleic acid molecules is by induction of senescence.

23. A method according to claim 22 wherein the induction of senescence is by growing said plant in reduced light conditions.

24. A method according to claim 22 wherein the induction of senescence is by altered day-length.

25. A method according to claim 22 wherein senescence is induced by chemical treatment.

26. A method according to claim 21 wherein the genome of said transgenic plant cell is yet further modified by transfection with a nucleic acid sequence selected from the group consisting of:
i) SEQ ID NO: 25, 27, 29, 31, 33, 35, 37, 39, 41, 43, 45, 47, 49, 51, 54, 56 and 58;
ii) a nucleic acid sequence that hybridises under stringent hybridisation conditions to SEQ ID NO: 25, 27, 29, 31, 33, 35, 37, 39, 41, 43, 45, 47, 49, 51, 54, 56 or 58 and which encodes a plant cell modifying polypeptide; and
iii) a nucleic acid sequence that encodes a polypeptide comprising SEQ ID NO: 26, 28, 30, 32, 34, 36, 38, 40, 42, 44, 46, 48, 50, 53, 55, 57 or 59.

27. A method according to claim 21 wherein the genome of said transgenic plant cell is yet further modified by transfection with a nucleic acid sequence selected from the group consisting of:
i) SEQ ID NO: 62, 64, 66, 68, 70, 72, 74, 76, 78 and 80;
ii) a nucleic acid sequence that hybridises under stringent hybridisation conditions to SEQ ID NO: 62, 64, 66, 68, 70, 72, 74, 76, 78 or 80 and which encodes an expansin polypeptide; and
iii) a nucleic acid sequence that encodes a polypeptide comprising SEQ ID NO: 63, 65, 67, 69, 71, 73, 75, 77, 79 or 81.

28. A method according to claim 21 wherein the genome of said transgenic plant cell is yet further modified by transfection with a nucleic acid sequence selected from the group consisting of:
i) SEQ ID NO: 82, 84, 86, 88, 90, 92, 94, 96, 98, 100, 102, 104, 106, 108, 110 and 112;
ii) a nucleic acid sequence that hybridises under stringent hybridisation conditions to SEQ ID NO: 82, 84, 86, 88, 90, 92, 94, 96, 98, 100, 102, 104, 106, 108, 110 or 112 and which encodes a plant cell wall hydrolase polypeptide; and
iii) a nucleic acid sequence that encodes a polypeptide comprising SEQ ID NO: 83, 85, 87, 89, 91, 93, 95, 97, 99, 101, 103, 105, 107, 109, 111 or 113.

29. A method according to claim 21 wherein said extracted plant material fraction is further processed by saccharification to sugar.

30. A method according to claim 29 wherein said sugar is used as a feedstock in the production of ethanol by microbial fermentation.

31-61. (canceled)


SEQUENCE LISTING (144 sequences)

SEQ ID NO: 1 - DNA, 4722 bp, Arabidopsis thaliana
ctctctctctctctatctctatctctcgatttgggggagttccgtcacggtggactagtacgtctcgttgccgttggtggcgtagtcggaattaatttcctcggcgttgagattcacatggtctagaattctagctaagtggttgttgttgttgttacgatttccgatttctcgagttttttttttatatttagcttctgtttcgtttatccctcccggagacactccttggtcgaatctctcatgctgaggtgttttggacacttgttgtcaagaagaaaccagttttggttctgattaatcgttggttggaaaatatactcaattccaggccatgccttcacttcaactattgcagttaactgagcggggtcggggtcttgtagcgtcaagacggaaatctatactgcttgcggctgggattgtagctgctggtggaactgctgtttacctgaaatcaagggtcgcttcccggaggcctgattcttcgcgtctttgcaatggtcagagtgatgatgatgagactttggaaaagctgactgcaactgatcaaaatgcaaagataaccacgaaaaagaagaaaggaggaggattgaagtctcttcaggttctgactgctattcttctctctcagatgggaaaaatgggtgccagggatcttttggcactagtcgccaccgtggttttcagaacagctttgagcaatagattggcaaaagtgcaaggtttccttttccgtgctgctttcttaaggcgtgcgccactgtttctacggctcatctccgagaatattatgttgtgtttcatgctatcaacattgcactctacttcaaagtacataactggggcattgagtttgcgattcagaaagatattgaccaagattatccattcacactattttgagaatatggtatattacaaaatatcacacgtggatggtcggattacgcaccctgaacaaagaattgccagcgatgtaccaagattctcctcagagttgagcgatcttatactggatgatttgacggcggttactgatggaattttgtatgcatggcgcctgtgttcatatgctagtccaaaatacatcttctggatactggcctatgtactgggggctgggacggcgataagaaacttttctccttcttttgggaaattgatgtccaaggaacagcagttagaaggagagtaccggcaacttcattcacgcttaaggactcattcggaaagcatagcattctatggtggggaaaccagggaagaatctcatatacaacaaaagttcaagaatcttgttagccatatgagtcacgtgcttcatgatcactggtggtttggtatgatccaagattttctgctgaagtatcttggggccacagttgcagttattctgattatcgaaccattcttctctgggcatctaagacctgacgactcgaccttaggaagagctgagatgcttagcaatataagatatcacactagtgtcattatatctctctttcaggcgttgggaacactttctataagttccaggcggctcaaccgactcagtggttatgctgaccgaatccatgagttgatggctgtctcaagagaactcagtggtgatgataaatcgtctttccagagaaatagaagcagaaattatctaagtgaagctaattatgtagagttttccgatgtcaaggttgttactccaaccggaaatgttttggtggaggatctcacccttcgagttgagcaagggtctaatcttctgattacaggtcctaatggaagtggcaagagttcccttttccgagtattaggaggtctatggcccctggtgtctggacatattgtgaagccaggagttggttctgatcttaacaaggagatcttctatgtgccgcaacggccttatatggcagtaggaacacttcgtgaccagttaatatatcctcttacttctggccaagagagtgaactgctcactgagattggaatggtggagctattgaaaaatgttgatctagaatatttattggatcgctaccaacctgaaaaagaggttaattggggtgatgaattatctcttggagagcaacagagattgggtatggccagactattctaccacaaacccaaatttgcaattctagatgaatgcacaagtgctgtcacaactgatatggaagaacgctttgccgctaaggttcgagctatgggaacttcttgcataacaatctcccatcgtccagcgcttgttgcattccatgatgttgttctgtcattagacggtgaaggaggatggagtgttcattacaagagggatgactctgcccttctgacggatgctgaaattgattcagtgaaaagttcagatacagatcggcaaaatgatgcgatggttgttcaacgagcgtttgctgcagctagaaaggaatctgctactaattcaaaggctcagtcgtaccagacacagttaattgcaagatcacctgttgtagataaaagtgtagtgttgcctcgttttcctcaacctcaaacatcccaaagggctttaccatcaagagtagctgcaatgttaaacgtgttgatacccactatatttgacaagcaaggagctcaactgcttgctgttgcttgccttgtcgtctcaagaacgctgatctctgaccgaatagcctctttgaatgggaccactgtgaagtatgtcttggagcaagataaggcagcctttgttcgtttgattggtttgagtgttctccaaagtggtgcatcttctataattgctccttcactaaggcatttaacgcaaaggctagcgttagggtggaggattcgtttgactcaacatctgctaaggaactatttgagaaataatgcgttttacaaggttttccacatgtcaggcaatagtattgatgcggaccagagactcactcgtgacctggaaaagttaaccgctgacttgtctggacttcttactggaatggtaaagccatcggttgacattctctggttcacctggaggatgaagttactgactggtcagaggggagttgccatactttacacatatatgttacttggtcttggttttctgagacgtgttgctcccgatttcggtgatctagccggtgaagaacagcagcttgaagggaagtttcggtttatgcacgagaggctgaacactcatgctgaatctattgcattctttggaggtggagctcgagaaaaggctatggttgacaaaaaattcagggccctactggatcattctctcatgctcttgaggaagaaatggttgtatggcatacttgatgattttgtgacaaagcaacttcccaataatgtgacgtggggattgagtttattgtatgccctagaacacaagggagacagagcacttgtctccactcaaggtgaattggcacatgcattgcggtatctagcttctgttgtctcccaaagctttatggcttttggcgatattcttgaactacacaagaagttcctggagctctctggtggtattaacagaatttttgagctcgatgagtttttggatgcttctcagtcaggtgttacctcagaaaatcaaacaagtcgtttggattctcaagatctactttccttttcggaggtggatatcattacccctgctcagaaattgatggctagcaagttgtcgtgtgaaatagtttcagggaaaagcctgctcgtcacaggtccaaatggtagtggaaagacttcagtatttagagtccttagagatatctggcccactgtatgtggaagacttaccaaaccatcattggatatcaaagaacttgggtcagggaatggcatgttttttgtcccgcagcgaccttatacatgtttagggacactgagagatcaaattatataccctctatctaaagaagaagcagagaaaagggcagcaaagttgtacaccagtggagagagctcaacagaagctggaagcattctggattctcatttgaaaaccattctggagaatgttcggttagtttatctcttggaaagagacgtaggtggttgggatgctactaccaattgggaagacatattatctcttggagagcaacagagattaggcatggcacgtttattctttcacaggccgaagtttggagtccttgatgaatgcacaaatgcgacgagtgttgatgttgaggaacagctctatagagttgcacgagacatgggagtcactttcataacctcatcacaacggccggctctgatcccattccattccttggagctaaggctgattgatggagaaggaaactgggagctccgttcgatcgaacagacaacagagtgaactcagcaaaacatttttagaaaggtctatatagttgttaaagaaaaaagtaataaagttaaagccattagacgatgcaagctatatggtatgtagtatatggattcttcctcgatcgcaaggagtggaagagaatgcgtcgatgctagtgcttttgttagaattggaggatttgatttgattctagatatatataaatgtaggcgattgaattggtggagcattttgagttctcctatggagtatggtcttagctttgaacaaacaaagaatatagtgatcactcaaataatgtacagttcgtttcaatttcctttgttgggattagtttttctatcttataattaaaagaatgaaattgaagtggttgaatc

SEQ ID NO: 2 - protein, 1337 aa, Arabidopsis thaliana
MPSLQLLQLTERGRGLVASRRKSILLAAGIVAAGGTAVYLKSRVASRRPDSSRLCNGQSDDDETLEKLTATDQNAKITTKKKKGGGLKSLQVLTAILLSQMGKMGARDLLALVATVVFRTALSNRLAKVQGFLFRAAFLRRAPLFLRLISENIMLCFMLSTLHSTSKYITGALSLRFRKILTKIIHSHYFENMVYYKISHVDGRITHPEQRIASDVPRFSSELSDLILDDLTAVTDGILYAWRLCSYASPKYIFWILAYVLGAGTAIRNFSPSFGKLMSKEQQLEGEYRQLHSRLRTHSESIAFYGGETREESHIQQKFKNLVSHMSHVLHDHWWFGMIQDFLLKYLGATVAVILIIEPFFSGHLRPDDSTLGRAEMLSNIRYHTSVIISLFQALGTLSISSRRLNRLSGYADRIHELMAVSRELSGDDKSSFQRNRSRNYLSEANYVEFSDVKVVTPTGNVLVEDLTLRVEQGSNLLITGPNGSGKSSLFRVLGGLWPLVSGHIVKPGVGSDLNKEIFYVPQRPYMAVGTLRDQLIYPLTSGQESELLTEIGMVELLKNVDLEYLLDRYQPEKEVNWGDELSLGEQQRLGMARLFYHKPKFAILDECTSAVTTDMEERFAAKVRAMGTSCITISHRPALVAFHDVVLSLDGEGGWSVHYKRDDSALLTDAEIDSVKSSDTDRQNDAMVVQRAFAAARKESATNSKAQSYQTQLIARSPVVDKSVVLPRFPQPQTSQRALPSRVAAMLNVLIPTIFDKQGAQLLAVACLVVSRTLISDRIASLNGTTVKYVLEQDKAAFVRLIGLSVLQSGASSIIAPSLRHLTQRLALGWRIRLTQHLLRNYLRNNAFYKVFHMSGNSIDADQRLTRDLEKLTADLSGLLTGMVKPSVDILWFTWRMKLLTGQRGVAILYTYMLLGLGFLRRVAPDFGDLAGEEQQLEGKFRFMHERLNTHAESIAFFGGGAREKAMVDKKFRALLDHSLMLLRKKWLYGILDDFVTKQLPNNVTWGLSLLYALEHKGDRALVSTQGELAHALRYLASVVSQSFMAFGDILELHKKFLELSGGINRIFELDEFLDASQSGVTSENQTSRLDSQDLLSFSEVDIITPAQKLMASKLSCEIVSGKSLLVTGPNGSGKTSVFRVLRDIWPTVCGRLTKPSLDIKELGSGNGMFFVPQRPYTCLGTLRDQIIYPLSKEEAEKRAAKLYTSGESSTEAGSILDSHLKTILENVRLVYLLERDVGGWDATTNWEDILSLGEQQRLGMARLFFHRPKFGVLDECTNATSVDVEEQLYRVARDMGVTFITSSQRPALIPFHSLELRLIDGEGNWELRSIEQTTE

SEQ ID NO: 3 - DNA, 2456 bp, Arabidopsis thaliana
aaaaagcttaattgttcttccaactactctgcaattaaatcggtagagtgaggcattgtccttctttttctccctctgttttttttttttttttttcctatcatctctgagagttttctcgagaaacttttgagtgtttagctactagattctgaattacgaatcatggaaggaattgatcacctcgccgatgagagaaacaaagcagagttcgacgttgaggatatgaagatcgtctgggctggttcccgccacgcttttgaggtttccgatcgaattgcccgccttgtcgccagcgatccggtatttgagaaaagcaatcgagctcggttgagtaggaaggagctgtttaagagtacgttgagaaaatgtgcccatgcgtttaaaaggattatcgagcttcgtctcaatgaggaagaagcaggaagattgaggcactttatcgaccagcctgcctatgtggatctgcactggggaatgtttgtgcctgctattaaggggcagggtacagaggagcagcagaagaagtggttgtcgctggccaataagatgcagattattgggtgttatgcacagactgagcttggtcatggctcaaatgttcaaggacttgagacaactgccacatttgatcccaagactgatgagtttgtaattcacactccaactcagactgcatccaaatggtggcctggtggtttgggaaaagtttctactcatgctgttgtttacgctcgtctcataactaacggaaaagactacggtatccatggattcatcgtgcaactgcgaagcttagaagatcattctcctcttccgaatataactgttggtgatatcgggacaaagatgggaaatggagcatataattcaatggacaacgggtttcttatgtttgatcatgttcgcattcctagagatcaaatgctcatgaggctgtcaaaagttacaagagaaggagaatatgttccatcggatgttccaaagcagctggtatatggtactatggtgtatgtgagacaaacaattgtggctgatgcttccaatgcactatctcgagcagtttgcatagctacaagatacagtgcagtgcggaggcaatttggcgcacataatggtggcattgagacacaggtgattgattataaaactcagcagaacaggctatttcctctgctagcatctgcatatgcatttcgatttgttggagagtggctaaaatggctgtacacggatgtaactgaaagactggcggctagtgatttcgcaactttgcctgaggctcatgcatgcactgcaggattgaagtctctcaccaccacagccactgcggatggcattgaagaatgtcgtaagttatgtggtggacatggatacttgtggtgcagtgggctccccgagctgtttgctgtatatgttcctgcctgcacatacgaaggagacaatgttgtgctgcaattacaggttgctcgattcctcatgaagacagtcgcccagctgggatctggaaaggttcctgttggcacaactgcttatatgggccgggcagcacatcttttgcaatgtcgttctggtgttcaaaaggctgaggattggttaaaccctgatgttgtactggaagctttcgaagctagggctctcagaatggctgttacgtgtgccaaaaatctcagcaagtttgagaatcaggaacaaggattccaagagctcttggctgatttggttgaggccgctattgctcattgccaattgattgttgtttccaagttcatagcgaaactggagcaagacataggtggcaaaggagtgaagaaacagctgaataatctgtgttacatttatgctctttatctcctccacaaacatctcggcgatttcctctccactaactgcatcactcccaaacaagcctctcttgctaacgaccagctccgttccttatacactcaggtccggcctaatgcggttgcacttgtggacgccttcaattacaccgaccattacttgaactcggttcttggccgttacgacggtaatgtgtacccaaagctctttgaggaagcgttgaaggatccattgaacgactcggtggttcctgatgggtacaagaataccttcgacctgtgcttcagcagcaacttcgtaccgctaggctctgaagagttttctttgcttgatactcgatatggttaatcacattagacttgcttcgtccttcttcttcgtcttcttcttcttctcgctttgaataatttcgcagtttaaaaactggcgatgcccttatttatatgtagcaatgtaatagttaatgtacgatcgtcatatggcggaattttagtactatttttcgttttcaatgcaacattaatacaattgatcgtttctactctgagaaggatatgtgggatatttgtaatcttcgtatatagtccaatcacatatattgacatttctct

SEQ ID NO: 4 - protein, 675 aa, Arabidopsis thaliana
MEGIDHLADERNKAEFDVEDMKIVWAGSRHAFEVSDRIARLVASDPVFEKSNRARLSRKELFKSTLRKCAHAFKRIIELRLNEEEAGRLRHFIDQPAYVDLHWGMFVPAIKGQGTEEQQKKWLSLANKMQIIGCYAQTELGHGSNVQGLETTATFDPKTDEFVIHTPTQTASKWWPGGLGKVSTHAVVYARLITNGKDYGIHGFIVQLRSLEDHSPLPNITVGDIGTKMGNGAYNSMDNGFLMFDHVRIPRDQMLMRLSKVTREGEYVPSDVPKQLVYGTMVYVRQTIVADASNALSRAVCIATRYSAVRRQFGAHNGGIETQVIDYKTQQNRLFPLLASAYAFRFVGEWLKWLYTDVTERLAASDFATLPEAHACTAGLKSLTTTATADGIEECRKLCGGHGYLWCSGLPELFAVYVPACTYEGDNVVLQLQVARFLMKTVAQLGSGKVPVGTTAYMGRAAHLLQCRSGVQKAEDWLNPDVVLEAFEARALRMAVTCAKNLSKFENQEQGFQELLADLVEAAIAHCQLIVVSKFIAKLEQDIGGKGVKKQLNNLCYIYALYLLHKHLGDFLSTNCITPKQASLANDQLRSLYTQVRPNAVALVDAFNYTDHYLNSVLGRYDGNVYPKLFEEALKDPLNDSVVPDGYKNTFDLCFSSNFVPLGSEEFSLLDTRYG

SEQ ID NO: 5 - DNA, 2403 bp, Arabidopsis thaliana
aattcgttagcgaagagaaaattttcaactcattgaaatggaatcgcggcgagagaagaatccgatgacggaggaggaatctgatgggctaattgcggcgaggcggatccaacgattgtctttacacctatctccctccttgacgccgtcgccgtcgttaccgttggtgcagacggagacgtgttcggcgaggtcgaagaagctggatgtcaacggcgaagctttgtcgctgtatatgagaggaaaacatatagatatacaagagaaaattttcgactttttcaattcccgacccgatttgcagacgccgatcgagatctccaaggatgatcatcgggaattgtgtatgaatcagcttatagggcttgttagagaagctggggtaaggccgtttaggtatgttgctgatgatcctgagaagtatttcgcaatcatggaagctgttggaagtgttgatatgtcccttgggattaagatgggcgttcaatacagtctctggggaggctctgtgatcaatttagggactaagaagcatagagacaagtattttgatggcattgacaatcttgattacaccggttgctttgccatgactgaattacaccatgggtcaaatgtgcaaggtcttcagaccacggcgacattcgatccacttaaagacgaatttgtgatcgatacacctaatgatggagctatcaaatggtggattggaaatgctgcagttcatgggaagtttgccactgtttttgccaggcttatacttccaactcatgattccaaaggagtctcggatatgggtgttcacgccttcattgttccgataagggatatgaaaacacaccagacactccctggtgttgaaatccaagattgtggacataaagtgggacttaatggagtggataatggtgcgttgagattccgttctgtgagaataccccgtgataatcttctcaatcgttttggagatgtgtcccgagatgggacgtatacaagtagtttgccaacaatcaataaaagatttggtgcaacactcggtgagcttgtaggtggtcgagttggccttgcctatgcatctgttggcgtccttaaaatctctgcaacgattgccattcgttattctcttctaagacaacaattcgggcctccaaagcaacctgaggtcagtattctcgattaccagtctcaacaacacaagctcatgccgatgttagcctccacctatgcataccattttgcaactgtataccttgtggagaaatattcagagatgaagaagactcacgatgagcaattggttgctgatgtccatgcactctctgctgggctcaaatcttatgtgacgtcttacaccgccaaggcgctctcggtctgcagagaagcctgtggaggtcatggttacgcagctgttaaccgatttggaagcttgagaaatgatcatgacattttccaaacatttgaaggagacaacactgtacttctgcaacaggtggctgctgatttattgaagcgttataaagaaaagttccaaggcgggacattgacagttacatggagctacttgagagaatcaatgaacacttatttgtctcagccaaatcccgttacagcgcgttgggaaggtgaagatcatctaagagatcctaaattccaactagatgctttccggtatcgaacatcgcgattgctacaaaatgtggcagcgagattgcagaagcattcaaagactcttggtggtttcggggcatggaacagatgcttgaatcatcttttaacgcttgcagaatctcacattgaaacagtcattcttgccaagttcatcgaagctgttaaaaactgcccggacccaagtgcaaaagctgctctgaaactagcatgtgatctttacgcattggaccgaatctggaaagatataggaacgtaccgtaacgtggattatgtggcgcctaacaaagctaaggtttgttttcttgtataatcgtaacccacagattatatcagtgacttacttctcgattaaattggtttaaaacaaaacaaaaatcgggtttttgtttgcaggcgattcataaactgacagagtatttgagttttcaagtaaggaatgtggccaaggaactagtggatgcgttcgagctccctgatcatgttactcgagcaccaattgctatgcagtccgatgcttattcccagtatactcaagttgttggattctaaaaacacaagaacaaaacatatatcatcacaatgatcttttaattcgaagcaaaaaaaagatgaaaaacaatttacagggggtacaaaagaatctgcagtcctttgtatgtgtttttagttgttgtttgttttcacaggagaataaaaaaaaacagagtaataaaaatgtcatttttttcgtaaacc

SEQ ID NO: 6 - protein, 646 aa, Arabidopsis thaliana
MESRREKNPMTEEESDGLIAARRIQRLSLHLSPSLTPSPSLPLVQTETCSARSKKLDVNGEALSLYMRGKHIDIQEKIFDFFNSRPDLQTPIEISKDDHRELCMNQLIGLVREAGVRPFRYVADDPEKYFAIMEAVGSVDMSLGIKMGVQYSLWGGSVINLGTKKHRDKYFDGIDNLDYTGCFAMTELHHGSNVQGLQTTATFDPLKDEFVIDTPNDGAIKWWIGNAAVHGKFATVFARLILPTHDSKGVSDMGVHAFIVPIRDMKTHQTLPGVEIQDCGHKVGLNGVDNGALRFRSVRIPRDNLLNRFGDVSRDGTYTSSLPTINKRFGATLGELVGGRVGLAYASVGVLKISATIAIRYSLLRQQFGPPKQPEVSILDYQSQQHKLMPMLASTYAYHFATVYLVEKYSEMKKTHDEQLVADVHALSAGLKSYVTSYTAKALSVCREACGGHGYAAVNRFGSLRNDHDIFQTFEGDNTVLLQQVAADLLKRYKEKFQGGTLTVTWSYLRESMNTYLSQPNPVTARWEGEDHLRDPKFQLDAFRYRTSRLLQNVAARLQKHSKTLGGFGAWNRCLNHLLTLAESHIETVILAKFIEAVKNCPDPSAKAALKLACDLYALDRIWKDIGTYRNVDYVAPNKAKVCFLV

SEQ ID NO: 7 - DNA, 2028 bp, Arabidopsis thaliana
atgtcggataatcgtgcactccgacgagctcatgttctcgccaatcacatactccaatcaaatcctccatcttcgaacccgtccctgtcgcgcgagttatgtttgcagtactctccaccggagctcaacgagagctatggattcgatgtcaaggagatgagaaaattacttgacggacacaacgtggtggatcgggactggatttatggactcatgatgcagagcaatctgtttaatcggaaggagagaggaggtaagattttcgtgtcgccggattacaatcagacgatggagcagcagcgtgagatcacaatgaaacggatctggtacttgcttgagaatggggttttcaaaggatggttgacggagacaggtcctgaggccgagctcaggaaattagctctgcttgaggtttgcgggatttatgatcactccgtctccatcaaagttggtgtgcatttcttcctgtggggtaatgctgtaaagttttttggaacaaagcgtcaccatgaaaagtggctgaagaacaccgaagattatgttgtcaagggctgttttgctatgactgagctaggccatggaagtaatgtacggggaattgaaacagtgacaacttatgacccaaaaactgaagagtttgtgataaatactccttgtgaatctgctcagaagtattggattggtggggcagctaatcatgcaacccacacaattgtgttttcacagcttcatatcaacggaaccaaccagggggtccatgcctttatcgcccaaatcagggatcaagatggcagcatatgtccaaatatccgcattgctgactgtggacacaaaattggtctaaatggtgttgacaatggccggatctggtttgataatcttcgaattccaagagagaatttgttgaatgcagttgctgatgtttcgtctgatgggaagtatgttagctcaattaaagatcctgatcagagatttggagcattcatggcccctttgacttctggccgagtcacaattgcatcaagtgcaatttattctgcaaaggtcggattatctattgctataaggtactcattatcgagaagagccttctctgttacagctaatggtcctgaagtcctcctccttgattacccaagccatcaaaggcgactgctaccactcctagcaaagacatatgctatgagttttgctgcaaatgaattgaagatgatttacgtgaagagaacaccggagaccaacaaagccatccacgttgtttcaagtgggttcaaagctgttctcacctggcacaatatgcacacacttcaggaatgtcgagaagctgtcggagggcaaggtgtgaaaacagaaaatctagttggtcagttgaaaggtgaatttgatgtgcagactacatttgagggtgacaataatgtattgatgcagcaggtgagcaaggcgcttttcgctgaatatgtatcgtgtaagaagagaaacaaacctttcaagggactgggattggagcacatgaacagtccacgtcctgtattaccgactcaactcacatcatcaaccctcagatgcagccaattccagacaaatgtgttctgcttaagagagcgagatcttctggagcaatttacttctgaagttgcacagcttcaagggagaggagaaagtcgagaattctctttcctcctgagtcatcaacttgctgaagacttaggtaaagctttcacagagaaagcaatacttcaaaccattttggatgctgaggcaaaactacctactggctcagtaaaggatgtgttgggtcttgtaagatcaatgtacgcattgatcagcttggaagaagatccatcgttgctgcgatatggttacctatctcaggataatgttggagatgtgaggagagaagtttcaaagctctgtggagagcttagaccacacgcgcttgcactcgtcacttcattcggcattccagactccttcttgagtccaattgcattcaactgggtcgaagccaatgcttggtcttcagtttag

SEQ ID NO: 8 - protein, 675 aa, Arabidopsis thaliana
MSDNRALRRAHVLANHILQSNPPSSNPSLSRELCLQYSPPELNESYGFDVKEMRKLLDGHNVVDRDWIYGLMMQSNLFNRKERGGKIFVSPDYNQTMEQQREITMKRIWYLLENGVFKGWLTETGPEAELRKLALLEVCGIYDHSVSIKVGVHFFLWGNAVKFFGTKRHHEKWLKNTEDYVVKGCFAMTELGHGSNVRGIETVTTYDPKTEEFVINTPCESAQKYWIGGAANHATHTIVFSQLHINGTNQGVHAFIAQIRDQDGSICPNIRIADCGHKIGLNGVDNGRIWFDNLRIPRENLLNAVADVSSDGKYVSSIKDPDQRFGAFMAPLTSGRVTIASSAIYSAKVGLSIAIRYSLSRRAFSVTANGPEVLLLDYPSHQRRLLPLLAKTYAMSFAANELKMIYVKRTPETNKAIHVVSSGFKAVLTWHNMHTLQECREAVGGQGVKTENLVGQLKGEFDVQTTFEGDNNVLMQQVSKALFAEYVSCKKRNKPFKGLGLEHMNSPRPVLPTQLTSSTLRCSQFQTNVFCLRERDLLEQFTSEVAQLQGRGESREFSFLLSHQLAEDLGKAFTEKAILQTILDAEAKLPTGSVKDVLGLVRSMYALISLEEDPSLLRYGYLSQDNVGDVRREVSKLCGELRPHALALVTSFGIPDSFLSPIAFNWVEANAWSSV

SEQ ID NO: 9 - DNA, 1759 bp, Arabidopsis thaliana
gaaaaaaacattatcttacaaaaatccaaatactccgacttaaccgggtcggatcctggtgagtactagtatctatctcattacaattcatatccttccttcaacattcgatcatcacgaagccaaagaacaatttctccgtcactatcataaccatggcggtgctttcatctgcagatcgagctagtaatgagaagaaggtgaagagttcatactttgatttgccacctatggaaatgtctgtagcatttcctcaagcaactccagcctctacatttccaccttgtacttcagactattatcatttcaatgatctactgactccggaagaacaagctatccggaagaaagtgagggaatgcatggagaaagaagttgctccaataatgacagagtactgggagaaagcagaatttccattccatatcactccaaagcttggggctatgggtgttgctggtggctcgatcaagggttatggatgtcctggtctctccatcaccgccaatgcaattgccacagcagaaatagctagagttgatgcaagttgttcgactttcattttggtgcattcttctttgggcatgctcactattgcactctgtggatcagaagcacagaaggagaagtatttgccttctttggctcaattgaatactgtggcttgttgggctttgacagagcccgacaatggaagcgatgcaagtggtctaggaacgactgcaacaaaggttgaaggaggttggaaaattaatggacaaaagcgttggattggaaacagcacctttgcagatctgttgatcatctttgcgaggaatacaacaactaaccaaatcaacggattcatagtcaagaaagatgcgcctggcctaaaggctactaagatcccaaataaaataggtttacgtatggttcaaaatggagatattctactacagaatgtctttgttccagatgaggatcggttacctggggtaaattcttttcaggacaccagcaaggttctggctgtctcacgtgtaatggtggcctggcaaccaatcggcatatcaatgggaatctacgatatgtgccacaggtatctgaaggagaggaaacagtttggagcaccgttggctgctttccagttaaaccaacagaagcttgtgcagatgctgggtaacgttcaagcgatgtttctaatgggttggcgtctctgcaagctgtatgagacgggtcagatgactccaggtcaagccagtttaggaaaggcatggatttcatcaaaagcgagagaaactgcttcgctaggtcgggaattacttggtgggaatggaattctagcagattttctggtagcaaaggctttctgtgaccttgaacccatttatacatacgaagggacttatgatataaacaccttagtaacagggagggaagtaacgggtattgcgagtttcaaaccggctacacgtagccgtctctaagttaaaaggttgtccattgtttgttgttgtctgttggttagtattgatattgtgatgtggggtttacatctacaaatgtgccaaataatctgacccaaaaagatttagggtccaatgggggaataatatcgttgttgcaaaacttaatccagttattgcttcttgcaatatggattcgtcttgtggcttgtactgggggaaataccatttgttaaccttttattaattcaaattgccaatgtcaataacttttggtgaatgtcaaagtgtcaattaagttgtgtttctttgtt

SEQ ID NO: 10 - protein, 436 aa, Arabidopsis thaliana
MAVLSSADRASNEKKVKSSYFDLPPMEMSVAFPQATPASTFPPCTSDYYHFNDLLTPEEQAIRKKVRECMEKEVAPIMTEYWEKAEFPFHITPKLGAMGVAGGSIKGYGCPGLSITANAIATAEIARVDASCSTFILVHSSLGMLTIALCGSEAQKEKYLPSLAQLNTVACWALTEPDNGSDASGLGTTATKVEGGWKINGQKRWIGNSTFADLLIIFARNTTTNQINGFIVKKDAPGLKATKIPNKIGLRMVQNGDILLQNVFVPDEDRLPGVNSFQDTSKVLAVSRVMVAWQPIGISMGIYDMCHRYLKERKQFGAPLAAFQLNQQKLVQMLGNVQAMFLMGWRLCKLYETGQMTPGQASLGKAWISSKARETASLGRELLGGNGILADFLVAKAFCDLEPIYTYEGTYDINTLVTGREVTGIASFKPATRSRL

SEQ ID NO: 11 - DNA, 2377 bp, Arabidopsis thaliana
accaatttttttgttacctttattacattagtctctctttctgttcaaacttgtaatcctaaaaatagaacaaaatcctctgttttttctgtaacgaattctttcttattgaatcacccaaaggtaagagcttttttagtggcttctctttctttgatttatccaatttttcacacacactcgagaatctgagacaatagagagaacaatggagagagttgatcaccttgccgatgagaggaacaaagcagagttcaacgtcgacgatatgaagatcgtctgggccggttcccgccacgctttcgatgtttcaaatcgtatgtctcgcctcgtcgctaatgatccggtctttgagaaaagcaaaagagctgtgatgagtaggaaagagttgttcaagaacacgttgaggaaaagtgttcacgcttggaagttgattaacgagcttcgtctctcagatgaggaaggactcaaattgagatctttcatggatcaaccaggcttcttggatctgcattggggaatgtttgtgcctgcaattaaaggacaaggcacagagcaacaacaacaaaagtggttgtctttagctactaagatgcagataattggatgttatgctcaaactgagcttggtcatggctctaatgttcaaggccttgagacaaccgctacttttgatccaaagacagatcagtttatcattcacagtccaactcagacatcatccaaatggtggcctggtgggttaggaaaagtttctactcatgctgttatttatgctcgtctaataaccaatggcaaagaccatggtgtacatggattcatcgtgcagctgcgtagtttggatgatcattctcctcttccgggtataaccgttggtgatatcggaatgaagtttggaaacggggcatataactcaatggacaatggttttcttatgtttgatcattttcgcattcctagagatcaaatgctcatgagactgtcaaaagttacaagagaaggaaaatatgtagcatcagatgttccaaggcaattggtgtatggtactatggtgtatgtgagacagtctattgtgtcaaatgcttccaccgcgctggctcgggcagtttgcattgctactaggtacagtgctgttcgaaggcagtttggctcacatgatggtggcattgagacacaggtgattaattataaaactcagcagaacaggttgtttcctctgctggcatctgcatatgcatttcggtttgtaggggaatggctgaagtggctctacactgatgtaacaaaaagactagaggccagtgatttcgcaacattgcctgaagctcatgcatgcactgctggattgaagtctatgactacctcagccacctctgatgggattgaagaatgtcgtaagttatgtggtggacatggatacttgtggtgtagtgggcttcctgaattgtttgctgtatatgttcctgcttgcacatacgagggagacaatgttgtgttgcagttacaggttgctagatttctgatgaagacagtttcacagttgggttctggaaaggctccttctgggacaactgcttatatgggcagagcaaaacaccttttgcaatgcagttccggagttcgaaatgctagggactggttaaaccctggtatggtgttggaagcttttgaagcaagagctttgagaatggctgttacttgtgctaacaatctaagcaagtttgagaatcaagaacaaggattctcagaactcttggctgatcttgttgaggctgctactgctcattgccagttaattgttgtttctaagtttatagccaaagtagagggagatattgaaggaaaaggagtgaagaaacagctcaagaatctatgctacatctacgcactctatctccttcacaaacaccttggtgatttcctcagtacaaactctgttactcctgaacaagcctcacttgcgaaccagcagcttcgatcactctactctcaggttcgaccaaacgcagtggccctagtggacgccttcgactacacggaccagtatcttggctctgtcttaggccgctatgacggaaacgtttatccaaagcttttcgaggaagcgttgaaggatccactcaatgactcggtggttcctgacggctaccgtgagtacatccgaccgttgattaagcaacgcttccgctctgccaaactctaactctttctttgtgcggctaaaaggggcaacactgttttatttatgttatattttattcatgtttttaacaaatgtgaagtaaactacaaatatcttaagttatatgggtgataaattaatatttgtaaaatatcacatttatttatctatgaagaaataaaacagattaattgc

SEQ ID NO: 12 - protein, 664 aa, Arabidopsis thaliana
MERVDHLADERNKAEFNVDDMKIVWAGSRHAFDVSNRMSRLVANDPVFEKSKRAVMSRKELFKNTLRKSVHAWKLINELRLSDEEGLKLRSFMDQPGFLDLHWGMFVPAIKGQGTEQQQQKWLSLATKMQIIGCYAQTELGHGSNVQGLETTATFDPKTDQFIIHSPTQTSSKWWPGGLGKVSTHAVIYARLITNGKDHGVHGFIVQLRSLDDHSPLPGITVGDIGMKFGNGAYNSMDNGFLMFDHFRIPRDQMLMRLSKVTREGKYVASDVPRQLVYGTMVYVRQSIVSNASTALARAVCIATRYSAVRRQFGSHDGGIETQVINYKTQQNRLFPLLASAYAFRFVGEWLKWLYTDVTKRLEASDFATLPEAHACTAGLKSMTTSATSDGIEECRKLCGGHGYLWCSGLPELFAVYVPACTYEGDNVVLQLQVARFLMKTVSQLGSGKAPSGTTAYMGRAKHLLQCSSGVRNARDWLNPGMVLEAFEARALRMAVTCANNLSKFENQEQGFSELLADLVEAATAHCQLIVVSKFIAKVEGDIEGKGVKKQLKNLCYIYALYLLHKHLGDFLSTNSVTPEQASLANQQLRSLYSQVRPNAVALVDAFDYTDQYLGSVLGRYDGNVYPKLFEEALKDPLNDSVVPDGYREYIRPLIKQRFRSAKL

SEQ ID NO: 13 - DNA, 2070 bp, Arabidopsis thaliana
aatccctagaaatgtccgaaaatgttgaactccggcgagcccatattctcgctaatcacatactccgttcacctcggccttcttcaaacccctccctcacgccggaagtatgttttcagtactctccaccggagctcaacgagagctatggattcgaagtcaaggagatgagaaaactactcgacggacacaacttagaggaaagagactggctttatgggctcatgatgcagagcaatctgtttaacccgaaacagagaggaggtcagattttcgtctcgcccgattacaatcaaacaatggagcaacagcgccagatcagtatgaagcggatcttttacttgctcgaaaaaggggtctttcaaggatggctaacggagacaggtcctgaagctgagctcaagaagtttgctctctacgaagtttgcgggatttatgattactccctctccgccaaactcggtgttcacttcttgttgtggggtaatgctgttaagttctttgggacaaagcgtcaccatgaaaagtggctgaaagacactgaagattatgttgtcaagggctgctttgcaatgactgagctaggccatggaactaatgtacggggaattgaaacagtgactacttatgacccaacaactgaagagtttgtgataaatactccttgtgaatctgctcagaagtattggatcggtgaggcagctaaccatgcaaaccatgcaattgtgatttctcagcttagtatgaatgggaccaaccaggggattcatgtctttattgctcaaatcagagatcatgatggcaacacatgtcctaacgtccgcatagctgactgtggacacaaaattggtttgaatggtgttgacaatggccgtatatggtttgacaatcttcgaattccaagagagaatttattgaactcagttgctgatgttttagctgatgggaagtatgttagctcaattaaagatcctgatcagagatttggagcattcttggcccctttgacatctggtcgtgtcacaattgcttcaagtgcaatttattctgcaaagctcggattagctgttgctattagatactcactatcgagaagagccttctcagttgcagccaatggtcctgaagttctccttcttgattatccaagccatcaaagacgacttttaccactcctagcaaagacatatgctatgagttttgctgtaaatgacttgaagatgatttacgtgaagaggacacctgagaccaacaaagccatccatgtcgtttcaagtggattcaaagctgttctcacttggcacaatatgcgaacgcttcaggaatgtcgtgaagctgttggaggccaaggtttgaaaacagaaaaccgagtaggtcatttaaaaggagaatatgatgtgcagactacatttgagggtgacaataatgtactcatgcagctggtgagcaaggcactttttgctgaatatgtatcgtgtaagaagagaaataaacctttcaaaggattgggattggagcacatgaacagtccacgtcctgtattgccgactcaactcacatcatctacactaagatgcagccagttccagaaaagtgtgttttgcttaagagagcgagatcttctagaacggtttacttctgaagttgcagagcttcaagggagaggagaaagcagagagttcttattccttttgaatcatcaactttctgaagacttaagtaaagctttcacagaaaaagcaatactgcaaaccgttttggatgctgaggccaaattgcctcctggctcagttaaggatgtgttgggtcttgtaagatcaatgtacgcattgatcagcttggaagaagatccatcgctgctaagatatggtcacctctcaagggataatgttggagatgtgaggaaagaagtttcaaagctctgtggagagcttagacctcacgcacttgcactcgtggcttcattcgggattccagacgccttcttgagtccaattgcattcaactgggtcgaagccaacgcttggtcttcactttagttacttgctagtaataatctcttccactaat

SEQ ID NO: 14 - protein, 675 aa, Arabidopsis thaliana
MSENVELRRAHILANHILRSPRPSSNPSLTPEVCFQYSPPELNESYGFEVKEMRKLLDGHNLEERDWLYGLMMQSNLFNPKQRGGQIFVSPDYNQTMEQQRQISMKRIFYLLEKGVFQGWLTETGPEAELKKFALYEVCGIYDYSLSAKLGVHFLLWGNAVKFFGTKRHHEKWLKDTEDYVVKGCFAMTELGHGTNVRGIETVTTYDPTTEEFVINTPCESAQKYWIGEAANHANHAIVISQLSMNGTNQGIHVFIAQIRDHDGNTCPNVRIADCGHKIGLNGVDNGRIWFDNLRIPRENLLNSVADVLADGKYVSSIKDPDQRFGAFLAPLTSGRVTIASSAIYSAKLGLAVAIRYSLSRRAFSVAANGPEVLLLDYPSHQRRLLPLLAKTYAMSFAVNDLKMIYVKRTPETNKAIHVVSSGFKAVLTWHNMRTLQECREAVGGQGLKTENRVGHLKGEYDVQTTFEGDNNVLMQLVSKALFAEYVSCKKRNKPFKGLGLEHMNSPRPVLPTQLTSSTLRCSQFQKSVFCLRERDLLERFTSEVAELQGRGESREFLFLLNHQLSEDLSKAFTEKAILQTVLDAEAKLPPGSVKDVLGLVRSMYALISLEEDPSLLRYGHLSRDNVGDVRKEVSKLCGELRPHALALVASFGIPDAFLSPIAFNWVEANAWSSL

SEQ ID NO: 15 - DNA, 2354 bp, Arabidopsis thaliana
tatctttctctctttcggatcattctctcctctgattccgaatcgggtaaattattatggattcttcttcttcgtcttcctccgccgccgcacgccgccgtatcaacgctatccactctcacctcgtcacctcttctcgctcttcccctctcctccgctccaatcccaccgccggcgagttctgtcttgataatggctatagtgttgttcttcccgagaaactgaatactggcagttggaacgtctacagatctgcaaaatctccgttcaagctcgttagcagattcccagatcatcctgacatcgctactctccatgacaattttgagcatgctgttcatgattttcgagattacaagtatttaggaactcgtgttcgtgtcgacggaactgttggagactacaaatggatgacatatggagaagctggtacagcaagaactgctttaggttctggtttggttcatcacggaatccccatgggatcttctgttggaatttacttcatcaatcgcccagagtggctcattgttgatcatgcttgttcttcttattcttatgtgtctgttcctttgtatgatactcttggtcctgatgctgtgaaatttattgtcaatcatgcaactgtgcaagccatattttgtgtggcagagactttaaactctttacttagctgtttgtctgagatgccaagtgtacgcctggtggtggttgttggagggttaattgaatctttaccctcgcttccctcatcatcaggagtgaaagttgtatcctattcggtgttactgaatcagggtcgtagtaaccctcagcgattttttccaccaaaacccgatgatgttgcaaccatatgctatacaagcggaacaactgggacacccaagggagtcgtattaactcatgcaaacttgattgccaatgttgctggctccagctttagtgtgaagtttttctcttcagatgtttacatttcgtatcttccacttgctcacatttacgaacgagctaatcagatcctaacagtgtactttggagttgctgttggattctaccaaggggacaatatgaaactactggatgatttggctgctctgagacctactgtatttagcagtgtccctcgattatacaatagaatatatgctggtatcattaatgcagtaaaaacctctggtggtctgaaagagagactcttcaatgctgcctataatgcaaagaagcaggctctcttgaatggaaagagtgcttctcccatatgggacaggttggtatttaataaaataaaggacagacttggagggcgggttcgttttatgacgtctggtgcttcacctctctctcctgaagtgatggaatttttgaaagtatgctttggaggaagggtaacagagggatatggaatgactgaaacatcttgtgttataagtggaatggacgagggtgataacctcactggacatgttggctctcctaatccagcttgtgaagtaaagcttgtggatgtcccagaaatgaactatacatcagcggatcagccccatccccgtggcgaaatatgtgttaggggtcctatcatttttacaggctattacaaagatgaaattcaaacgaaagaggtgattgatgaagatggatggcttcacactggagatataggtctgtggctgccgggaggacgtctaaaaattattgacagaaagaagaacatcttcaaattggcgcagggggagtatatagctccagagaaaattgaaaacgtctatgccaaatgcaaatttgtgggccagtgcttcatatatggtgatagctttaattcatcattggtagctgttgtatcggttgatccagatgtgctgaaaagctgggcagcttcagaaggcattaagggaggagatctgagagaattgtgtaataatccgagagtgaaagcagcagtactatctgacatggacactgttggaagagaagctcagttgagaggcttcgagtttgcaaaggctgtgacattggtgctggaaccatttactctggaaaatggcttgttgactccgacgttcaagattaagagaccacaagcaaaggaatatttcgcagaagcaataacaaacatgtacaaggagcttggtgcttctgatccctctgctaatagaggtttgtgatgatggtataatcgtgtgattaataaaaagcctacgcaaaagttgtttctcccacaaattgtattcttttttgttttcttgctacatatttattgaatagtaaaggtaataaaggtgctacattacattaatgaaactttcttacagtacaatgttttccctctgtaataaaaaggcaaaacattcccaatt

SEQ ID NO: 16 - protein, 701 aa, Arabidopsis thaliana
MDSSSSSSSAAARRRINAIHSHLVTSSRSSPLLRSNPTAGEFCLDNGYSVVLPEKLNTGSWNVYRSAKSPFKLVSRFPDHPDIATLHDNFEHAVHDFRDYKYLGTRVRVDGTVGDYKWMTYGEAGTARTALGSGLVHHGIPMGSSVGIYFINRPEWLIVDHACSSYSYVSVPLYDTLGPDAVKFIVNHATVQAIFCVAETLNSLLSCLSEMPSVRLVVVVGGLIESLPSLPSSSGVKVVSYSVLLNQGRSNPQRFFPPKPDDVATICYTSGTTGTPKGVVLTHANLIANVAGSSFSVKFFSSDVYISYLPLAHIYERANQILTVYFGVAVGFYQGDNMKLLDDLAALRPTVFSSVPRLYNRIYAGIINAVKTSGGLKERLFNAAYNAKKQALLNGKSASPIWDRLVFNKIKDRLGGRVRFMTSGASPLSPEVMEFLKVCFGGRVTEGYGMTETSCVISGMDEGDNLTGHVGSPNPACEVKLVDVPEMNYTSADQPHPRGEICVRGPIIFTGYYKDEIQTKEVIDEDGWLHTGDIGLWLPGGRLKIIDRKKNIFKLAQGEYIAPEKIENVYAKCKFVGQCFIYGDSFNSSLVAVVSVDPDVLKSWAASEGIKGGDLRELCNNPRVKAAVLSDMDTVGREAQLRGFEFAKAVTLVLEPFTLENGLLTPTFKIKRPQAKEYFAEAITNMYKELGASDPSANRGL

SEQ ID NO: 17 - DNA, 2313 bp, Arabidopsis thaliana
atggaatttgcttcgccggaacaacgtcgtctcgaaaccattcgatctcacatcgatacttctccgaccaacgatcaatcatcatctctattcctcaacgccaccgcttcttctgcttcacctttctttaaagaggatagctacagtgttgtgcttccagaaaagcttgatactggaaaatggaatgtctacagatctaaaagatcgcctacgaaactcgttagtaggttcccggatcatcctgaaatcgggactttacatgacaattttgtacatgctgttgaaacatatgctgaaaacaagtatcttggtacacgagttcggtccgatggaaccattggagagtattcatggatgacatatggagaagcagcgtctgagcgacaagccattggttcaggactcttgtttcatggagttaaccaaggagattgcgttggactctattttattaacagaccagagtggttggttgtggatcatgcttgtgcagcatattcatttgtctctgttcctttatatgatacacttggtccagacgctgttaagtttgtggtgaatcatgctaatctgcaagctatattttgtgtaccacaaaccttgaatattttgctaagcttcctagcggaaatcccatccattcgtctcattgtggtggtgggaggggctgatgagcatttgccatcacttcctcgaggaactggagtcacaattgtatcataccaaaagctattgagtcagggtcgaagtagcttacatccattttcgcctccaaagccagaagacattgcaaccatatgctacacaagtggaaccacaggaacaccaaagggtgttgtgttgactcatggaaacttgatcgcgaatgtcgctggttccagtgtggaagcagaattctttccttcagatgtttacatatcatatcttcctttggcgcacatatatgaacgtgcaaatcagattatgggggtgtatggtggtgttgctgtcggtttctatcagggggatgtcttcaagctgatggatgattttgctgtgttaagaccaacaatattctgtagtgtccctcgcttatataatcgaatatatgatggcattacaagtgccgtaaaatcatctggggttgtgaaaaaaaggcttttcgaaattgcctataactcaaagaagcaagcgatcattaatgggcggactccttctgcattttgggacaagctggtgttcaacaaaataaaagaaaagcttggtggacgggttcggtttatggggtctggtgcttctcctttgtcacctgatgtcatggatttcttgagaatatgctttggatgttcggtgcgtgaagggtatggtatgaccgagacttcttgtgtcataagtgctatggatgatggtgacaatttatctggccatgtcggttcccctaatccagcttgcgaggtaaaacttgtggatgttcccgaaatgaattacacatcagacgatcaaccatacccacgtggtgaaatctgtgtaagaggaccaatcatcttcaaaggctactacaaagatgaagaacaaacgagagaaattcttgatggagatggctggctacacacaggagatatcgggttgtggttacctggtggtcggctcaagatcatagacaggaagaagaacatatttaagttggcgcaaggagaatatatagcaccagagaagatcgaaaatgtttataccaaatgtagattcgtttcgcagtgtttcattcacggtgatagcttcaattcctctctagtagctatagtttcagtcgaccccgaagttatgaaagattgggctgcatcagaaggcatcaagtatgagcatctaggacagctctgtaacgatccaagagtgcgaaagactgttcttgctgagatggatgaccttggaagagaagctcagttgagagggtttgagtttgcaaaggctgtgactttggtgccagaaccattcaccttggagaatggacttctcacaccaacattcaagataaagagacctcaagcaaaagcctactttgcagaagcaattagcaaaatgtatgcggaaatcgcagcctcgaaccccattccttctaaactgtgatgctttttgctttgcttaaaagtgtttctttttttttgtattacaaaggtaattgtagaactgttaaaagatcggattaggatgaggtagatcgatcatttgaaataaaacagattcatggtgtaattttctttgatactgttttccagaataaaggaagcagactatgtcctaatcctgaaatggatgatgagtgtggtcataagtttg

SEQ ID NO: 18 - protein, 700 aa, Arabidopsis thaliana
MEFASPEQRRLETIRSHIDTSPTNDQSSSLFLNATASSASPFFKEDSYSVVLPEKLDTGKWNVYRSKRSPTKLVSRFPDHPEIGTLHDNFVHAVETYAENKYLGTRVRSDGTIGEYSWMTYGEAASERQAIGSGLLFHGVNQGDCVGLYFINRPEWLVVDHACAAYSFVSVPLYDTLGPDAVKFVVNHANLQAIFCVPQTLNILLSFLAEIPSIRLIVVVGGADEHLPSLPRGTGVTIVSYQKLLSQGRSSLHPFSPPKPEDIATICYTSGTTGTPKGVVLTHGNLIANVAGSSVEAEFFPSDVYISYLPLAHIYERANQIMGVYGGVAVGFYQGDVFKLMDDFAVLRPTIFCSVPRLYNRIYDGITSAVKSSGVVKKRLFEIAYNSKKQAIINGRTPSAFWDKLVFNKIKEKLGGRVRFMGSGASPLSPDVMDFLRICFGCSVREGYGMTETSCVISAMDDGDNLSGHVGSPNPACEVKLVDVPEMNYTSDDQPYPRGEICVRGPIIFKGYYKDEEQTREILDGDGWLHTGDIGLWLPGGRLKIIDRKKNIFKLAQGEYIAPEKIENVYTKCRFVSQCFIHGDSFNSSLVAIVSVDPEVMKDWAASEGIKYEHLGQLCNDPRVRKTVLAEMDDLGREAQLRGFEFAKAVTLVPEPFTLENGLLTPTFKIKRPQAKAYFAEAISKMYAEIAASNPIPSKL

SEQ ID NO: 19 - DNA, 1852 bp, Arabidopsis thaliana
caaagcggtgagtcatcttaccttataaatgatcacaaacccgaggactgtgagttttcgtgtagaacaaatccttctctgagaaaaaacaacagatccgaattttatctttaatcagccggaaaaaatggagaaagcgatcgagagacaacgcgttcttcttgagcatctccgaccttcttcttcttcttcgcacaattacgaggcttctctatctgcttctgcttgcttggctggggacagtgctgcatatcagaggacctctctctatggagatgatgttgtcattgtcgcggcacataggactccactatgcaagtccaaacgtggcaatttcaaggatacatatcccgatgatttgctcgcacctgttttgagggcattgatagagaagacgaatctaaacccaagtgaagtaggtgacattgttgtgggtactgttttggcacctggatctcagagagccagtgaatgcaggatggctgcgttctatgctggtttccctgaaaccgtggctgtcagaactgtgaatagacagtgctcatctgggcttcaggctgttgctgatgtagccgctgccattaaagcgggattttatgacattggtatcggggctggtttggagtccatgactaccaatccaatggcatgggaagggtcagtcaacccagcggtgaagaagtttgcacaagcgcagaattgtcttcttcctatgggtgttacgtcagaaaatgtagcacaacgctttggtgtctcaaggcaggagcaagatcaagctgctgttgactcgcacagaaaggcagctgctgctactgctgctggtaaattcaaggatgagatcattcctgttaagaccaagcttgttgacccgaagactggtgatgagaaacccattacagtttctgttgatgatggtatccgaccaaccacaactcttgcttctcttgggaagctgaagccagtgtttaagaaggatggcaccactactgctggaaattccagccaagtaagtgatggtgcaggagcggttctcctaatgaagagaagtgttgcaatgcaaaaaggacttcccgttcttggtgtattcaggacatttgctgcagttggtgttgaccctgcaatcatgggtatcggtccagcagttgccattcctgctgcagttaaggcggctggtttagaacttgatgacatcgacttgtttgagatcaatgaggcatttgcatctcagtttgtttattgccgtaacaaattgggacttgacccagagaaaatcaatgtcaacggaggtgcaatggccataggccatcctttgggcgctacaggagcgcgttgtgttgctacattgttgcacgagatgaaacgccgtggtaaagactgccgttttggagtagtgtcaatgtgcattgggacggggatgggtgcagcagctgtgtttgagagaggagatggagttgatgagcttcgcaacgcaaggaaagttgaagcgcaaggtcttttgtccaaggacgctcgctagagaggaccatgcaccaaaaccgtttttcacctcacctctctcttttctggtcttcttgtcactttcttcagatattattatagtttcgaataaagcacacaaccaatgtttgcctgagtcttgtgttgttcttgaccaggtcatgtgtttagtattttttttaagtgaagggggataatctttaaatcttttatatgtaagaatattgttcttctcgaatttcatcagttgttgcttctagaaattttagattttttgggtggcagccatttctatatttcgatggataagtcaaacgttcataaagtcaagggttctgccttttgtttgtgtttc

SEQ ID NO: 20 - protein, 462 aa, Arabidopsis thaliana
MEKAIERQRVLLEHLRPSSSSSHNYEASLSASACLAGDSAAYQRTSLYGDDVVIVAAHRTPLCKSKRGNFKDTYPDDLLAPVLRALIEKTNLNPSEVGDIVVGTVLAPGSQRASECRMAAFYAGFPETVAVRTVNRQCSSGLQAVADVAAAIKAGFYDIGIGAGLESMTTNPMAWEGSVNPAVKKFAQAQNCLLPMGVTSENVAQRFGVSRQEQDQAAVDSHRKAAAATAAGKFKDEIIPVKTKLVDPKTGDEKPITVSVDDGIRPTTTLASLGKLKPVFKKDGTTTAGNSSQVSDGAGAVLLMKRSVAMQKGLPVLGVFRTFAAVGVDPAIMGIGPAVAIPAAVKAAGLELDDIDLFEINEAFASQFVYCRNKLGLDPEKINVNGGAMAIGHPLGATGARCVATLLHEMKRRGKDCRFGVVSMCIGTGMGAAAVFERGDGVDELRNARKVEAQGLLSKDAR

SEQ ID NO: 21 - DNA, 2535 bp, Arabidopsis thaliana
tctctttttcattaattccgaaaattttgactcgcaaaatctcaacgaagctcgaaaatggattcacgaaccaaggggaagacggtgatggaagttggtggcgatggcgttgccgtcataacactcatcaatcctcccgtcaattctctatccttcgacgtgctatacaatctcaaaagtaattacgaggaggccttgagcaggaatgatgttaaagctattgttattactggtgcaaaggggaggttctctggtggctttgatatatctggttttggtgaaatgcagaaggggaatgtcaaagagccaaaggctggttacatttcgattgacatcataaccgacttgcttgaagctgctaggaaaccgtctgtcgctgccattgatggacttgccttgggaggaggattagaacttgctatggcttgtcatgctaggatatcagctcctgctgcacagttaggcttgcctgagctgcaacttggtgttattcctggttttggaggaacgcagcgtctcccgcgtcttgttggtctcaccaaagcccttgaaatgattttgacatctaagccagttaaagctgaggaaggtcattctttgggtcttattgatgctgtggtgccacctgcggagttagtaaccactgctcgtcgctgggcccttgacatagtcgggaggagaaaaccatgggtatctagtgtttccaagactgataagttacctcctcttggagaggcaagggagatactgacgtttgccaaggcacagacactcaagcgtgcccccaatatgaaacaccctttaatgtgccttgacgctattgaagtaggtattgtttctggtccaagggctggtttagaaaaggaagctgaagtcgcctcacaagtggtaaaactggataccaccaaaggcttgatccacgtcttcttttctcagcgaggaactgctaaggttcctggagttactgatcgtgggttggtgccaaggaagattaagaaggtagccataattggaggcgggttgatgggatctggaatagctactgcgttgatcctaagtaactatccagtgattctcaaggaggtaaatgagaaattcctggaggctggaattggcagagttaaagctaatctccagagccgtgtaaggaagggaagcatgtctcaggaaaagtttgagaaaaccatgtctctccttaagggttctcttgattatgaaagctttagagatgtggacatggtcattgaggctgttattgagaatatatctttgaagcaacaaatttttgctgatctggagaaatactgtcctcagcattgtatccttgctagcaacacatcgaccattgacttgaacaaaattggggagcggaccaagtctcaggatcgaattgttggagcacattttttcagtccagcacatatcatgccgctacttgaaatagttcggaccaatcatacctctgcccaagtaattgttgacctgttagatgttgggaagaagattaagaaaacaccagttgtggtgggaaactgcacagggtttgcagtgaataggatgttcttcccttacacacaggcagctatgttccttgttgagtgtggagcagatccatatctaatcgacagggcaatcagcaagtttggaatgccaatgggtcccttcagactgtgtgacctggttggatttggtgtggcgattgcaaccgcaacgcagtttatcgagaacttctcagaacggacttacaaatcaatgattattccacttatgcaagaggacaagagagctggtgaagccactcgcaaaggtttctatttgtatgatgataagcgcaaggcgaaacctgatcctgagctaaagaaatatatcgaaaaggcaagaagcatatctggagtaaagcttgaccctaagttggcgaatttgtcggagaaggacattattgaaatgacattcttcccagtagtaaacgaggcgtgtagggtttttgctgaaggtatcgctgtcaaagcagcagaccttgacattgctggcataatgggaatgggttttccaccttacagaggaggaatcatgttctgggctgattccatcggatcgaaatacatttactcgaggctggatgagtggtcgaaggcttatggtgaattcttcaagccttgtgcttttttggctgagaggggatctaaaggagttcttttgagcgctcccgtgaaacaagccagctcacggttgtaaggtcagcgtttcagtggaaccatttctccattggtgcatcttctgtgttttctttatgttcacattttacttagccgggaagtgggtatcttttggcctcagaagcttttaaataaagtctcatctcagacatatgaatctgggaggaagtagggattaagttgtagtagcaataagatttgttttatgttgtgctttgtcatacaacaaagttgtcagatacatttgttgttataaagctatatggagtaagttctatgatttaatgataatgttgtcatttgacaaatcagttaaaat

SEQ ID NO: 22 - protein, 725 aa, Arabidopsis thaliana
MDSRTKGKTVMEVGGDGVAVITLINPPVNSLSFDVLYNLKSNYEEALSRNDVKAIVITGAKGRFSGGFDISGFGEMQKGNVKEPKAGYISIDIITDLLEAARKPSVAAIDGLALGGGLELAMACHARISAPAAQLGLPELQLGVIPGFGGTQRLPRLVGLTKALEMILTSKPVKAEEGHSLGLIDAVVPPAELVTTARRWALDIVGRRKPWVSSVSKTDKLPPLGEAREILTFAKAQTLKRAPNMKHPLMCLDAIEVGIVSGPRAGLEKEAEVASQVVKLDTTKGLIHVFFSQRGTAKVPGVTDRGLVPRKIKKVAIIGGGLMGSGIATALILSNYPVILKEVNEKFLEAGIGRVKANLQSRVRKGSMSQEKFEKTMSLLKGSLDYESFRDVDMVIEAVIENISLKQQIFADLEKYCPQHCILASNTSTIDLNKIGERTKSQDRIVGAHFFSPAHIMPLLEIVRTNHTSAQVIVDLLDVGKKIKKTPVVVGNCTGFAVNRMFFPYTQAAMFLVECGADPYLIDRAISKFGMPMGPFRLCDLVGFGVAIATATQFIENFSERTYKSMIIPLMQEDKRAGEATRKGFYLYDDKRKAKPDPELKKYIEKARSISGVKLDPKLANLSEKDIIEMTFFPVVNEACRVFAEGIAVKAADLDIAGIMGMGFPPYRGGIMFWADSIGSKYIYSRLDEWSKAYGEFFKPCAFLAERGSKGVLLSAPVKQASSRL

SEQ ID NO: 23 - DNA, 2849 bp, Arabidopsis thaliana
aataaagctttcatttttcaatctcatcagagagaaattaagctatggcgaagaagattggagtaacgatggaagttgggaacgatggtgttgctgtcatcaccatttctaaccctcctgttaactctttagccagtccaattatttctgggttgaaggagaagtttcgtgacgccaatcaaaggaatgatgttaaggccatcgtgttgatcgggaacaatggaagattttcgggtggttttgatatcaatgtctttcagcaagttcataagactggggatttatcgcttatgcctgaagtatctgttgagcttgtgtgtaacctgatggaagactccaggaagccggttgttgctgcagttgaaggattagctcttggtggtggtttggaacttgcaatggcgtgtcatgccagagttgctgctccgaaagctcaattaggcttaccagaactgactcttggagtgattcctggttttggaggaacccaacgtcttccaaggttagtaggccttgcaaaagcaacggatatgatactgctttctaagtcaatatcctcagaagaggggcacaaactgggtctcattgatgccttggtgccacctggagatgtgttgagcacatccagaaagtgggctctagacattgcagagggacgtaaaccctttctgcagtcactgcacagaaccgacaaaattggttctttatctgaagctcgtgctatattgaagaattcaagacagctagccaagaagattgccccaaatatgcctcagcaccatgcttgcattgaggtgattgaagaaggaatcatccatggaggatacagtggggttctcaaggaagcagaagttttcaagcaactagtactgtcagacactgcaaagggtcttgttcatgttttctttgcacaacgtgcaacatcaaaagtgcctaatgtaactgacgttggattgaaaccaaggccgataaaaaaagttgcagttattggtggcggtctgatgggttcaggcatcgctacggctctacttctaagcaatataagagttgtgctcaaagaaataaactcagaattccttatgaagggaataaaatcagttgaagcaaatatgaaaagcttggtatctagggggaaactgacacaagataaagcaggaaaggccctctctttgttcaagggagtacttgattacacagaattcaatgatgtggacatggtcatagaggcagtgattgaaaacattcaattgaaacaaaacatattcaaagaaatcgaaaaggtttgttcaccccactgcattttggcgagtaatacatctactatcgacctcgatgtaattggtgaaaagactaactcaaaagatcgcattgttggcgcacatttcttcagtccggcccatctgatgccccttctcgagattgttcgttcaaagaatacttctgctcaggtgattctggatctcatggcggttggaaaggccataaagaaagttcccgtggtggttggaaactgcataggctttgcagtgaataggacattctttccttattcacaagctgcacatatgttggccaatctaggtgttgacttgttcagaattgacagcgttattacctctttcggcttgccattgggccctttccagcttggtgatttggctggacacgggattggattagcagttgggccgatatatgccaaggtctatggtgaccgcatgttcagatctccaatgacagagcttcttcttaagagcgggcgaaatggaaaaatcaacggaagaggatactatatatacgaaaagggaagcaaaccaaaacctgatccatcagtgctttcaattgttgagaaatcaaggaaactgaccaatatcatgcctggtgggaagcctatatcagtaactgataaagaaattgtagagatgatcttattccctgttgtcaacgaggcatgccgcgtcctagatgaaggagttgtgatccgagcctcagacttggacattgcgtctgtccttggaatgagttttccttcttaccggggaggaattgttttctgggcagacactgttggaccaaagtacatatatgagaggctcaagaaattgtcggagacttatggcagctttttcaaaccatcgaggtatctggaggaaagagcaatgaatggaatgcttttgagtgaatcgaaatcgtcgaggtccaaattgtgaagcgtttaattaagtatatgcagaaggagaggctgaaggtgttagtcactaaatgaaaataaatcagtctctcttgttgtttcaaagttcaaagttaaaatgctattaaggtgtggttgttgttaacactcttaaaataaacaattttctgctcttaataaatgtttgtagtgacgtcagtttcataaagttatgatttgttctagccagagagaaaaatatgaagaaatttacatctttttttttataatcaaattaaggatttattatatattactttcaaatcctacagaagctgattgagaaacatctcagatcataatccgacacagtaaacaagggtgttccacagctaaaccatcaacataaaaagtgatactgaacacaaaaaaaaaactaacaaaaaattcgtgtagtaaagaaacaaagctcaccaaaatctacaccagccacaaggggctttaaagaggagactttataagaaaagaaagagaaaagtgagaagaaagcttaacgacacaataaaagcttgtgtctattactctctctaatcacctgccaatgcgttgaagccgcaacaaccgccactgagaaagaaaaattacggtgggattacctgataatatagggcggtggtga

SEQ ID NO: 24 - protein, 721 aa, Arabidopsis thaliana
MAKKIGVTMEVGNDGVAVITISNPPVNSLASPIISGLKEKFRDANQRNDVKAIVLIGNNGRFSGGFDINVFQQVHKTGDLSLMPEVSVELVCNLMEDSRKPVVAAVEGLALGGGLELAMACHARVAAPKAQLGLPELTLGVIPGFGGTQRLPRLVGLAKATDMILLSKSISSEEGHKLGLIDALVPPGDVLSTSRKWALDIAEGRKPFLQSLHRTDKIGSLSEARAILKNSRQLAKKIAPNMPQHHACIEVIEEGIIHGGYSGVLKEAEVFKQLVLSDTAKGLVHVFFAQRATSKVPNVTDVGLKPRPIKKVAVIGGGLMGSGIATALLLSNIRVVLKEINSEFLMKGIKSVEANMKSLVSRGKLTQDKAGKALSLFKGVLDYTEFNDVDMVIEAVIENIQLKQNIFKEIEKVCSPHCILASNTSTIDLDVIGEKTNSKDRIVGAHFFSPAHLMPLLEIVRSKNTSAQVILDLMAVGKAIKKVPVVVGNCIGFAVNRTFFPYSQAAHMLANLGVDLFRIDSVITSFGLPLGPFQLGDLAGHGIGLAVGPIYAKVYGDRMFRSPMTELLLKSGRNGKINGRGYYIYEKGSKPKPDPSVLSIVEKSRKLTNIMPGGKPISVTDKEIVEMILFPVVNEACRVLDEGVVIRASDLDIASVLGMSFPSYRGGIVFWADTVGPKYIYERLKKLSETYGSFFKPSRYLEERAMNGMLLSESKSSRSKL

SEQ ID NO: 25 - DNA, 1273 bp, Arabidopsis thaliana
attcatttcatacatatttgtctctcttaaatgtgtttatttccccttttgtagtaaccactcaaaaggttttacttcactcaaaaatggctttgtctcttatctttctagctctcttagtgttgtgccctagtagtggtcacagccaacgttctccttcaccgggatactacccgagttcccgagtaccgacttcaccttttgatcgtgaattccgtactttatggggctctcaacaccaacgtagagagcaagacgttgtcactctttggctcgacaaatccactgggagtgggttcaagtctcttcgtccataccggtcgggctactttggtgcttccattaagctccaaccaggcttcactgctggagtggatacatccctctatctctcaaacaaccaagagcatcctggagaccacgatgaggtcgatatcgagtttctagggacaacgccagggaagccttattcccttcaaacgaatgtcttcgttaggggaagtggcgaccgaaatgtcattggaagagaaatgaaatttaccttgtggtttgaccctactcaagattttcaccattacgcaattttgtggaaccctaaccaaattgtattctttgtagacgatgtaccgatacgtacatataatagaaagaatgaagctatattccccacaagaccgatgtgggtttacggatcgatatgggatgcatcggactgggccacagaaaatggaaggatcaaagccgactatcgataccaaccatttgtggctaagtacaaaaactttaagctagcgggatgcacagcggatagctctagctcatgcagaccgccatcgcctgcacccatgcgcaaccgcgggttgagccggcagcagatggcggcattgacatgggcacagaggaacttcttggtctataactattgccatgatccgaaaagagaccatacccaaacaccagaatgttaaaacaaacaaacaaaagaaaggttataataattttattttgcattatcaaacatacatgaataattggaggtcttgtgacgggtccacactggaaactatattgaagcagttgagcgtccggtcctctatgatttgagattaaaaagaaaaggttttgtttttcaatactttgtaatgtttccatttggtatctttcttataattgtaatattgtgtgatcacatatattaatatctggtattgtattgttgtgtgaaccaaaaaagaaagaaaaagaatggaagttgttctttcgaaggtggctt

SEQ ID NO: 26 - protein, 293 aa, Arabidopsis thaliana
MALSLIFLALLVLCPSSGHSQRSPSPGYYPSSRVPTSPFDREFRTLWGSQHQRREQDVVTLWLDKSTGSGFKSLRPYRSGYFGASIKLQPGFTAGVDTSLYLSNNQEHPGDHDEVDIEFLGTTPGKPYSLQTNVFVRGSGDRNVIGREMKFTLWFDPTQDFHHYAILWNPNQIVFFVDDVPIRTYNRKNEAIFPTRPMWVYGSIWDASDWATENGRIKADYRYQPFVAKYKNFKLAGCTADSSSSCRPPSPAPMRNRGLSRQQMAALTWAQRNFLVYNYCHDPKRDHTQTPEC

SEQ ID NO: 27 - DNA, 1149 bp, Arabidopsis thaliana
atctctcccccttcttctctcttcctcacattcctcaccaaaccctctccaaaacacacccacacgtacgcacacacacaaagacaatgtctcctttcaaaatattcttcttcacgactcttctcgtggcggcgttttcagtgtcggctgctgatttcaacactgacgtcaacgtagcttggggaaatggccgtgggaagatactcaacaacggccagcttcttactctctccttagacaaatcctctggttccggttttcaatccaaaacagagtatttgtttggaaagattgatatgcagattaagcttgttcctggtaactctgcaggaacagtcacaactttttacctaaaatccgaaggatccacttgggatgagattgattttgagttcttgggtaatatgagtggagatccttatactttacacactaatgtttacactcaaggtaaaggtgacaaagagcaacaattccatctctggttcgacccaaccgccaatttccacacttactcaatcctctggaaccctcaaagaatcatattgaccgtcgatgacacacccattagagagtttaaaaactatgagtctctcggtgtcttgtttccaaagaacaagccgatgaggatgtacgcgagtttatggaacgcagacgattgggcaacaagaggcggtcttgttaaaactgattggtctaaagctccattcatggcttcttacagaaacattaagattgactcgaaaccaaactccaattggtacactcaagaaatggattcaacaagccaagctagactcaaatgggttcagaagaattacatgatctacaattattgtactgaccataggaggtttccacagggagctcctaaggaatgcacaacaagctcatagaatctcaaattatattctatttatttatctacacttcctctctttcttttatgtgaaaattgtgaatgctctgtttatagcttgtctattatgtccgagaatttctttttctgtttttgattcttttcgttgtatatctttgtccaataaaggaaatgatgtgtctttactcttatagatatgtataaaaagatgtccctgttttatttgttaaaaaaaattgtttatgataatgatagttctttcttcttct

SEQ ID NO: 28 - protein, 269 aa, Arabidopsis thaliana
MSPFKIFFFTTLLVAAFSVSAADFNTDVNVAWGNGRGKILNNGQLLTLSLDKSSGSGFQSKTEYLFGKIDMQIKLVPGNSAGTVTTFYLKSEGSTWDEIDFEFLGNMSGDPYTLHTNVYTQGKGDKEQQFHLWFDPTANFHTYSILWNPQRIILTVDDTPIREFKNYESLGVLFPKNKPMRMYASLWNADDWATRGGLVKTDWSKAPFMASYRNIKIDSKPNSNWYTQEMDSTSQARLKWVQKNYMIYNYCTDHRRFPQGAPKECTTSS

SEQ ID NO: 29 - DNA, 1359 bp, Arabidopsis thaliana
atattcactctctcttcttcattagcctttcgtctccttcttctcttctctatctctctttagcttctctacgcaagcttattatctctttctcttaaagctttttctatatacaaacagaagcgtttgatactgtaaaaaagatttagaaatggagactctgagtcgtttattggttttcatgtctctgttttccggtttagtttctggatttgctctgcaaaatcttccaatcacatcttttgaagaaagttacactcaactttttggtgacaagaacttatttgttcatcaagatggcaaatctgtccggttaacgctcgatgaaagaaccggttccggttttgtttcaaatgattattacttgcatggattcttcagtgcttcaattaaattaccttctgattatacagctggagttgttgttgccttttatatgtctaatggagatatgtatgagaagaatcatgatgagatagattttgagtttcttggtaacattagagaaaaagaatggagagttcagacaaacatttacggtaatggaagtactcattcaggaagagaagagagatataatctctggtttgatcctactgaagattttcatcaatacagtatcctctggtctgattctcacatcatattctttgtagacaatgttcctattagagaggtcaaacgtactgcggaaatgggtggtcactttccatcaaagccgatgtctctctacacaacaatatgggacggttctaaatgggcaactaacggtggaaagtacggtgtaaactacaaatatgcgccttacattgcgcggttctcggatctagtcctgcacggctgccccgtggaccctatcgagcagtttccgaggtgcgatgaaggcgcggctgaggatatgcgtgcggcgcaagagattactccttcacaaaggagtaaaatggatgttttcagacggagactcatgacatattcatattgctatgatcgggctaggtacaatgttgctttatcggagtgtgtggtgaatcccgctgaggctcaaaggcttagggtttatgatccggtcagatttggcggcattccgaggcgccaccgcaatggaaagcaccggagcaagagaagccgggttgatggaaccgagtcgatatgataacatatataggcaaaatagtatgggtgagatttgggggttctataataagtccattattttggttatttatgattcaatgcattttcacatatagaggaggcatttttgtcatttgttagttgtatatcattataagaaaagaaaaaataattaatttatttatttgtgatattttttttatatatgataaattgtttcttgtt

SEQ ID NO: 30 - protein, 333 aa, Arabidopsis thaliana
METLSRLLVFMSLFSGLVSGFALQNLPITSFEESYTQLFGDKNLFVHQDGKSVRLTLDERTGSGFVSNDYYLHGFFSASIKLPSDYTAGVVVAFYMSNGDMYEKNHDEIDFEFLGNIREKEWRVQTNIYGNGSTHSGREERYNLWFDPTEDFHQYSILWSDSHIIFFVDNVPIREVKRTAEMGGHFPSKPMSLYTTIWDGSKWATNGGKYGVNYKYAPYIARFSDLVLHGCPVDPIEQFPRCDEGAAEDMRAAQEITPSQRSKMDVFRRRLMTYSYCYDRARYNVALSECVVNPAEAQRLRVYDPVRFGGIPRRHRNGKHRSKRSRVDGTESI

SEQ ID NO: 31 - DNA, 1182 bp, Arabidopsis thaliana
ctcaccatcatcatcatcatcatcatcatcatttctctctatctctttctaaaacacaaccatgactgtttcttcatctccatgggctctcatggctctctttctaatggtttcttcaacaatggtaatggctattcctccacgcaaggccattgatgtaccatttggtcgtaactacgtcccaacttgggcttttgaccaccagaaacagttcaatggcggttccgaacttcagcttatcctcgacaaatacactggcacaggatttcaatcaaaggggtcatatttgtttggacattttagtatgcacataaagcttccagctggtgacacagccggagttgtcacagctttctatctatcatctaccaacaatgagcatgacgagatagactttgagttccttggaaacagaacaggacaaccagctatattacagacaaatgtattcacaggaggaaagggaaacagagaacaacgaatctatctctggtttgatccttctaaggcttatcacacttactcaatcctttggaacatgtaccagatcgtattctttgttgacaacataccaatccgaacgttcaagaatgctaaggatctaggagtacgtttcccattcaaccaaccaatgaagctttactcaagcctttggaacgcggatgattgggccacgagaggcggtttagagaagaccaattgggccaatgcacctttcgttgcatcttacaaaggattccacatagatggttgccaagcttctgtggaagccaagtactgtgccacacaaggccgcatgtggtgggatcagaaagagttccgtgaccttgacgctgaacaatggcgtcgtctcaaatgggttcgtatgaagtggaccatctacaactactgtaccgaccggactaggttcccggttatgccagctgaatgtaaaagggacagagacgcataagttactactcttgagggttttaatgaatttatgctatcattattatttgaattatgcttgttcaagagattgatatatgtattgtttgttggcccatgatgtttatgctatatttgggcctaaaattacatgttataattcatatatgtattgattagctatgtattttcatatgttatgatctacttacaagttttattctttaacgtggtaaaagtgcatttggtatt

SEQ ID NO: 32 - protein, 296 aa, Arabidopsis thaliana
MTVSSSPWALMALFLMVSSTMVMAIPPRKAIDVPFGRNYVPTWAFDHQKQFNGGSELQLILDKYTGTGFQSKGSYLFGHFSMHIKLPAGDTAGVVTAFYLSSTNNEHDEIDFEFLGNRTGQPAILQTNVFTGGKGNREQRIYLWFDPSKAYHTYSILWNMYQIVFFVDNIPIRTFKNAKDLGVRFPFNQPMKLYSSLWNADDWATRGGLEKTNWANAPFVASYKGFHIDGCQASVEAKYCATQGRMWWDQKEFRDLDAEQWRRLKWVRMKWTIYNYCTDRTRFPVMPAECKRDRDA

SEQ ID NO: 33 - DNA, 1127 bp, Arabidopsis thaliana
atcaacaatatacatctgagatatcaaaactcctagagagatctaataaaaatgaagtcttttacgttcttgattctctttctctttgcagcacaatctatcagcgtctatgcaggcagcttccacaaagacgttaagatacattggggtgatggtcgtggaaagattcacgataatcaagggaaacttctatctctctcgcttgacaaatcctctggttcaggtttccaatctaatcaagagtttctctatggtaaagccgaagttcaaatgaaacttgtccctggtaactctgctggaaccgtcacaacattctatcttaaatctccgggaactacatgggatgagattgatttcgagtttttggggaacataagtggtcatccatatactctccatactaatgtttacacaaaaggctcaggagacaaagaacaacaatttcatctatggtttgacccaaccgctaactttcacacttattgcatcacatggaatccacaaagaattatttttaccgttgatgggattccaattagagagtttatgaatgccgagtcacgtggagtcccattcccaacaaagcaaccaatgaggctatatgcgagtctatgggaagcagagcattgggctacaaggggaggattagagaaaacagattggtcaaaagctcctttcaccgcttattacagaaactacaatgtcgagggatgtgtatgggttaatggaaagtcagtttgtcccgcaaattcccaatggttcactcaaaaactcgattcgaatggccagacaagaatgaaaggggtacagagtaaatacatggtttacaactattgctccgataaaaaaaggtttcctcgaggtgttcctccagagtgcagctaaatgattgattctttgattcataaataagttacttgacgttgtctttgtaactcaatggttcttcttatctataatttttcaattcatttgttaccaaatatatgcgtgtttgtgtaagatcttttttacaatgaacaatgagttcttccattctaaaaaccttttaaaacatgtcgttactatatacgtacaacgatacttggggataaattgaataatatataaccttacttgatggataa

SEQ ID NO: 34 - protein, 277 aa, Arabidopsis thaliana
MKSFTFLILFLFAAQSISVYAGSFHKDVKIHWGDGRGKIHDNQGKLLSLSLDKSSGSGFQSNQEFLYGKAEVQMKLVPGNSAGTVTTFYLKSPGTTWDEIDFEFLGNISGHPYTLHTNVYTKGSGDKEQQFHLWFDPTANFHTYCITWNPQRIIFTVDGIPIREFMNAESRGVPFPTKQPMRLYASLWEAEHWATRGGLEKTDWSKAPFTAYYRNYNVEGCVWVNGKSVCPANSQWFTQKLDSNGQTRMKGVQSKYMVYNYCSDKKRFPRGVPPECS

SEQ ID NO: 35 - DNA, 1048 bp, Arabidopsis thaliana
atcaacaatacatttgagatatcaatacacctccaaaatatatacaaaaatgaagctttcttgtggtacaagttttgcgttcttgattatgtttctctttgcggcacaatctatgcatgtctatgcgggtagcttccacaaagatgttcagatacattggggtgacggccgtggaaaggtgcgcgacagagatggaaagcttctatctctctcgctcgacaaatcctctggatccggttttcagtccaaccaggagtttctctatggcaaagccgaggttcaaatgaaacttgtccctggtaactcagctggaacagtaacaacattctatcttaagtctccgggaactacgtgggatgagattgatttcgagttcttaggaaacctaagtggtcatccgtatactctccatactaatgtttacacaaaaggctcaggagacaaagaacaacaatttcatctatggttcgacccaactgttaactttcacacttattgcatcacatggaatccccaaaggattatttttacagttgatggaattcctattagagagttcaagaactccgagtcaattggagttccgttcccaacgaagcaaccaatgaggctttacgcgagtctctgggaagccgagcattgggctacaaggggagggttagagaaaacagattggtcaaaggctcctttcaccgctttctacagaaactacaatgttgaaggatgtgtatgggctaatggaaaatcatcttgtcccgcaaattcctcatggttcactcaacaactcgattcaaacggccagacaagaatgaaaggggtacagagtaagtacatggtctacaactattgtaacgacaaaagaaggtttcctcgaggtgttcctgtagagtgcagttaaattgttgattctttgatacatgaataagttacttggcattgccttagtaactcaattgagttcttacatatttttatcattcatttgttgctcatatgtatgcggtgtttgtgtaagatgattttaagaagtaataaaaataaattatgg

SEQ ID NO: 36 - protein, 282 aa, Arabidopsis thaliana
MKLSCGTSFAFLIMFLFAAQSMHVYAGSFHKDVQIHWGDGRGKVRDRDGKLLSLSLDKSSGSGFQSNQEFLYGKAEVQMKLVPGNSAGTVTTFYLKSPGTTWDEIDFEFLGNLSGHPYTLHTNVYTKGSGDKEQQFHLWFDPTVNFHTYCITWNPQRIIFTVDGIPIREFKNSESIGVPFPTKQPMRLYASLWEAEHWATRGGLEKTDWSKAPFTAFYRNYNVEGCVWANGKSSCPANSSWFTQQLDSNGQTRMKGVQSKYMVYNYCNDKRRFPRGVPVECS

SEQ ID NO: 37 - DNA, 1067 bp, Arabidopsis thaliana
acaaaccaatctaactcaatacaagaatatctacaatctctagaaaatggcgatcacttacttgcttcctctgtttctttctcttatcatcacctcctctgtttcagctaatttccaaagagacgttgagatcacttggggtgatggtcgtggacagatcaagaacaatggagagcttctcactttatctctagataaatcctctggttctggattccaatccaaaaacgagtacttgtttggtaaagtctccatgcaaatgaagcttgtccctggaaactccgcaggaacagtcacaacactttacttgaaatcacctggaacaacatgggacgagatagatttcgagtttttagggaattcaagtggagaaccttacacacttcacacaaatgtctacacacaaggcaaaggagacaaagaacaacaattcaaactctggtttgatccaacagctaatttccacacttacactattctctggaacccacaaagaatcattttcaccgtcgatggaactccgatcagagaattcaagaacatggagtctctaggcactctgtttcccaagaacaaaccaatgagaatgtactcgagtctttggaacgctgatgattgggcaacgagaggtggtttggtcaaaaccgattggtctaaagctcctttcactgcttcttaccgtggctttcaacaagaagcttgtgtttggtcaaacggcaagtcttcttgtcctaatgcctcgaaacaggggactactactggctcgtggttgtcacaagagcttgactcaacagctcaacaaaggatgagatgggtgcagaggaactacatgatctataattattgtacggatgcgaagaggttccctcaaggtcttcctaaagagtgcttagctgcatagagagagtaaagagttgagagaggaacaagattttatttttctttgtggttataaaattctattcattttattgtagatcacgtgaattttattgatttgttttgtagtatactctatagttcgttaaagttataatattctctttgttacaatgtgcttttttgtt

SEQ ID NO: 38 - protein, 284 aa, Arabidopsis thaliana
MAITYLLPLFLSLIITSSVSANFQRDVEITWGDGRGQIKNNGELLTLSLDKSSGSGFQSKNEYLFGKVSMQMKLVPGNSAGTVTTLYLKSPGTTWDEIDFEFLGNSSGEPYTLHTNVYTQGKGDKEQQFKLWFDPTANFHTYTILWNPQRIIFTVDGTPIREFKNMESLGTLFPKNKPMRMYSSLWNADDWATRGGLVKTDWSKAPFTASYRGFQQEACVWSNGKSSCPNASKQGTTTGSWLSQELDSTAQQRMRWVQRNYMIYNYCTDAKRFPQGLPKECLAA

SEQ ID NO: 39 - DNA, 1386 bp, Arabidopsis thaliana
cccctccttctcccgttctccggtcctcatgcctgccggaatcagctggtcggtttggactctcaatggagagtgtggaagcttccaagcaaatatggtggtatctggtgtcaaatagtgtttctttattatcttctgcccatagatcttaaacgtaaaagcatccaaagatgcattaattaacgataacatggggatgaggtccatgtgcctctgcaacacctcttatgcaatcacattctcttgcttccctaagttactgctgccaacattgaaagaaacacaaagtgcagtgattttaataatcaaatattatgttaagacgtaagagcaagctgattgatggctataaatagaaatgtccgcatagtgtttgtttggtgtgatcagagtgagaatgggtcgtctttcatcaactctctgcctcacgtttctgattttggccacagttgcatttggtgtacctcctaaaaagtccattaatgttccatttgggaggaactacttccccacttgggcttttgatcacattaagtatctcaatggtggttctgaagtgcatcttgtcctcgacaagtacactggcactggctttcagtccaaaggttcctacttgtttggccacttcagtatgcacataaagatggttgctggtgactctgctggaactgtgaccgccttctatttgtcgtcccagaactcagaacatgatgagatagactttgagtttctagggaacagaactggccagccttacattttgcagactaacgtgtttacaggaggcgccggaaaccgtgagcaacgaatcaacctctggtttgatccttccaaggactatcattcctattcagttctctggaacatgtatcagattgtgttctttgtggatgatgtgccgatacgagtgttcaagaatagcaaagacgttggggtgaagtttccattcaaccaaccaatgaagatatattcgagtctgtggaatgcagacgactgggccacaaggggagggttggagaagaccaactgggaaaaagctccttttgtcgcgtcctacagaggcttccacgtcgatggatgtgaagcttcagtaaatgctaagttctgcgagacgcagggaaaacgttggtgggatcagaaagagttccaggacttggacgctaaccagtacaagcgtctcaaatgggttcgtaagagatacaccatctacaactattgcactgaccgcgtccgatttcccgtacctcctccagagtgccgcagagaccgcgacatatagtgatttttcagattttatctagattgttttcccactgtatgacaatatttcctacttttgtaatgtttattcttcttattacaatttaatctagaaaagtttggatt

SEQ ID NO: 40 - protein, 293 aa, Arabidopsis thaliana
MGRLSSTLCLTFLILATVAFGVPPKKSINVPFGRNYFPTWAFDHIKYLNGGSEVHLVLDKYTGTGFQSKGSYLFGHFSMHIKMVAGDSAGTVTAFYLSSQNSEHDEIDFEFLGNRTGQPYILQTNVFTGGAGNREQRINLWFDPSKDYHSYSVLWNMYQIVFFVDDVPIRVFKNSKDVGVKFPFNQPMKIYSSLWNADDWATRGGLEKTNWEKAPFVASYRGFHVDGCEASVNAKFCETQGKRWWDQKEFQDLDANQYKRLKWVRKRYTIYNYCTDRVRFPVPPPECRRDRDI

SEQ ID NO: 41 - DNA, 1073 bp, Arabidopsis thaliana
ttcattgagtcaagtgctcgagactgtagagtgaagtttgatttatttagagagataagttaaaaaaaaaatggtcggtatggatttgttcaaatgtgtaatgatgataatggtgttggttgtctcttgtggtgaagctgtttctggagctaagttcgatgagctttatcggtcaagctgggctatggatcattgtgtcaacgaaggcgaagtcacaaaactcaagcttgacaattactctggagctgggtttgaatcaagaagcaaatacttgtttggtaaagtctctatccagattaagcttgttgagggtgattcagcaggaacagtcactgctttctacatgtcttcagatggtccgaaccacaacgaattcgattttgagttcttagggaacactaccggcgagccttacatagtccagaccaatatctacgtgaacggagttggaaacagagaacaaagactcaacctttggttcgatcccaccactgagtttcacacttactcaatcctctggagtaaacgcagtgttgtattcatggtagacgaaacaccgattcgagttcagaagaatcttgaagagaaaggtatcccatttgctaaagatcaagcaatgggtgtttacagctccatttggaatgcagatgattgggctacacaaggaggtcttgttaaaactgattggagtcacgctcctttcgttgcttcttacaaagaattccaaatcgatgcatgtgagattccaacaactactgatctaagcaagtgtaatggagaccagaagttttggtgggatgaaccaactgtctctgagcttagccttcatcagaatcatcagcttatttgggttcgagctaatcatatgatttatgattattgttttgatgctactaggtttcctgttactcctcttgagtgccaacatcatcgtcatttgtagtttagagattagtcaagaataaaaatggatcaagagaacacatgtggttactctttgatgattctgttttaatgtataaataatcaatcttctattgattttggtgaagtttttaagttcggattttaac

SEQ ID NO: 42 - protein, 290 aa, Arabidopsis thaliana
MVGMDLFKCVMMIMVLVVSCGEAVSGAKFDELYRSSWAMDHCVNEGEVTKLKLDNYSGAGFESRSKYLFGKVSIQIKLVEGDSAGTVTAFYMSSDGPNHNEFDFEFLGNTTGEPYIVQTNIYVNGVGNREQRLNLWFDPTTEFHTYSILWSKRSVVFMVDETPIRVQKNLEEKGIPFAKDQAMGVYSSIWNADDWATQGGLVKTDWSHAPFVASYKEFQIDACEIPTTTDLSKCNGDQKFWWDEPTVSELSLHQNHQLIWVRANHMIYDYCFDATRFPVTPLECQHHRHL

SEQ ID NO: 43 - DNA, 1296 bp, Arabidopsis thaliana
aattttctcccccacaccaaaagataaacctctctctctctatctatcttatgtcaaaagacagatttattaacaaacacaaatcataaatctttttcttcttccgagaaagaatgtctaaatcatcgtataatcacatcttcatcttgatcttgtgtttatgtttgagatcatcatcagccttcacgaatctcaacaccttaagctttgaagaatcactttctcctctctttggagatgccaatctcgtccgttcacctgatgatctctccgttcgtcttctcctcgatcgctataccggttctggtttcatatcatcgaatatgtatcaacatggattttacagctccatgattaagcttcccgctgattatactgccggcgtcgtcgtcgccttttatacatcaaacggagacgtgttcgagaaaacacacgacgagttagacatagagtttctagggaacataaaaggaaagccgtggaggtttcagacaaatctatacggaaatggaagtacacatagaggtcgtgaagagagatatcgtctctggtttgatccttctaaggagtttcatcgttatagcattctctggactcctcacaagatcatattttgggtagatgatgtgccaataagagaagtgatcagaaacgatgcaatgggagcagattatccggcaaagccaatggctctttacgccaccatttgggacgcttctgattgggctacttccggcggcaaatacaaagctaattacaaatttgcccctttcgtagctgaattcaaatctttctctctagacggttgctccgttgatcctattcaagaagtccccatggattgttccgactcagtcgatttccttgagtctcaagattactcttccattaattcccatcaacgcgccgccatgcgaagatttcgacaacgtttcatgtactattcttactgttacgatacacttaggtatcctgagcctctaccggaatgtgtgattgttccggctgagaaagataggtttaaggaaacgggaaggttgaagtttggtggtacggaggcgcgtgaacggcggagaaatcgccggcagcaacgtcggcctgagattgagattgagagtgatcctgacgacagaaaacttttataagatttgtttgtgccattctttttacttaaaattcgtttgtgagtataataaaataaagagtaaagatttgaaaaagaaagaaagaaatagaggataaatttgataggcctttgggctttcatgaattgtgaatgatttaagctcattcatt

SEQ ID NO: 44 - protein, 343 aa, Arabidopsis thaliana
MSKSSYNHIFILILCLCLRSSSAFTNLNTLSFEESLSPLFGDANLVRSPDDLSVRLLLDRYTGSGFISSNMYQHGFYSSMIKLPADYTAGVVVAFYTSNGDVFEKTHDELDIEFLGNIKGKPWRFQTNLYGNGSTHRGREERYRLWFDPSKEFHRYSILWTPHKIIFWVDDVPIREVIRNDAMGADYPAKPMALYATIWDASDWATSGGKYKANYKFAPFVAEFKSFSLDGCSVDPIQEVPMDCSDSVDFLESQDYSSINSHQRAAMRRFRQRFMYYSYCYDTLRYPEPLPECVIVPAEKDRFKETGRLKFGGTEARERRRNRRQQRRPEIEIESDPDDRKLL

SEQ ID NO: 45 - DNA, 1078 bp, Arabidopsis thaliana
aacaaacctaagctcaaagcccaccaaaaagaaaacaatcacttagcaatggcgatgatcagttactccaccattgtggttgctctgcttgcttcttttatgatttgctctgtttccgccaattttcaacgagacgtagagataacttggggagacggacgtggacagatcacaaacaacggcgatcttcttacactctctctcgacaaagcttcaggctcaggattccaatccaagaacgaatatttgttcggtaaaatcgacatgcagatcaaactcgttgccggaaactccgctggaactgtcaccgcttactatttgaaatcccctggatctacatgggacgagattgactttgagttcttgggaaacctaagtggtgatccttacacacttcatactaatgtctttacgcaaggaaaaggcgatagagaacaacaattcaaactctggttcgatcccactagtgatttccacacttactctatcctctggaacccacaacgcatcatattctccgtggatggaactcccataagagaattcaagaacatggaatctcaaggaactctgtttcctaagaaccagccaatgagaatgtactctagtctatggaacgctgaagaatgggccacaaggggtggtctcgtcaaaaccgactggtctaaagctcccttcactgcttcctaccgcggcttcaacgaagaagcttgcgtcgtgatcaacggccagtcttcatgccctaacgtgtcaggacaagggagtactggttcgtggttgtctcaggagctagactcgacgggtcaagaacagatgagatgggtacagaataactacatgatttacaattactgtacggacgctaaaaggttccctcaaggtcttccacgcgagtgcttagctgcgtaaagcttttaaacaaatgatccaaagccatgaaaagtaatactattctttgattcatcttataaagtatctgatgtttggttacatctgttttttatttattctatgtaagtatatttgtattcgaatattgatcaatcactttaccaaaataaaacatttgaacgtacga

SEQ ID NO: 46 - protein, 286 aa, Arabidopsis thaliana
MAMISYSTIVVALLASFMICSVSANFQRDVEITWGDGRGQITNNGDLLTLSLDKASGSGFQSKNEYLFGKIDMQIKLVAGNSAGTVTAYYLKSPGSTWDEIDFEFLGNLSGDPYTLHTNVFTQGKGDREQQFKLWFDPTSDFHTYSILWNPQRIIFSVDGTPIREFKNMESQGTLFPKNQPMRMYSSLWNAEEWATRGGLVKTDWSKAPFTASYRGFNEEACVVINGQSSCPNVSGQGSTGSWLSQELDSTGQEQMRWVQNNYMIYNYCTDAKRFPQGLPRECLAA

SEQ ID NO: 47 - DNA, 1074 bp, Arabidopsis thaliana
atctcacatcacaattcacatctcctcgaacaaacaaattataaacccattttccttcataaatttctaaaataaaaccccttaaactttcattcacatcatccaacccccaatgggtcgaatcttgaaccgtaccgtgttaatgactcttctagtcgtaacaatggccggaacagcattctccggtagcttcaacgaagagtttgacttaacttggggtgaacacagaggcaaaatcttcagtggaggaaaaatgttgtcactctcactagaccgggtttccgggtcgggttttaaatccaagaaagaatatttgttcggaagaatcgacatgcagcttaaactcgtcgccggtaactccgctggaaccgtcactgcctactacttgtcatcggaaggaccaacacacgacgagatagactttgagtttcttggtaatgaaacagggaagccttatgttcttcacactaatgtatttgctcaaggcaaaggaaacagagaacaacagttttatctctggtttgatccaaccaagaacttccacacttattctcttgtctggagaccacaacacatcatatttatggtagataatgttccaatcagagtattcaacaatgcagagcaacttggtgttccatttcccaagaaccaaccaatgaagatatactcgagtttatggaatgcagatgattgggctacaagaggtggtttggttaagacagattggtctaaagctcctttcacagcttactacagaggctttaacgctgcagcttgtactgtttcttcagggtcatctttctgtgatcctaagtttaagagttcttttactaatggtgaatctcaagtggctaatgagcttaatgcttatgggagaagaagattaagatgggttcagaagtattttatgatttatgattattgttctgatttaaaaaggtttcctcaaggattcccaccagagtgtaggaagtctagagtctaaaaaccaatgattctctctttgttgttgtttagtgcaaattaaattctctttgttgtttctttaataaattgatttgatttttcttc

SEQ ID NO: 48 - protein, 291 aa, Arabidopsis thaliana
MGRILNRTVLMTLLVVTMAGTAFSGSFNEEFDLTWGEHRGKIFSGGKMLSLSLDRVSGSGFKSKKEYLFGRIDMQLKLVAGNSAGTVTAYYLSSEGPTHDEIDFEFLGNETGKPYVLHTNVFAQGKGNREQQFYLWFDPTKNFHTYSLVWRPQHIIFMVDNVPIRVFNNAEQLGVPFPKNQPMKIYSSLWNADDWATRGGLVKTDWSKAPFTAYYRGFNAAACTVSSGSSFCDPKFKSSFTNGESQVANELNAYGRRRLRWVQKYFMIYDYCSDLKRFPQGFPPECRKSRV

SEQ ID NO: 49 - DNA, 1037 bp, Arabidopsis thaliana
aatgtgattaagtaaagtgagggagaagcaacgagagatagagatagagagatcaagatgagaggatctgatcagaagattctactgatggttatggtggttgtagcagttgtagctgcggctcaaggccaagaagagactactggattcgtgacatggggtaacaattactaccagacatggggacatcaagctttggttattaataaaacctctgagctccagctcaccctcgataaaaactctgggtccgggtttgaatcccaattgatttacggatcaggatacttcaacgtaagaatcaaggcacctcaaactacgtctacgggagtcattacttccttctatttaatctcccgttcaagccgccatgatgagctctgtttccagatcttgggaaagaatgggccaccgtatttgctgaatacgaatatgtatctgtacggtgaaggaggcaaagatcagaggtttcgtctctggtttgatccaacaaaagattaccattcctacagttttctttggaaccctaatcaacttgtgttttatgttgacgatacaccgatcagggtgtacagcaagaatccagatgtttactacccatcggtgcagacaatgttcctaatgggaagtgtgcaaaacggatcgataattgacccgaagcagatgccttacattgctaagttccaggcatcaaagattgaagggtgtaagactgagttcatgggaatagataaatgcactgatcctaagttctggtggaatcgcaaacagctaagctctaaagagaagacactgtacctaaacgcaaggaagacgtacctggattatgactattgttctgacagacagcgatatccaaaggtgcctcaagaatgtggaagttacacttagagaaagatcaatacctgtcacagggttaagcctacaaaacaattatcttgaaaaaataacttatgttactgtaagatgctttatgataagccttttctatcaaataacaacattattgtcatcaatgctagatacattactatgtc

SEQ ID NO: 50 - protein, 277 aa, Arabidopsis thaliana
MRGSDQKILLMVMVVVAVVAAAQGQEETTGFVTWGNNYYQTWGHQALVINKTSELQLTLDKNSGSGFESQLIYGSGYFNVRIKAPQTTSTGVITSFYLISRSSRHDELCFQILGKNGPPYLLNTNMYLYGEGGKDQRFRLWFDPTKDYHSYSFLWNPNQLVFYVDDTPIRVYSKNPDVYYPSVQTMFLMGSVQNGSIIDPKQMPYIAKFQASKIEGCKTEFMGIDKCTDPKFWWNRKQLSSKEKTLYLNARKTYLDYDYCSDRQRYPKVPQECGSYT

SEQ ID NO: 51 - DNA, 1042 bp, Arabidopsis thaliana
aaaacacaacatgaagattatgtgggaaacagcagttgtgttttgtctctgttcgttgtctttggtttcttcacacagcaggaaattcacaacaccaaacgtgacacgcctcacagatcaattcagtaagatcgccattgaaaatggcttctccagacgtttcggagctcacaatattcaggtcaatggctccctcgctaagctcactctcgacaaatcctccggagctgggttggtgtcaaagaacaagtatcactatggtttcttcagtgcaagactcaagcttcccgctggatttgcctctggtgttgtggttgctttctatttatcaaacgcagagacttatccgaaaagccacgacgagatagacatagaattgttgggtagaagtaggagagatgattggacgatccagacgaatgtatatgcaaatggaagtactagaacgggaagagaagagaagttttatttttggttcgatccaacgcaagcctttcacgactataccctcatttggaactcccaccatactgtatttttggtagacaacattccggttaggcagtttccaaaccggggagccttcacaagcgcgtacccgtctaaaccgatgtctctatacgtcaccgtttgggacggttcagagtgggccactaaaggtggtaagtaccccgtcaactacaagtacgcccctttcgtggtttccgttgctgacgtggagttaagtggctgttccgttaacaacggctcctctaccgggtccgggccatgtaccaagtcgggcgggtcgatttcgagtctggaccctgttgatggtcaggattttgccacgttgtcgaagaatcagatcaatgccatggattgggctaggagaaagctgatgttctattcttattgcagtgataagccaagatacaaagtcatgcctgctgagtgcaactgatttttttttttgttaaaacacacattgttactttctattgttttatgatataattttacgtaggatattagaggtttaaagtatgtttcaaacttcgcc

SEQ ID NO: 52 - DNA, 1042 bp, Arabidopsis thaliana; identical to SEQ ID NO: 51

SEQ ID NO: 53 - protein, 310 aa, Arabidopsis thaliana
MKIMWETAVVFCLCSLSLVSSHSRKFTTPNVTRLTDQFSKIAIENGFSRRFGAHNIQVNGSLAKLTLDKSSGAGLVSKNKYHYGFFSARLKLPAGFASGVVVAFYLSNAETYPKSHDEIDIELLGRSRRDDWTIQTNVYANGSTRTGREEKFYFWFDPTQAFHDYTLIWNSHHTVFLVDNIPVRQFPNRGAFTSAYPSKPMSLYVTVWDGSEWATKGGKYPVNYKYAPFVVSVADVELSGCSVNNGSSTGSGPCTKSGGSISSLDPVDGQDFATLSKNQINAMDWARRKLMFYSYCSDKPRYKVMPAECN

SEQ ID NO: 54 - DNA, 1064 bp, Arabidopsis thaliana
atctcacaccaaaacacaaagctctcatcttcttttagtttccaaactcacccccacaactttcatttctatcaaccaaacccaaatgggtccaagttcgagcctcaccaccatcgtggcgactgttcttcttgtgacattgttcggttcggcctacgcaagcaacttcttcgacgagtttgacctcacttggggtgaccacagaggcaaaatcttcaacggaggaaatatgctgtctttgtcgctggaccaggtttccgggtcaggtttcaaatccaaaaaagagtatttggtcggtcggatcgatatgcagctcaaacttgtcgccggaaactcggccggcaccgtcactgcttactacttgtcttcacaaggagcaacacatgacgagatagactttgagtttctaggtaacgagacagggaagccttatgttcttcacaccaatgtctttgctcaagggaaaggagacagagagcaacagttttatctctggttcgacccaaccaagaacttccacacttactccattgtctggagaccccaacacatcatattcttggtggacaatttacccattagagtgttcaacaatgcagagaagctcggcgttcctttcccaaagagtcaacccatgaggatctactctagcctgtggaatgcagacgattgggccacgagaggtggtctagtcaagactgactggtccaaggctcctttcacagcttactacagaggattcaacgctgcggcttgcacagcctcttcaggatgtgaccctaaattcaagagttcttttggtgatggtaaattgcaagtggcaaccgagctcaatgcttatggcaggaggagactcagatgggttcagaaatacttcatgatctataattattgctctgatctcaaaaggttccctcgtggattccctccagaatgcaagaagtccagagtctgatgaacacatattacctcatatttctctgcttgtttgatgcaattcttaaattcctctgttattccattgtacattgtcaagatcaataaagcattcctggtttcaaaat

SEQ ID NO: 55 - protein, 289 aa, Arabidopsis thaliana
MGPSSSLTTIVATVLLVTLFGSAYASNFFDEFDLTWGDHRGKIFNGGNMLSLSLDQVSGSGFKSKKEYLVGRIDMQLKLVAGNSAGTVTAYYLSSQGATHDEIDFEFLGNETGKPYVLHTNVFAQGKGDREQQFYLWFDPTKNFHTYSIVWRPQHIIFLVDNLPIRVFNNAEKLGVPFPKSQPMRIYSSLWNADDWATRGGLVKTDWSKAPFTAYYRGFNAAACTASSGCDPKFKSSFGDGKLQVATELNAYGRRRLRWVQKYFMIYNYCSDLKRFPRGFPPECKKSRV

SEQ ID NO: 56 - DNA, 1003 bp, Arabidopsis thaliana
atgaagctttcttgtggtacaagctttgcgttcttacttctgtttcttcttgcggcacaatctgtgcatgtctatgcgggtagcttccacaaagacgttcagatacattggggtgatggccgcggaaagattcacgacagagatggaaaacttctttctctctcgctcgacaaatcctctggatccggttttcagtctaaccaggagtttctctatggcaaagccgaggttcaaatgaaacttgttcctggtaattcagctggaacagtgacaacattctatctaaaatctccgggaactacgtgggatgagatcgatttcgagttcttgggaaacataagtggtcatccatatactctccatactaatgtttacacaaaaggcacaggagacaaagaacaacaatttcatctatggttcgacccaaccgttaactttcacacttattgcatcacatggaatcctcaaaggattatttttacggttgatggaattcctattagagagttcaagaaccccgaggcgattggagtcccgttcccaactagacaaccaatgagactctacgcgagtctttgggaagccgagcattgggcaacaagaggaggattagagaaaacagattggtcaaaagctccttttactgctttttatagaaactacaatgtcgatggatgtgtatgggctaatggaaaatcatcttgttccgcgaattccccatggttcactcaaaaactcgattcgaacggccagacaagaatgaaaggtgtgcaaagtaaatacatgatctacaactattgcaccgataaaagaaggtttcctcgaggagttcctgccgagtgcacttaaattcttgattctttgatacatgaatatgttacttggcattgcctttgtaactcaatcaagtttttatttatttttatcatttatttgttgctcatatatacagtatgtggtgtttgtgtaagatgttttaagattgaagtaataaaaataaatt

SEQ ID NO: 57 - protein, 282 aa, Arabidopsis thaliana [sequence omitted]

SEQ ID NO: 58 - DNA, 1059 bp, Arabidopsis thaliana
gaatgatttcaattctctatcttattgacacataaacatggaccggtccactttcatcttatctctactcttcacactcactgtctcgactacgaccctcttctctccggttttcgcaggaacgttcgacacagaattcgatatcacttggggtgatggtcgtggcaaggttcttaacaacggagagcttctcactctctccctcgacagagcctccggctctggttttcaaaccaagaaagagtatttgttcgggaaaatcgacatgcagctcaaactcgtccctggaaactccgccggaaccgtcactgcctactacttgaagtcaaaaggtgatacgtgggacgaaatcgatttcgagtttcttggtaatctaactggtgatccgtataccatgcatacgaatgtgtatactcaaggcaaaggtgatagagaacaacaatttcatctctggttcgatccaaccgctgatttccacacttactctgttttatggaaccctcatcacatcgttttcatggttgatgatattccggtaagagaattcaagaatctacaacatatgggtatccagtatccaaaattgcagccgatgaggttatactcgagtctatggaacgcggaccagtgggctacaaggggaggtctcgttaagactgactggtccaaggcaccattcaccgcttcttacagaaactttagagcggatgcatgtgtttcctcgggcggaagatcatcttgtcctgccggttcacctagatggttctctcaaaggcttgatctaacagcggaagataagatgagagtggtgcagaggaagtacatgatctataactattgtacggacaccaaaaggtttcctcaaggatttccaaaggagtgtagacactagctacgagaattaatgtgtgatccgaaactccaaagctagtattgtataggttcgtctaaattatatatgtaatgtttatgtttatttaggttctgtaataagcaatttcccctgtttctatgtagatattctcataataaaatgggcgtgagttatgttggttcaat

SEQ ID NO: 59 - protein, 282 aa, Arabidopsis thaliana
MDRSTFILSLLFTLTVSTTTLFSPVFAGTFDTEFDITWGDGRGKVLNNGELLTLSLDRASGSGFQTKKEYLFGKIDMQLKLVPGNSAGTVTAYYLKSKGDTWDEIDFEFLGNLTGDPYTMHTNVYTQGKGDREQQFHLWFDPTADFHTYSVLWNPHHIVFMVDDIPVREFKNLQHMGIQYPKLQPMRKSLWNADQWATRGGLVKTDWSKAPFTASYRNFRADACVSSGGRSSCPAGSPRWFSQRLDLTAEDKMRVVQRKYMIYNYCTDTKRFPQGFPKECRH

SEQ ID NO: 60 - DNA, 1059 bp, Arabidopsis thaliana; identical to SEQ ID NO: 58

SEQ ID NO: 61 - protein, 284 aa, Arabidopsis thaliana
MDRSTFILSLLFTLTVSTTTLFSPVFAGTFDTEFDITWGDGRGKVLNNGELLTLSLDRASGSGFQTKKEYLFGKIDMQLKLVPGNSAGTVTAYYLKSKGDTWDEIDFEFLGNLTGDPYTMHTNVYTQGKGDREQQFHLWFDPTADFHTYSVLWNPHHIVFMVDDIPVREFKNLQHMGIQYPKLQPMRLYSSLWNADQWATRGGLVKTDWSKAPFTASYRNFRADACVSSGGRSSCPAGSPRWFSQRLDLTAEDKMRVVQRKYMIYNYCTDTKRFPQGFPKECRH

SEQ ID NO: 62 - DNA, 978 bp, Arabidopsis thaliana
actactcatccccttttccacttcttacttcctcctctttagcccatcagcaaatatcatgaatcttacagaatattcccatattttgtttctttcactatgcaccctcaacttctgcttgtactccataaactccgacgacaacggaggctgggagagaggccatgctaccttctatggtggagctgatgcatccggcacaatgggtggtgcttgtgggtacggtaacttacacagccaaggctatgggctacaaaccgcggctttgagcacggctttgttcaatagtgggcagaaatgtggggcctgctttgagctacagtgtgaggatgatcctgagtggtgcatccctggttccatcatcgtctcggctacaaacttctgtcctccaaactttgccttagctaatgataatggtggttggtgcaatcctcctcttaagcactttgacttggccgagcctgccttcctccagatcgctcagtaccgagctgggatcgttcctgtcgcattcagaagggttccatgtgagaaaggtggagggataaggtttacgataaacgggaatccatatttcgacctcgtgctaatcacgaatgtgggtggtgctggagatataagggccgtctctttgaaaggctcaaagacagatcagtggcaatccatgtcaagaaactggggacagaattggcaaagcaacacttacctcagaggtcaaagcctttctttccaagtcactgatagtgatggtcggactgttgtgagctacgatgttgtgcctcatgattggcagttcggtcagacttttgaaggcggacaattttagatttatgatcaagtgaaactaatagacgttatatccaagaaaagtcaatgattgtgtttaagataaaactctgcttttcattgatcataaccactcttcgatatataatagtcataagaaagaaatacaatgaaacaattaattttgtttgg

SEQ ID NO: 63 - protein, 255 aa, Arabidopsis thaliana
MNLTEYSHILFLSLCTLNFCLYSINSDDNGGWERGHATFYGGADASGTMGGACGYGNLHSQGYGLQTAALSTALFNSGQKCGACFELQCEDDPEWCIPGSIIVSATNFCPPNFALANDNGGWCNPPLKHFDLAEPAFLQIAQYRAGIVPVAFRRVPCEKGGGIRFTINGNPYFDLVLITNVGGAGDIRAVSLKGSKTDQWQSMSRNWGQNWQSNTYLRGQSLSFQVTDSDGRTVVSYDVVPHDWQFGQTFEGGQF

SEQ ID NO: 64 - DNA, 989 bp, Arabidopsis thaliana
acatgtaaattcacatatctctctctcttatttgatcataaagatattattgcttcttcaagtaaacaaaaaatgggaagctttctcttcctcatcgtagtcatcttcctcttctcttcatccgttaacgcttgtgatcgatgtcttcaccgttctaaagcagcttatttctcctctgcctctgctctctcttctggagcttgtgcttatggctctatggctacgagtttcttcgccggacatatcgctgcagctatcccttctatctacaaagacggtgctggctgtggagcttgctttcaagtcagatgcaagaaccctaagctgtgtagcactaaaggaaccattgtgatgatcacagacttaaacaagagtaaccaaaccgatcttgtccttagtagcagagcttttagagctatggctaaacctattgttggtgctgacaaagaccttctcaaacaaggcattgtcgacatcgaataccaaagagttccttgcgattacggcaacaagaacatgaacgtgagagtagaagaagcaagcaagaaaccaaactacttagagataaagcttttataccaaggaggtcaaacagaagtagtatccatcgacattgctcaagtcggttcatcgccaaattggggttacatgacaagaagccacggagctgtttgggtaactgacaaagtacccaccggagctatccagttcaggttcgtagtaaccggtggatacgacggtaaaatgatttggtcacagagtgttcttccatccaattgggaagctgggaaaatttacgacgccggtgttcaaatcactgacattgctcaagaaggttgtgatccttgcgatgctcacatctggaactaagttatttagtggcacacaaacaaacatctttcataggtttatatataatgtgtgacagaggttattaatgaacatagttaaagaatgcttaatctaatgattctcatgattctacattg

SEQ ID NO: 65 - protein, 265 aa, Arabidopsis thaliana
MGSFLFLIVVIFLFSSSVNACDRCLHRSKAAYFSSASALSSGACAYGSMATSFFAGHIAAAIPSIYKDGAGCGACFQVRCKNPKLCSTKGTIVMITDLNKSNQTDLVLSSRAFRAMAKPIVGADKDLLKQGIVDIEYQRVPCDYGNKNMNVRVEEASKKPNYLEIKLLYQGGQTEVVSIDIAQVGSSPNWGYMTRSHGAVWVTDKVPTGAIQFRFVVTGGYDGKMIWSQSVLPSNWEAGKIYDAGVQITDIAQEGCDPCDAHIWN

SEQ ID NO: 66 - DNA, 1105 bp, Arabidopsis thaliana
ccatattattcacgattctcatcaaatcatctccgatactcacaaccgaaataactaacccctcctcaacaaaaaacaacaaaacatgtacactccatcatacttaaaatattcaataatctcaattatatccgtattattcctccaaggaactcatggagacgacggaggttggcaaggtggtcacgccacgttttacggcggcgaagatgcttccggcaccatgggcggagcttgtggctatggaaatttgtatggccaaggttacgggacgaacacggcggctttaagtacggctctattcaacaacggactcacgtgtggcgcgtgctatgagatgaagtgtaacgatgacccgaggtggtgtctcgggtcaaccatcaccgtcacagctacaaacttttgcccacctaaccctggcctctccaacgataatggaggttggtgcaatcctcctcttcagcatttcgacctcgccgagccagcttttcttcagatcgctcagtatcgtgccggcattgttcctgtctctttccgaagagtaccatgtatgaagaaaggaggaataaggtttacgatcaacggacactcatacttcaacctcgttctgatctccaacgtaggaggagcaggagacgtacacgccgtctcaatcaaaggctcaaaaacacagtcgtggcaagcgatgtctagaaactggggacaaaactggcagagcaattcatacatgaacgaccaaagcctttccttccaggtaacgaccagcgatggtcgcacactcgttagcaacgacgtggctccttctaattggcagttcggacaaacctaccaaggtggtcagttctgatccaaaccatcatccacatctctctgttttgggtgctgacgtggctgcatattgctgaggtggctcgtaagcacccgcttaattagcttagccttttttttctcttatttacgaattattgcttcaatggttgtattttcattgtgcctacaaaaaagcaaggtttttttacatgtttattggatttttttcttctctttataagccaatatcgacgcccaaaagaatgaaattacacatgaattgatttaataattt

SEQ ID NO: 67 - protein, 253 aa, Arabidopsis thaliana
MYTPSYLKYSIISIISVLFLQGTHGDDGGWQGGHATFYGGEDASGTMGGACGYGNLYGQGYGTNTAALSTALFNNGLTCGACYEMKCNDDPRWCLGSTITVTATNFCPPNPGLSNDNGGWCNPPLQHFDLAEPAFLQIAQYRAGIVPVSFRRVPCMKKGGIRFTINGHSYFNLVLISNVGGAGDVHAVSIKGSKTQSWQAMSRNWGQNWQSNSYMNDQSLSFQVTTSDGRTLVSNDVAPSNWQFGQTYQGGQF

SEQ ID NO: 68 - DNA, 1396 bp, Arabidopsis thaliana
ccaattctaaaccaaacaacagattctcataatcatctcttcttttttcctctttacgaaaagaagaaagatcaaaccttccaagtaatcattttctttctctctctcacacacacacattcactagttttagcttcacaaaatgtgatctaacttcatttacctatatgcaggtttacacaaaaagaaaaaagaacgatggctcttgtcaccttcttgtttattgctacccttggagcaatgacgtcacatgtcaatggttacgccggaggaggttgggtcaacgcacacgccacattctacggtggtggtgatgcttccggcacaatgggaggtgcttgtggatacggaaacctatatagccaaggctatggaaccaacacggcggcgctaagcacggctctattcaataatggtctaagttgtggtgcttgcttcgagataagatgtcaaaacgatggaaaatggtgtcttcctggctcaattgtcgtcacagccacaaacttttgccctcctaacaacgccttaccgaacaacgcaggaggttggtgtaaccctcctcagcagcattttgatctctctcagcccgtatttcaacgcatcgctcaatacagagccggcattgtccccgtcgcttaccgaagagtgccgtgcgtgagaagaggaggaataaggtttacgataaacggacactcttacttcaacctagttctgatcactaacgtcggaggagccggagatgttcactcagcgatggttaaaggttcaagaactggatggcaagcgatgtcaagaaactggggacagaactggcagagtaactcttaccttaacggacaatctctctcattcaaagttacaacaagcgatggccaaaccattgtctctaacaacgttgctaacgcaggctggtctttcggccagaccttcacaggtgcgcagctacgttaggaagagtgattcggtgaaaattcatctcattgatcgtgtggtattgacgtgttgtagtagaagcagttagagagaggggcatgatagtaatttggtcctttctttcaattgaggttaacctaaaaagaagtggtgcttcgagtgcttgattttgcacgaggccttgatgatgtcatcttttgggaaccttttcttatctttcttcatttttattggtaaggttttatgttatactgatgcagaggtggtattgagttgaagtaccacccgctagtagtagtagtagtctctcgtgtcatttgtatcccttctcgaagcgagagggagagttttagattttattaatctcgttaaagtcatttgtatgttgtaaatttttcaatttctacaagtaagaaatattggagatttgtttgttatatactttgtgtaataaattatattc

SEQ ID NO: 69 - protein, 250 aa, Arabidopsis thaliana
MALVTFLFIATLGAMTSHVNGYAGGGWVNAHATFYGGGDASGTMGGACGYGNLYSQGYGTNTAALSTALFNNGLSCGACFEIRCQNDGKWCLPGSIVVTATNFCPPNNALPNNAGGWCNPPQQHFDLSQPVFQRIAQYRAGIVPVAYRRVPCVRRGGIRFTINGHSYFNLVLITNVGGAGDVHSAMVKGSRTGWQAMSRNWGQNWQSNSYLNGQSLSFKVTTSDGQTIVSNNVANAGWSFGQTFTGAQLR

SEQ ID NO: 70 - DNA, 1366 bp, Arabidopsis thaliana
aaacccaacaacataatttcacatatctctctttctttctcttgaaggaaagacgaagatctccaagtcccaagttgttaacacaagacgtaaacatgggtcatcttgggttcttagttatgattatggtaggagtcatggcttcttctgtgagcggctacggtggcggttggatcaacgctcacgccactttttacggtggtggtgatgcttccggcacaatgggtggtgcttgtggatatggtaatctatatagccaaggctacgggacgagcacggcggctctaagcacagctctcttcaacaatggacttagctgtggttcttgctttgagataagatgtgaaaacgatggtaaatggtgtttacctggctcaatcgttgtaaccgctacaaacttctgcccgccaaataacgcgttagcgaacaataatggcggttggtgtaatcctcctcttgaacactttgaccttgctcagcctgtttttcaacgcattgctcagtacagagctggaatcgtccctgtttcctacagaagggttccttgcaggagaagaggaggaataagattcacgataaacggccactcatacttcaaccttgtgctgatcacaaacgtcggtggtgccggagacgttcactcggcggcgatcaagggttcaagaacagtgtggcaagctatgtcaaggaactgggggcaaaattggcaaagcaactcttacctcaacggtcaagcactttcctttaaggtcaccaccagcgacggccgcacagttgtctccttcaacgccgctcctgccggctggtcttatggccagacttttgccggtggacagttccgttaaaaagggcaagttggttaatctctcttccatttatctaaagtaaactcatttgtgtggttatattggtctcttgaaaaaactcggttattgagagagtgatgcgtcgagggctcggttttgcagaaggccttgatgacgtctaatctttttttggacctctttatttttctttcttgaaactagtttttgttaagaaagaaaaaacaagttatagtagttaatgtattactgatgcagaggtggagttttaactaccacccgctagtagtagttatgagttttttattttaaggtgtgagagagagatggattatcaagatttgtcaattttattatgtttgtttgtaataatacaattctttactccagttaatgaaaattgggggattgatcacttttttttgcattttttttcacttttcatatcaacataccaaattttacaaccattatagactattttgggaactttatctttcgtttcaaatttttacaaacattattgaatgctcatttagctt

SEQ ID NO: 71 - protein, 249 aa, Arabidopsis thaliana
MGHLGFLVMIMVGVMASSVSGYGGGWINAHATFYGGGDASGTMGGACGYGNLYSQGYGTSTAALSTALFNNGLSCGSCFEIRCENDGKWCLPGSIVVTATNFCPPNNALANNNGGWCNPPLEHFDLAQPVFQRIAQYRAGIVPVSYRRVPCRRRGGIRFTINGHSYFNLVLITNVGGAGDVHSAAIKGSRTVWQAMSRNWGQNWQSNSYLNGQALSFKVTTSDGRTVVSFNAAPAGWSYGQTFAGGQFR

SEQ ID NO: 72 - DNA, 1249 bp, Arabidopsis thaliana
acattcctctcttctctctctcttttcccaaaaaccaaaaccttttttccgtaccaaggtcaacaaatggcggcgaaagtaattacatttatggcggttatggtggttactgcctttacggccaacgccaaaatccccggagtttacaccggtggtccttggatcaatgctcacgccaccttctacggtgaagctgacgcttccggcactatgggtggtgcgtgtgggtacgggaatttgtatagccaaggttacggagtgaacacggcggctttaagcactgccttattcaacaatggtttgagttgtggctcttgctttgagcttaagtgtatcaatgatccaggatggtgtcttcctggaaacccatcaattcttatcaccgccactaatttctgccctcctaactttaatcaagctagcgacaatggtggttggtgtaatcctcctcgtgagcactttgatctcgctatgcctatgtttctttccatcgctaagtataaggctggtatcgtccccgtctcttaccgcaggatcccatgtaggaagaagggaggaatcagattcacaatcaatggattcaagtacttcaacttggtgctggtcactaacgtagccggagctggagacgtcattaaggtgagtgtgaaaggatccaacacacaatggttagatctgagccggaactggggacaaaactggcaatccaacgcacttcttgtcggccaatctctttctttccgagtcaaaacctctgatggccgaagctccacctccaacaacattgctcccagtaactggcaatttggccagacctactccggcaagaacttccgcgtctgatttctagaatatgaaaaagagtcgtgtggcggaagatactaaagcttttttttatatcaaatatatatatttacgtgtctttgatttagtttacgatttggaattggggttttaccatttttcccttattttctcagggaaagtattgggattgcttggtttgcaagttagggtttactttaaagattagggctatattgttttttttgttttttttggtgtgttgtgtcttgtttggtatgtagtcttgtagagaggctgaagcggctgcagaaagaaagggttgggataaagattcatgtagcccgcagctctcttttacgttatgattgtattaaagtatatatatatcatgaagaaattaaacatggtttcttcgatttatataaataaataattggatttt

SEQ ID NO: 73 - protein, 258 aa, Arabidopsis thaliana
MAAKVITFMAVMVVTAFTANAKIPGVYTGGPWINAHATFYGEADASGTMGGACGYGNLYSQGYGVNTAALSTALFNNGLSCGSCFELKCINDPGWCLPGNPSILITATNFCPPNFNQASDNGGWCNPPREHFDLAMPMFLSIAKYKAGIVPVSYRRIPCRKKGGIRFTINGFKYFNLVLVTNVAGAGDVIKVSVKGSNTQWLDLSRNWGQNWQSNALLVGQSLSFRVKTSDGRSSTSNNIAPSNWQFGQTYSGKNFRV

SEQ ID NO: 74 - DNA, 1202 bp, Arabidopsis thaliana
gtccctatccaatacacatctctctctctcttattaactcaatgacggcgactgcgtttagggtcggcttgtggttggccgttacggcttcctttctcttaaccgcaacaaacgccaaaatccccggcgtttacagtggcggaccgtggcagaatgcacacgccactttctacggtggcagtgacgcctccggcacaatgggcggcgcgtgtgggtacgggaacttgtacagccaaggatacggtgtgaacacggcggcgttgagcactgctttgttcaacaacggattcagctgtggtgcttgttttgagattaagtgtactgatgatccgagatggtgtgttccgggaaatccatctattcttgtgacggcgacgaacttttgtccgccgaattttgctcagccgagtgacgacggagggtggtgcaatccgccgcgcgagcattttgatctcgccatgcctatgttcctcaagatcggtctataccgtgcaggcattgtccccgtctcctatcgcagggtaccttgtcggaagataggagggataagattcacagtaaacggattcagatacttcaatcttgttctggtaactaacgttgccggcgccggagatattaacggagttagcgtaaagggatcaaagacagattgggtgaggatgagtcggaactggggacagaactggcaatccaacgccgttctcatcggccaatcactctctttccgagtcaccgcctctgaccgacgttcttctacctcatggaacgttgctcctgccacgtggcagtttggtcagactttctccggcaaaaactttcgagtctgaaatctgaaaaaccacccaaaagatgttttttaattttattttttacaatggaagaagacacaaacaagaagagagatgagacattttccagccctgggttttccctttaattttcttgggaatattccggtggcttgtggaaaatatttccgtttttagtattattatcagcaaaatttcttttaatctttgcgatttacgagtgatttgtttttctcttggttattttgttcctttaattatttcgttggtggcatcttcgttctgctaataagccaaagagtgtcgaacagtcagtgatcgagtcgaatggaccgtgtaatgggccattgggccaagttttcttgatataaaatctgaaatactactaaa

SEQ ID NO: 75 - protein, 262 aa, Arabidopsis thaliana
MTATAFRVGLWLAVTASFLLTATNAKIPGVYSGGPWQNAHATFYGGSDASGTMGGACGYGNLYSQGYGVNTAALSTALFNNGFSCGACFEIKCTDDPRWCVPGNPSILVTATNFCPPNFAQPSDDGGWCNPPREHFDLAMPMFLKIGLYRAGIVPVSYRRVPCRKIGGIRFTVNGFRYFNLVLVTNVAGAGDINGVSVKGSKTDWVRMSRNWGQNWQSNAVLIGQSLSFRVTASDRRSSTSWNVAPATWQFGQTFSGKNFRV

SEQ ID NO: 76 - DNA, 1142 bp, Arabidopsis thaliana
aaaacatgtcagagcaatcttttgtcgcatccccacaagtcttgaaaagcggagcctgatgatcaaaaggaacccccaaaactctctcaaacgcatcacttttgtctgccacctgtgactgactgtgaggatcccaacaacacttcttgttcatggattctgggcttcagcaactcgcattgtgcctcttcttcattctctgccgcctctttcaagccactgccgaagatgactggaagatagccacagccacgctttctagagacagagacggctcctcctccgtcgctactggaggcgcttgtgggtatggagatctgaggcagagcagctttgccggctacagcgcaggcctgagcgggaagctgttcaacaggggaagcagctgcggagcttgtctagaagtgcggtgcgtgaaccacatccgttggtgtcttcaaggcagcccctccgtggtggtcaccgccaccgatttctgtcctcccaattcgggactctcctccgattacggaggttggtgcaacttcccaaaggaacacttggaactatctcatgccgccttcacagggatcgcagaaaccagagctgagatgatacctatacagtataggagggtcaagtgtgggcggagaggcgggttgagattcagcttgagcgggagctcccactttttccaggtgttgataagcaatgtgggcctcgacggggaagtggttggtgtgaaagtgaagggccacacaacggcttggatcccaatggccagaaactggggacagaactggcactcctctctcgatctcatcggacagtctctctctttcgaggttactctcaaaggcggcaaaaccattgcctcttatgatgtggctcctccgtattggcgcttcggaatgacataccaaggaaagcagttccactcctgactcctttatcttcttcaaatcatgattcactttcggtgactttattatgactttggagtcattagatatcatcacttcatttcaaggaggtataaatgactttccacgagtggctcatctttgtcttctcttttcttttttctttttatataatatatccttcacaatccttacttggaccgcatttagctgtgtgagagtatattatgcacattgatc

SEQ ID NO: 77 - protein, 256 aa, Arabidopsis thaliana
MDSGLQQLALCLFFILCRLFQATAEDDWKIATATLSRDRDGSSSVATGGACGYGDLRQSSFAGYSAGLSGKLFNRGSSCGACLEVRCVNHIRWCLQGSPSVVVTATDFCPPNSGLSSDYGGWCNFPKEHLELSHAAFTGIAETRAEMIPIQYRRVKCGRRGGLRFSLSGSSHFFQVLISNVGLDGEVVGVKVKGHTTAWIPMARNWGQNWHSSLDLIGQSLSFEVTLKGGKTIASYDVAPPYWRFGMTYQGKQFHS

SEQ ID NO: 78 - DNA, 1358 bp, Arabidopsis thaliana
cactcccatcaaagcaaaactatctttctccttctcattcctttttctctcactctcctccattaaagctctgcactttctcaaagagaatgttcatgggtaagatgggtcttttgggaattgctctgttttgttttgctgcaatggtgtgctctgttcatggctatgacgctggatgggtcaatgctcatgctaccttctatggtggaagtgatgcttcaggaacaatgggtggagcttgtggctacgggaacctctacagtcaaggttacgggaccaacacggcggcgttgagcactgctctgttcaacaacggtcttagctgcggggcttgttttgagatcaagtgtcagagcgacggcgcgtggtgtttacctggtgctatcattgtcacagccaccaatttctgtcctcctaacaacgctcttcccaataacgctggtggttggtgtaaccctccgcttcatcatttcgatctctctcagcctgtttttcaacgcattgctcagtacaaagctggtgttgtccctgtttcctacagaagggttccgtgtatgagaagaggaggtataagattcacaatcaacggtcactcttacttcaaccttgtcttggtgaccaatgttggtggtgctggagatgttcattcggttgcggttaaaggttctagaacaaggtggcaacaaatgtcaagaaactggggacagaactggcaaagcaacaatctcttaaacggtcaagcattgtcatttaaggtgactgctagtgatggtcgtaccgtcgtctctaacaacattgctccagctagttggtcctttggacaaaccttcaccggccgtcaattccgttaaaattgagtcaagttcggttttatatagttttagggtttgtgtagtagttggttgaggaaagagtagagaaagagagagggtttaaggctttttagggtttttaagtggagagccttgaaacttcttctttgactcttgagggttaaaatggagaaaagagcttgttttataagggctcttttagtcatgttaaggattagggttttagtaagtgggtgttgagtagtagagtggtgtgtcttaagggacctctatgtttcaccaatgggttcctttgttttatcacttttttattttgttttatcttcttttttcaagcttctgtcttgtggtttaaaaagcagaagtgggtaggggcagaggaggaatttcaccacccgcctatgtgtttttctttttgggtcgttttggtttatgttatgaagttgtaattgtaatgtagatcaaccaatgggtttgataagtgttttgttaccatactatgaattttatctgttttgtgtgatt

SEQ ID NO: 79 - protein, 253 aa, Arabidopsis thaliana
MFMGKMGLLGIALFCFAAMVCSVHGYDAGWVNAHATFYGGSDASGTMGGACGYGNLYSQGYGTNTAALSTALFNNGLSCGACFEIKCQSDGAWCLPGAIIVTATNFCPPNNALPNNAGGWCNPPLHHFDLSQPVFQRIAQYKAGVVPVSYRRVPCMRRGGIRFTINGHSYFNLVLVTNVGGAGDVHSVAVKGSRTRWQQMSRNWGQNWQSNNLLNGQALSFKVTASDGRTVVSNNIAPASWSFGQTFTGRQFR

SEQ ID NO: 80 - DNA, 1174 bp, Arabidopsis thaliana
aaaccaaaattaaatcatcaaaaatcaaaatctctaaaatcaaaaattcgcattttctctctcgccggagagatgcaacggtttcttctacctttactcttccttgcactatcgccgccggcgatttgtcattactcttcctcaacctcttcaccttcttcgtcctccgtttcttccgacgcctcagaatggcgtccagctcgagccacctactacgccgctacgaatcctcgagacgcggtgggcggtgcgtgtggatacggagatctcgtcaaatctgggtacggtatggctactgttggtctaagcgagactctgtttgagcgtggtcagatctgtggtgcttgtttcgagctcagatgtgttgatgatctccgttggtgtatccctggaacttcaattatactcaccgctacgaatttctgtgctcctaattacggatttgatcccgacggtggtggtcattgtaatccacctaacaaacatttcgtgcttccgatcgaagcgtttgagaagatcgctatttggaaagctgggaacatgccggtgcagtatcgaaggatcaactgtagaaaggaagggagcatgcggtttacagtcgatggtggaggcattttcatttcggttttgatcaccaatgttgcagggtccggtgatatagctgctgtgaagatcaaagggtcgagaaccgggtggttacctatgggtcgtaattggggacaaaattggcatattaatgctgatctcaggaaccaagctctctcatttgaagtaacttctagtgacaggtcaaccgtgacatcttacaatgtctccccgaaaaactggaattatggacagaccttcgaagggaaacaattcgagactccgtgagacagagacggacacattgagatacaatcccatgattctttttttccgggtcagaattttttgatggcaagtatatcgatcttggtctaaggttatctttagggttgaaaaggaaagaacttgggtcagacttttgcatttcatagttctctctctcccacgcataggtacaaaaactcaccaatgtactaagaacagtgatcacacaaacatcactattgtatctatttgtttacattactaaggagcgaaagcttccttcaagtgcttaaagctaatgatgagtactgagtcttatcgt

SEQ ID NO: 81 - protein, 266 aa, Arabidopsis thaliana
MQRFLLPLLFLALSPPAICHYSSSTSSPSSSSVSSDASEWRPARATYYAATNPRDAVGGACGYGDLVKSGYGMATVGLSETLFERGQICGACFELRCVDDLRWCIPGTSIILTATNFCAPNYGFDPDGGGHCNPPNKHFVLPIEAFEKIAIWKAGNMPVQYRRINCRKEGSMRFTVDGGGIFISVLITNVAGSGDIAAVKIKGSRTGWLPMGRNWGQNWHINADLRNQALSFEVTSSDRSTVTSYNVSPKNWNYGQTFEGKQFETP

SEQ ID NO: 82 - DNA, 2360 bp, Arabidopsis thaliana
acatttcttcacttccacacacttttacttctttctctcttctcttctcttctccagatctgatcccaaacctttgattcattgttgttgttctctgctgctttatcagagagcatcatcatgtacggaagagatccatggggaggtccattggagataaacactgcagattccgccaccgacgatgatcgtagtcggaatttaaacgatttggatcgtgcggctctttcacgtccactagatgagacgcagcagagttggttacttggtccaacggagcagaagaagaagaagtacgtcgatctcggttgtattatcgttagccgcaagatcttcgtctggactgttggtactcttgttgccgccgcgttactcgccggattcattaccttgatcgttaaaactgtgccgcgtcatcatcctaagactccgccgccggataattatactatagctctacacaaagctcttaagttcttcaatgctcagaaatctgggaaattgccaaagcataataacgtgtcatggagaggtaattctgggcttcaagatgggaaaggtgaaacaggaagcttctataaagatttggtgggaggttattatgatgctggtgatgctatcaagttcaatttccccatggcttatgctatgactatgttgagctggagtgttattgaatatagtgctaaatacgaagctgctggtgagctcactcatgttaaggagcttatcaaatggggaactgattactttctcaagactttcaatagtactgctgattccattgatgatcttgtgtcacaggttggatcagggaatactgatgatggaaatacagatcctaatgaccattactgttggatgcgacctgaggatatggactataaaaggcccgtgactacttgtaatggtggatgttcggatctcgctgcagagatggcagctgctctggcttcagcatctattgtattcaaggataacaaggaatattctaaaaagcttgtccatggtgctaaggtggtgtatcagtttggaaggacgaggagagggagatatagtgcaggcactgcggaatctagcaagttctataattcaagtatgtattgggatgagttcatttggggtggtgcttggatgtattatgctaccggaaatgtaacgtatctcaatctaatcacccaacctactatggccaagcatgctggtgccttctggggtggcccttactatggtgtatttagctgggacaacaagcttgctggtgctcagttgctgttgagccggttgaggttgtttctgagtcctggatatccatatgaagaaattctaaggaccttccacaatcagaccagcatagtcatgtgctcatacttgcctattttcaacaaatttaacagaaccaatggaggtttaatagagttgaatcatggagctccacagccgctgcaatattctgtaaatgcagctttcttagcgactctatacagtgattatctggatgctgctgatactcctggatggtactgtggacctaatttctattcgacaagtgtgctacgtgactttgctagatcccagattgattatatactgggtaaaaaccctcggaaaatgagttatgtcgttggttttggcacaaaatacccaagacatgtgcatcacagaggagcttcgatacccaagaacaaagtcaagtataactgcaaaggaggatggaaatggagagacagcaagaaaccaaacccaaacacgattgaaggagccatggttgctggtcctgacaagcgcgacgggtaccgtgatgtccgtatgaactacaactacactgaaccgactcttgcaggcaatgctggtctagtcgcagctcttgtggcattatcgggtgaagaagaagccaccggtaagatagacaaaaacactattttctcagctgttcctcctttgttccctactccaccacctccaccagcaccatggaaaccttgagaaagctagacttgtgtgattctgtcgctgctgccaaaaaaaatgaatgaggtaagaaggatttgggtgtgagaccagaagattagaagctaaacacaagtcagccataaccaaactactaaggatttcatttggctttactagatacaaacacggggtgggttactttaccacaagcattgtctttcttttctttttttgggttgctgttttgttcttgtgagatatcatatatatctatgcgttttactctgtatatgtttgataccaaacttgtattctttgataaacaatttaatgaactgtattaaacttttaactatgttttattgtgcaagtgtgagatcaacctggaataacaactgtagtctact

SEQ ID NO: 83 - protein, 621 aa, Arabidopsis thaliana
MYGRDPWGGPLEINTADSATDDDRSRNLNDLDRAALSRPLDETQQSWLLGPTEQKKKKYVDLGCIIVSRKIFVWTVGTLVAAALLAGFITLIVKTVPRHHPKTPPPDNYTIALHKALKFFNAQKSGKLPKHNNVSWRGNSGLQDGKGETGSFYKDLVGGYYDAGDAIKFNFPMAYAMTMLSWSVIEYSAKYEAAGELTHVKELIKWGTDYFLKTFNSTADSIDDLVSQVGSGNTDDGNTDPNDHYCWMRPEDMDYKRPVTTCNGGCSDLAAEMAAALASASIVFKDNKEYSKKLVHGAKVVYQFGRTRRGRYSAGTAESSKFYNSSMYWDEFIWGGAWMYYATGNVTYLNLITQPTMAKHAGAFWGGPYYGVFSWDNKLAGAQLLLSRLRLFLSPGYPYEEILRTFHNQTSIVMCSYLPIFNKFNRTNGGLIELNHGAPQPLQYSVNAAFLATLYSDYLDAADTPGWYCGPNFYSTSVLRDFARSQIDYILGKNPRKMSYVVGFGTKYPRHVHHRGASIPKNKVKYNCKGGWKWRDSKKPNPNTIEGAMVAGPDKRDGYRDVRMNYNYTEPTLAGNAGLVAALVALSGEEEATGKIDKNTIFSAVPPLFPTPPPPPAPWKP

SEQ ID NO: 84 - DNA, 2247 bp, Arabidopsis thaliana
agcagcagcagcacacttctcaagatctctctctgtctttgtcatcaccactctatcgtctactagagggaagagatcatcattctacagtttcaagaagataccatttccaagacttgaaactgtggaagaagcaaaaatggggactttgagtaaggtgctatgtctcatgctgctttgctgtatcgtggctgctgctgagggtactttgaagtacaaggacccaaagcagcctttgggtgcaagaatcagagatttgatgaaccgtatgactctacaagagaagattggtcagatggttcagatcgaacgcagcgtcgctacacctgaagtcatgaagaaatacttcattgggagtgttttgagtggtggaggaagtgtgccctcggaaaaagctactcctgaaacttgggtgaacatggtcaatgagattcaaaaggcgtctctttcgacccgccttgggatccctatgatctacgggattgatgctgttcacggtcacaacaatgtgtatggcgccaccattttcccgcacaatgtaggccttggagtcaccagggatcctaaccttgttaagaggattggggcagcaaccgcgcttgaagtcagagcgactggaatcccatatgcctttgccccgtgtattgcagtttgtagggatccaagatggggaagatgctacgagagctacagcgaggattatagaattgtccaacagatgactgagattatacccggtttgcaaggcgaccttcctaccaagcgaaagggtgttccctttgttggtggaaagacaaaagttgcagcttgtgcaaagcattttgttggagatggaggtacagtgagaggaattgatgagaacaacacagtgattgactcgaagggcctgttcggaattcacatgcctggatattacaatgctgtaaacaagggtgttgcaacaataatggtgtcttactctgcctggaacggtttgagaatgcatgctaacaaggaacttgtcacgggtttcctcaagaacaagttgaagttcaggggttttgtcatctctgattggcaggggattgataggatcacgactcctcctcatcttaactattcatactctgtttacgcgggaatcagtgctggaattgacatgattatggttccgtacaactacactgagttcatagacgaaatcagcagtcagatacagaaaaagcttattccgattagcaggatcgatgacgccctgaagagaatcttaagggtcaaattcacaatgggactctttgaggaaccactggctgatctcagctttgccaaccagcttggtagcaaggaacatagggaactagctcgtgaagccgtgaggaagtctctggtgctgctcaagaatggtaagacaggtgctaaacctctgcttcctctgcctaagaaatcagggaagatccttgtcgcgggagcacatgctgataacttgggatatcaatgtggtggctggaccatcacctggcaaggccttaatggcaacgaccataccgttggtacaacaatcctagcggctgtgaagaatacagtggctcctaccacacaagtcgtctacagccaaaaccccgatgcgaactttgtgaagtcgggtaaatttgactatgccattgtggtagtcggggagccaccttatgctgaaatgtttggggacaccacgaacctgaccataagtgatcctggtccgagcataataggaaacgtgtgcggatcagtgaaatgtgtggtggttgtggtctctggccgtcccgtggtgattcagccttatgtttcgaccattgacgctctcgtggcggcttggcttccgggaacagaaggtcaaggagtggctgatgctctgtttggtgattacgggttcacaggaaagttggcccggacgtggtttaagtcggtgaagcagctaccgatgaatgttggagatcggcattacgacccattgtacccattcggtttcgggttgaccacaaaaccatacaaaatgtagactcccttttagttattcggcaaggagaggacaatattaagggacaaaacgaaataacttcgatgcacaattttgtgggttctagctctcaagttaaggttgaggctttgagattgtctgtctacaaaactagtatgttataaacagtgatgatatgaatcagcttttgttatttgcccgtatgattctgtattttgttccattttatgagatatctaacatttgggcctctc

SEQ ID NO: 85 - protein, 624 aa, Arabidopsis thaliana
MGTLSKVLCLMLLCCIVAAAEGTLKYKDPKQPLGARIRDLMNRMTLQEKIGQMVQIERSVATPEVMKKYFIGSVLSGGGSVPSEKATPETWVNMVNEIQKASLSTRLGIPMIYGIDAVHGHNNVYGATIFPHNVGLGVTRDPNLVKRIGAATALEVRATGIPYAFAPCIAVCRDPRWGRCYESYSEDYRIVQQMTEIIPGLQGDLPTKRKGVPFVGGKTKVAACAKHFVGDGGTVRGIDENNTVIDSKGLFGIHMPGYYNAVNKGVATIMVSYSAWNGLRMHANKELVTGFLKNKLKFRGFVISDWQGIDRITTPPHLNYSYSVYAGISAGIDMIMVPYNYTEFIDEISSQIQKKLIPISRIDDALKRILRVKFTMGLFEEPLADLSFANQLGSKEHRELAREAVRKSLVLLKNGKTGAKPLLPLPKKSGKILVAGAHADNLGYQCGGWTITWQGLNGNDHTVGTTILAAVKNTVAPTTQVVYSQNPDANFVKSGKFDYAIVVVGEPPYAEMFGDTTNLTISDPGPSIIGNVCGSVKCVVVVVSGRPVVIQPYVSTIDALVAAWLPGTEGQGVADALFGDYGFTGKLARTWFKSVKQLPMNVGDRHYDPLYPFGFGLTTKPYKM

SEQ ID NO: 86 - DNA, 1875 bp, Arabidopsis thaliana
ggaggcgcgtgtgaaggaccttctctctcgcatgactttgccggagaagattggtcagatgactcagatagaacgctccgtcgcttctcctcaagtcattactaattctttcatcggcagtgtacagagcggcgccggaagttggccgttggaggatgctaagtcgtcggattgggctgatatgatcgacggttttcagcgttcagcgttagcgtcgcgtttgggaattccaataatctacggcactgacgccgttcacggcaacaataacgtctacggcgccaccgtcttcccccacaacattggactcggcgccaccagagacgcagatttggtcaaaagaattggtgctgcaacagcacttgaaataagggctagtggcgttcactggacgtttgctccttgtgtggccgttttgggagatccgaggtggggacgatgctatgaaagttatagtgaagctgctaaaattgtatgtgagatgagtttgcttatctcgggcctacaaggcgagccacctgaagaacacccttacggttacccttttcttgcaggaagaaacaacgtgatcgcgtgtgctaaacactttgttggagatggtggtactgagaaaggcttaagcgaagggaacactataacatcatatgaagacttggagaaaatacatgttgctccctatcttaattgtattgctcagggagtgtcgaccgttatggcctcattttccagttggaatggaagtagactccactccgactatttccttttgactgaagttcttaaacagaaactcggtttcaagggatttctagtttcagactgggatggtttggagacgataagcgagccagaaggttccaactaccgtaactgcgtcaaactcgggattaatgctggaatcgacatggtgatggtgcctttcaagtacgaacagttcatacaggacatgacagatttggtggagtcaggggaaataccaatggctcgggtcaatgatgctgttgaaagaatactaagagtgaagtttgttgctggtctctttgaacatcctcttgcggaccgatctttgttgggaactgttggctgcaaggaacatagagaagtggcacgtgaagcggttagaaagtcattagtgttgctgaagaatggtaaaaatgctgatacaccatttctaccactagaccgcaatgccaagaggattctagtggttggaatgcatgcaaacgatcttggaaatcagtgtggaggatggacaaagataaaatccggtcaaagcggcagaatcacaattggcacgacgcttttggattcaataaaagcagcagttggagacaaaactgaagtcatcttcgagaaaactccaacaaaggaaaccttagcatcaagtgatggcttctcatacgccattgttgcagtgggggaacctccatacgcagaaatgaagggagataactcggaactcactatacctttcaatggtaacaacattataaccgcggttgcagagaaaatcccgactctagtgattttgttctcaggacgtccaatggttttagagccaacggttcttgaaaagactgaagctttggtcgctgcttggttccctggaactgaagggcaagggatgagtgatgtgattttcggagattatgacttcaaagggaagttaccagtgagctggttcaaacgcgttgaccagttgccgttaaacgctgaagcaaattcgtatgacccattgtttcctcttggtttcggtctcacttccaatttcggttaactggtttaacttcaccggttaacttgtattaaaaatatgcgattaccatatgaaccacacgtttaatttaataagttaatcaaattgatgaatttttt

SEQ ID NO: 87 - protein, 581 aa, Arabidopsis thaliana
MTLPEKIGQMTQIERSVASPQVITNSFIGSVQSGAGSWPLEDAKSSDWADMIDGFQRSALASRLGIPIIYGTDAVHGNNNVYGATVFPHNIGLGATRDADLVKRIGAATALEIRASGVHWTFAPCVAVLGDPRWGRCYESYSEAAKIVCEMSLLISGLQGEPPEEHPYGYPFLAGRNNVIACAKHFVGDGGTEKGLSEGNTITSYEDLEKIHVAPYLNCIAQGVSTVMASFSSWNGSRLHSDYFLLTEVLKQKLGFKGFLVSDWDGLETISEPEGSNYRNCVKLGINAGIDMVMVPFKYEQFIQDMTDLVESGEIPMARVNDAVERILRVKFVAGLFEHPLADRSLLGTVGCKEHREVAREAVRKSLVLLKNGKNADTPFLPLDRNAKRILVVGMHANDLGNQCGGWTKIKSGQSGRITIGTTLLDSIKAAVGDKTEVIFEKTPTKETLASSDGFSYAIVAVGEPPYAEMKGDNSELTIPFNGNNIITAVAEKIPTLVILFSGRPMVLEPTVLEKTEALVAAWFPGTEGQGMSDVIFGDYDFKGKLPVSWFKRVDQLPLNAEANSYDPLFPLGFGLTSNFG

SEQ ID NO: 88 - DNA, 2015 bp, Arabidopsis thaliana
aacgaagaagaagagtaaagaaaatggtcgtcgaggagtcatcatgcgtttacaagaacggagacgcgccagtggaggcgcgtgtgaaggaccttctctctcgcatgactttgccggagaagatcggtcagatgactcaaatcgaacgcagagtcgcttctccctccgcctttacagatttcttcatcggaagtgtactaaacgctggaggtagtgtaccgttcgaggacgctaagtcatcggattgggctgatatgattgatggttttcagcgatcagcgttagcgtcgcgtttgggaattccaattatctatggcactgacgctgtccacggcaacaataatgtctacggcgccactgtcttccctcacaacattggcctcggagccaccagagacgcagatttggtcagaagaattggagcagcaactgcacttgaagtaagagcgagcggggttcattgggcattttctccttgcgtggccgttttgagagatccgaggtggggaagatgctatgagagttatggagaagatcctgaacttgtctgtgagatgacttctcttgtctcgggcctacaaggtgtgccaccagaagaacacccaaatggttatccttttgttgcaggaagaaacaacgtcgtcgcgtgtgtcaaacactttgttggagatggtggtactgacaagggcataaatgaagggaacaccatcgcttcatatgaagaattagagaagatacatattcctccttatctgaagtgtcttgctcagggagtttccacggttatggcgtcttactccagttggaatggaactagactgcacgccgaccgttttctcttgacagaaattctaaaggaaaaacttggtttcaagggatttctagtctcagactgggaaggtttggaccggcttagtgagcctcagggatcaaactaccgctactgcattaaaactgcggttaatgctggcattgacatggttatggtaccatttaagtacgaacagttcatacaggacatgacagatctggtagagtcaggggaaataccaatggctcggatcaatgatgctgttgaaagaatactcagagtgaaatttgtcgctggtctttttggacatcctctcacagatcgatctttgttgcctactgttggttgcaaggaacatagagaattggcgcaggaagcagttagaaagtcgttagttctgctaaagagtggcaaaaatgctgataaaccgtttctgccactagatcgcaacgctaagaggattctagtcactggaactcacgcagatgatcttggatatcagtgcggaggatggacaaagacatggttcggtctaagcggcaggatcacaattggcactacacttttggacgccataaaagaagcagttggagacgaaaccgaagtaatctatgagaaaactccatcaaaggaaaccttagcctcaagtgaaggattctcttacgccattgttgcagtcggggaaccgccctatgcagagacgatgggggataactcggaactcagaataccttttaatggtactgacattgtcaccgcagttgcagagataatcccgactctggtgatattgatctcaggacgacctgtggttttggagccgacggttcttgaaaagacagaggctctggttgctgcttggctgcctggaacagaagggcaaggggtggctgacgttgtttttggagattatgacttcaaggggaagttgccagtgagctggttcaaacacgttgagcatttgccactagatgctcatgccaattcttatgacccattgttcccgtttggtttcggtctcaattcaaaaccagtgtaacttcactggtctgattgatgtacaggaacatcggggaattatgttatgacatatattacacgcaatgtcgtatttgtataagcaaaccatgcaaaaacaatgtattgttgatgtgcttttatgaggaaaatatatgagaaggaaaacaaacattggtgttgatgt

SEQ ID NO: 89 - protein, 608 aa, Arabidopsis thaliana
MVVEESSCVYKNGDAPVEARVKDLLSRMTLPEKIGQMTQIERRVASPSAFTDFFIGSVLNAGGSVPFEDAKSSDWADMIDGFQRSALASRLGIPIIYGTDAVHGNNNVYGATVFPHNIGLGATRDADLVRRIGAATALEVRASGVHWAFSPCVAVLRDPRWGRCYESYGEDPELVCEMTSLVSGLQGVPPEEHPNGYPFVAGRNNVVACVKHFVGDGGTDKGINEGNTIASYEELEKIHIPPYLKCLAQGVSTVMASYSSWNGTRLHADRFLLTEILKEKLGFKGFLVSDWEGLDRLSEPQGSNYRYCIKTAVNAGIDMVMVPFKYEQFIQDMTDLVESGEIPMARINDAVERILRVKFVAGLFGHPLTDRSLLPTVGCKEHRELAQEAVRKSLVLLKSGKNADKPFLPLDRNAKRILVTGTHADDLGYQCGGWTKTWFGLSGRITIGTTLLDAIKEAVGDETEVIYEKTPSKETLASSEGFSYAIVAVGEPPYAETMGDNSELRIPFNGTDIVTAVAEIIPTLVILISGRPVVLEPTVLEKTEALVAAWLPGTEGQGVADVVFGDYDFKGKLPVSWFKHVEHLPLDAHANSYDPLFPFGFGLNSKPV

SEQ ID NO: 90 - DNA, 2011 bp, Arabidopsis thaliana
caacaaacaacgagaaaaatccctaatacatgagtatgcgtgtttaacactaccttaaatgagattaatgcttttttcccaaaccgttatgattaattattattagtccataaataccccactcaaagacaagccataaagagtgtaagaaagaagagagcacacaagaacaacaaaacagaggaagaagaagaagaagatgaagcttctggctctgtttccatttctagcgatcgtgatccaactcagctgttgggagctaggaacagatgcattaccgagcggtgggttcgtgaggacgaaaggtgttcagtttagtctcaatggctatccatattacgctaatggcttcaatgcctactggctcatgtacgtagcctccgatccatcccaacggtctaagatctccaccgctttccaagatgcttctcgccatggattgaccgttgctcgaacctgggctttcagcgatggcggttacagggctcttcagtattcccctggctcctacaacgaggatatgtttcagggtttggattttgcgttagctgaggcaagaaggcatggtataaagataatactcagctttgccaataactacgagagcttcggagggaggaagcaatatgtggattgggctcgaagcagaggccgtcccgtttcttctgaagacgacttcttcactgactctcttgttaaagatttctacaagaaccatatcaaggctgtgctgaacagattcaatacctttaccaaagttcattacaaagatgacccaaccattatggcttgggagctcatgaacgagccccgttgcccctctgatccttccggaagagccattcaggcttggattactgaaatggctgctcatgtgaaatcactagacagaaaccatctgcttgaagctggcctcgaaggtttctatggtcagtcttcacctcaaagcaagactcttaacccacctggccagtttggaaccgatttcatcgccaataaccgcattcccggcattgatttcgtcacggttcactcttaccctgatgaatggtttccagactcaagcgagcaatcccaaatggatttcttgaacaaatggctagacgcacacatccaagacgcacagaacgttcttcacaaaccaataatattagcagagtttggtaaatcaatgaagaaaccaggttataccccagcgcagagagacatcgtcttcaacaccgtgtacagcaagatttacgggtctgcaaaacgaggaggtgcagcagcaggaggattgttctggcaacttctggtaaacggaattgataattttcaagatgggtatgggatcatacttagccaaagctcgtcgaccgttaacgtcatttcacagcaatcgcggaagttgactttgattaggaaaatcttcgctaggatgatcaatgtggagaaatggaagagagcgagaggtcagggacaagttgggaaacgaggtcacaaaatcaataactgaaatgatactaattaaaccacttttttatagcgaatggaacgatctcagctcgtccacgaaagttatagtgatagatttcataatatatagttttttggccggaatgaatgaactttattagtgcgacgaaaattattagtaataacccgtcaaaactcaaaaggttggttatttagtaatgggctttacttattaggcccactatgaaatacaagggcctagaatttctccagccacatgtgagttataacatgggcaaaaccattcaacaatgtttgttttggattctgtgaaagagatgactatcgttgataatttgaaggatgaagaagattcagaacaataaaacgtcgtcggcgtttgatttcagacatcgccggagagaacaagactctccatttgttacttccatggcttatgtgggtaggtcttggcgtaaacttgtattttatattctcgatttgtacggtttaatgattaaagaagaaaatagacaaaaaaaaattatttagcttc

SEQ ID NO: 91 - protein, 431 aa, Arabidopsis thaliana
MKLLALFPFLAIVIQLSCWELGTDALPSGGFVRTKGVQFSLNGYPYYANGFNAYWLMYVASDPSQRSKISTAFQDASRHGLTVARTWAFSDGGYRALQYSPGSYNEDMFQGLDFALAEARRHGIKIILSFANNYESFGGRKQYVDWARSRGRPVSSEDDFFTDSLVKDFYKNHIKAVLNRFNTFTKVHYKDDPTIMAWELMNEPRCPSDPSGRAIQAWITEMAAHVKSLDRNHLLEAGLEGFYGQSSPQSKTLNPPGQFGTDFIANNRIPGIDFVTVHSYPDEWFPDSSEQSQMDFLNKWLDAHIQDAQNVLHKPIILAEFGKSMKKPGYTPAQRDIVFNTVYSKIYGSAKRGGAAAGGLFWQLLVNGIDNFQDGYGIILSQSSSTVNVISQQSRKLTLIRKIFARMINVEKWKRARGQGQVGKRGHKINN

SEQ ID NO: 92 - DNA, 1994 bp, Arabidopsis thaliana
aactactaaaatcggatctataacttttgttctttgagagacttatttctctctcggtttcttactcttttcggatcagaaatttaaaagcttttaacttttgtcttgctctgtttcatggacactgatttctcaattggtctctgtttttgttgagtgggttgagaagagagatggcgagaagtagatcgattagtggttatgggatatggaaatatttgaatcctgcgtattatcttagaagaccgagacgtttggctttgcttttcattgtcttcgtctctgtttctatgcttgtctgggatcgtattaatcttgcccgagaacatgaggttgaagtttttaagctaaatgaagaagtttcacggttggagcagatgttagaagagcttaatggtggtgttggcaataagcctttgaagactctgaaggatgccccagaagatccagttgataaacagcgaaggcagaaagtaaaagaggcaatgatccatgcttggagctcttatgaaaagtatgcatgggggaaagatgagcttcagcctcggacaaaagatggcactgatagctttggtggccttggagcaactatggtagattctttagatacactctatataatgggtctagatgagcagtttcaaaaagccagagagtgggttgcaagctcattggatttcgacaaggattatgacgccagtatgtttgagacaaccataagagttgtaggcggacttcttagtgcgtatgatctttctggggacaaaatgttccttgaaaaggctaaggatattgcagacagattattgcctgcatggaatactccaacgggtataccttacaatattatcaacttgagaaatggaaatgctcacaatccttcatgggcggcagggggagacagtattctcgcagactccggcactgagcagctcgagtttattgccctttcccaaaggacaggggacccaaaatatcagcagaaggtagagaaggttattacagaactgaataagaactttcctgctgatggtttacttcccatctatataaatccggataatgctaatccatcgtactctaccacaacatttggtgccatgggagatagcttttatgagtatttgctcaaagtttgggtgcaagggaacaaaacatctgccgtgaaaccctatagagatatgtgggagaaatcaatgaaaggtttgttaagcttggtcaagaaatcaacaccttcatcatttacgtatatatgtgagaagaacggaaataatttgattgataagatggatgaattggcgtgctttgctcctggaatgttggctttaggagcttcaggttatggccctgatgaagaaaaaaagtttctttcacttgctggagagcttgcctggacttgttataacttttaccaatcgacaccaacgaaacttgctggagagaactatttcttcactgcagggcaggacatgagtgttggcacatcttggaacattttaagaccagaaaccgttgaatcactgttttacctctggcgattaactgggaacaagacatatcaagagtggggatggaatatatttcaagcatttgagaagaactctcgcgtagaatctggatatgtaggcttgaaggatgtcaatacaggtgctaaagacaacaagatgcaaagcttcttcttagctgagactcttaagtatctatatcttctcttttcgccttcatctgttatttcattagacgagtgggttttcaacacagaagcccatccgcttaagattgtggcacggaatgatccgcgtaagccaactatagcactacgccagaggaagtttggtcatcagattaacgtttagtagacagtttctgctcaaatggaatttgatattattatatattaccacaaagtttgaggcacattgttgtatcagtttaaagaacagatggacaacgtttttgccacagcaatttgtagaggtactttactgtttcct

SEQ ID NO: 93 - protein, 560 aa, Arabidopsis thaliana
MARSRSISGYGIWKYLNPAYYLRRPRRLALLFIVFVSVSMLVWDRINLAREHEVEVFKLNEEVSRLEQMLEELNGGVGNKPLKTLKDAPEDPVDKQRRQKVKEAMIHAWSSYEKYAWGKDELQPRTKDGTDSFGGLGATMVDSLDTLYIMGLDEQFQKAREWVASSLDFDKDYDASMFETTIRVVGGLLSAYDLSGDKMFLEKAKDIADRLLPAWNTPTGIPYNIINLRNGNAHNPSWAAGGDSILADSGTEQLEFIALSQRTGDPKYQQKVEKVITELNKNFPADGLLPIYINPDNANPSYSTTTFGAMGDSFYEYLLKVWVQGNKTSAVKPYRDMWEKSMKGLLSLVKKSTPSSFTYICEKNGNNLIDKMDELACFAPGMLALGASGYGPDEEKKFLSLAGELAWTCYNFYQSTPTKLAGENYFFTAGQDMSVGTSWNILRPETVESLFYLWRLTGNKTYQEWGWNIFQAFEKNSRVESGYVGLKDVNTGAKDNKMQSFFLAETLKYLYLLFSPSSVISLDEWVFNTEAHPLKIVARNDPRKPTIALRQRKFGHQINV

SEQ ID NO: 94 - DNA, 2103 bp, Arabidopsis thaliana
agttgaaaaactcaagaatctcctcggacaagagcagagatccagagtgatgtctgagaagattcattcaacacattaacgaaactccccaaaatagcttttattgatggactcaaatttcaagtggcttttgtttgccatcttgatctctctgacattttcgggttttgttcttcaccatggagttctcgctgaaagtgttaagcctgatgaagctaaacagcttagagacgaggtacgtgggatgttttatcatgcctttgatggatatatgaacaatgcgtttccactcgatgaattgagaccgttatcttgccaaggagaagatactcttggaggctatgcgttgactctgattgactcgttggatacattagctttacttggtgaccgagagcgcttcacttcttctgttgaatggattggtaaaaatcttcagtttaatataaataaaacagtctctgtgtttgagacaactatccgagtccttggggggttactatctgctcatctgattgcaagtgattatgcaacgggcatgagaattccatcttacaacaatgaattactagttttggctgagaatttggcacggagaatgcttcctgcatttgatacaccgactggaatcccatttggatctgtgaatttgatgtacggtgttgacaaacatgaaagcaagataacatcaactgccggtggtggtactttgtctttagagtttggcgtgcttagtcgtttaacaaatgatcctgttttcgaacaagttgcaaagaatgcggtgaggggactatgggcacgtcgttcaaatctcgacttggttggtgctcacatcaatgtctttacaggtgaatggacacagaaggatgctggtataggaacaagcattgattccttctatgagtatctactcaaggcttatatactatttggggatgaggaatatctatacatttttcaagaagcttatagatctgcaatgcagtaccttcacaaggatccttggtatgtagaagtcaatatggattccgcagctattgtctggccagtatttaacagccttcaagctttctggccaggacttcaggttttagctggagacgttgatccagcaataagaactcacactgccttctttagcgtctggaaacgatatggtttcacccctgagggttttaatcttgctacacttagtgtccagtatgggcaaaagagttatcccttaaggccggaattaatcgagagtacatactggttgtacaaagctaccagagatcccaggtatcttgatgcaggacgtgactttgtggctagtttgcaatacggggcaaaatgtccttgcggttattgtcacatcacagatgtagaactgcataaacaagaagatcacatggagagcttcttcctcgcagaaacggtgaagtacttatggctactctttgacttagctgttgactcggataacctcgtagacaatggcccttacaagtacatcttcagtactgagggtcatctcttaccaataacaccacaaatatctctagctcgggaacattgctcttactttggtggatactgtccaagcaattccacaaaactagaacaagaagtccttggagaagacagtagtaacgatgatcacagtaacgattatccttaccacgaatcttttccagttaccggtttgataaagggtttatgtccaggactaacacatgctcagaaatatggtttttcgtatgttctaccggagaagacagatcgtgaagatgttaatcaacctaaaccggtagttacaagcagttcaattgtgttaatctcggatcaaacagtggagaagagaccacaggaagaagaaggatttacttctcagtctgaaccaataatgacaatctctggtggtagtagcaacgaccaaacaggtcaagagctaaccttgttagagtcagaaactgatgatcaaagatcatactcttcttaaatctctcatttatatatatgttccacagaagaagattacaaaaacttttgttagaaatatagattcagttttccaatctcatcaagaatcttgaatccttcttttgttgatcgattgttgtc

SEQ ID NO: 95 - protein, 624 aa, Arabidopsis thaliana
MDSNFKWLLFAILISLTFSGFVLHHGVLAESVKPDEAKQLRDEVRGMFYHAFDGYMNNAFPLDELRPLSCQGEDTLGGYALTLIDSLDTLALLGDRERFTSSVEWIGKNLQFNINKTVSVFETTIRVLGGLLSAHLIASDYATGMRIPSYNNELLVLAENLARRMLPAFDTPTGIPFGSVNLMYGVDKHESKITSTAGGGTLSLEFGVLSRLTNDPVFEQVAKNAVRGLWARRSNLDLVGAHINVFTGEWTQKDAGIGTSIDSFYEYLLKAYILFGDEEYLYIFQEAYRSAMQYLHKDPWYVEVNMDSAAIVWPVFNSLQAFWPGLQVLAGDVDPAIRTHTAFFSVWKRYGFTPEGFNLATLSVQYGQKSYPLRPELIESTYWLYKATRDPRYLDAGRDFVASLQYGAKCPCGYCHITDVELHKQEDHMESFFLAETVKYLWLLFDLAVDSDNLVDNGPYKYIFSTEGHLLPITPQISLAREHCSYFGGYCPSNSTKLEQEVLGEDSSNDDHSNDYPYHESFPVTGLIKGLCPGLTHAQKYGFSYVLPEKTDREDVNQPKPVVTSSSIVLISDQTVEKRPQEEEGFTSQSEPIMTISGGSSNDQTGQELTLLESETDDQRSYSS

SEQ ID NO: 96 - DNA, 2107 bp, Arabidopsis thaliana
ggcggagactggcggagatgatgaattcataaattccttcttctcctttcctctcttttcatttgattggcttcttacacagcttcgtcatcatgtcttgtcctatccatcctaggcgtctcttcctctgcttgcttatttctctcactttcttcgtcgtcgacccttcatcgcagcatatcgaggtcaagaagaaacaaatgcgagagaaagtacgcgagatgttctatcatgcgtatgacaattacatgacttatgcatttccgcatgatgagctaaagcctctaactaaaagtttcacagactccctcagtgagcttggaaatcttaagcttgaacacctgccaacagattataatggatcagctgttactcttgttgaatcattatccagccttgctatattggggaacagtacagaatttgaaaagggggttctctggctctcagaaaatcttactttcgatattgatgcccgggtcaacctttttgagtgcaatataagagttcttggaggacttatctctgctcatcttcttgcaattgatccaaataataggttgattcagggttcctacaacaatcagcttcttcgattagctgaagaccttggaaaacgttttttacccgcgtttgaaacacctacaggattaccatatgcatggattaatttgaagaatggagtaatggagaatgagacaactgaaacaagcacttcaggatgtggttctctcgttcttgaaatgggagcattgtcacggctcactggtgacccgaggtttgaatcagctgcactacgtgcgcttcgtcagctatggaggatgcgaagttcgttagatctgcttgggacaacattggatgtggtaactggggaatggatagagtactcctctagcattggagctggggttgactctttctatgaatacctcttgaaggcttatattctttttggaaaagaagactactggcgaatgtttcattctgcttatctggcatctcagaagtacttcagacatgggccttggtaccatgaagctaatatgtggagtggaaaaccaacttactggcagctcacaagtcttcaggcgttttggcctggtctgcaggttcttgttggagatattgcagctgcaaattcatcgcatcgtgagtttttccatgtatgggaaaagtttggtgtattacctgagaggtatctactcgatcatcaaataatacatccgacaatgaagtactatccactacgtcctgaattagcagaatccacgttctacctttaccaagctacaaaagatccatggtatctagatgttggtgaatcaatggtaaaatctcttaatctctacacaaaggtgccagggggatttgcaagtgttagagatgtgacgaccatgcaattggaagatcatcagcacagtttctttctcgctgaaacgtgcaagtacctatatctcctcttcgatgactcatttgtggccaaaaggaattatatatttacaaccgagggccatcctatacaggttgtgagctcctggcatgagaaactaccagaaacttatttctcaggcaactggaccctttcaaagagtggagcatgggaaagtcgagctagcgcattgtcactacaagtctgtccactgatatctcttaactctagacatcctgagcaacaaagagagagtgcttgccatgttcttgacgagcaaatcaatcataagtgttggagcaacaaagagtgcggagttgatgccactacttgtagactaagaacctgcagcggggttggatactgcggcttatggaatcccttataacttttgacacaaaacagtggggtcttgggttttaataaaggaaagagaagaggtggagtttacgaactgaaactgtaaattaatgcgaaatgacaagttttggtagacagaaaatggatagttgttgcatgaggctgttgtatctttttctttgattcatcagtcctgacatggatatatagttatatactatgggaggatcaacctcgatcaaaggaatattatgtttttcttgtttttttcttgacgtatagctttatgcctttatcaaatggtaattctaggacctt

SEQ ID NO: 97 - protein, 574 aa, Arabidopsis thaliana
MSCPIHPRRLFLCLLISLTFFVVDPSSQHIEVKKKQMREKVREMFYHAYDNYMTYAFPHDELKPLTKSFTDSLSELGNLKLEHLPTDYNGSAVTLVESLSSLAILGNSTEFEKGVLWLSENLTFDIDARVNLFECNIRVLGGLISAHLLAIDPNNRLIQGSYNNQLLRLAEDLGKRFLPAFETPTGLPYAWINLKNGVMENETTETSTSGCGSLVLEMGALSRLTGDPRFESAALRALRQLWRMRSSLDLLGTTLDVVTGEWIEYSSSIGAGVDSFYEYLLKAYILFGKEDYWRMFHSAYLASQKYFRHGPWYHEANMWSGKPTYWQLTSLQAFWPGLQVLVGDIAAANSSHREFFHVWEKFGVLPERYLLDHQIIHPTMKYYPLRPELAESTFYLYQATKDPWYLDVGESMVKSLNLYTKVPGGFASVRDVTTMQLEDHQHSFFLAETCKYLYLLFDDSFVAKRNYIFTTEGHPIQVVSSWHEKLPETYFSGNWTLSKSGAWESRASALSLQVCPLISLNSRHPEQQRESACHVLDEQINHKCWSNKECGVDATTCRLRTCSGVGYCGLWNPL

SEQ ID NO: 98 - DNA, 2107 bp, Arabidopsis thaliana; identical to SEQ ID NO: 96

SEQ ID NO: 99 - protein, 574 aa, Arabidopsis thaliana; identical to SEQ ID NO: 97

SEQ ID NO: 100 - DNA, 2584 bp, Arabidopsis thaliana
aagtctttcatcacatttccatttttctctcttccataaaacccaaaagaaactaggaagaggagtaaataatatttgttttaaagaaatgattctccacaaaatggcgttcttggccgttattctcttcttcttgataagcagcagcagcgtttgcgttcatagccgtgaaacgtttgcttgcgatacaaaggacgcagcaacagctacactgagattctgccagctttcagttcctataccggagagagtcagagatttgatcggacggttgacattggccgagaaagtgagcttgttagggaacactgcggcggcgataccacgtctaggaatcaaagggtacgagtggtggtcggaggctttacacggcgtttcaaatgtgggacccggtactaagttcggtggggtttaccctgcagccaccagtttccctcaagtcatcaccaccgttgcttctttcaatgcctccttgtgggaatccatcggacgggttgtgtcaaatgaggccagggccatgtacaacggtggagttggtgggcttacgtattggagcccaaacgttaacatattgagggacccacgttggggacgtggacaggaaactcccggtgaagatccagtagtagccggtaaatacgcagcgagctacgtcagagggttacagggaaacgaccgtagccggttaaaagtagctgcttgttgcaaacatttcacagcttacgatctcgataactggaacggcgtcgacagattccatttcaacgctaaggtaagcaagcaagacatagaagacacgttcgacgtaccgttccgtatgtgtgttaaagaaggtaacgttgcgagcattatgtgttcgtacaatcaagttaatggtgttcctacatgtgctgatcctaatctcctcaagaagaccatacgcaatcaatggggtctcaacgggtatatcgtgtctgattgtgactctgtcggtgttttgtacgatacccaacattacactggtactcctgaagaagctgccgctgattccatcaaagctggcttggatttagattgtgggccatttctaggagcccatacaatcgatgcggtgaagaaaaacttgttgcgtgagtccgatgttgataatgccttaatcaacacgctaacagtccaaatgagactaggaatgtttgatggcgatatagcggctcaaccgtacggacaccttggaccggcacacgtgtgtacaccggttcacaaaggactagctctcgaagcagctcaacaaggaatcgtcctactcaaaaatcacggctcgtctctacctctctcaagccaacgtcaccgaactgtcgccgtaattggacctaattcagacgctacggtcacaatgattggtaattatgcaggggttgcttgtggatataccagtccggttcaaggtattaccggttatgctcgaaccattcatcaaaagggttgcgtggacgtacactgcatggatgatagattgttcgatgccgcggttgaagcggctcgtggagctgatgcgacggttcttgtgatgggtttggatcagtctattgaagcggagttcaaggacagaaacagtttgcttttgcctgggaaacaacaagagcttgtctctagagttgctaaggccgctaaaggcccagttatcttagtattgatgtctggtgggcctatcgatatatcttttgctgagaaggatcggaaaattccagcgattgtttgggccgggtatccgggtcaagaaggtggtaccgcaatcgccgatatcttattcggcagtgctaatcccggaggaaagcttccgatgacttggtatccgcaagattatttaaccaatttaccaatgacagaaatgtcgatgcggccggtccattcgaagcggatcccgggtcggacttaccggttctacgacggtccagttgtttacccgttcgggcatggtttgagttacacgcgctttactcacaacatagccgacgcgccaaaagtgattcctatagctgttcgtggaagaaacggcaccgtttcagggaaatcaatccgtgtgacgcacgctaggtgtgatcgtctctctctcggagtccacgtggaagttactaacgttggctcgagagatgggacgcacacaatgcttgtgttctcggctccgccgggtggagaatgggctccgaagaaacagctggttgcttttgagagagtacacgtggcggttggggagaagaagcgtgtgcaggtgaatatacacgtgtgtaagtatttaagtgtagtggaccgagccgggaaccgaaggattccgatcggtgatcatgggattcatattggagatgagagtcatacggtgtcgcttcaagcttctactcttggagtcatcaagtcttgactctgtttttttcttttcacttttcttgttgttcccaaaatatttttaagagattttaatgtttctaacgaaacgaatttgaaaaaggaaatacaaaactagaagaaaatctgtttcttataattcaaaagatgtatttaaaattgaattgtatggcctcggattttttaaaataaaggttgttttcgg

SEQ ID NO: 101 - protein, 768 aa, Arabidopsis thaliana
MILHKMAFLAVILFFLISSSSVCVHSRETFACDTKDAATATLRFCQLSVPIPERVRDLIGRLTLAEKVSLLGNTAAAIPRLGIKGYEWWSEALHGVSNVGPGTKFGGVYPAATSFPQVITTVASFNASLWESIGRVVSNEARAMYNGGVGGLTYWSPNVNILRDPRWGRGQETPGEDPVVAGKYAASYVRGLQGNDRSRLKVAACCKHFTAYDLDNWNGVDRFHFNAKVSKQDIEDTFDVPFRMCVKEGNVASIMCSYNQVNGVPTCADPNLLKKTIRNQWGLNGYIVSDCDSVGVLYDTQHYTGTPEEAAADSIKAGLDLDCGPFLGAHTIDAVKKNLLRESDVDNALINTLTVQMRLGMFDGDIAAQPYGHLGPAHVCTPVHKGLALEAAQQGIVLLKNHGSSLPLSSQRHRTVAVIGPNSDATVTMIGNYAGVACGYTSPVQGITGYARTIHQKGCVDVHCMDDRLFDAAVEAARGADATVLVMGLDQSIEAEFKDRNSLLLPGKQQELVSRVAKAAKGPVILVLMSGGPIDISFAEKDRKIPAIVWAGYPGQEGGTAIADILFGSANPGGKLPMTWYPQDYLTNLPMTEMSMRPVHSKRIPGRTYRFYDGPVVYPFGHGLSYTRFTHNIADAPKVIPIAVRGRNGTVSGKSIRVTHARCDRLSLGVHVEVTNVGSRDGTHTMLVFSAPPGGEWAPKKQLVAFERVHVAVGEKKRVQVNIHVCKYLSVVDRAGNRRIPIGDHGIHIGDESHTVSLQASTLGVIKS

SEQ ID NO: 102 - DNA, 2477 bp, Arabidopsis thaliana
ctctgtcactctcttacactcatcactttctcatctcttttccgcatttcgaagccatggctaaacaactgcttcttcttctccttctcttcatcgtccacggcgttgaatcagctcctccgccgcactcctgtgacccttcaaatccaaccacgaaactctaccagttctgccgcactgacctcccaattggcaagcgagcgcgtgatctcgtctcgaggctaaccatagacgagaagatctcgcagctggtgaatacagctccagggataccgcgtcttggagttccggcgtacgaatggtggtcagaagctttacacggcgtagcctacgccggtccaggaatccggtttaacggaacggttaaagctgctaccagctttcctcaagtcatcttgaccgctgcttcattcgactcctatgaatggttccgcattgctcaagttataggaaaggaagcaagaggagtgtacaacgcagggcaagcgaatggaatgacattttgggcaccaaacattaacatatttagagatccacggtggggaagaggccaagaaactcccggcgaggatccgatgatgaccggaacttacgcggtggcttacgttagaggactacaaggtgactcttttgacggtcggaaaacactctccaatcatctccaagcctccgcttgttgtaagcattttacagcgtacgatcttgatcgttggaagggtattactcgctacgtcttcaatgctcaggtgagtttggctgacttggcggagacgtaccaaccaccgttcaagaaatgcatagaagaaggtcgagccagtggcatcatgtgtgcttataaccgagtcaacggcattccttcttgcgctgacccgaatcttttgactcgaaccgctcgtggtcaatgggctttccgcggatacattacttcagattgtgacgcagtctcaatcatctacgatgctcaaggctacgctaaaagccctgaagacgctgttgctgatgtcctcaaagctggcatggacgtaaactgtggatcatacttacaaaaacacaccaaatcagctcttcaacagaagaaagtgtctgaaacagacattgacagagctctactcaatcttttctctgttaggattcgtctcggtctctttaacggcgatccaaccaaattaccttacggaaacattagtcctaacgaagtttgttcaccggctcatcaagctttagcacttgatgctgctcgtaatggtatagttctcttgaaaaacaatcttaaactccttcctttctccaaacgcagcgtttcgtctttagctgtgatcggtccaaacgctcacgtcgttaaaacgcttcttggaaactacgctggtccgccttgtaagactgtgactcctcttgatgccttacgtagttatgtcaaaaacgctgtttaccaccaaggatgtgattccgtggcttgctctaacgctgcgattgatcaagcggttgcaattgcaaaaaatgctgatcatgtggttttgattatgggactagaccagactcaagagaaagaagattttgaccgtgtggaccttagtcttcccgggaagcaacaagaactcataacaagcgttgcaaatgctgcaaagaaaccggtggtccttgttctgatatgtggcggtcctgtcgatatttcatttgctgcgaataacaataagataggaagcattatatgggctggttatcctggagaagctggagggattgctatttctgaaatcatattcggagatcacaatcccggagggagattaccagtgacatggtatcctcagagctttgtgaatatacagatgacagacatgagaatgcggtctgcaactggatatcctggaaggacttacaaattctataaaggacctaaagtgtatgagtttggtcatggtcttagctactcagcttactcttaccggttcaagactctggctgaaaccaatctctacttgaaccaatctaaagctcaaaccaactcggactctgttcgatacactctggtttcggaaatggggaaagaaggttgcgatgtagccaagactaaggtcactgtagaggtggaaaatcaaggggagatggcagggaaacatccggtgctgatgtttgcgaggcacgagcgaggaggagaagacggtaaacgagcagagaaacagcttgttgggtttaagagcattgtgttaagcaatggagagaaagcagagatggagtttgagattggtctttgtgagcatttgagcagagctaatgaatttggagtaatggttcttgaagaaggaaagtatttcttaaccgttggagattcagagctccctcttattgttaatgtctgaatctgaaaaacataaacaagaaaaaagacattctccttttgaattggctttttcatatatatttgaaataacatgaaatgaatgtgatttgtgtatctactacatatttaaactgtt

SEQ ID NO: 103 - protein, 767 aa, Arabidopsis thaliana
MAKQLLLLLLLFIVHGVESAPPPHSCDPSNPTTKLYQFCRTDLPIGKRARDLVSRLTIDEKISQLVNTAPGIPRLGVPAYEWWSEALHGVAYAGPGIRFNGTVKAATSFPQVILTAASFDSYEWFRIAQVIGKEARGVYNAGQANGMTFWAPNINIFRDPRWGRGQETPGEDPMMTGTYAVAYVRGLQGDSFDGRKTLSNHLQASACCKHFTAYDLDRWKGITRYVFNAQVSLADLAETYQPPFKKCIEEGRASGIMCAYNRVNGIPSCADPNLLTRTARGQWAFRGYITSDCDAVSIIYDAQGYAKSPEDAVADVLKAGMDVNCGSYLQKHTKSALQQKKVSETDIDRALLNLFSVRIRLGLFNGDPTKLPYGNISPNEVCSPAHQALALDAARNGIVLLKNNLKLLPFSKRSVSSLAVIGPNAHVVKTLLGNYAGPPCKTVTPLDALRSYVKNAVYHQGCDSVACSNAAIDQAVAIAKNADHVVLIMGLDQTQEKEDFDRVDLSLPGKQQELITSVANAAKKPVVLVLICGGPVDISFAANNNKIGSIIWAGYPGEAGGIAISEIIFGDHNPGGRLPVTWYPQSFVNIQMTDMRMRSATGYPGRTYKFYKGPKVYEFGHGLSYSAYSYRFKTLAETNLYLNQSKAQTNSDSVRYTLVSEMGKEGCDVAKTKVTVEVENQGEMAGKHPVLMFARHERGGEDGKRAEKQLVGFKSIVLSNGEKAEMEFEIGLCEHLSRANEFGVMVLEEGKYFLTVGDSELPLIVNV

SEQ ID NO: 104 - DNA, 1490 bp, Arabidopsis thaliana
aaagccttcgcttcaacaacattttttctttagtcgtaaacatattcattcatgattttaacgctttttcttcaaaatatatgaagaagaagacttggttcttgaatttctcactgttctttcttcaaatcttcacatcttcgaatgcattggatgttactcaattcggagcggttggagatggagttacagacgattcacaggcgttcttgaaagcttgggaagctgtgtgtagcggaacaggagatgggcagtttgtcgttccggcagggatgacatttatgttacagcccctgaagtttcaagggtcttgcaaatccacccctgtttttgttcagatgttaggcaagctggttgcaccgagtaaaggaaattggaaaggggacaaagatcaatggattctttttacagacattgaaggacttgtgattgaaggtgacggtgaaattaacggccagggttcgagctggtgggaacacaaaggctctagacctaccgcattgaagttcaggagctgcaacaaccttagattgagtgggttaacgcatttagatagtccaatggctcacattcacataagcgagtgcaactatgtgaccatctcaagcctacgaataaatgcacctgaatcaagtcctaacaccgatggaatcgacgtaggggcttcatccaacgttgtcatccaggattgcatcatcgcaaccggtgatgattgcattgcgattaattcggggacgtctaacatccacatttccggtatagattgcggaccaggccatgggataagcataggaagcttgggaaaagatggagagacagctacagtggagaatgtatgtgtccaaaactgtaactttagaggaactatgaatggagctcggatcaaaacttggcagggcggatcgggttacgcgagaatgattactttcaatggaattactctagacaatgttgaaaatccaatcataatcgatcagttctataacggtggcgattccgataatgccaaagatcgtaagtcgtcggcagtggaagtgagcaaagtggtgtttagtaatttcattgggacgtcaaagtcagagtacggtgttgacttccgatgcagcgagagagttccatgcacagagattttcttgagagacatgaaaatagaaacggcatcatcaggatcaggacaagtcgcacaaggacagtgtttaaacgtgagaggtgcgtctacaattgctgtaccaggtctagaatgtttagaactttccacagatatgttttcatcggcgcaattgctggaacaaacttgcatgtcggcacaatcagtgcaaccaagaacaacaacacaaccgatgcaagatccaatatgggtttttcaaagcagagggaaacaacttagagtttacaatattgccatattagtttcatttatctctttggttacatatattttagctagataggttacatatattatgcgagtaaaatttgtaatctaattg

SEQ ID NO: 105 - protein, 456 aa, Arabidopsis thaliana
MKKKTWFLNFSLFFLQIFTSSNALDVTQFGAVGDGVTDDSQAFLKAWEAVCSGTGDGQFVVPAGMTFMLQPLKFQGSCKSTPVFVQMLGKLVAPSKGNWKGDKDQWILFTDIEGLVIEGDGEINGQGSSWWEHKGSRPTALKFRSCNNLRLSGLTHLDSPMAHIHISECNYVTISSLRINAPESSPNTDGIDVGASSNVVIQDCIIATGDDCIAINSGTSNIHISGIDCGPGHGISIGSLGKDGETATVENVCVQNCNFRGTMNGARIKTWQGGSGYARMITFNGITLDNVENPIIIDQFYNGGDSDNAKDRKSSAVEVSKVVFSNFIGTSKSEYGVDFRCSERVPCTEIFLRDMKIETASSGSGQVAQGQCLNVRGASTIAVPGLECLELSTDMFSSAQLLEQTCMSAQSVQPRTTTQPMQDPIWVFQSRGKQLRVYNIAILVSFISLVTYILAR

SEQ ID NO: 106 - DNA, 1417 bp, Arabidopsis thaliana
acaaggacaaaaaattcacaaacatcaaacattctccacctactgcccaaaaacgacagcattccttttccagcaacaaagaaaaaacacatttccttgccaatggcccgttgtaccaaccttgttaccgttttcctattatgggctcttttgatgttctcatggtgtaaagcttcaagaatcagccctaatgtatatgaccattcttataaaaggttcaaatccgatagcttaatcaagcgaagagaggacatcacgggattgagaagttttgtaagagcctctttgcggactccaaccaccgttagtgtttctgattttggagctaaaggagatggaaaaaccgatgacacgcaggcgttcgtgaatgcgtggaagaaagcatgttcttcaaatggagctgttaatctcctagttcctaaagggaatacttatctccttaagtctattcaattaactggtccatgcaattctattctcaccgttcagatattcggaacgttatccgcatctcaaaaacgatcggattacaaagatatcagcaaatggataatgtttgatggcgttaacaatctatcagtcgatggaggcgacactggggttgtggacggaaacggcgaaacgtggtggcaaaactcatgcaaacggaacaaagctaagccatgcacaaaagccccaacggctcttactttttacaactcgaaaagtctgatagtgaagaatctgaaggtgagaaatgcacagcagatccagatttcgattgaaaaatgctccaacgttcaggtctctaatgtcgtggtaactgcgcctgcggatagtcctaacaccgatggtattcatatcactaacacccaaaacattcgagtctccgaatccatcattggaacaggcgatgattgcatatctattgaaagtggatcacaaaatgttcaaatcaatgatataacttgcggtcccggtcacggtatcagtattgggagccttggagatgacaattcaaaggcttttgtctcaggcgtgactgtggatggtgctaagctttccggtacagacaatggagtaagaatcaaaacttaccagggagggtcaggaactgctagcaatattatatttcaaaacattcagatggataatgttaagaatccgatcataatcgaccaagactactgcgacaagagcaaatgcactacagagaaatccgcggtccaagtgaagaacgtggtgtaccgggacataagtggcactagcgcatcggaaaacgcaataacgtttaactgcagcaagaactatccatgccaaggaattgtgcttgacagagtgaacattaaaggaggaaaagcaacttgcaccaatgctaatgtggttgataaaggagctgttctgcctcagtgcaactccacttagttacttagatcgc

SEQ ID NO: 107 - protein, 433 aa, Arabidopsis thaliana [sequence omitted]

SEQ ID NO: 108 - DNA, 1473 bp, Arabidopsis thaliana
atgatgggaagcatattattgttactattattcttttctctggtccaatcacgttcagacacgtcatattcaaaaattcaacttcccggcgattctctgaccctctccgttaccgatttcggtgctaccggcgatggtatcaactacgatacttctgcgattcagtcaaccattgacgcctgcaatcgtcactacacatctttttcttccatctgccgcgtcgttttcccttccggcaactatttgaccgctaagctccatctccgatctggcgtcattctcgatgtgacggagaacgccgtgcttctcggtggaccgagaatcgaggattattatccggcggagacttcgtcggattggtacgtggtggtggcgaataacgctacggatgttggaatcactggcggaggagcgattgatggccaaggatcgaagttcgtggtcagattcgacgagaagaagaacgtgatggtgagctggaaccaaaccggagcttgcttgggtgatgagtgtagacccaggcttgttggattcgttgattctataaacgttgagatctggaacatcacactacgagagcctgcgtattggtgtttgcacattgtgagatgtgagaacacatcagtccatgatgtatcgatccttggggacttcaatactccaaataacgatggaatcgacattgaagattccaacaacactgttataactcggtgtcatatcgatactggagatgatgcaatctgtcccaagacttacactggtccactttacaacttaaccgctacagactgttggatccggaccaaatcctcagccattaaactcggtagtgcgagctggtttgatttcaaaggtcttgtctttgataacatcactatttttgaatctcacagaggacttggcatgcaaatacgcgatggaggaaatgtgagtgacgttacattttcaaacataaacattagtacaagatactacgatccttcttggtggggaagagcagaaccaatctatataacaacttgccctcgtgactcatctgcaaaggaaggctcaatctcgaatctcctattcgtaaacataacaatcgattccgaaaacggagtctttttatcaggttcaccaaacggactactctcagatataaagttcaagaacatgaaccttactttcagaagatggagtaattacagtgcagggcttgtggactatagacctggatgtcaaggtctagtgaaccatagagccacgtctggaatcattatggagcatgtgaacgggtttcgcgttgagaatgttgacctgaaatggtcagatgatgatgatgtgaatgctgcttggaatgttcctctcgaatttagaccttccactgtgaataatgtctcgtttgttggtttcacttctggtctttacacgaaattgtttgagtctgattacgttatggttggtgaaaacaacattgcttttgcttag

SEQ ID NO: 109 - protein, 490 aa, Arabidopsis thaliana
MMGSILLLLLFFSLVQSRSDTSYSKIQLPGDSLTLSVTDFGATGDGINYDTSAIQSTIDACNRHYTSFSSICRVVFPSGNYLTAKLHLRSGVILDVTENAVLLGGPRIEDYYPAETSSDWYVVVANNATDVGITGGGAIDGQGSKFVVRFDEKKNVMVSWNQTGACLGDECRPRLVGFVDSINVEIWNITLREPAYWCLHIVRCENTSVHDVSILGDFNTPNNDGIDIEDSNNTVITRCHIDTGDDAICPKTYTGPLYNLTATDCWIRTKSSAIKLGSASWFDFKGLVFDNITIFESHRGLGMQIRDGGNVSDVTFSNINISTRYYDPSWWGRAEPIYITTCPRDSSAKEGSISNLLFVNITIDSENGVFLSGSPNGLLSDIKFKNMNLTFRRWSNYSAGLVDYRPGCQGLVNHRATSGIIMEHVNGFRVENVDLKWSDDDDVNAAWNVPLEFRPSTVNNVSFVGFTSGLYTKLFESDYVMVGENNIAFA

SEQ ID NO: 110 - DNA, 2234 bp, Arabidopsis thaliana
ctaacgcagtctctctctctctctctctctctctctctctctctctctctctctctgggcttcggtggtgatcacttgggtcgaatactgatgaagaaactggctgatcggctccttcatcgttttactaatcatgaaaccgctttaaacgccggccaccactgctcagaggggacagatcaaggtacaagtggtcttagtcataggaaggatcacaggatgtcctctcatggtgtccacctgcatgtccatgatcgatatgttgtgatggacaatgggatcctccaagttacactgtcaaagccaggtggaattatcactgggatagagtataacggtattgacaatgtgctcgaagttcgtaacaaggagactaacagagggtactgggacctgcactggaatgaacctggaggcaagggaatatttgatgtcatcagtggagtgactttcagggtcatagtcgagaccgaagaacaggttgagatctcatttctaagaacatgggatccatcccttgagggcaagtacattcccttgaatatcgataaaaggtttataatgctccgtggctcttctggagtgtactcatacggcatttatgaacatcttaaggattggcctggctttgaacttggagaaactagaattgccttcaagctcagaaaagacaaattccattacatggctgtggcagatgacagaaaaaggataatgccttttccagatgatctatgcaaaggaagatgccaaactctagattaccaggaagcttctctgctcactgctccttgtgatccacgcctacaaggcgaagtagatgataaataccaatattcgtgtgagaataaggatctgagagtacatggatggatatccttcgatccaccagtaggattttggcaaattacgcccagtaatgagttccgctcaggcggaccactcaaacaaaacctgacttcacatgttggcccaaccactcttgcagtgtttcacagtactcattatgctggaaaaaccatgatgcctcgttttgaacacggtgagccttggaagaaagtctatggccctgttttcatttacctaaattccacagccaatggagatgatccactttgcttatgggacgatgctaagataaagatgatggctgaggttgaaaggtggccttatagctttgtggcatctgacgactatccaaagtctgaagaacgtggcacagcccgtggtagattacttatccgtgacaggttcataaacaatgatttgatttcagcaagaggtgcttatgttggtttggctccgcctggtgattctggttcttggcaaattgaatgcaagggataccaattttgggctattgcggatgaggctggctatttctcgatagggaacgtgcgtcctggcgagtataatctctatgcttgggtccccagtttcattggagattatcacaacggcacaattgttagagtgacttcaggttgcatgattgagatgggtgatatcgtttatgaacctccaagagatggacctacattatgggaaatcggtatccctgaccgaaaagcttctgagttctttatcccagatcctgatcccactcttgtaaacagggtgttagtccatcaccaagacaggttcaggcaatatgggttatggaagaaatacacagatatgtatccaaatgatgaccttgtttacactgtcggtgtgagtgattaccgcagagactggttctttgctcatgttcccaggaagaaaggagatgtgcatgaaggaacaacttggcagattatatttaatcttgaaaacatcgatcaaaaggccaattacaaactgcgagtcgccatagcatctgcaaccttagccgagttgcagattcgaatcaatgatgcggaagcaatccgccctctcttcacaaccggactcattgggagagacaactcaatagcgaggcacgggatccacggggtttacatgctgtatgcggtgaacataccgggtaaccggcttgtgcaaggtgataatactatattcctgaaacagccaagatgcaatggtccctttcaagggataatgtatgactacattcgtcttgaaggccctccttcttagcttagaatgatatgtgatatcattgtttatgtatagatatataataaaaccaaaagataatggtaacaactgaatcatattgatatgcaaactccccaaaccaaaattctgacaac

SEQ ID NO: 111 - protein, 675 aa, Arabidopsis thaliana
MKKLADRLLHRFTNHETALNAGHHCSEGTDQGTSGLSHRKDHRMSSHGVHLHVHDRYVVMDNGILQVTLSKPGGIITGIEYNGIDNVLEVRNKETNRGYWDLHWNEPGGKGIFDVISGVTFRVIVETEEQVEISFLRTWDPSLEGKYIPLNIDKRFIMLRGSSGVYSYGIYEHLKDWPGFELGETRIAFKLRKDKFHYMAVADDRKRIMPFPDDLCKGRCQTLDYQEASLLTAPCDPRLQGEVDDKYQYSCENKDLRVHGWISFDPPVGFWQITPSNEFRSGGPLKQNLTSHVGPTTLAVFHSTHYAGKTMMPRFEHGEPWKKVYGPVFIYLNSTANGDDPLCLWDDAKIKMMAEVERWPYSFVASDDYPKSEERGTARGRLLIRDRFINNDLISARGAYVGLAPPGDSGSWQIECKGYQFWAIADEAGYFSIGNVRPGEYNLYAWVPSFIGDYHNGTIVRVTSGCMIEMGDIVYEPPRDGPTLWEIGIPDRKASEFFIPDPDPTLVNRVLVHHQDRFRQYGLWKKYTDMYPNDDLVYTVGVSDYRRDWFFAHVPRKKGDVHEGTTWQIIFNLENIDQKANYKLRVAIASATLAELQIRINDAEAIRPLFTTGLIGRDNSIARHGIHGVYMLYAVNIPGNRLVQGDNTIFLKQPRCNGPFQGIMYDYIRLEGPPS

SEQ ID NO: 112 - DNA, 1710 bp, Arabidopsis thaliana
atggtggctcatgagaggaggatccataatcttcaaaaaccaacgtgcatttgcattatctggttttgcctcttggtttctctctctcaccatggaagagcatcatcaacctcagcatctatcttcaatctttctctcccacaccaacacccattccctgaacatgttgttcttaatgttcaaagaaaactcaacgattctctctcaagaagacaacttctcacttaccaacaagacgacggcaccacggcgtcgtcaccaataccttcttgcatcaccggaaacccaatcgacgattgttggcgctgcgacccaaactggtcggcaaaccgccaaagactcgccgattgctcaattggtttcggacaaggaacactcggaggaaaaggcggtcagttttacctcgtcaccgattcctccgacaacgacgcagcgaatccaattcccggaactcttcgacacgccgtgattcagccagaaccactatggatcatcttctccagcgacatgggaatcaaactcaaacacgagctcatcatcggaagctacaagacaatcgatggaagaggcacgaatatccaaatcaccggccatggttgcctcacgattcagcaggttagccacgtcatcatccacaacgttcacattcaccactgtaaaccctccggcaacactttggtcgcttcgtcgcctacacatgttggatttcgaggagtctccgacggagacggaatctcagtatcagcttctcatcacatttgggtcgatcactgttctctcggttactgtgcggacggactcatcgacgtcatcctcgcttccaccgccgtcacaatctccaacaactatttctctcatcacgacgaggttatgctcctaggtcacgacgaccggtacacggctgataaggggatgcaagtaacaattgcattcaaccatttcggagaagggcttgttcagaggatgccgcggtgtaggcacggttatatccacgtggtgaacaacgacttcacggcatgggaaatgtatgcaatcggcggaagtgctagccctacaattaatagtcagggtaaccgttacaccgcacctattgatcccaatgccaaagaggtaacgaagcgcgtggactcaaacgagaaacattggtcgggatggaactggagaacggaaggtgacgttatggttaacggagcttttttcgtgccgtcaggtgacggagtgagcccagcgtatgccagagccaccagtgttcagcctaaagccgccgcgatcattgaccagctcacggtcaatgccggcgttttcggcgatccaagtgggagaaacggccaaggaggaagctttcccggaattacgaacggtggtgggaccatcacgcgcggttacagtaaaagtggacctgccggcggtggcagcggcagtgacagtgatgatgggcttttcacattgatattcggtaacaatagcggtgcggtggctctaaggccgggacaggtctggtcgattctattgattatcattttatattggtatattccacaccatacgagatcatagtttcattattggttcatagttacaaaaaaaaaatgaaaagacaaaaaaacgaatatacagatatataggtacacagcacttgcaagttgtaaaaatatgggaaatgtataagatattataaatcttgtttgatgcctaaactattttggtccc

SEQ ID NO: 113 - protein, 518 aa, Arabidopsis thaliana
MVAHERRIHNLQKPTCICIIWFCLLVSLSHHGRASSTSASIFNLSLPHQHPFPEHVVLNVQRKLNDSLSRRQLLTYQQDDGTTASSPIPSCITGNPIDDCWRCDPNWSANRQRLADCSIGFGQGTLGGKGGQFYLVTDSSDNDAANPIPGTLRHAVIQPEPLWIIFSSDMGIKLKHELIIGSYKTIDGRGTNIQITGHGCLTIQQVSHVIIHNVHIHHCKPSGNTLVASSPTHVGFRGVSDGDGISVSASHHIWVDHCSLGYCADGLIDVILASTAVTISNNYFSHHDEVMLLGHDDRYTADKGMQVTIAFNHFGEGLVQRMPRCRHGYIHVVNNDFTAWEMYAIGGSASPTINSQGNRYTAPIDPNAKEVTKRVDSNEKHWSGWNWRTEGDVMVNGAFFVPSGDGVSPAYARATSVQPKAAAIIDQLTVNAGVFGDPSGRNGQGGSFPGITNGGGTITRGYSKSGPAGGGSGSDSDDGLFTLIFGNNSGAVALRPGQVWSILLIIILYWYIPHHTRS

SEQ ID NO: 114 - DNA, 1588 bp, Arabidopsis thaliana
agtttgggcttcaaaataatcaaacgaaaacaatggaaaaagcaacggagagacaaaggatactgcttcgtcatcttcaaccttcgtcatcttccgacgcctctctctctgcctcagcttgcttgtccaaagacagtgctgcatatcaatatggagatgatgttgtcattgtcgcggcacaaaggactgcactttgcaaggcaaaacgtggcagcttcaaggatacatttccagacgagttgcttgcctctgtattgagagcattgatagagaaaactaatgtaaacccaagtgaagttggtgacattgtagtgggtactgttttgggaccaggatctcagagagccagtgaatgcaggatggctgcgttctatgctggtttccccgaaactgttcccatcagaaccgtgaacagacagtgttcatctgggcttcaggctgttgctgatgttgccgctgccataaaagctggtttttatgacattggtattggagctgggctggagtccatgacaactaatccaaggggatggaaaggatcagtcaacccaaatgtgaagaagtttgaacaagctcacaattgccttcttccaatgggtattacttcagaaaatgtagcacaccggtttaatgtttcaagggaggagcaggatcaagctgctgttgattctcacagaaaggctgcttctgctactgcttccggaaagtttaaggatgagataacccctgtaaaaaccaagattgttgacccaaagacaggtgatgagaaacccataacagtttctgtggatgatgggattcgacctaacacaaccctttccggacttgcaaagctgaagccagtgtttaaggaagacggaaccacaactgctgggaattctagccaattaagtgacggtgctggagctgttctccttatgaggagaaatgtcgcaatgcagaaaggccttcccattcttggtgtattcaggacattttctgcagttggtgtggacccagccatcatgggggttgggccagccgttgccattcctgctgcagtcaaggcagctggtttagaactcaatgacgtcgacttgtttgagattaacgaggcatttgcatctcagtttgtttattgtcggaacaagctcgggctagacgcggaaaagatcaatgtcaatggaggagccatagccattggacatcccttgggcgctacaggagccagatgcgttgcgacgctgctgcatgagatgaaacgacgtggtaaagactgtcgttttggcgtagtgtcaatgtgtataggttcgggaatgggagcagccgctgtgtttgagagaggaggcggtgtggatgagctctgtgatgtccggaaagtctaatgacaataaggccttttgaccaaggaccctagctaaggaccaaattagaacacagtactacaaataaacattatcacaaataaatgcgttctagatgaataaatcataacgatagtacaatacatgagggaaaacttcttgttattttttaactctcttttgttatatggttggaatatatacagatactctttgagaacatatcataatctatttggtttgtc

SEQ ID NO: 115 - protein, 443 aa, Arabidopsis thaliana
MEKATERQRILLRHLQPSSSSDASLSASACLSKDSAAYQYGDDVVIVAAQRTALCKAKRGSFKDTFPDELLASVLRALIEKTNVNPSEVGDIVVGTVLGPGSQRASECRMAAFYAGFPETVPIRTVNRQCSSGLQAVADVAAAIKAGFYDIGIGAGLESMTTNPRGWKGSVNPNVKKFEQAHNCLLPMGITSENVAHRFNVSREEQDQAAVDSHRKAASATASGKFKDEITPVKTKIVDPKTGDEKPITVSVDDGIRPNTTLSGLAKLKPVFKEDGTTTAGNSSQLSDGAGAVLLMRRNVAMQKGLPILGVFRTFSAVGVDPAIMGVGPAVAIPAAVKAAGLELNDVDLFEINEAFASQFVYCRNKLGLDAEKINVNGGAIAIGHPLGATGARCVATLLHEMKRRGKDCRFGVVSMCIGSGMGAAAVFERGGGVDELCDVRKV

SEQ ID NO: 116 - DNA, 1726 bp, Arabidopsis thaliana
tgagaacgatcgatcaaaaatctctcccacgataaaaatgaggaccctgaagctcattgtacttaacgtcttcccctttccagaaaaatcaaccaaacccatttcagtttccccggaaagtttttgtctttcaatcaccattagaattcagctgcaacacaagtgtttgcaacagagagaatcatggagagagctatggaaagacaaaagatattgcttcgtcatctcaatccagtttcttcttctaattcttctcttaaacatgaaccttctcttctgtctcctgtgaattgtgtttctgaagtttccccaatggctgcttttggagatgacattgtgattgtagcggcatatcgtaccgccatttgcaaagcgagacgtggaggtttcaaagacactcttcctgatgatcttcttgcttctgttcttaaggctgtagtggaaagaacatctttggatccaagtgaagttggtgatatcgttgttggtaccgtgatagcgcctggttctcagagagcaatggagtgtagagttgcagcttattttgctggttttcctgactccgtgccagttagaactgtcaatagacaatgctcatcaggactacaagcagttgctgatgttgctgcttccattagagctggttattacgacattggtattggtgctggagtggaatcaatgtcaactgatcatattcctggaggcggctttcatggctctaatccaagagcacaggatttcccaaaagcccgtgattgtttgcttccaatgggaattacttctgaaaacgttgcagaaaggttcggtgtcacaagagaagagcaagatatggctgcggtggagtctcacaaacgcgctgcagctgcaatcgcgtctggtaaactcaaggatgaaatcattcctgttgctactaagattgtggaccctgagactaaagcagagaaggcaatcgtcgtatctgttgatgacggtgtacgtccaaactcaaacatggcagatttggcaaagctgaagactgtctttaaacagaacggttccaccacagctggcaatgctagtcagatcagtgatggtgctggagcagtactgctaatgaagagaagtttggctatgaagaagggacttcccattcttggagtattcaggagttttgctgttactggtgtggaaccatctgtaatgggtattggtccagctgttgccattcccgctgcaactaagctcgcagggctcaacgtcagcgatattgatctattcgagatcaatgaggcatttgcatctcagtatgtgtactcttgcaagaagttagagctggatatggaaaaggtcaatgttaatggaggagccattgctattggccatcccctgggtgctacaggagctcgatgtgttgcgacattgttgcacgagatgaagcggagaggaaaagattgccgctttggagtaatctcaatgtgcataggcactggaatgggagctgcagctgtttttgagaggggagactctgttgataacttgtccaacgctcgtgtggctaacggggatagtcattagaacatcgaagagagcttgaataagtagaagtaatgatgcattgagtctaataaatatgatgctttagctctttcacattgctgaacaatgaaaacttttgtcattctgagtttaaaatcaactacttttctctgaacaaagtgttaaatctaaaaccaaaagttacatc

SEQ ID NO: 117 - protein, 457 aa, Arabidopsis thaliana
MERAMERQKILLRHLNPVSSSNSSLKHEPSLLSPVNCVSEVSPMAAFGDDIVIVAAYRTAICKARRGGFKDTLPDDLLASVLKAVVERTSLDPSEVGDIVVGTVIAPGSQRAMECRVAAYFAGFPDSVPVRTVNRQCSSGLQAVADVAASIRAGYYDIGIGAGVESMSTDHIPGGGFHGSNPRAQDFPKARDCLLPMGITSENVAERFGVTREEQDMAAVESHKRAAAAIASGKLKDEIIPVATKIVDPETKAEKAIVVSVDDGVRPNSNMADLAKLKTVFKQNGSTTAGNASQISDGAGAVLLMKRSLAMKKGLPILGVFRSFAVTGVEPSVMGIGPAVAIPAATKLAGLNVSDIDLFEINEAFASQYVYSCKKLELDMEKVNVNGGAIAIGHPLGATGARCVATLLHEMKRRGKDCRFGVISMCIGTGMGAAAVFERGDSVDNLSNARVANGDSH

SEQ ID NO: 118 - DNA, 1470 bp, Arabidopsis thaliana
catattgtctacttttagattttgatatctattggaactcttactagctcattcatccgtcggcctgtgaattgtgtttctgaagtttccccaatggctgcttttggagatgacattgtgattgtagcggcatatcgtaccgccatttgcaaagcgagacgtggaggtttcaaagacactcttcctgatgatcttcttgcttctgttcttaaggctgtagtggaaagaacatctttggatccaagtgaagttggtgatatcgttgttggtaccgtgatagcgcctggttctcagagagcaatggagtgtagagttgcagcttattttgctggttttcctgactccgtgccagttagaactgtcaatagacaatgctcatcaggactacaagcagttgctgatgttgctgcttccattagagctggttattacgacattggtattggtgctggagtggaatcaatgtcaactgatcatattcctggaggcggctttcatggctctaatccaagagcacaggatttcccaaaagcccgtgattgtttgcttccaatgggaattacttctgaaaacgttgcagaaaggttcggtgtcacaagagaagagcaagatatggctgcggtggagtctcacaaacgcgctgcagctgcaatcgcgtctggtaaactcaaggatgaaatcattcctgttgctactaagattgtggaccctgagactaaagcagagaaggcaatcgtcgtatctgttgatgacggtgtacgtccaaactcaaacatggcagatttggcaaagctgaagactgtctttaaacagaacggttccaccacagctggcaatgctagtcagatcagtgatggtgctggagcagtactgctaatgaagagaagtttggctatgaagaagggacttcccattcttggagtattcaggagttttgctgttactggtgtggaaccatctgtaatgggtattggtccagctgttgccattcccgctgcaactaagctcgcagggctcaacgtcagcgatattgatctattcgagatcaatgaggcatttgcatctcagtatgtgtactcttgcaagaagttagagctggatatggaaaaggtcaatgttaatggaggagccattgctattggccatcccctgggtgctacaggagctcgatgtgttgcgacattgttgcacgagatgaagcggagaggaaaagattgccgctttggagtaatctcaatgtgcataggcactggaatgggagctgcagctgtttttgagaggggagactctgttgataacttgtccaacgctcgtgtggctaacggggatagtcattagaacatcgaagagagcttgaataagtagaagtaatgatgcattgagtctaataaatatgatgctttagctctttcacattgctgaacaatgaaaacttttgtcattctgagtttaaaaaaaaaaaaaaaaaaa

SEQ ID NO: 119 - protein, 414 aa, Arabidopsis thaliana
MAAFGDDIVIVAAYRTAICKARRGGFKDTLPDDLLASVLKAVVERTSLDPSEVGDIVVGTVIAPGSQRAMECRVAAYFAGFPDSVPVRTVNRQCSSGLQAVADVAASIRAGYYDIGIGAGVESMSTDHIPGGGFHGSNPRAQDFPKARDCLLPMGITSENVAERFGVTREEQDMAAVESHKRAAAAIASGKLKDEIIPVATKIVDPETKAEKAIVVSVDDGVRPNSNMADLAKLKTVFKQNGSTTAGNASQISDGAGAVLLMKRSLAMKKGLPILGVFRSFAVTGVEPSVMGIGPAVAIPAATKLAGLNVSDIDLFEINEAFASQYVYSCKKLELDMEKVNVNGGAIAIGHPLGATGARCVATLLHEMKRRGKDCRFGVISMCIGTGMGAAAVFERGDSVDNLSNARVANGDSH

SEQ ID NO: 120 - DNA, 672 bp, Arabidopsis thaliana
atggcacggtttgagccatataactataataatggtcatgatcctttctttgcacacattaaccaaaatccagagctaataaatctggacttaccagcttctaccccttccagtttcatgcttttctccaatggagctttagttgatgccaatcacaataattctcacttcttcccaaatttattgcacggtaatacgagaagaaaaggaaataaagaagagagtgggtcgaagagaagaagaaagaggtcggaagaggaagaagccatgaatggagatgagactcagaagccaaaagatgttgttcatgtccgagctaagagaggtcaagctactgatagccatagtttggctgaaagggtacgaagagagaagatcaatgaaaggctgaaatgcttacaagaccttgttccaggatgctacaaggcaatgggaatggcagtgatgcttgatgtcatcatagattatgtacgatcactccagaatcaaatcgagtttttgtccatgaaactctcagcggcaagtgcatgttacgaccttaattctttggatattgagccaacggatatatttcagggagggaatattcatagtgcagcagagatggaaaggattttaagagaaagcgttggaacacagcctcctaatttcagttcaacattacccttttga

SEQ ID NO: 121 - DNA, 1164 bp, Arabidopsis thaliana
atgggaggtggtggtcgcatgtctactgtcataaccagcaacaacagtgagaagaaaggaggaagcagccaccttaagcgagcgccgcacacgaagcctcctttcacacttggtgacctcaagagagccatcccaccccattgctttgaacgctcttttgtgcgctcattctcctatgttgcctatgatgtctgcttaagttttcttttctactcgatcgccaccaacttcttcccttacatctcttctccgctctcgtatgtcgcttggctggtttactggctcttccaaggctgcattctcactggtctttgggtcatcggccatgaatgtggccatcatgcttttagtgagtatcagctggctgatgacattgttggcctaattgtccattctgcacttctggttccatatttttcatggaaatatagccatcgccgccaccattctaacataggatctctcgagcgagacgaagtgttcgtcccgaaatcaaagtcgaaaatttcatggtattctaagtactcaaacaacccgccaggtcgagttttgacacttgctgccacgctcctccttggctggcctttatacttagctttcaatgtctctggtagaccttacgatcgctttgcttgccattatgatccctatggcccaatattttccgaaagagaaaggcttcagatttacattgctgacctcggaatctttgccacaacgtttgtgctttatcaggctacaatggcaaaagggttggcttgggtaatgcgtatctatggggtgccattgcttattgttaactgtttccttgttatgatcacatacttgcagcacactcacccagctattccacgctatggctcatcggaatgggattggctccggggagcaatggtgactgtcgatagagattatggggtgttgaataaagtattccataacattgcagacactcatgtagctcatcatctctttgctacagtgccacattaccatgcaatggaggccactaaagcaatcaagcctataatgggtgagtattaccggtatgatggtaccccattttacaaggcattgtggagggaggcaaaggagtgcttgttcgtcgagccagatgaaggagctcctacacaaggcgttttctggtaccggaacaagtattaa

SEQ ID NO: 122 - protein, 387 aa, Arabidopsis thaliana
MGGGGRMSTVITSNNSEKKGGSSHLKRAPHTKPPFTLGDLKRAIPPHCFERSFVRSFSYVAYDVCLSFLFYSIATNFFPYISSPLSYVAWLVYWLFQGCILTGLWVIGHECGHHAFSEYQLADDIVGLIVHSALLVPYFSWKYSHRRHHSNIGSLERDEVFVPKSKSKISWYSKYSNNPPGRVLTLAATLLLGWPLYLAFNVSGRPYDRFACHYDPYGPIFSERERLQIYIADLGIFATTFVLYQATMAKGLAWVMRIYGVPLLIVNCFLVMITYLQHTHPAIPRYGSSEWDWLRGAMVTVDRDYGVLNKVFHNIADTHVAHHLFATVPHYHAMEATKAIKPIMGEYYRYDGTPFYKALWREAKECLFVEPDEGAPTQGVFWYRNKY

SEQ ID NO: 123 - DNA, 1164 bp, Arabidopsis thaliana
atgggaggtggtggtcgcatgtctactgtcataaccagcaacaacagtgagaagaaaggaggaagcagccaccttaagcgagcgccgcacacgaagcctcctttcacacttggtgacctcaagagagccatcccaccccattgctttgaacgctcttttgtgcgctcattctcctatgttgcctatgatgtctgcttaagttttcttttctactcgatcgccaccaacttcttcccttacatctcttctccgctctcgtatgtcgcttggctggtttactggctcttccaaggctgcattctcactggtctttgggtcatcggccatgaatgtggccatcatgcttttagtgagtatcagctggctgatgacattgttggcctaattgtccattctgcacttctggttccatatttttcatggaaatatagccatcgccgccaccattctaacataggatctctcgagcgagacgaagtgttcgtcccgaaatcaaagtcgaaaatttcatggtattctaagtacttaaacaacccgccaggtcgagttttgacacttgctgccacgctcctccttggctggcctttatacttagctttcaatgtctctggtagaccttacgatcgctttgcttgccattatgatccctatggcccaatattttccgaaagagaaaggcttcagatttacattgctgacctcggaatctttgccacaacgtttgtgctttatcaggctacaatggcaaaagggttggcttgggtaatgcgtatctatggggtgccattgcttattgttaactgtttccttgttatgatcacatacttgcagcacactcacccagctattccacgctatggctcatcggaatgggattggctccggggagcaatggtgactgtcgatagagattatggggtgttgaataaagtattccataacattgcagacactcatgtagctcatcatctctttgctacagtgccacattaccatgcaatggaggccactaaagcaatcaagcctataatgggtgagtattaccggtatgatggtaccccattttacaaggcattgtggagggaggcaaaggagtgcttgttcgtcgagccagatgaaggagctcctacacaaggcgttttctggtaccggaacaagtattaa

SEQ ID NO: 124 - DNA, 1733 bp, Arabidopsis thaliana
gcataaaaggaaaatggagcagaaaaatctctcttttccgagcattttaataagttttctgcttgttttaatcttagtagtagtcatgaggttgtggaagaaacagaatccacctccagggccatggaagtttcctatcataggtaatcttcctcatttattactcacttctgatctaggccatgaacgttttagagccttggctcaaatttatggacctgttatgagtcttcaaattggccaagtttcagctgttgtcatttcttcagctgaagcagccaaagaggttatgaaaactcaggctgatgccttcgcccaacgccctatcgtcttggacgcacagattgtgttttataatcggaaagatgtcttgtttgcttcatatggagatcactggaggcagatgaagaaaatttggatacttgaatttctgagtgccaaaaaagttcaatcctccaggttaatccgagaggaagaaatggaggatgccatcacattcctccgttcgaaagccggatctccggtcaatattacaaagatcatttatggcattataatttcgatcatgataagaacatccgttggtaattgtaagcaaaaagaaagattgctgagtgttgccgatgcagtcaatgaggcagcgacgagttttggcaccgcagacgcttttccgacgtggaaattacttcactatatcattggagctgagtcaaaacccaggcgtttgcatcaggagattgacgatatacttgaagagattcttaatgaacacaaagccaataagccttttgaagcggataacttaatggatgttctattgaatcttcaaaaaaatggaaacgttccagtgccagtgacaaacgaaagcatcaaagcatccgttttgcaaatgtttactgccgggagcgaaacaacttcgaaagctacagaatgggtaatggcagagctgatgaaaaatccaactgaactaagaaaagcacaagaagaagttagacaagtatttggtgaaatgggaaaagttgatgaatcaagatttcatgatttgaaattcttcaagttagtggttaaagaaactctaagattacatcctccggttgtcttgattccgagggagtgtagagaaacaacacgaattgatggatatgaaattcatccgaacactcgaattgttgtgaatgcttgggcgataggaagagatcctaatacttggtcggaacctggaaagtttaacccagaaaggtttaaagattgtgcaattgattataaagggacgacatttgaactggtaccatttggtgcaggaaaaagaatatgtcctggcattacttcagctattaccaatttggagtatgtcattataaatctattatatcattttaattgggaactggccgatggaattacacctcaaacacttgatatgactgaagctattggcggtgctctcaggaaaaaaatagatcttaagttgattcctattccatatcaagttagcttaggctcaaatatttcttgattacataggagggttgaaatatatataataaactttaattaacgatgttctaatatggtttgggtgagttataataggttttccaccgatcatataagtagccttctttgatggatgggttagattataatgagttgtgggttggatttttagatgggttaaatgatttggatggataataataaattgaaatgttttctttttcaaatccgaaaaa

SEQ ID NO: 125 - DNA, 1358 bp, Arabidopsis thaliana
gagaagttgaccataaatcatttatcaacatgggtgccggcggtcgtggtcggacatcggaaaaatcggtcatggaacgtgtctcagttgatccagtaaccttctcactgagtgaattgaagcaagcaatccctccccattgcttccagagatctgtaatccgctcatcttactatgttgttcaagatctcattattgcctacatcttctacttccttgccaacacatatatccctactcttcctactagtctagcctacttagcttggcccgtttactggttctgtcaagctagcgtcctcactggcttatggatcctcggccacgaatgtggtcaccatgcctttagcaactacacatggtttgacgacactgtgggcttcatcctccactcatttctcctcaccccgtatttctcttggaaattcagtcaccggaatcaccattccaacacaagttcgattgataacgatgaagtttacattccgaaaagcaagtccaaactcgcgcgtatctataaacttcttaacaacccacctggtcggctgttggttttgattatcatgttcaccctaggatttcctttatacctcttgacaaatatttccggcaagaaatacgacaggtttgccaaccacttcgaccccatgagtccaattttcaaagaacgtgagcggtttcaggtcttcctttcggatcttggtcttcttgccgtgttttatggaattaaagttgctgtagcaaataaaggagctgcttgggtagcgtgcatgtatggagttccggtattaggcgtatttacctttttcgatgtgatcaccttcttgcaccacacccatcagtcgtcgcctcattatgattcaactgaatggaactggatcagaggggccttgtcagcaatcgatagggactttggattcctgaatagtgttttccatgatgttacacacactcatgtcatgcatcatttgttttcatacattccacactatcatgcaaaggaggcaagggatgcaatcaagccaatcttgggcgacttttatatgatcgacaggactccaattttaaaagcaatgtggagagagggcagggagtgcatgtacatcgagcctgatagcaagctcaaaggtgtttattggtatcataaattgtgatcatatgcaaaatgcacatgcattttcaaaccctctagttacgtttgttctatgtataataaaccgccggtcctttggttgactatgcctaagccaggcgaaacagttaaataatatcggtatgatgtgtaatgaaagtatgtggttgtctggttttgttgctatgaaagaaagtatgtggttgtcggtcaaaaaaaaaaaaaa

SEQ ID NO: 126 - protein, 374 aa, Arabidopsis thaliana
MGAGGRGRTSEKSVMERVSVDPVTFSLSELKQAIPPHCFQRSVIRSSYYVVQDLIIAYIFYFLANTYIPTLPTSLAYLAWPVYWFCQASVLTGLWILGHECGHHAFSNYTWFDDTVGFILHSFLLTPYFSWKFSHRNHHSNTSSIDNDEVYIPKSKSKLARIYKLLNNPPGRLLVLIIMFTLGFPLYLLTNISGKKYDRFANHFDPMSPIFKERERFQVFLSDLGLLAVFYGIKVAVANKGAAWVACMYGVPVLGVFTFFDVITFLHHTHQSSPHYDSTEWNWIRGALSAIDRDFGFLNSVFHDVTHTHVMHHLFSYIPHYHAKEARDAIKPILGDFYMIDRTPILKAMWREGRECMYIEPDSKLKGVYWYHKL

SEQ ID NO: 127 - DNA, 1356 bp, Arabidopsis thaliana
ctccgaacggccctttcgctattcagattttaagagagaaagaggaggaagtccaaagcttcattaaggtcattgaacaatgggtgccggtgggagaatgcaagatccgtcaaatgacaaaactaccaccgaacgcgtcccataccaaaaaccgccatttacagtcggcgaactgaaaaaagcgatcccaccccattgcttcaatcgatccgtaatccgctcattctcatacgtcgtttacgacctcacaatcgcctccatcttctactacatcgcgacaaattacatccataccctcccccaaccactcacctacttcgcctggcccatatactgggccctacaaggctgcgtcctcaccggcgtctgggtcatcgcccacgaatgcggccaccatgcattctccgaccaccaatggctcgacgacaccgtcggcctaatcctccactccttcctcctcgttccgtacttctcatggaaatacagccaccgccgccaccattccaacaccggatctatcgaacacgacgaagtcttcgtccctaaactcaaatccgacctccgatcaaccgcgaaatacctaaacaaccctccggggagaatcctaacccttttggtaaccctaaccctaggctggccgttatacctaatgttcaacgtctcaggccgttactacgaccgattcgcttgccatttcgacccaaacagtccgatctactccaaccgtgaacgcgcacaaatcttcatctccgacgccggaatcttcgccgttctgtacggacttttccgtctagcaacaatcaaaggtctcggttgggttctggcaatgtacggtggtccgttacttgtggtgaacggtttccttgttttgattacctttttacagcatacacacgcgtcgttaccccattatgactccaccgaatgggactggttacgtggggctttagccaccatagatcgtgactacgggattctgaacaaggttttccataacattacagatacgcatgtgactcatcatttgttttcgactatgcctcattaccatgcgatggaggcgacgaaagcgataaagccgattttgggggattattatcagtttgatgggacgtcggtttttaaggccatgtatagggagacgaaggagtgcatttatgttgataaggatgcagaggtgaaagatggggtttactggtaccggaataagattgagtaagatgtaagagagtggtgaatggtagttaatgttattttagcgtatgttttggttttgatgccgtttgaatggacatggaaattgtagtcggatgaataagttatgtaatcggacttggtttttgtaaaaaaaaaaaa

SEQ ID NO: 128 - protein, 379 aa, Arabidopsis thaliana
MGAGGRMQDPSNDKTTTERVPYQKPPFTVGELKKAIPPHCFNRSVIRSFSYVVYDLTIASIFYYIATNYIHTLPQPLTYFAWPIYWALQGCVLTGVWVIAHECGHHAFSDHQWLDDTVGLILHSFLLVPYFSWKYSHRRHHSNTGSIEHDEVFVPKLKSDLRSTAKYLNNPPGRILTLLVTLTLGWPLYLMFNVSGRYYDRFACHFDPNSPIYSNRERAQIFISDAGIFAVLYGLFRLATIKGLGWVLAMYGGPLLVVNGFLVLITFLQHTHASLPHYDSTEWDWLRGALATIDRDYGILNKVFHNITDTHVTHHLFSTMPHYHAMEATKAIKPILGDYYQFDGTSVFKAMYRETKECIYVDKDAEVKDGVYWYRNKIE

SEQ ID NO: 129 - DNA, 1435 bp, Arabidopsis thaliana
tttcactattaatcatttatcaacaagatgggtggcggtggccgtggtcggacttcgcaaaaacccctcatggaacgtgtctcagttgatccacccttcaccgtgagtgatctcaagcaagcaatccctccccattgcttcaagcgatctgtaatccgttcctcttactacatagtccacgatgctattatcgcctacatcttctacttccttgccgacaaatacattccgattctccctgcccctctagcctacctcgcttggcccctttactggttctgtcaagctagcatcctcaccggcttatgggtcatcggtcacgaatgcggtcaccatgccttcagcgactaccagtgggttgacgacactgtgggcttcatcctccactcgtttctcatgaccccgtatttctcctggaaatacagccaccggaaccaccatgccaacacaaattcgcttgacaacgatgaagtttacatccccaaaagcaaggccaaagtcgcgctttactataaagttctcaaccacccacctggccgactgttgattatgttcatcaccttcaccctaggcttccctctatacctctttaccaatatttccggcaagaagtatgaaaggtttgccaaccatttcgaccccatgagtccgattttcaaagagcgtgagcggtttcaggtcttgctatcggatcttggccttcttgctgtgctttacggagttaaacttgcggtagcagcgaaaggcgccgcctgggtgacgtgcatttacggaattccagttttaggcgtgtttatctttttcgatatcatcacctacttgcaccacacccatctgtcgttgcctcattatgattcatctgaatggaactggctcagaggggctttgtcaacaatcgatagggactttgggttcctgaatagtgtgctccatgatgttacacacactcacgttatgcatcatctgttttcatacattccacactatcatgcgaaggaggcaagggatgcaatcaacacagtcttgggcgacttttataagatcgataggactccaattctgaaagcaatgtggagagaggccaaggaatgcatcttcatcgagcctgaaaaaggtagggggtccaagggtgtatattggtacaataaattctgatcatatggaagatgcatctgcattttcaaacccaccctttagtttttgtttctatgttttttttttttgaaccagatcttagttctatgtgtaataaaccgccggtacaatggttttctatgcctaggccaggcgaaataattaagtactgcccctatctatatttgtaaggtattacatttcgcccatgaaataaaaaagatctggttttgttaaaaaaaaaaaaaaaaaaaaaaaaaaaaaaaaaaaaaaaaaaaaaaaaaaaaaaaaaaaaaaaaaa

SEQ ID NO: 130 - protein, 375 aa, Arabidopsis thaliana
MGGGGRGRTSQKPLMERVSVDPPFTVSDLKQAIPPHCFKRSVIRSSYYIVHDAIIAYIFYFLADKYIPILPAPLAYLAWPLYWFCQASILTGLWVIGHECGHHAFSDYQWVDDTVGFILHSFLMTPYFSWKYSHRNHHANTNSLDNDEVYIPKSKAKVALYYKVLNHPPGRLLIMFITFTLGFPLYLFTNISGKKYERFANHFDPMSPIFKERERFQVLLSDLGLLAVLYGVKLAVAAKGAAWVTCIYGIPVLGVFIFFDIITYLHHTHLSLPHYDSSEWNWLRGALSTIDRDFGFLNSVLHDVTHTHVMHHLFSYIPHYHAKEARDAINTVLGDFYKIDRTPILKAMWREAKECIFIEPEKGRGSKGVYWYNKF

SEQ ID NO: 131 - DNA, 2040 bp, Arabidopsis thaliana
ctcaggcaggtctcagttgatgagacgctgagttctgaatcctttgagctgtgtcaggctcggcacttgtgggatggtgaaggagtgatcgatcaggagtgcaggagctgcattagtttctcagggtcgatcaggttattctgaaaaaggctgcgtctgtgagcagtttgcaaaaatggccctcgttaccgactttctgaactttctgggcacgacatggagcaagtacagcgtgtacacccatagctatgctggaaactatgggcctactttgaagcacgccaaaaaggtttctgctcaaggtaaaactgcgggacagacactgagacagagatcggtgcaggacaaaaagccaggcacttactctctggccgatgttgcttctcacgacaggcctggagactgctggatgatcgtcaaagagaaggtgtatgatattagccgttttgcggacgaccaccctggagggacggtaattagcacctactttgggcgggatggcacagacgttttcgcaacattccatccacctgccgcatggaagcaactcaatgactactacattggagaccttgctagggaagagccccttgatgaattgcttaaagactacagagatatgagagccgagtttgttagagaagggcttttcaagagttccaaggcctggttcctgcttcagactctgattaatgcagctctctttgctgcgagcattgcgactatctgttacgacaagagttactgggctattgtgctgtcagccagtttgatgggtctcttcgtccaacagtgtggatggcttgcccatgatttccttcatcaacaggtctttgagaaccgtaccgcgaactccttctttggctatttgttcggcaattgcgtgcttggctttagtgtatcatggtggaggacgaagcacaacattcatcatactgctccgaatgagtgcgacgaacagtacacacctctagacgaagacattgatactctccccatcattgcctggagcaaggaaattttggccaccgttgagagcaagagaattttgcgagtgcttcaatatcagcactacatgattctgcctctattgttcatggcccggtacagttggacttttggaagtttgctcttcacattcaatcctgatttgagcacgaccaagggattgatagagaagggaacagttgcttttcactacgcctggttcagttgggctgcgttccatattttgccgggtgtcgctaagcctcttgcgtggatggtagcaactgagcttgtggccggtttgttgttgggattcgtgtttacgttgagtcacaatggaaaggaggtttacaatgaatcgaaggacttcgtgagagcccaggttattaccacccgtaacaccaagcgaggctggttcaacgattggttcactgggggactcgacacccagattgagcatcacctgtttccaacaatgcccaggcacaactaccccaagatcgcacctcaggtcgaggctctttgcaagaagcacggcctcgagtacgataatgtctccgtcgttggtgcctctgtcgcggttgtgaaggcgctcaaggaaattgctgatgaagcgtcaattcggcttcacgctcactaagaaatcgtcgaactttgactattcatttttttcgcctggctacctcaaatgttcgggagcaggtgcttggcagtgtgttcaaccggagcgcactgaaaatgtgcagaatccatttccagaaattaccattcctagctaaatcttctttttaccaggtcggatatatgaaacttttttgatgcaacaagtagcattcaattgaagacattgttcgagatataattcgcagtgtttctattcagcgggcatacgtactagtccatatcggcggttgccgagagtttacattattagttggcacaacgagtagatctagtgtaaatttctatttccgcatgtaatattactctgaatatataccgttatctattttcctaaaaaaaaaaaaaaaaaaaaaaaaaaaaaaaaaaaaa

SEQ ID NO: 132 - protein, 483 aa, Arabidopsis thaliana
MALVTDFLNFLGTTWSKYSVYTHSYAGNYGPTLKHAKKVSAQGKTAGQTLRQRSVQDKKPGTYSLADVASHDRPGDCWMIVKEKVYDISRFADDHPGGTVISTYFGRDGTDVFATFHPPAAWKQLNDYYIGDLAREEPLDELLKDYRDMRAEFVREGLFKSSKAWFLLQTLINAALFAASIATICYDKSYWAIVLSASLMGLFVQQCGWLAHDFLHQQVFENRTANSFFGYLFGNCVLGFSVSWWRTKHNIHHTAPNECDEQYTPLDEDIDTLPIIAWSKEILATVESKRILRVLQYQHYMILPLLFMARYSWTFGSLLFTFNPDLSTTKGLIEKGTVAFHYAWFSWAAFHILPGVAKPLAWMVATELVAGLLLGFVFTLSHNGKEVYNESKDFVRAQVITTRNTKRGWFNDWFTGGLDTQIEHHLFPTMPRHNYPKIAPQVEALCKKHGLEYDNVSVVGASVAVVKALKEIADEASIRLHAH

SEQ ID NO: 133 - DNA, 2160 bp, Arabidopsis thaliana
cggaggtctcttgtcgttcttggagtctgtgtcgagcttggaatgcggtaggcgcggccgtttcgtggttttggcgttggcattgcgcgagggcggacagtgggagtgcgggaggtctgtttgtgcatgacgaggtggttgtaatcttcgccggcagaatggtgtcccagggcggcggtctctcgcagggttccattgaagaaaacattgacgttgagcacttggcaacgatgcccctcgtcagtgacttcctaaatgtcctgggaacgactttgggccagtggagtctttccactacattcgctttcaagaggctcacgactaagaaacacagttcggacatctcggtggaggcacaaaaagaatcggttgcgcgggggccagttgagaatatttctcaatcggttgcgcagcccatcaggcggaggtgggtgcaggataaaaagccggttacttacagcctgaaggatgtagcttcgcacgatatgccccaggactgctggattataatcaaagagaaggtgtatgatgtgagcaccttcgctgagcagcaccctggaggcacggttatcaacacctacttcggacgagacgccacagatgttttctctactttccacgcatccacctcatggaagattcttcagaatttctacatcgggaaccttgttagggaggagccgactttggagctgctgaaggagtacagagagttgagagcccttttcttgagagaacagcttttcaagagttccaaatcctactaccttttcaagactctcataaatgtttccattgttgccacaagcattgcgataatcagtctgtacaagtcttaccgggcggttctgttatcagccagtttgatgggcttgtttattcaacagtgcggatggttgtctcacgattttctacaccatcaggtatttgagacacgctggctcaatgacgttgttggctatgtggtcggcaacgttgttctgggattcagtgtctcgtggtggaagaccaagcacaacctgcatcatgctgctccgaatgaatgcgaccaaaagtacacaccgattgatgaggatattgatactctccccatcattgcttggagtaaagatctcttggccactgttgagagcaagaccatgttgcgagttcttcagtaccagcacctattctttttggttcttttgacgtttgcccgggcgagttggctattttggagcgcggccttcactctcaggcccgagttgacccttggcgagaagcttttggagaggggaacgatggctttgcactacatttggtttaatagtgttgcgttttatctgctccccggatggaaaccagttgtatggatggtggtcagcgagctcatgtctggtttcctgctgggatacgtatttgtactcagtcacaatggaatggaggtgtacaatacgtcaaaggacttcgtgaatgcccagattgcatcgactcgcgacatcaaagcaggggtgtttaatgattggttcaccggaggtctcaacagacagattgagcatcatctatttccaacgatgcccaggcacaaccttaataaaatttctcctcacgtggagactttgtgcaagaagcatggactggtctacgaagacgtgagcatggcttcgggcacttaccgggttttgaaaacacttaaggacgttgccgatgctgcttcacaccagcagcttgctgcgagttgaggcatcgcagcactcgtcgaaacatttttgtctgttatagtgttcatatgtgatcgaggggaaaaggtcccatgctctgatctattcttctgtagccaatatttttcaattgaaaggaggttcctcacttatcttccatctatcgttgcacatcctgcatcagagttagcgttggagtaatgttaagcacttgtagattatgcccaccattgccacatttctgttcggttacaatcgtttgattccatgctatcctccgtgttcatctcgttgttataagcaagcttgaaaaaacatgctacgagattggcagacgttgtcttggcagctgtagaggttggttccattcattgtgtagtacagaactctctcgtccctgtttctctacattacttgttacatagtgactttcattcacagcaaaaaaaaaaaaaaaaaa

SEQ ID NO: 134 - protein, 520 aa, Arabidopsis thaliana
MVSQGGGLSQGSIEENIDVEHLATMPLVSDFLNVLGTTLGQWSLSTTFAFKRLTTKKHSSDISVEAQKESVARGPVENISQSVAQPIRRRWVQDKKPVTYSLKDVASHDMPQDCWIIIKEKVYDVSTFAEQHPGGTVINTYFGRDATDVFSTFHASTSWKILQNFYIGNLVREEPTLELLKEYRELRALFLREQLFKSSKSYYLFKTLINVSIVATSIAIISLYKSYRAVLLSASLMGLFIQQCGWLSHDFLHHQVFETRWLNDVVGYVVGNVVLGFSVSWWKTKHNLHHAAPNECDQKYTPIDEDIDTLPIIAWSKDLLATVESKTMLRVLQYQHLFFLVLLTFARASWLFWSAAFTLRPELTLGEKLLERGTMALHYIWFNSVAFYLLPGWKPVVWMVVSELMSGFLLGYVFVLSHNGMEVYNTSKDFVNAQIASTRDIKAGVFNDWFTGGLNRQIEHHLFPTMPRHNLNKISPHVETLCKKHGLVYEDVSMASGTYRVLKTLKDVADAASHQQLAAS

SEQ ID NO: 135 - DNA, 1089 bp, Arabidopsis thaliana
atggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcaatgacacctgaggatcacgcgtacgtgggttcatccgatgatcaggtgagctttaacgactttgagtggtggtga

SEQ ID NO: 136 - protein, 362 aa, Arabidopsis thaliana
MDNFLPFPSSNANSVQELSMDPNNNRSHFTTVPTYDHHQAQPHHFLPPFSYPVEQMAAVMNPQPVYLSECYPQIPVTQTGSEFGSLVGNPCLWQERGGFLDPRMTKMARINRKNAMMRSRNNSSPNSSPSELVDSKRQLMMLNLKNNVQISDKKDSYQQSTFDNKKLRVLCEKELKNSDVGSLGRIVLPKRDAEANLPKLSDKEGIVVQMRDVFSMQSWSFKYKFWSNNKSRMYVLENTGEFVKQNGAEIGDFLTIYEDESKNLYFAMNGNSGKQNEGRENESRERNHYEEAMLDYIPRDEEEASIAMLIGNLNDHYPIPNDLMDLTTDLQHHQATSSMTPEDHAYVGSSDDQVSFNDFEWW

SEQ ID NO: 137 - DNA, 936 bp, Arabidopsis thaliana
aattattttataaagaacaaaaaaaaaaaaagacggcagagaaacaatggaacgtggagctcccttctctcactatcagctaccaaaatccatctctgaattgaacttggaccagcacagcaacaacccaaccccaatgaccagctcagtcgtagtagccggcgccggtgacaagaacaatggtatcgtggtccagcagcaaccaccatgtgtggctcgtgagcaagaccaatacatgccaatcgcaaacgtcataagaatcatgcgtaaaaccttaccgtctcacgccaaaatctctgacgacgccaaagaaacgattcaagaatgtgtctccgagtacatcagcttcgtgaccggtgaagccaacgagcgttgccaacgtgagcaacgtaagaccataactgctgaagatatcctttgggctatgagcaagcttgggttcgataactacgtggaccccctcaccgtgttcattaaccggtaccgtgagatagagaccgatcgtggttctgcacttagaggtgagccaccgtcgttgagacaaacctatggaggaaatggtattgggtttcacggcccatctcatggcctacctcctccgggtccttatggttatggtatgttggaccaatccatggttatgggaggtggtcggtactaccaaaacgggtcgtcgggtcaagatgaatccagtgttggtggtggctcttcgtcttccattaacggaatgccggcttttgaccattatggtcagtataagtgaagatggaattattcttcatttttatatctgttcaaaacatgtgtttggatagatattttatttttatgtcttatcaataacatttctatataatgttgcttctttaaggaaaagtgttgtatttcaatactttatgagaaactgatttatatatgcaaatgatttaacccaaa

SEQ ID NO: 138 - protein, 238 aa, Arabidopsis thaliana
MERGAPFSHYQLPKSISELNLDQHSNNPTPMTSSVVVAGAGDKNNGIVVQQQPPCVAREQDQYMPIANVIRIMRKTLPSHAKISDDAKETIQECVSEYISFVTGEANERCQREQRKTITAEDILWAMSKLGFDNYVDPLTVFINRYREIETDRGSALRGEPPSLRQTYGGNGIGFHGPSHGLPPPGPYGYGMLDQSMVMGGGRYYQNGSSGQDESSVGGGSSSSINGMPAFDHYGQYK

SEQ ID NO: 139 - DNA, 1380 bp, Arabidopsis thaliana
aatctctgattgcccagcgtccattttaagcctctgtttcgatctgcacaacacacatacacacaacacaacacaaaccacagtctctctttctctctctatctatcttctctttctctctctatctctatcactgaaacccaaagagatccaccatttgttcttttttccttcacacagagaactgttttcttccacacttcctttttactaggcagtgttaaccaattgagagagaaaaatgatggttgatgaaaatgtggaaaccaaggcctctactttagtggcaagtgttgatcatgggtttggatccgggtcgggtcatgatcatcatgggttatcggcgtctgtgcctcttcttggtgttaactggaagaagagaaggatgcctagacagagacgatcttcttcttcctttaaccttctctctttccctcctcctatgcctcctatttcccacgtgccaactcctctccccgcacgtaaaattgacccaagaaagctaagattcctcttccaaaaggaactcaagaacagtgacgtcagctctctccgacgtatgatactcccgaagaaagccgcggaggctcacttgccggcacttgaatgcaaggaagggattcctataagaatggaagatttggacggttttcacgtttggaccttcaagtataggtactggccaaacaacaatagcagaatgtacgtgctagaaaacacaggcgattttgtgaatgctcatggtctgcagctaggtgacttcatcatggtttaccaagatctctactcaaacaattacgttatacaagcaagaaaagcatcggaagaagaagaagtagacgtaatcaatcttgaagaagacgacgtttacacaaacttaacaaggatcgaaaacactgtggttaacgatcttctcctccaagattttaatcatcacaacaacaacaacaacaacaacagcaacagcaacagcaacaaatgttcttactattatccagtcatagatgatgtcaccacaaacacagagtcttttgtctacgacacgacggctcttacctccaacgatactcctctcgattttttgggtggacatacgacgactactaataattattactccaagttcggaacattcgatggtttgggctccgttgagaatatctctctcgatgacttctactagataatcaatcgatgggctcatggtattcttgatggtgatcagctatttaatatccttataatatatataagaattaaatgcaatttgcatatatattatcaagtgttgaatataacattacagtttatatgaaaatcatagctagatgtgagtcatgaaggtgtgttgtatttgatgaataaatgttatttagtgtt

SEQ ID NO: 140 - protein, 313 aa, Arabidopsis thaliana
MMVDENVETKASTLVASVDHGFGSGSGHDHHGLSASVPLLGVNWKKRRMPRQRRSSSSFNLLSFPPPMPPISHVPTPLPARKIDPRKLRFLFQKELKNSDVSSLRRMILPKKAAEAHLPALECKEGIPIRMEDLDGFHVWTFKYRYWPNNNSRMYVLENTGDFVNAHGLQLGDFIMVYQDLYSNNYVIQARKASEEEEVDVINLEEDDVYTNLTRIENTVVNDLLLQDFNHHNNNNNNNSNSNSNKCSYYYPVIDDVTTNTESFVYDTTALTSNDTPLDFLGGHTTTTNNYYSKFGTFDGLGSVENISLDDFY

SEQ ID NO: 141 - DNA, 2987 bp, Arabidopsis thaliana
gaaagaaagagagagtcttcttgttggagtaaacccaaacggttttagattacttattagctgttcatcagttcttcctctctaaaagagtaaaacctaaacatctctctctgttctattagaaccaaagaccaatctttgtgaacaaaacacatctcgtatacttcagatctagactcgaaaattttagacctctttacaattggtctttgttcatctgaagttggagaaaatagttagcttaggtcggatcttttcatatgctttggatcctccttcgtctcttttgtataattttaaccttatcaagagttctttttgaatctcaaaagattatatagtagtatagaaggtttatatgtatatgtatagccagatagtttatgttgtttaaagattcgatgatagccaagttgggttaactttctttttccttgcctccttactcacatacaaaccctatctgtccgtacaaaatactaaaaaccctaacttttctctctccaccaatctagtttattgtttcatttccacttcaacgatgaaaagcttgcatgtggcggccaacgccggagatctggctgaggattgtggaatactcggtggagacgctgatgatactgttttgatggatggaattgatgaagttggtagagagatctggttagatgaccatggaggagataataatcatgttcatggtcatcaagatgatgatttgattgttcatcatgacccttcaatcttctatggagatctcccaacgcttcctgatttcccatgcatgtcgtcttcatcatcgtcttcaacatctccagctcctgtcaacgcaatcgtctcctcagcctcttcttcttcggcagcttcttcctccacttcctcagctgcttcttgggctatattgagatcagatggagaagatccgactccaaaccaaaaccaatacgcatcaggaaactgtgacgactcttctggtgcattgcaatccacagcttccatggagattccattagacagcagtcaaggttttggttgcggcgaaggcggtggtgattgcattgatatgatggagactttcgggtacatggatctacttgatagcaacgagttctttgacacctcagctatatttagccaagacgacgacacgcaaaaccctaacttgatggaccaaacccttgagagacaagaagaccaggtcgttgttccgatgatggagaataacagtggtggagacatgcaaatgatgaattcttccttggaacaggacgatgatctcgctgctgtgtttttggagtggctaaagaacaacaaggagactgtgtcggctgaggatttgaggaaagtaaagataaagaaagctacgattgaatcagcggcaagaagactaggcggtggtaaagaagcgatgaagcagcttttaaagctgattcttgaatgggtccaaactaatcacttacaaagaagacgcaccaccaccaccaccaccaacctctcttatcaacaatcattccaacaagatccatttcaaaaccctaaccctaataacaacaacctaatcccaccgtccgaccaaacctgtttctcaccttcaacatgggttcctccaccaccacaacaacaagcttttgtctcggacccgggttttggatacatgcctgctccaaactatccgccacagccagagttccttcctttacttgaatctccaccgtcatggccaccaccaccacagtctggtcccatgccacatcaacaattccccatgccgccaacctcgcagtataatcaatttggagatccaacaggtttcaatggatacaacatgaatccgtaccaatatccttatgttcctgcaggacaaatgagagatcagagattactccgtttgtgttcctcagcaactaaagaggcaagaaagaaacggatggcgagacagaggaggttcttgtctcatcaccacagacataacaacaacaacaacaacaacaacaataatcagcagaaccaaacccaaatcggagaaacctgtgccgcggtggctccacaacttaaccccgtggccacaaccgccacgggagggacctggatgtattggcctaatgtcccggcagtgccgcctcaattaccgccagtgatggagactcagttacctaccatggaccgagctggctcagcttctgctatgccacgtcagcaggtggtaccagatcgccggcagggatggaaaccagaaaagaatttgcggtttctcttgcagaaagtcttgaagcaaagcgacgtgggtaacctcggaaggatcgttttgccaaaaaaagaagctgagacacacttgccggagctagaggcaagagacggcatctctctggccatggaagacatcggaacctctcgtgtttggaacatgcgctacaggttttggcctaacaacaaaagcaggatgtatctcctcgagaacaccggcgattttgtgaaaaccaatgggctccaagaaggtgatttcatagtcatatactccgacgtcaaatgtggcaaatatttgatacgaggggttaaagtaagacaaccgagcggacaaaagccggaggccccaccgtcgtcagcagctacgaagagacaaaacaagtcgcaaaggaacataaacaataactctccgtcggcgaatgtggtggtcgcttcaccaacttctcaaactgttaaatgaaaaacagagacaaaaagaaacaatataaatattattatgtaccaaataagaaagagggcaaaaggaaaaaatggcagcgtacccgagtgtgccacttctcgtgcatgcatgggatcttgaagacaaatggagggtcatgattaaagctgtttggtcggggtccgggtttttactccattttttgctttttcttgtcgagtcggttcttttataactctttactctttttaccttcaggatattgtagagatgattaattctggaaatggtgtttgtgttatat

SEQ ID NO: 142 - protein, 720 aa, Arabidopsis thaliana
MKSLHVAANAGDLAEDCGILGGDADDTVLMDGIDEVGREIWLDDHGGDNNHVHGHQDDDLIVHHDPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMMENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKCGKYLIRGVKVRQPSGQKPEAPPSSAATKRQNKSQRNINNNSPSANVVVASPTSQTVK

SEQ ID NO: 143 - DNA, 1580 bp, Arabidopsis thaliana
cagggtttatttaacttgccctttctcgtttcctcctttttttcttaaaccactctgcttcctcttcctctgagaaatcaaatcactcacactccaaaaaaaaatctaaactttctcagagtttaatgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaaggtttcgagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctgagagagagagctttgccttctagtttgaatttctatttcttccgcttcttcttcttttttttcttttgttgggttctgcttagggtttgtatttcagtttcagggcttgttcgttggttctgaataatcaatgtctttgccccttttctaatgctccaagttcagat

SEQ ID NO: 144 - protein, 356 aa, Arabidopsis thaliana
MDWEIRGSSLGQKLLEFDSEQERQTRFRAYDSEEAAAHTYDLAALKYWGPDTILNFPAETYTKELEEMQRVTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVNQANHQEGILVEAKQEVETREAKEEPREEVKQQYVEEPPQEEEEKEEEKAEQQEAEIVGYSEEAAVVNCCIDSSTIMEMDRCGDNNELAWNFCMMDTGFSPFLTDQNLANENPIEYPELFNELAFEDNIDFMFDDGKHECLNLENLDCCVVGRESPPSSSSPLSCLSTDSASSTTTTTTSVSCNYLV